SEQUENCE LISTING (275 sequences)

SEQ ID NO: 1 - DNA, 3278 bp, H. sapiens
aggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 2 - DNA, 26001 bp, H. sapiens
atgccctccataggctttcagaccttcttcaaggccaaagggaaggccttcatggaaatataattatgtgaaacaccccagaattttttcacaaactttcctgcctataaacgccaggtcctaccactgtccagtgtcccacttcccactgtccagtgggaagactccctccaggacaaaaccaccctttccttcagggatgtgacgtgctgtgctttcgttgacagtcatgcagctgctagacatgtcacttcctagctctccattcgggtctgtgtcccaggacctaaagaaagctaaaagaagccgggcgtggtggctcatgcctgtcatcccagcattttgggaggctgaggctggcggatcactggagatcaggagtttgagaccagtctgaccagcatggtgaaaccctgtctctactaaaaatacaaaaaaagaaaagaaaaaagaaaaaaaaataagctgggcgtggtggcgcgggcctgtagtcccagctactccggaggcggaggcaggacaatcacttgaacctggaaggcggaggttgcagtgagctgagatcgcgtcattgcactccagcctgtgcgacgagagactctgtctcaaaagaaagaaagaaagagagaaagaaagaaagaaagaaaagaaagaaagaaagaagaaagaaagaaaagaaagaaagtaagaagcaagcaagctgaaatagcttatttttctgttttaaaaaatagacttttagtgttcacaagtaagaataaaaaaaaaaaaagcttggccttaggaggaatcctatctgcttaggcctctgagaggcagcgtcacctgaagggaaactgactgggcaagagccaggctcttaggagctgtctgtactttgcttcccatccgtccgctctcccccagcagccaaagtctcctccctccattatattgcaaatcaatgaatccattagactttccatgttttctttttagactttgagattcaaggtcagctagaattaatggttaacagctgacaggtgaaagtgtggttaaatgcagctttggctagcaggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggtacagtttcagaacttactatttaacattcctctcaagcaaatacgccttgaaatgctttttttaaatcataggaatttaaaaacactttacaatagagaatgattgatttttaaaatgtgtctgatttagctttgtagagatgttccgctaatatccataactaatctgagaggaaatgtggaacaacagaagagtaacagtgtctactcagtaacaagcgttttacgagttaaaaagacatccaaatgcagtactgaaaaatcagaagtcttgatttgtctcactgatgactccgtttttcctagagcagtctgtttaatgcttactggagataaatagatttataggtgaccaagacaatcgattaatgtatcagccacagacttttttaaatagaaaattttctaagtaggaaatcattcatagctctttgaaagatatagggagaggcctcaaggaaagaaagaaaggaaaaaaattgggaaaggaaacaaagatgaaaaattggggtggggagagcggtcagatggtggccatgagaaggatctgaacacagagagcggcggggccggcggggaaggagggaggaggggagagcgctgcttccctgtgggttccggcttctgcagagctgtaagagttgaatgccacacacagtcacactaaggaatgctccaggattgggaaagaaaattcaacattataatgagaacactgtgaatgctattgaattaactactcccctctctccctatttcttgtaagtcttagtgtcagtaaactaattataaatttacattttatgttctaaaagcatgcacctttttctcattgtaggatgattttcttatatcaagtggtacatttcattttatttacttcagtttctggtggtaagtagagtgttatcttaactatgggctgggagagggaaatcacactgcaatctccacacatgtgggagaatcccacaccatttatgccgggaaggaaataaaatgtttttattaacttcctgcctgaggctccagaggttttcaaagcagggtaggaattgaggtgaaaaaattgtttgtactggtaggaatctgtgtctatatgtgtaccatatctacatccatgcctacatacatcattcatttaaatgatatttaaaagcaatattaaaaagaagaaatcttgatttgcctcactaattagttggtttttcataaagcagtcagttcaatggaacatacacacacacataaaataggaattttgtactgaaaaatgttatgtaactattcatacacagtttatgcatttcaataatttaactaccctgttaattctccaccctatgtcacatctgctaaatttgtttataaattacttagccaatattaagtaggatatatcagtacaggtattgtttagttccatgaccattctttatttaaaatgaaagctgaagcattcctttagaaaaatagcttatgtctacatgttatatttaatttctgatactattgctagcattttaagcatagtaaattctttttgctggcctatgtgaaaaaaggcaaacctaggtgaagatattaaaagtaaattccacttaatatattaaattacacagctcataatttcaattttctctgtgacaagactactgctcaggttataatattcctttgttataattatctggaaattgtattgttatttcattgataaatatgtcagcgctaaataatttgatatattttgaacactggacccaataactacataaacaattgaccctgaatcaggggttccacatccatggatttaaacaacattgaagtgaaattttttaaaaaaaattatatctgcactcaacatgtgcagagttttttcttgtcattattcccgaaacaacacagtataacaacaatttgcatagtatttacattgtactaggtattataagtaatctagagatgatttaaagtacacaggaggctgtgagtaggttatatgtaaatactatgccattttctatcagggacttgagcgttcatggattttggtatctgcaggaggtctggcagccaatccctcatagatatcaaggaatgatagtgtacatatttaggtcctttatacatgcacatggaacatttacaaatattgaccacacacaagcctctgaaacaaggctcaacaaattttaaggggttaaaattatactgatcatgttattcaatcaccgtgaatgggagctagaataataataaaaagggaattgaaaatctgcaaacgtttgaaaattgagtatttataatcaccgtgaatgggagctagaataataataaaaagggaattgaaaatctgcaaatgtttgaaaattgagtatttatacctttttagattaagatgaatcagagatgaaatcacaatggtaattagaaaaggaattgtacaataatgaaagtacagtatatcaaaactttgagatgtaaccgaatcagtgttgagatcaaatgtatagatttacatgtatacattagaatacagaaaagggaaaaattaatgatataagatgacaagaagatagagaatagcaacttaagtgctaagaaattagaaggcagaaagaagaaatcgcagaaattaattaaatggaagacacacatatttgagaggtcaacataacccctttctagcccattttataaggtcagggtaatcttgacaccaaaatctgataggaaaattctgaggaaaaaaagtcacaggtcttttatcttatgaacatatgtacaaatgtcctaaacaaaatattatccaacttaacccagtagattgttaaaaaaattaaaggatgattacattgtgatattgtgacaaagttgggtttattccaggaatgaaaagttgatttaacattcaaaaatcaatagatgtgtgacttcggggtacataccgaaaaaaaagaagggaaagcaggaactcacacagatatacttgtacacccatctcatagcagcattattcacactaacccaaaggtggaagcagcccatgtgtttatcaacaggtgactggataaagtgtggtacatacatacaaaggaatattactcaaccctaaaaagaaaggaaattctgacacatgctatgacatggatgaaccttgaggacattacgctaagtgaaataagccagtcacaaaagaacaaatactgtgggatctcactgataggaggtacttagggtagtcaatttcatcaagacggaaattagaacagaggttgccaggggccgaggggccaggggaagggctgagggttggtgtttaatgggcacagagcatcagctggcgaagttgaaaagttctggaggtggatggtgctgatggttgcacagcaatgtaaatacccttagtgccacagaactgtacactcaagatggctacatggcacatggtatgctatctgtcataataaaaaataaaaataattttaaatgttaatatgtgccgaaaaatgcttgctaaaattctattaatgatcaaaactttaaataaactggaaatggaagagaactttcatcagctattaaacgatattttaaacaaaagcaaacaaaaatccaaaccaaaaatcttcaagaaacataatacgtaatagcggaatattgaaagctttcccaagggattgagaataggacaagaatacttcccattatttccatttactcttgactggaggtactcgttggtgcggtaaggaagggaaataaaaggaaaacgattaggaggaacataagctctcgtttttcacagatgatgtgattgagtacctagaaaaatccaaaattaaccatcacataaattatttgagccaattaattaaatgagtaagaagtctcgatacaacgtcaatatacaacagcaagcacttgctcgttcgcaaaagcagatgaactagtttttactgacactagaagcagcatgtgttccatgtgttccacatccatgcactcaaacaaccttgacgtgaaatattttttaaaaaattgtatctgtactcaacgtgtgcagagtttttttccaaaattgttttccaaaaatttttaaatgtttatatgtttagggggtgtaagtgcagatttcttacatacatatattatgcggcggtgaagtgccggcttttggtgtacccatcacccaagtagtgaacacagcctccaacaggtaatttttcaacgctcaccccactcccatcctcccatctagtggcatattgaaaatcaatttgtcttgtaaaattaaataatccaattaggagggggatatattctaaggaaattagtgcatgatgcacacacacacacacacacagaacacgtgtgtgcgcatgtgcacatgagagagagtgagagagaaactgggtcttgctctgtcgcccaggctggattgcagtggtaaaatcacagctcactgcagcctcaaactcccaggctcaggagatcctcctacctcagcctcccgagtagctgggattacaggtggaaacaaccatgcccagctagtatttttttttttttttgtattttttatagagacagggtcttgccatgttgcccaggctggtcttgaactcctcagctcaagcaatctacctaccttagcctcccaaagtgctgggattacacgcatgagccactgcgcccactccgcattattaaatatagaacatttatttgattcatcagttaatattcttcttaaaagtactattttaatgtagcaagattgctttccaccaaaaggtggggtttccacggtggggtttccaaattattctcaatggggtgaggatgtgtgttatcacacccccgagccatcagatgctgtcagaaagtgatcactctgaagtctttgtttcaaataagcacagggtttggataaagagacgcaattaggaaaggaaaaagcagaaggctcgttccagacctggatgagatcctaaaaagcagcagcttttgccagtaaagatccttgaaatgattcaattaccctcaaagcactccttgtctccaagacaatcactcataagcacaattccattgaagccaacgtaccattttgtgattttcgtttccacctgaggctgttcattcaataaactcacataaaagtgtttattgccttgatttccaaattcaggcgtatttcctggtaagtagagctacttgccttgcctttatgagattaccacctaactagatgtatgcccagtaaaatccaacataacgcatgccatgtactacatcacagaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtaaatgcttatgtttctacatcgaggagacagatttttaaagggagattgctattcttaacacatttccatctaacattttataaaatttaacattaacaactggaagataaattgtctttcagttgaaatattgttacagaaagaagtgatggtgtttacgcaatttagaaaagaataaatatgcctccaagtgtagactttccagcctctcctatagtctcatattaatggtatgtttcttctgtttgtctcaatttttacacttcttaaacatttcacactttgcttttctatcgattattaatttttgtcgtgcttctcaacaaactggaacttctcccaactattttattagaaaaaaataaaatatttaaaaagaaaatttgaaaaaaagtacagtacagtgatccccctgccaccaccaaaaccgcaatgtttgctatatttgtacagcaatatacaatatacaatacctatatttgtatacatgtttaaccgtttgaaataattttaaaacatgacactttacccctaaatacttcagcatgcactatcctacacaaaagacatacgaaatttaacaagaattcctttatattatttcagttttccctcaaatataatttatagtaattaaccagaatcttaccaagagtcactcactgcattgggtggtttgtcaggtttaaaacattttaaacagtccaccaaccatttgtattcccatcctgagcttgaaaatttaataataagcctttctgtagaattaagttttcaacatctttattattgctacattcacaggcatttatgtagcacccagaacttataaaatttactattccagaacctagagcagggattggcaaatgtcttcttaataacgcagagtaaatatgttaggctttgtgggcaaaacccacagtaaagccaaggatattatttaagtatttatgtcaccacttaaaatgtaacaatttgaaaatataaaaatcattttgtatagctaacaggctaaacagaaacacacagatttttggttgcattttaccaacaggtcctagttgacacattcctgtttgttcctattagaaaggagtattacatgcagtctcttaagtgtagggatattgaagtaaaaaacaaactcagaatcttgctaagaaaatatttgttttggcatgagataaagtagtttgtttccttctttttggctttctgtgtgctgacttttaagatccattattttaaaaacataaattcctattcattaatatgtattttttaaaaaaacaggtttacttgtgtcctgaaagacagtgttacagaaacactgccaagagtgaataggacagcagcgatttctgggtattctttcaagcaatgctcacaccaaataagcggtaagatatgttctcagaatcaacaaataccagctgtgatgtacacatatcgccacatcggatgtggttttaaggctatgaaatgaaacactgctatgtggaaataaacccccttaatgaagttctttcagtgtagagtataaactagtatacatacatgcctgccctccaacacactgtaaaaacctctttacctcatagaaagacatatcttactacctcacttcccatcatttatttatattctttctatttcccagcctaaaatcttaaatgaaagtctttttttttttgagacagggtctcactctgttgtccaggctggagtgcagtggtgcaatcacagctcactccagcctcaacctcctgggttcaagtgatcttcctgccttagcctctggagtagctgggaccagaggcatgcaccaacattcccagctaatttgttcatttttcctagagacagggtctcactgtattgcccaggctggtctcaaactcttggcctcaagtgatctgcccgcctcggccttccagagtgccgggattccatggtgcccagtcgaaattctttattaaacgtattaatccaaattgaaaggagcaaatataaaggttgaagtgacacttgtcttaatagtgaatagatacttgaatcagttaattagcgaaataatcagtgcagttagggagaagagaggctaggtcagaaaatcaaaatgtgaatttacaagtctaaaattgttacagtgtaagaaggacattggcattcttttactgcttccattcaagaataagaattttgcagattaatataacgaaagacctctgaggaaaggtgggtgaaaaagttgaaaggatgagtcaggagggacagttgcttaggtcattgcccctagaatctggaaggtactcatgtcttctgcttttatttccagcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcggtgagtgagtcccaggacattcgagtggtcgatgaaaaacagaatcgtgatttactaaaaagcttttgccatcaactttatgccagaatttattttgaacccctaaaagacatttctataatagtactcctagttttcttcatgaaaaatacacttaaagcctaatttggatgcatttcatttatggtaaggagtctatcttttaataacactgtcagaaaaatatatatacttggctaatttcaaaagcgctacacttttaaattggcacttttgaaacagctgcaattggtatgattgtcagtgcccttcccagtctaaaaaatgttacagtctaacagaataaaaataaaaacctactctctctctctctaaataacagttccttacctaagacaaaatactcatgtaaaaagtcttatcctgctccatactggattttgaaatatttcaaggataaatctatcacataaggatttaaaaattatctgatctctaataaccaaatctgtgttctcatctttaaaaatttactagggaaatagattattaatttgtatattcagaaatatttgagatgatttagattttcatagtaaactgcatttatctggaatcaacagaaaagtgaaaaacattcaaattactaatacttgcgttttaacattggattttaacattctgctctccacattcacaaagaggagtgaacagaaagcaaacaaagcatcaacgagttatttcaaaaacaacagtggtgaaaaacacacacaccaaacccctaaattcatgatttgacttgtaaggcttatctttagctcagctcagacgacagcttttatgtctaagacttaacagaatgtgaactgcaagacaagaaattggaggtttctaagcaagataaagttaagtcattaaaagtaagaaggacttagccaggcgcggtggctcacacctgtaatcccagcactttgggaggccgaggcgggcagatcacctgaggtcaggagttcgacaccagcctgaccaatatggtgaaaccccgtctctactaaaaaagaatacaaaaattagccaggcgcggtggtgggcgcttgtaatcccagctacttgggaggctgagacaagagaatcgcttgaacccaggaggcggaggttgcagtgagccaagatcgtgccattgcactccaacctgggcaacagagtgagactccgtctcaaataaaaaaaaacaaaaaatgagaagggcttgagaagtcattcattcatgcactctccttcttcatgtggtcactctctcaagctgtcattatactgaagaagaaataaacttacacaattcacaggtgcttagcaacactgctgggaccatgcccagccattcagcctcccagatggatgcttcggggtctcgcaggtcctctctccaaaggggactttcttaatatctcatgttttttcctccttgcagttggaagaataagacacttttcctttttctttttattcagtaacatttgtctactgaagcacacccaaacagggacaccaaccagaataacgaagctcgataaagtggtgtctggattttcactgaaatcctgtgcactttctaatctgggtaattatcgacttcttgatgatgtaattcaaccattaaatatgctgatgattacagtagatctcactcaggataccagcttatgctcacgatgaaacggacccaaagatctttaccttcttcatgtgatagatttcatcatgtcctatacagttagatcctctatttaaatttccagtttaaaataatcatgccattttcttctaaataaaaaaaaattaaaagatcttgggatacacttaaattttttaatatggaatttacacatactgtgaccggaattttcctgatagctggtgaattgagtccctgacatagttcttccgtcgcgcagcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaaggtaaggagttaacaagtaaggataatttgttatcttctaaaaatagctgatcaaaatccatcattaaaaattccaagtaactaaaaatttactctaaatgtcagtataggataaaagttgcaaagaatttctagcccctctccctttctattccccacctacttaccacaaacccaacattaccgaggactcttttttttttttttttttttttgagatggagtctcgctctgctgcccaggctggagtgcggtggcatgatttcagctcactgcaaccttcgcctcccaggtccaagcgattctcctgcctcagcctcctgagtagctgggactacaggcatgggacaccacgcccagtaattttttttgtatttttagtagagatggggtttcaccatgttggccaggctggtctcaaactcctgacttcaggtgatccacctgcctcggcctcccaaagtgctgagattacagggttgggccaccgtgcccggccagtaaattttaaaataaatataaatattacttcacctaaataaattttaggtacaggtacagttgtgttacatggatatactgtgtagtggtgaagtctgggctttcagtgtagccaaatagtatacattattcccattagataatttctcctgcctcaccctcctctatcctcccaactctctgagtctccaatagctttcattccactgtctctgtgcctgtgtacactttatttagctcccacttaaaagtgagaatctacaatatttgactttctgtttctgagttgtttcacttgagataatagcctccagttctatccatgttgctgcaaaagatatgattttatgatttttttatggctaagtaacattctatagtatattctatacaccacattttctttatccattcatttgctgatagactcttaggttgatccatatctttgttattgtgaatagtgctgcagtaaacatgtgagtgtaggtatctttgtgacatgatttttttttctttcttttcctttggctatgtacccagtagtgggattgctggaggtctccaccttggcaaaggggcagttgtctagttctaaatgaaatgaagagattccatttccatttcatgacaactaaaagacaattcagtccaatgctttgtttaaaataattgaaccaggaacagaaagagctgaaaatgtcagtgaaatgtcaaacccaaagtggagagagagagagaggtggaaatgaatgtctccatcaagtgggttagggtggtggtggagggaggtttgagaattgagtccctgtttccttaccaatggaaattaacataggacatcagaaacagcatggaaaatttctttgattatcaaaagtacactagctaaggttgctgtccctcttttatttatttatttatttgagacagggtcttgctctgtcactcagatttggttgcactgggtgtgatctcagcccactgcagcctccacttcctaggctcaagcaatccacccgtctcatcctcccaagttgctgggaccacaggtgtgcaccaccacccccagctaatgtttgtttttttgtagagacagagtttcgccatgttggccaggctggtctagaactcctggcctcaagcaatccacctgccttggtctcccacagtgcagggattacaagcctgagccactatgcccagcttgctattcctcattgacaacattcacttaaacaagcaaacaaatatccgtaaaattaagtcagctttaaaacctggcttgtatatatctctgaattggaactctcagagccctcagcacttgcctgattggcctcctgttgatgaaaagttgtcctccagacaactcagccaagggaggccctgtgtgccttgcctatcacatgagcctcactttccactgagtgaggctgtcatttcagaagcaccgggtctgtcacatgaaaatatatctgttaccatcacttactaaacaaatttagtagaatttgtttggtgcttattatgtatcaggcattgttctgaaggctggggataccatttagtgaactaaatcgacaaaaactttgcctattggaccagagtggagatgagagagaagtgaccagatcgatctgtgttatcagcagacagccaataagatttgctgataggttggaccacggattgtgaaggagtcagtgatagcaccaagacttttggcccaaggaactggaaagatggaaatgtcaatgacagaatgtggaggatttcgcaagcagcagagtgtaggagaagcaatgcccttggctcatctagggcaaagggttgaattggcaactggaaatagaagtcaaattcagaagagaggtttatgtggacatctgtgtttaggtgtcaccaatatacagctgatactttttaaaaatctagtgaaattatcaaagggttaaatgcagagagggaagaaagtaggtccaaagatcaagccttgggacattggaagtttagaaataagaaagatgtcattgtcacttgtaattttgtgctagtcactgctctttttctttgtcttattaccttactgaccaattcctagaataggaataacacatttgatctttaatacagtatgtgataggaacatggcttctataagcccaaacttggcaatttaaatttaaatttattaaaattaaataaaactggctgggtgcagtggctcacgcctgtaatcccagcactttgggaggccgaggcggtgaatcacgaggtcaggagtacgagaccatcctggccaacatggtgaaaccccgtctctactaaaaatacaaaagttagccaggtgtggtggcatgtgcctgtaatcccagctactcaagaggctgaggcaggagaatcacttgaacccgggaggcggaggttgcagtgagccaagatcgcgacactgcactccagcctgggtgacaagagaaagactatgtctcaaaaaaaaaaaaaattttaaataaaactaaaaattcaccttcccagctgtgttagccacatttcaagtgcccaatagccacatgtagttggcagttactgtattggatggcacaggcatagaatatttccatcactgcagaaagatctatggacagtcttgctctagactgtgattttgtctacttaggaaaagtcactcttttccaggaagatgcttccagggtgtggagtaaacgacggacttcacccatctccttataaacctattgcaaccctggataggaggtttctaggtagcatgaagactcttgaatctttaagataggctgggagtgttgaaaggaaggaaatgaaaagggaagatactaggaagactgacaatagagcaagctcagaaaatttttatggaggtgatttagttagaaaatttgtctgcaacctaagggccatggagtgtgactccatggtttatgggtgtaactccgtggtttatgaagagtactttcaaaataggaaaatcttcacaactaagtgctagcatgagctgactttactttctctaggtgctgtaaaaatgtttttatgtgtttgatatgatatatttctacttcccttttgtttttgttagaaatctttgtctccttaaaacatctgagagtggattgcccagtacacgcattaaaaagagcaaagctctttctggtttcagtctacaaagctgcaggcacagcatcccaggtaaactgagagttctgcattctggctgagagtgaccagccccgaggaggctgatacatgctgagggagggtctcactctgacatgtggtctgctgtctagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagtaagccatatgaagggttatgcagacacccttgtcccgtctgcctgtgaggtgcattatgtttataccgttttgtttccaactgcaggggcaagtgttacttaaagctttcttcaaacggatctccaactaaaatacttcacgggagaggaggcatctctggatacacattaaggttgtgtaaaatggataatggtgagtataatgtcacttgaaaaaatatagctgaaggaattattccatgcttcatacatcacaatcaagactgtcagttatagccacagaagggagaacattcaggaaataacaaattttgcaattttctattattttcactcctgtcactcaagctgaccatgttttaaaggtaaatattgaggcttgactaaactgtacattgcctagtattaactaaatatatgctttaatttgacacatttatacacctggcatttctgttttctttctttcttttttttttttttttttttgagacacagtctcactctgttgcccaggctggagtgcagtgatgtggtcagtgttcactgcagcctctggcttgaactcctggactcgaacaatcctcccaccttagctccctgagtagctggggctacaggcgtgcaccacaacactcggctaatttgttgtagagatgggatcttgccatgttgcccaggctggtcttgaactcctgggctcaagcaatcctcccgctttggcctcccaaagttctgggattacaggcttcagccactgcacccagccacaactggcatttcctaatgagacccagactctgccaacactcctgttatcaaggccagtaatctttcctattatcctttgtaagggaattatcgatcagcactttggttagtcaaattactaattttcttccaaaaattgtgtatctattcaagaaatactggagcacctcctttttagggtctttattcagattccatgcagggttctggagacttagggattggcaaaggttaaggtaaaactttactagtaacaatgagtgtggtaggatggaaataagtgtttagattacacgagacctgtaataacataaaagttacaataaaaatccaaccagcagtgtttccatcccagtggccaatttctgagcatagtcacgagagatgctttgtaggcaaacagaattgttctaagggacaaaatccccaacaggaaagaacacaccacaaacactcctgcttaaattaccatagtaacttaggatggctttactatatattgatttacataaagctgcatgttcttatactgtttattgccaaatgtccaatattacaatacactataaggtgcaactgagatttaatgaataaaatggaagtaacaatcctgcctcgtgatagttttagaagcacaaaaacattctgtgtcagattatctgctgtaccgagaaggcgaatcaatccttaatttctgagaacttgttttgtagaacataaagacgttatattgcctccaacactggtatcctaactaacagactatgccttcctagagcttagaggcgctccgatgaaaatctctggatggctcaagacttcttaaaaagcaagtcaattacgtcgtatctcatacattctgttttcctcacaataaatttccctaagacaagaaggagcattcggcaccattctgttgtctttcttctacttctaagcgttagaagggacacttagcaaatgttgctgttaagtaatgttgacatggtttaataaaatgggaatgagcacgtatacctcaatacattgcagactgcattttcccccttccttcttcattatggttttctctgttggatttatagactctggcctgtagaagttacagaatatgccaggtatagattgatagctacaggagaaaatgtaagataaaggaaaataaagtcttacgtcttttcagtgcaactttggagggagtgaattagataactaggtttttactgcgctgtatttgatgaaataaccccctaatgtgaaagggaatagctgcgtgagatatttatggtgccttgtctgtcactggtctacaatgtaacttaactttctgaagatagatagcagcaccattaaaataaacatttcttaccacaaaatatgattctaaacacatattttcagcatttcgtttaaactgagaaacagcataggatgaacctcaaggcctctcacctggaccttgagttatttctaaaatatcttagttactatttacctattaattttcctaaaatttacctttatgacgcttcccaccttgcagaaattccagaatagatggccctccaaaatgaatgttcacccttccggctctaaaatgagagcctctgttcaggcttccgaagtcacatcgtgctcgttctcacctcagttgctgttagctgctgtttccttccgaactccttctctcatcctctcctcctatttgaaatctgccccagaattatacactcattttcctaccaaggaaaaaaaggcctagaaaggttgttttacacccacaaaactagtgaatggaccttctaggacccggcttctcatcagtgattcttctgttaacttagactcctcccttagctcaggacgcggagccttctgagcacctgagcctggttattctaaatgtgatctgggcacagcacattgacatcacctgggagcttgttagaaagaatttcaggacccacacagatgttactgagtcagaatctgcattttgaaagctacacaggcaacccacaggcacataaattttgagtcgcataggtgtgtgcgtgtgtgtgcgcatgtatgtgtgcgtgtgtttgtgtgcacgtgtgtgtgtgtgtgtctagaatactgctgtctacgaacaccctattcccatccatctgtgttccatggctccaaccgggagggtgggttcttgtgtcgggcatccagtaagtagaaatagagggcactgtcctgtctaggcagccccaagagaaaagaaacagagggatgagcctgagtcaaagtccctgaaaagtaccaaggaccccagagaatcccaaactgtcaacaaggccaaaggtcagaggaagttcatagcagatcagtcttactttggacgtgggtggagcaggagtgactgggatcatggtcagcagagtcactgggacagggcaggaactggacaatggctgcagcctgcgggcaaggtgcttgccttttctttctaagagcagttctcaaacgccagcagggctggttcaaacacagatggctgagcttccaggctggagtttctcattcactggatctgaggttgggctgaagaatgtgcatttctaacacgttcccaggtgacgctgttggtctggagactgcacttgacaaccactggtttaaaaacaccattcacgttatcatttgaaggagggtaagacagccttgtagtaccacacaaggagggctacattcttaggggtgtgtaattacaagatgacttagtcaattccatttttcatgtgcatgttttgctttggcagcttgattataaagtctctgtaactcagggtcatgataaactattgacttgaggaaaggttttcttcttgttcctgaaggagcataattactgatggaaaggaagatgtaggaagctgctcatcacaatgcttctgttgcagagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatgggtcagtaccacggctgtttttattagttcatcttcttcacacatttataaaaaatattactagcatgttaggaaataaatactttaaccaattagattgtcttatttgcaaaattaattaattgcttcagtggtaaaaaacgcaaaaaggaagagctcatggtctcccagcatcagaacaggtgcaggtacaaggctgcttgactgcctgctattccgcttcccatttaaccgcattcacatccccaagggccttcatgttattccctgcaagagcatacctccctctgtgcctcgctctgtgcactgtgcccggaactaaactcacagaggatttaccattgtctgaatcaaatttctaatatgtgtgtgtgtgtgtgtgcgtgtgtgtttaaatacagaaagtggccaggtgtggtggttcacgcctgtcatctcagcactttgggtggctgaggtgggaggattgctcaatgccaggagtctgaggtcagcctgggcaacatagtgagacctcgtctctaaaaaatatttaaaaactagcctggcatggtgttttgtgtgcctgtagcaccagctgctcagaagtctgaggtaggaggattgcttgagcccaagagttcaagggtgcagtgagttactacagtgccactgtgctccagcctgagcaacagagcaagaccctatctctaagtaaataaataaaatacagaacgagttcggtatgcatcctcacattggattccttacttaggtcactttcagcgttggccaaacaaaaaggctccagctgggggtatatatattccagggaagttaagttggccaaactttccgtttgctacttcagtatcctccgagttgtttccagacacagttttgtgtgctttttagttttggttttcttttttaatcaatctgcagcacactcatgttaaactcaagaacacatgtgaggccaagagttcccgttacctgtcacatatgggaatggagtagcagaaagcctagtttctgtcacagctagttactggcgagattgagactgagtccagcggtcttcgtgtgtgtgtgtgcgtgtgtgtctgtgcagtgtgcgtgtgtgtgcgtgtctgtgtgtgcgtgtgcgggtgtgtgcgtgtgtgtgtgttggagcacggagggagtgctcattcattttttgtgtataatggattttctttatagggtgaatatgttttttatcccgaaaaatcttaggataaaatcacttttttctacctaaatgtccatcattggcagaaaatattagtaataattaaacagccacacacttcacaatgtctgggaattatttttagtaaaggaaatttctttccctctgttgtttgctccttagggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacaggtacggagaattttatccggaaagttgtctccaatggtgaactggataaaatgtttaacactactagacttacggcctgaccctgccaatctctccatgcgttatcatcatgaaagggagagggcctggaatgctagtcattcactctgctaaggctgacacactttcctggctattgaaacttattttgggaatgtgggtaaagagatacgttttcctgagtcttcttcaggtgcatagaatgacataatttcataatactttggaatagtaaagataatttagtctaaagataatttattaaagataatttagggatgaaggattgaaggttagaacaattaagcaacttgtgcaggatcaaagtgagttggatgaggagttagcggtgagggtgaggcttgtctctctctcgccctctcatcctggcacatgtgcgatatcgtgctgaacctgagggaggaaaatacacgacaacaaggcaaaaaatgaatatagtaaacaaagaaaacacagataatgtacagtggaagaagagtctcttctggaaaagaggatatattttgcgtctcatatttaaaccacgattttttaaatttagattctcaacgacccatatgcctgccttccaaaggagatagaaatgtaatatacactgattgctgggtgactggatgggggtacagaaaactaagaggtaaaaatgatgttgttatatgtgctccatcctagaaatgaagagcggaaccttttctgccctgtcaagtcatgtagctgaagcacaactcgagtcacactactcagttgcaggaagcggattaataaagatggagaggcaaaaatcacccaagtgaggctggtgcctcatatgtttgattggaaattttaaatgtgactaaatctctttaaagactaattatatttaatgaagtttaatgtgaagcctagcacttttcagtaaatgttctagcctgctatccaattactttcttgggaagtcattccagttagagtcataattaatttttgaacttaattaacattaacaaaatggtacacgcaatagtgggaataatgtcttcttcatacttgtaattataaaaggtctgtgaagtaaatctaacattttttccttctagatttttatatagacatgagttttgtgttgttgttgttttgagatggactctcgctctgtcgcccaggctggagtgcagtggcacgatctcggctcactgttacctccacctcccgggttcaagtgattctcctgcctcagcctcccgagtagctgggattataggtacccaaccaccaccccaagctaattttttgtatttttagtagagacggggtttcatcatgttagccaggatggtctcaatctcctgacctcgtgatccacctgcctcggccttccaaagtgctggaattacaggcgtgagcccccacacccgtccatgatttttattttaaatatatgtggcccagcaccactggtggctcacgcctgtaatcccagcactttgggaggccaagatgggtggatcacttgaggtcaggagttcaagactggcctggccaacatggtgaaaccctgtctctactaaaaatacaaaaattagctgggcatggtggtgtgtgcctgtaatcccagctactcgggaggctgaggcaagataatcgcttgaacttgggaggtggaggtagcagtgagctgagattgcaccactgcactccagcctgggcgacagaaagagactccgtctcaattaaaaatatatatatatatatatttatatgtatgcatatatgtttatgtgtattgtgtatggttattctacaaacgaaccaaaaaaatttttttcagacaaaatacaaaatactctccagaaagccaagatacccttagtgaccaacgaagagtgccagaagagatacagaggacataaaataacccataagatgatctgtgccggctacagggaaggagggaaggacgcttgcaaggtaacagagtgttcttagccaatggaatatatgcaaattggaatgcttaatgcgttggggtttttttgtttgttttgttttttttgtttgtttttttttgagacagagtctcgctctgttgcccaggctggagtgcagtggctcgatctcagctcactgcaagctctgcctcccaggttcacgccattctcctgcctcagcctcccaaatagctgggactacaggcgccagctaccaagcccagctagcgtcttttttttttttagttttagtagagacggggtttcaccatgttggccaggatggtctcgatctcctgacctcatgatctgcctgcctgggcctcccaaagtgctgggattacaggcgtgagccaccgcgccgggccgcttaatgcattttaaaaagcagtcttctgccaatgagcagggaacacagtgtatttgtttgacttagactgaaatcaaaagcaaggagattgactggatgaacgcaagcacccaggttctctgcagtatattaaggggccaagacaacattttaggcaaaatcagcctgagcaagatgtgctgaagatgggaagcgtctgagttgatctgtgcaccttttcttgtctcccctcgttctagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtgattttttttttttttttttgagatgaagtctcaccctgttgcccaagctggagtgcagtggtgtgatctcggctcactgtaaactctacctcctggattcaagcgattgtcctgcctcagtctcccaagtagctgagattacaggcacatgccaccatgcccggctaatttttgtatttttagtagagacggggtttcactatgttggccaggctggtcttgaactcctgacctcgtgatctgcccacctcggcctctcaaagtgctgggattacaggtgtgagtcactgcgtctggccatggaaaatatttattgagcacaattatgtgagagcatcatgctgagctttgaagatacagtggtgagcaaacatatatcctggcttcatgaagattatactctagttaacatgagcaacaaaataaaataatcacacaaaatatataggttcaagctgaaatgagtggctgcaccagattctatgagataagaaaggaagaaggacatttttcaccaagttcaaagactgggatacaaaggaatttgtcctgacaaaggcaaaacaaaaacaacaacaaacaaaaaacccaaaagagcaaaatgacagtagaacataacggggccagatcaaaaatgctgacaggttcccaaaagaataaaatgacggtaggacatgacggggccagatccaaaatgctgacaggttcaaacaaaattggaattgaaaatcagagtgcgttcaagagtatcaaacaatactatcttgttacttgcttattaccttagtagactggaagcaacacttcacacaaaaaaagggtttggatgtaatttcggataagaagagatgtttctgtaaagtctttcctgagaagcatattatttgagaaaaacacatatttctgtttttagtatttcactttgtataatgtcttaatttttgaagagctggtatattcctatgattcattaatgaaagttctataagatataaaatatacaatgaggagatctcctcttctgtaccagaagagtgcacattctacacactgcgtagcacctttctcacttacgttctgtctgggcacacttctgattgacacgcagagggctctctctgtctggggatatttctgatgggtaccgagagagcttcctctatcttgggttatttctgatgcgtagagaagggctgcctctgtccattatggaaggctggtgttc

SEQ ID NO: 3 - DNA, 20 bp, Artificial Sequence
  primer
cagcctggagcatcgtaaca

SEQ ID NO: 4 - DNA, 25 bp, Artificial Sequence
  primer
tttatcgagcttcgttattctggtt

SEQ ID NO: 5 - DNA, 29 bp, Artificial Sequence
  probe
ttgtctactgaagcacacccaaacaggga

SEQ ID NO: 6 - DNA, 2246 bp, Mus musculus
ggataccggctcacgtagaaaaaggacaagactataggaaagaaagcaaacactccgccgaggactacagcaaagacagaaagtatctgcaggatgacctcattacatcaggtgttatattttatcttttttgcctcagtttctagtgaatgcgttactaaggtcttcaaagacatcagctttcaaggaggtgacctgagtactgttttcacaccgagcgccacatactgccgcttggtctgcactcaccacccacggtgcttgctcttcacgttcatggctgagtcatcttcggatgatcctaccaaatggtttgcctgcatcctgaaggacagcgtcacagaaatattgccaatggtaaacatgacaggcgcgatctctggatattccttcaagcaatgccctcagcaattaagtacttgcagcaaagatgtgtacgtgaacctagacatgaagggcatgaactataacagctctgtcgtgaagaatgctcgagaatgccaggagagatgcacagacgatgcccactgccagtttttcacatacgcaacagggtattttcccagtgtggaccatcgtaaaatgtgtcttttgaagtacacccgaacggggacgccaaccacaataacgaagctcaatggcgtggtatctggattttcactgaagtcctgtggactttcaaacttggcttgtatcagggacattttccctaacacggtgctggcagaccttaacattgacagcgtggtggccccagatgcttttgtctgtcgtcgcatctgcacgcatcaccccacttgtttgttcttcacattcttttcccaagcatggccgaaagaatctcagagacatctttgtctccttaaaacctctgaaagtggattaccaagcacacgcattacaaagagccacgccctttcgggcttcagtctccagcactgcaggcacagtgtcccagtattctgccatccgtccttttacaacgacactgatttcttgggagaagagctggacatcgtcgatgtgaaaggccaagaaacctgtcagaaaacgtgtaccaataacgcccgctgccagttctttacctactatccatcgcacagactgtgcaatgagaggaaccgcaggggcagatgttacctaaagctttcctccaatggatctccaacgagaatacttcatgggaggggaggcatctctggatactcactgaggctgtgcaaaatggataatgtgtgcacaactaaaatcaaccccagagtggtaggaggagctgcgtctgttcacggtgagtggccatggcaggtgactctgcacatcagccagggacacctgtgtggaggctccatcattggaaaccaatggatactgacagcagctcattgtttctctgggatagagacacctaaaaagctgcgtgtctacggtggcattgtaaatcaatcagaaataaatgaagggactgctttcttcagggttcaagaaatgataattcatgatcagtatacgacagcagaaagtgggtatgatattgccctgttaaaactggaatcagccatgaattacacagattttcagcggccaatatgcctgccttccaaaggagatagaaacgcagtgcacacagaatgctgggtgactggatgggggtacacagcactaagaggtgaagtacaaagtactcttcagaaagccaaggttccattggtgtcaaatgaagaatgtcagacaagatacagaagacacaaaataaccaataagatgatctgtgcaggctacaaagaaggagggaaggatacgtgcaagggagattctggagggcccctgtcctgcaaatacaatggggtctggcacttggtgggcatcacaagctggggtgaaggctgtggtcagaaggagagaccgggggtctacacgaacgtggccaagtacgtggactggattctggagaaaactcaaacagtctgaaagagttcaactggtatcactttgtggccctggaagattattccatagaaatgagcttgacgtctctgatgaagacactgggatactgactcttccactgtaaccaattgaatggccttgatgtacgtaagaacacccagaaagaaaactattattttcagaattcctgatctgggagaaccactggttgttttctgcatccagctactactcaaggaaacaaatacagcaaggagattttaaaaataaaaacacatcagatatataaggaaaatatc

SEQ ID NO: 7 - DNA, 20 bp, Artificial Sequence
  primer
acatgacaggcgcgatctct

SEQ ID NO: 8 - DNA, 25 bp, Artificial Sequence
  primer
tctaggttcacgtacacatctttgc

SEQ ID NO: 9 - DNA, 26 bp, Artificial Sequence
  probe
ttccttcaagcaatgccctcagcaat

SEQ ID NO: 10 - DNA, 20 bp, Artificial Sequence
  synthetic oligonucleotide
tcctggcattctcgagcatt

SEQ ID NO: 11 - DNA, 20 bp, Artificial Sequence
  synthetic oligonucleotide
tggtaatccactttcagagg

SEQ ID NO: 12 - DNA, 20 bp, Artificial Sequence
  synthetic oligonucleotide
aaggacctacactatggaat

SEQ ID NO: 13 - DNA, 20 bp, Artificial Sequence
  synthetic oligonucleotide
cctctgaaagtggattacca

SEQ ID NO: 14 - DNA, 20 bp, Artificial Sequence
  synthetic oligonucleotide
tcggaagcgactcttatatg

SEQ ID NO: 15 - DNA, 20 bp, Artificial Sequence
  synthetic oligonucleotide
ttcaaacaagtgacatacac

SEQ ID NO: 16 - DNA, 20 bp, Artificial Sequence
  synthetic oligonucleotide
tgagagaattgcttgctttc

SEQ ID NO: 17 - DNA, 20 bp, Artificial Sequence
  synthetic oligonucleotide
aaatataccttgagagaatt

SEQ ID NO: 18 - DNA, 20 bp, Artificial Sequence
  synthetic oligonucleotide
agtatgtcagaaatatacct

SEQ ID NO: 19 - DNA, 20 bp, Artificial Sequence
  synthetic oligonucleotide
ttaaaatcttagtatgtcag

SEQ ID NO: 20 - DNA, 20 bp, Artificial Sequence
  synthetic oligonucleotide
cagcatatttgtgaaagtcg

SEQ ID NO: 21 - DNA, 20 bp, Artificial Sequence
  synthetic oligonucleotide
tgtgtaggaaatggtcactt

SEQ ID NO: 22 - DNA, 20 bp, Artificial Sequence
  synthetic oligonucleotide
tgcaattcttaataagggtg

SEQ ID NO: 23 - DNA, 20 bp, Artificial Sequence
  synthetic oligonucleotide
aaatcatcctgaaaagacct

SEQ ID NO: 24 - DNA, 20 bp, Artificial Sequence
  synthetic oligonucleotide
tgatataagaaaatcatcct

SEQ ID NO: 25 - DNA, 20 bp, Artificial Sequence
  synthetic oligonucleotide
acacattcaccagaaactga

SEQ ID NO: 26 - DNA, 20 bp, Artificial Sequence
  synthetic oligonucleotide
ttcaggacacaagtaaacca

SEQ ID NO: 27 - DNA, 20 bp, Artificial Sequence
  synthetic oligonucleotide
ttcactcttggcagtgtttc

SEQ ID NO: 28 - DNA, 20 bp, Artificial Sequence
  synthetic oligonucleotide
aagaatacccagaaatcgct

SEQ ID NO: 29 - DNA, 20 bp, Artificial Sequence
  synthetic oligonucleotide
cattgcttgaaagaataccc

SEQ ID NO: 30 - DNA, 20 bp, Artificial Sequence
  synthetic oligonucleotide
ttggtgtgagcattgcttga

SEQ ID NO: 31 - DNA, 20 bp, Artificial Sequence
  synthetic oligonucleotide
aatgtctttgttgcaagcgc

SEQ ID NO: 32 - DNA, 20 bp, Artificial Sequence
  synthetic oligonucleotide
ttcatgtctaggtccacata

SEQ ID NO: 33 - DNA, 20 bp, Artificial Sequence
  synthetic oligonucleotide
gtttatgcccttcatgtcta

SEQ ID NO: 34 - DNA, 20 bp, Artificial Sequence
  synthetic oligonucleotide
ccgtgcatctttcttggcat

SEQ ID NO: 35 - DNA, 20 bp, Artificial Sequence
  synthetic oligonucleotide
cgtgaaaaagtggcagtgga

SEQ ID NO: 36 - DNA, 20 bp, Artificial Sequence
  synthetic oligonucleotide
agacaaatgttacgatgctc

SEQ ID NO: 37 - DNA, 20 bp, Artificial Sequence
  synthetic oligonucleotide
gtgcttcagtagacaaatgt

SEQ ID NO: 38 - DNA, 20 bp, Artificial Sequence
  synthetic oligonucleotide
tgcacaggatttcagtgaaa

SEQ ID NO: 39 - DNA, 20 bp, Artificial Sequence
  synthetic oligonucleotide
gattagaaagtgcacaggat

SEQ ID NO: 40 - DNA, 20 bp, Artificial Sequence
  synthetic oligonucleotide
ccgggatgatgagtgcagat

SEQ ID NO: 41 - DNA, 20 bp, Artificial Sequence
  synthetic oligonucleotide
aaacaagcaaccgggatgat

SEQ ID NO: 42 - DNA, 20 bp, Artificial Sequence
  synthetic oligonucleotide
tcctgggaaaagaaggtaaa

SEQ ID NO: 43 - DNA, 20 bp, Artificial Sequence
  synthetic oligonucleotide
attctttgggccattcctgg

SEQ ID NO: 44 - DNA, 20 bp, Artificial Sequence
  synthetic oligonucleotide
aaagatttctttgagattct

SEQ ID NO: 45 - DNA, 20 bp, Artificial Sequence
  synthetic oligonucleotide
aatccactctcagatgtttt

SEQ ID NO: 46 - DNA, 20 bp, Artificial Sequence
  synthetic oligonucleotide
aaccagaaagagctttgctc

SEQ ID NO: 47 - DNA, 20 bp, Artificial Sequence
  synthetic oligonucleotide
ggcagaacactgggatgctg

SEQ ID NO: 48 - DNA, 20 bp, Artificial Sequence
  synthetic oligonucleotide
tggtaaaatgaagaatggca

SEQ ID NO: 49 - DNA, 20 bp, Artificial Sequence
  synthetic oligonucleotide
atcagtgtcatggtaaaatg

SEQ ID NO: 50 - DNA, 20 bp, Artificial Sequence
  synthetic oligonucleotide
aacaatatccagttcttctc

SEQ ID NO: 51 - DNA, 20 bp, Artificial Sequence
  synthetic oligonucleotide
acagtttctggcaggcctcg

SEQ ID NO: 52 - DNA, 20 bp, Artificial Sequence
  synthetic oligonucleotide
gcattggtgcacagtttctg

SEQ ID NO: 53 - DNA, 20 bp, Artificial Sequence
  synthetic oligonucleotide
gcagcggacggcattggtgc

SEQ ID NO: 54 - DNA, 20 bp, Artificial Sequence
  synthetic oligonucleotide
ttgaagaaagctttaagtaa

SEQ ID NO: 55 - DNA, 20 bp, Artificial Sequence
  synthetic oligonucleotide
agtattttagttggagatcc

SEQ ID NO: 56 - DNA, 20 bp, Artificial Sequence
  synthetic oligonucleotide
atgtgtatccagagatgcct

SEQ ID NO: 57 - DNA, 20 bp, Artificial Sequence
  synthetic oligonucleotide
gtacactcattatccatttt

SEQ ID NO: 58 - DNA, 20 bp, Artificial Sequence
  synthetic oligonucleotide
gattttggtggtacactcat

SEQ ID NO: 59 - DNA, 20 bp, Artificial Sequence
  synthetic oligonucleotide
tcctgggcttgattttggtg

SEQ ID NO: 60 - DNA, 20 bp, Artificial Sequence
  synthetic oligonucleotide
ggccactcaccacgaacaga

SEQ ID NO: 61 - DNA, 20 bp, Artificial Sequence
  synthetic oligonucleotide
tgtctctgagtgggtgaggt

SEQ ID NO: 62 - DNA, 20 bp, Artificial Sequence
  synthetic oligonucleotide
gtttccaatgatggagcctc

SEQ ID NO: 63 - DNA, 20 bp, Artificial Sequence
  synthetic oligonucleotide
atatccactggtttccaatg

SEQ ID NO: 64 - DNA, 20 bp, Artificial Sequence
  synthetic oligonucleotide
ccatagaaacagtgagcggc

SEQ ID NO: 65 - DNA, 20 bp, Artificial Sequence
  synthetic oligonucleotide
tgactctaccccatagaaac

SEQ ID NO: 66 - DNA, 20 bp, Artificial Sequence
  synthetic oligonucleotide
cgcaaaatcttaggtgactc

SEQ ID NO: 67 - DNA, 20 bp, Artificial Sequence
  synthetic oligonucleotide
ttcagattgatttaaaatgc

SEQ ID NO: 68 - DNA, 20 bp, Artificial Sequence
  synthetic oligonucleotide
tgaaccccaaagaaagatgt

SEQ ID NO: 69 - DNA, 20 bp, Artificial Sequence
  synthetic oligonucleotide
tattatttcttgaaccccaa

SEQ ID NO: 70 - DNA, 20 bp, Artificial Sequence
  synthetic oligonucleotide
aacaaggcaatatcataccc

SEQ ID NO: 71 - DNA, 20 bp, Artificial Sequence
  synthetic oligonucleotide
ttccagtttcaacaaggcaa

SEQ ID NO: 72 - DNA, 20 bp, Artificial Sequence
  synthetic oligonucleotide
gaaggcaggcatatgggtcg

SEQ ID NO: 73 - DNA, 20 bp, Artificial Sequence
  synthetic oligonucleotide
gtcactaagggtatcttggc

SEQ ID NO: 74 - DNA, 20 bp, Artificial Sequence
  synthetic oligonucleotide
agatcatcttatgggttatt

SEQ ID NO: 75 - DNA, 20 bp, Artificial Sequence
  synthetic oligonucleotide
tagccggcacagatcatctt

SEQ ID NO: 76 - DNA, 20 bp, Artificial Sequence
  synthetic oligonucleotide
ccagatgccagacctcattg

SEQ ID NO: 77 - DNA, 20 bp, Artificial Sequence
  synthetic oligonucleotide
cattcacactgcttgagttt

SEQ ID NO: 78 - DNA, 20 bp, Artificial Sequence
  synthetic oligonucleotide
tggcacagtgaactcaacac

SEQ ID NO: 79 - DNA, 20 bp, Artificial Sequence
  synthetic oligonucleotide
ctagcattttcttacaaaca

SEQ ID NO: 80 - DNA, 20 bp, Artificial Sequence
  synthetic oligonucleotide
ttatggtaattcttggactc

SEQ ID NO: 81 - DNA, 20 bp, Artificial Sequence
  synthetic oligonucleotide
aaatattgccttatggtaat

SEQ ID NO: 82 - DNA, 20 bp, Artificial Sequence
  synthetic oligonucleotide
tatctgcctatatagtaatc

SEQ ID NO: 83 - DNA, 20 bp, Artificial Sequence
  synthetic oligonucleotide
gccactacttggttattttc

SEQ ID NO: 84 - DNA, 20 bp, Artificial Sequence
  synthetic oligonucleotide
aacaaatctatttatggtgg

SEQ ID NO: 85 - DNA, 20 bp, Artificial Sequence
  synthetic oligonucleotide
ctgcaaaatggtgaagactg

SEQ ID NO: 86 - DNA, 20 bp, Artificial Sequence
  synthetic oligonucleotide
gtgtagattcctgcaaaatg

SEQ ID NO: 87 - DNA, 20 bp, Artificial Sequence
  synthetic oligonucleotide
ttttcaggaaagtgtatctt

SEQ ID NO: 88 - DNA, 20 bp, Artificial Sequence
  synthetic oligonucleotide
cacaaatcatttttcaggaa

SEQ ID NO: 89 - DNA, 20 bp, Artificial Sequence
  synthetic oligonucleotide
tcccaagatattttaaataa

SEQ ID NO: 90 - DNA, 20 bp, Artificial Sequence
  synthetic oligonucleotide
aatgagataaatatttgcac

SEQ ID NO: 91 - DNA, 20 bp, Artificial Sequence
  synthetic oligonucleotide
tgaaagctatgtggtgacaa

SEQ ID NO: 92 - DNA, 20 bp, Artificial Sequence
  synthetic oligonucleotide
cacacttgatgaattgtata

SEQ ID NO: 93 - DNA, 20 bp, Artificial Sequence
  synthetic oligonucleotide
taccttgagagaattgcttg

SEQ ID NO: 94 - DNA, 20 bp, Artificial Sequence
  synthetic oligonucleotide
gtcagaaatataccttgaga

SEQ ID NO: 95 - DNA, 20 bp, Artificial Sequence
  synthetic oligonucleotide
atcttagtatgtcagaaata

SEQ ID NO: 96 - DNA, 20 bp, Artificial Sequence
  synthetic oligonucleotide
gagtcacacattcaccagaa

SEQ ID NO: 97 - DNA, 20 bp, Artificial Sequence
  synthetic oligonucleotide
gtgagcattgcttgaaagaa

SEQ ID NO: 98 - DNA, 20 bp, Artificial Sequence
  synthetic oligonucleotide
cttatttggtgtgagcattg

SEQ ID NO: 99 - DNA, 20 bp, Artificial Sequence
  synthetic oligonucleotide
acataaatgtctttgttgca

SEQ ID NO: 100 - DNA, 20 bp, Artificial Sequence
  synthetic oligonucleotide
ggtccacataaatgtctttg

SEQ ID NO: 101 - DNA, 20 bp, Artificial Sequence
  synthetic oligonucleotide
gtctaggtccacataaatgt

SEQ ID NO: 102 - DNA, 20 bp, Artificial Sequence
  synthetic oligonucleotide
tgcccttcatgtctaggtcc

SEQ ID NO: 103 - DNA, 20 bp, Artificial Sequence
  synthetic oligonucleotide
gttatagtttatgcccttca

SEQ ID NO: 104 - DNA, 20 bp, Artificial Sequence
  synthetic oligonucleotide
tcagtagacaaatgttacga

SEQ ID NO: 105 - DNA, 20 bp, Artificial Sequence
  synthetic oligonucleotide
tgggtgtgcttcagtagaca

SEQ ID NO: 106 - DNA, 20 bp, Artificial Sequence
  synthetic oligonucleotide
agccagattagaaagtgcac

SEQ ID NO: 107 - DNA, 20 bp, Artificial Sequence
  synthetic oligonucleotide
agcaaccgggatgatgagtg

SEQ ID NO: 108 - DNA, 20 bp, Artificial Sequence
  synthetic oligonucleotide
gccattcctgggaaaagaag

SEQ ID NO: 109 - DNA, 20 bp, Artificial Sequence
  synthetic oligonucleotide
ttgagattctttgggccatt

SEQ ID NO: 110 - DNA, 20 bp, Artificial Sequence
  synthetic oligonucleotide
actgaaaccagaaagagctt

SEQ ID NO: 111 - DNA, 20 bp, Artificial Sequence
  synthetic oligonucleotide
agaatggcagaacactggga

SEQ ID NO: 112 - DNA, 20 bp, Artificial Sequence
  synthetic oligonucleotide
tgtcatggtaaaatgaagaa

SEQ ID NO: 113 - DNA, 20 bp, Artificial Sequence
  synthetic oligonucleotide
aagaaatcagtgtcatggta

SEQ ID NO: 114 - DNA, 20 bp, Artificial Sequence
  synthetic oligonucleotide
ggtgcacagtttctggcagg

SEQ ID NO: 115 - DNA, 20 bp, Artificial Sequence
  synthetic oligonucleotide
ggacggcattggtgcacagt

SEQ ID NO: 116 - DNA, 20 bp, Artificial Sequence
  synthetic oligonucleotide
aactggcagcggacggcatt

SEQ ID NO: 117 - DNA, 20 bp, Artificial Sequence
  synthetic oligonucleotide
ccttaatgtgtatccagaga

SEQ ID NO: 118 - DNA, 20 bp, Artificial Sequence
  synthetic oligonucleotide
tggtggtacactcattatcc

SEQ ID NO: 119 - DNA, 20 bp, Artificial Sequence
  synthetic oligonucleotide
ggcttgattttggtggtaca

SEQ ID NO: 120 - DNA, 20 bp, Artificial Sequence
  synthetic oligonucleotide
aacgatcctgggcttgattt

SEQ ID NO: 121 - DNA, 20 bp, Artificial Sequence
  synthetic oligonucleotide
acaggtgtctctgagtgggt

SEQ ID NO: 122 - DNA, 20 bp, Artificial Sequence
  synthetic oligonucleotide
cactggtttccaatgatgga

SEQ ID NO: 123 - DNA, 20 bp, Artificial Sequence
  synthetic oligonucleotide
ctaccccatagaaacagtga

SEQ ID NO: 124 - DNA, 20 bp, Artificial Sequence
  synthetic oligonucleotide
ttaggtgactctaccccata

SEQ ID NO: 125 - DNA, 20 bp, Artificial Sequence
  synthetic oligonucleotide
agacacgcaaaatcttaggt

SEQ ID NO: 126 - DNA, 20 bp, Artificial Sequence
  synthetic oligonucleotide
tttcttgaaccccaaagaaa

SEQ ID NO: 127 - DNA, 20 bp, Artificial Sequence
  synthetic oligonucleotide
gtggtttccagtttcaacaa

SEQ ID NO: 128 - DNA, 20 bp, Artificial Sequence
  synthetic oligonucleotide
ttggctttctggagagtatt

SEQ ID NO: 129 - DNA, 20 bp, Artificial Sequence
  synthetic oligonucleotide
ggcacagatcatcttatggg

SEQ ID NO: 130 - DNA, 20 bp, Artificial Sequence
  synthetic oligonucleotide
gattcctgcaaaatggtgaa

SEQ ID NO: 131 - DNA, 20 bp, Artificial Sequence
  synthetic oligonucleotide
gcagagtgtagattcctgca

SEQ ID NO: 132 - DNA, 20 bp, Artificial Sequence
  synthetic oligonucleotide
atcatttttcaggaaagtgt

SEQ ID NO: 133 - DNA, 20 bp, Artificial Sequence
  synthetic oligonucleotide
gtgagacaaatcaagacttc

SEQ ID NO: 134 - DNA, 20 bp, Artificial Sequence
  synthetic oligonucleotide
ttagtttactgacactaaga

SEQ ID NO: 135 - DNA, 20 bp, Artificial Sequence
  synthetic oligonucleotide
ctgctttatgaaaaaccaac

SEQ ID NO: 136 - DNA, 20 bp, Artificial Sequence
  synthetic oligonucleotide
atacctagtacaatgtaaat

SEQ ID NO: 137 - DNA, 20 bp, Artificial Sequence
  synthetic oligonucleotide
ggcttgtgtgtggtcaatat

SEQ ID NO: 138 - DNA, 20 bp, Artificial Sequence
  synthetic oligonucleotide
tgggaaagctttcaatattc

SEQ ID NO: 139 - DNA, 20 bp, Artificial Sequence
  synthetic oligonucleotide
atggaattgtgcttatgagt

SEQ ID NO: 140 - DNA, 20 bp, Artificial Sequence
  synthetic oligonucleotide
tttcaagctcaggatgggaa

SEQ ID NO: 141 - DNA, 20 bp, Artificial Sequence
  synthetic oligonucleotide
gttggtaaaatgcaaccaaa

SEQ ID NO: 142 - DNA, 20 bp, Artificial Sequence
  synthetic oligonucleotide
tcaggacacaagtaaacctg

SEQ ID NO: 143 - DNA, 20 bp, Artificial Sequence
  synthetic oligonucleotide
tgcaagctggaaataaaagc

SEQ ID NO: 144 - DNA, 20 bp, Artificial Sequence
  synthetic oligonucleotide
tgccaatttaaaagtgtagc

SEQ ID NO: 145 - DNA, 20 bp, Artificial Sequence
  synthetic oligonucleotide
atatttcaaaatccagtatg

SEQ ID NO: 146 - DNA, 20 bp, Artificial Sequence
  synthetic oligonucleotide
ttctgaatatacaaattaat

SEQ ID NO: 147 - DNA, 20 bp, Artificial Sequence
  synthetic oligonucleotide
tttactatgaaaatctaaat

SEQ ID NO: 148 - DNA, 20 bp, Artificial Sequence
  synthetic oligonucleotide
ggtatcctgagtgagatcta

SEQ ID NO: 149 - DNA, 20 bp, Artificial Sequence
  synthetic oligonucleotide
ccagctatcaggaaaattcc

SEQ ID NO: 150 - DNA, 20 bp, Artificial Sequence
  synthetic oligonucleotide
aaagctattggagactcaga

SEQ ID NO: 151 - DNA, 20 bp, Artificial Sequence
  synthetic oligonucleotide
atggaatctcttcatttcat

SEQ ID NO: 152 - DNA, 20 bp, Artificial Sequence
  synthetic oligonucleotide
atggagacattcatttccac

SEQ ID NO: 153 - DNA, 20 bp, Artificial Sequence
  synthetic oligonucleotide
gctctgagagttccaattca

SEQ ID NO: 154 - DNA, 20 bp, Artificial Sequence
  synthetic oligonucleotide
ctgggaaggtgaatttttag

SEQ ID NO: 155 - DNA, 20 bp, Artificial Sequence
  synthetic oligonucleotide
tcaagagtcttcatgctacc

SEQ ID NO: 156 - DNA, 20 bp, Artificial Sequence
  synthetic oligonucleotide
tcagtttacctgggatgctg

SEQ ID NO: 157 - DNA, 20 bp, Artificial Sequence
  synthetic oligonucleotide
gacattatactcaccattat

SEQ ID NO: 158 - DNA, 20 bp, Artificial Sequence
  synthetic oligonucleotide
gtataaatgtgtcaaattaa

SEQ ID NO: 159 - DNA, 20 bp, Artificial Sequence
  synthetic oligonucleotide
gtaaagttttaccttaacct

SEQ ID NO: 160 - DNA, 20 bp, Artificial Sequence
  synthetic oligonucleotide
ccataatgaagaaggaaggg

SEQ ID NO: 161 - DNA, 20 bp, Artificial Sequence
  synthetic oligonucleotide
ttaagttacattgtagacca

SEQ ID NO: 162 - DNA, 20 bp, Artificial Sequence
  synthetic oligonucleotide
tgtgtgggtcctgaaattct

SEQ ID NO: 163 - DNA, 20 bp, Artificial Sequence
  synthetic oligonucleotide
atcttgtaattacacacccc

SEQ ID NO: 164 - DNA, 20 bp, Artificial Sequence
  synthetic oligonucleotide
gtacactctgcaacagaagc

SEQ ID NO: 165 - DNA, 20 bp, Artificial Sequence
  synthetic oligonucleotide
agggaataacatgaaggccc

SEQ ID NO: 166 - DNA, 20 bp, Artificial Sequence
  synthetic oligonucleotide
atccagttcaccattggaga

SEQ ID NO: 167 - DNA, 20 bp, Artificial Sequence
  synthetic oligonucleotide
ttttccagaagagactcttc

SEQ ID NO: 168 - DNA, 20 bp, Artificial Sequence
  synthetic oligonucleotide
gtcacatttaaaatttccaa

SEQ ID NO: 169 - DNA, 20 bp, Artificial Sequence
  synthetic oligonucleotide
ttaatatactgcagagaacc

SEQ ID NO: 170 - DNA, 20 bp, Artificial Sequence
  synthetic oligonucleotide
agaaatatccccagacagag

SEQ ID NO: 171 - DNA, 20 bp, Artificial Sequence
  synthetic oligonucleotide
aaatgtctttgttgcaagcg

SEQ ID NO: 172 - DNA, 20 bp, Artificial Sequence
  synthetic oligonucleotide
taaatgtctttgttgcaagc

SEQ ID NO: 173 - DNA, 20 bp, Artificial Sequence
  synthetic oligonucleotide
ataaatgtctttgttgcaag

SEQ ID NO: 174 - DNA, 20 bp, Artificial Sequence
  synthetic oligonucleotide
cataaatgtctttgttgcaa

SEQ ID NO: 175 - DNA, 20 bp, Artificial Sequence
  synthetic oligonucleotide
cacataaatgtctttgttgc

SEQ ID NO: 176 - DNA, 20 bp, Artificial Sequence
  synthetic oligonucleotide
ccacataaatgtctttgttg

SEQ ID NO: 177 - DNA, 20 bp, Artificial Sequence
  synthetic oligonucleotide
tccacataaatgtctttgtt

SEQ ID NO: 178 - DNA, 20 bp, Artificial Sequence
  synthetic oligonucleotide
gtccacataaatgtctttgt

SEQ ID NO: 179 - DNA, 20 bp, Artificial Sequence
  synthetic oligonucleotide
aggtccacataaatgtcttt

SEQ ID NO: 180 - DNA, 20 bp, Artificial Sequence
  synthetic oligonucleotide
taggtccacataaatgtctt

SEQ ID NO: 181 - DNA, 20 bp, Artificial Sequence
  synthetic oligonucleotide
ctaggtccacataaatgtct

SEQ ID NO: 182 - DNA, 20 bp, Artificial Sequence
  synthetic oligonucleotide
tctaggtccacataaatgtc

SEQ ID NO: 183 - DNA, 20 bp, Artificial Sequence
  synthetic oligonucleotide
tgtctaggtccacataaatg

SEQ ID NO: 184 - DNA, 20 bp, Artificial Sequence
  synthetic oligonucleotide
atgtctaggtccacataaat

SEQ ID NO: 185 - DNA, 20 bp, Artificial Sequence
  synthetic oligonucleotide
catgtctaggtccacataaa

SEQ ID NO: 186 - DNA, 20 bp, Artificial Sequence
  synthetic oligonucleotide
tcatgtctaggtccacataa

SEQ ID NO: 187 - DNA, 20 bp, Artificial Sequence
  synthetic oligonucleotide
cttcatgtctaggtccacat

SEQ ID NO: 188 - DNA, 20 bp, Artificial Sequence
  synthetic oligonucleotide
ccttcatgtctaggtccaca

SEQ ID NO: 189 - DNA, 20 bp, Artificial Sequence
  synthetic oligonucleotide
cccttcatgtctaggtccac

SEQ ID NO: 190 - DNA, 20 bp, Artificial Sequence
  synthetic oligonucleotide
gcccttcatgtctaggtcca

SEQ ID NO: 191 - DNA, 20 bp, Artificial Sequence
  synthetic oligonucleotide
atgcccttcatgtctaggtc

SEQ ID NO: 192 - DNA, 20 bp, Artificial Sequence
  synthetic oligonucleotide
tatgcccttcatgtctaggt

SEQ ID NO: 193 - DNA, 20 bp, Artificial Sequence
  synthetic oligonucleotide
ttatgcccttcatgtctagg

SEQ ID NO: 194 - DNA, 20 bp, Artificial Sequence
  synthetic oligonucleotide
tttatgcccttcatgtctag

SEQ ID NO: 195 - DNA, 20 bp, Artificial Sequence
  synthetic oligonucleotide
tccgtgcatctttcttggca

SEQ ID NO: 196 - DNA, 20 bp, Artificial Sequence
  synthetic oligonucleotide
atccgtgcatctttcttggc

SEQ ID NO: 197 - DNA, 20 bp, Artificial Sequence
  synthetic oligonucleotide
catccgtgcatctttcttgg

SEQ ID NO: 198 - DNA, 20 bp, Artificial Sequence
  synthetic oligonucleotide
tcatccgtgcatctttcttg

SEQ ID NO: 199 - DNA, 20 bp, Artificial Sequence
  synthetic oligonucleotide
gtcatccgtgcatctttctt

SEQ ID NO: 200 - DNA, 20 bp, Artificial Sequence
  synthetic oligonucleotide
accgggatgatgagtgcaga

SEQ ID NO: 201 - DNA, 20 bp, Artificial Sequence
  synthetic oligonucleotide
aaccgggatgatgagtgcag

SEQ ID NO: 202 - DNA, 20 bp, Artificial Sequence
  synthetic oligonucleotide
caaccgggatgatgagtgca

SEQ ID NO: 203 - DNA, 20 bp, Artificial Sequence
  synthetic oligonucleotide
gcaaccgggatgatgagtgc

SEQ ID NO: 204 - DNA, 20 bp, Artificial Sequence
  synthetic oligonucleotide
gattctttgggccattcctg

SEQ ID NO: 205 - DNA, 20 bp, Artificial Sequence
  synthetic oligonucleotide
agattctttgggccattcct

SEQ ID NO: 206 - DNA, 20 bp, Artificial Sequence
  synthetic oligonucleotide
gagattctttgggccattcc

SEQ ID NO: 207 - DNA, 20 bp, Artificial Sequence
  synthetic oligonucleotide
tgagattctttgggccattc

SEQ ID NO: 208 - DNA, 20 bp, Artificial Sequence
  synthetic oligonucleotide
tttgagattctttgggccat

SEQ ID NO: 209 - DNA, 20 bp, Artificial Sequence
  synthetic oligonucleotide
ctttgagattctttgggcca

SEQ ID NO: 210 - DNA, 20 bp, Artificial Sequence
  synthetic oligonucleotide
tctttgagattctttgggcc

SEQ ID NO: 211 - DNA, 20 bp, Artificial Sequence
  synthetic oligonucleotide
ttctttgagattctttgggc

SEQ ID NO: 212 - DNA, 20 bp, Artificial Sequence
  synthetic oligonucleotide
tttctttgagattctttggg

SEQ ID NO: 213 - DNA, 20 bp, Artificial Sequence
  synthetic oligonucleotide
cacagtttctggcaggcctc

SEQ ID NO: 214 - DNA, 20 bp, Artificial Sequence
  synthetic oligonucleotide
gcacagtttctggcaggcct

SEQ ID NO: 215 - DNA, 20 bp, Artificial Sequence
  synthetic oligonucleotide
tgcacagtttctggcaggcc

SEQ ID NO: 216 - DNA, 20 bp, Artificial Sequence
  synthetic oligonucleotide
gtgcacagtttctggcaggc

SEQ ID NO: 217 - DNA, 20 bp, Artificial Sequence
  synthetic oligonucleotide
tggtgcacagtttctggcag

SEQ ID NO: 218 - DNA, 20 bp, Artificial Sequence
  synthetic oligonucleotide
ttggtgcacagtttctggca

SEQ ID NO: 219 - DNA, 20 bp, Artificial Sequence
  synthetic oligonucleotide
attggtgcacagtttctggc

SEQ ID NO: 220 - DNA, 20 bp, Artificial Sequence
  synthetic oligonucleotide
cattggtgcacagtttctgg

SEQ ID NO: 221 - DNA, 20 bp, Artificial Sequence
  synthetic oligonucleotide
ggcattggtgcacagtttct

SEQ ID NO: 222 - DNA, 20 bp, Artificial Sequence
  synthetic oligonucleotide
cggcattggtgcacagtttc

SEQ ID NO: 223 - DNA, 20 bp, Artificial Sequence
  synthetic oligonucleotide
acggcattggtgcacagttt

SEQ ID NO: 224 - DNA, 20 bp, Artificial Sequence
  synthetic oligonucleotide
gacggcattggtgcacagtt

SEQ ID NO: 225 - DNA, 20 bp, Artificial Sequence
  synthetic oligonucleotide
cggacggcattggtgcacag

SEQ ID NO: 226 - DNA, 20 bp, Artificial Sequence
  synthetic oligonucleotide
gcggacggcattggtgcaca

SEQ ID NO: 227 - DNA, 20 bp, Artificial Sequence
  synthetic oligonucleotide
agcggacggcattggtgcac

SEQ ID NO: 228 - DNA, 20 bp, Artificial Sequence
  synthetic oligonucleotide
cagcggacggcattggtgca

SEQ ID NO: 229 - DNA, 20 bp, Artificial Sequence
  synthetic oligonucleotide
ggcagcggacggcattggtg

SEQ ID NO: 230 - DNA, 20 bp, Artificial Sequence
  synthetic oligonucleotide
tggcagcggacggcattggt

SEQ ID NO: 231 - DNA, 20 bp, Artificial Sequence
  synthetic oligonucleotide
ctggcagcggacggcattgg

SEQ ID NO: 232 - DNA, 20 bp, Artificial Sequence
  synthetic oligonucleotide
actggcagcggacggcattg

SEQ ID NO: 233 - DNA, 20 bp, Artificial Sequence
  synthetic oligonucleotide
tgcttgaaggaatatccaga

SEQ ID NO: 234 - DNA, 20 bp, Artificial Sequence
  synthetic oligonucleotide
tagttcatgcccttcatgtc

SEQ ID NO: 235 - DNA, 20 bp, Artificial Sequence
  synthetic oligonucleotide
tgttatagttcatgcccttc

SEQ ID NO: 236 - DNA, 20 bp, Artificial Sequence
  synthetic oligonucleotide
aatgtccctgatacaagcca

SEQ ID NO: 237 - DNA, 20 bp, Artificial Sequence
  synthetic oligonucleotide
gggaaaatgtccctgataca

SEQ ID NO: 238 - DNA, 20 bp, Artificial Sequence
  synthetic oligonucleotide
tgtgcagagtcacctgccat

SEQ ID NO: 239 - DNA, 20 bp, Artificial Sequence
  synthetic oligonucleotide
ttcttgaaccctgaagaaag

SEQ ID NO: 240 - DNA, 20 bp, Artificial Sequence
  synthetic oligonucleotide
tgaattatcatttcttgaac

SEQ ID NO: 241 - DNA, 20 bp, Artificial Sequence
  synthetic oligonucleotide
tgatcatgaattatcatttc

SEQ ID NO: 242 - DNA, 18 bp, Artificial Sequence
  synthetic oligonucleotide
agtttctggcaggcctcg

SEQ ID NO: 243 - DNA, 18 bp, Artificial Sequence
  synthetic oligonucleotide
cagtttctggcaggcctc

SEQ ID NO: 244 - DNA, 18 bp, Artificial Sequence
  synthetic oligonucleotide
acagtttctggcaggcct

SEQ ID NO: 245 - DNA, 18 bp, Artificial Sequence
  synthetic oligonucleotide
cacagtttctggcaggcc

SEQ ID NO: 246 - DNA, 18 bp, Artificial Sequence
  synthetic oligonucleotide
gcacagtttctggcaggc

SEQ ID NO: 247 - DNA, 18 bp, Artificial Sequence
  synthetic oligonucleotide
gtgcacagtttctggcag

SEQ ID NO: 248 - DNA, 18 bp, Artificial Sequence
  synthetic oligonucleotide
ggtgcacagtttctggca

SEQ ID NO: 249 - DNA, 18 bp, Artificial Sequence
  synthetic oligonucleotide
tggtgcacagtttctggc

SEQ ID NO: 250 - DNA, 18 bp, Artificial Sequence
  synthetic oligonucleotide
ttggtgcacagtttctgg

SEQ ID NO: 251 - DNA, 18 bp, Artificial Sequence
  synthetic oligonucleotide
attggtgcacagtttctg

SEQ ID NO: 252 - DNA, 18 bp, Artificial Sequence
  synthetic oligonucleotide
cattggtgcacagtttct

SEQ ID NO: 253 - DNA, 18 bp, Artificial Sequence
  synthetic oligonucleotide
gcattggtgcacagtttc

SEQ ID NO: 254 - DNA, 18 bp, Artificial Sequence
  synthetic oligonucleotide
ggcattggtgcacagttt

SEQ ID NO: 255 - DNA, 18 bp, Artificial Sequence
  synthetic oligonucleotide
cggcattggtgcacagtt

SEQ ID NO: 256 - DNA, 18 bp, Artificial Sequence
  synthetic oligonucleotide
acggcattggtgcacagt

SEQ ID NO: 257 - DNA, 18 bp, Artificial Sequence
  synthetic oligonucleotide
gacggcattggtgcacag

SEQ ID NO: 258 - DNA, 18 bp, Artificial Sequence
  synthetic oligonucleotide
ggacggcattggtgcaca

SEQ ID NO: 259 - DNA, 18 bp, Artificial Sequence
  synthetic oligonucleotide
cggacggcattggtgcac

SEQ ID NO: 260 - DNA, 18 bp, Artificial Sequence
  synthetic oligonucleotide
gcggacggcattggtgca

SEQ ID NO: 261 - DNA, 18 bp, Artificial Sequence
  synthetic oligonucleotide
agcggacggcattggtgc

SEQ ID NO: 262 - DNA, 18 bp, Artificial Sequence
  synthetic oligonucleotide
cagcggacggcattggtg

SEQ ID NO: 263 - DNA, 18 bp, Artificial Sequence
  synthetic oligonucleotide
gcagcggacggcattggt

SEQ ID NO: 264 - DNA, 18 bp, Artificial Sequence
  synthetic oligonucleotide
ggcagcggacggcattgg

SEQ ID NO: 265 - DNA, 18 bp, Artificial Sequence
  synthetic oligonucleotide
tggcagcggacggcattg

SEQ ID NO: 266 - DNA, 18 bp, Artificial Sequence
  synthetic oligonucleotide
ctggcagcggacggcatt

SEQ ID NO: 267 - DNA, 18 bp, Artificial Sequence
  synthetic oligonucleotide
tgcacagtttctggcagg

SEQ ID NO: 268 - DNA, 16 bp, Artificial Sequence
  synthetic oligonucleotide
cacagtttctggcagg

SEQ ID NO: 269 - DNA, 14 bp, Artificial Sequence
  synthetic oligonucleotide
acagtttctggcag

SEQ ID NO: 270 - DNA, 27 bp, Artificial Sequence
  synthetic oligonucleotide
gcgtttgctcttcttcttgcgtttttt

SEQ ID NO: 271 - DNA, 23 bp, Artificial Sequence
  primer
acacgcattaaaaagagcaaagc

SEQ ID NO: 272 - DNA, 26 bp, Artificial Sequence
  primer
cagtgtcatggtaaaatgaagaatgg

SEQ ID NO: 273 - DNA, 25 bp, Artificial Sequence
  probe
tgcaggcacagcatcccagtgttct

SEQ ID NO: 274 - DNA, 3275 bp, Macaca mulatta
  misc_feature (1)...(3275): n = A,T,C or G
aggcacacagacaaaatcaagctctacagctgtctgtgtgtatgtcacttgtttgaatatgaattaaaattaaaatataaattcaatgtattgagaaagcaagcaattctctcaaggtatatttctgacatactaagattttaacgactttcacaaatatgctgcaccgagagacaatgttacacaacactgagaaccagtacaagtaaatattaaagttaagtgaccatttcctacacacgctcattcagaggaggatgaggatcaatttggaagaagaaaaacacccttattaagaattgcagcaaataagcaaacgaggtcttttcaggatgattttcttatatcaaatggtacatttcattctatttacgtcagtttctggtgaatgtgtgactcggttgttcaaggacatccactttgaaggaggggacattgctacggttttcacaccaagcgccaagcactgccaggtggtctgcactcaccacccacgatgtctgctcttcactttcacgggggaatctgcatctgaggatcctacccagtggtttacttgtgtcctgaaggacagtgttacagaaacactgccaagagtgaataggacaggagcgatttctgggtattctttcaagcaatgctcacaccaaataagcgcttgcaacaaagacatttatgtggacctagacatgaagggcataaactataacagctcacttgccaagagtgctcaagaatgccaagaaagatgcacggatgacatccactgccactttttcacgtatgccacaaggcagtttcccagtctggagcatcgtaacatttgtctactgaagcacacccaaacggggacaccaaccggaataatgaagctcgataaagtggtgactggattttcactgaaatcctgtgcactttctaatctggcttgtatcagggacgttttccccaacacggtgtttgcggacagcaacatcgatagtgtcatggctccagatgcctttgtctgtcgccggatctgcactcatcatcccggttgcttgttttttaccttcttttcccaggaatggcccaaagaatctcaaagaaatctttgtctccttaaaacatctgagagtggattacccagtacacgcattaaaaagagcaaagctctttctggtttcagtctccaaagctgcaggcacagcatcccagtgttctgccattcttcattttaccatgacactgatttcttgggagaagaactggatattgttgctgtgaaaggtcacgaggcctgccagaaactgtgcaccaatgccgtccgctgccagttttttacctatgccccagctcaagcatcttgcaacgaagggaagggcaaatgttacttaaagctttcttcaaatggatctccaactaaaatacttcgcgggacaggaggcatctctggatacacattaaggctgtgtaaaatggataatgagtgtaccaccaaaatcaagcccaggatcgttggaggaactgcatctgttcgtggtgagtggccatggcaggtgactctgcacaccacctcacccactcagagacacctgtgtggaggctccatcattggaaaccagtggatattaacggccgctcactgtttctatggggtagagtcacctaagattttgcgtgtctacattggcattttaaatcaatctgaaataaaagaggatacatctttctttggggttcaagaaataataatccatgatcaatataaaatggcagaaagtgggtatgatattgccttgttgaaactggaaaccacagtgaattacacagattctcaacgacccatatgcctgccttcaaaaggagatagaaatgtgatatacactgactgctgggtgactggatgggggtacagaaaattaagagacaaaatacagaatactctccagaaagccaagatacccttagtgaccaatgacgagtgccagaagagatacagaggacataaaataacccataagatgatctgtgccggctacagggaaggagggaaggatgcttgcaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcttggtgctggtaggaaaatgccagaagaaaacaaactgtcacaagctgttatgtccaaagctcccgttctatgatcattgtagtttgtttcagcatccagtgtctttgtttctgatcacgcttctaaggagtccaagaattaccataaggcaatatttctgatgattactatataggcagatatatcagaaaataaccaagtagtggcagcagggatcaggtagaagaactgataaaagaaaccaccataaatagatttgttcaatgaaaaatgaaaactggaagaaaggataacaaagacagtcttcaccattttgcaggaatctacactctgcctgtgtgaacacatttctttgtaaagaaagaatttgattgcatttactggcagattttcagaatagtcaggaattcatgttatttccattttaaaacatgtttaaaaaaatcagtttgagtagacacaagctaagagtgaatgtgaaggtaccagaatttctgtatggaagagggtgacaagcagcaatgtacctggaagtggtaccttaggaccaatcttaaagatacactttcctgaaaaatgatttgtgatggatcgtatatttatttaaaatatcttgggagggagaggctgatggcgatagggaggcaagctgaagcctccataagacaagctgctactgcgactgtggcccccaaagagctacaccgcatatttatttgacaaaagtcaccattgactacatccgtactacagagaaaaaacaatttgggcacaaatggatggttacagtaaagtcttcagcaagcagctccctgtattctaagtactgggcttttctgtttggtgcaaatatttatctcattattgctgtgatctagtccagtaacctagaatttgatttgtcaccacatagctttcaacctgtgccaacaattatacaattcatcaagtgtg

SEQ ID NO: 275 - DNA, 20 bp, Artificial Sequence
  synthetic oligonucleotide
ccatagaacagcttcacagg

(19) United States
(12) Patent Application Publication
(10) Pub. No.: US 2012/0083522 A1
(43) Pub. Date: Apr. 5, 2012

(54) MODULATION OF INFLAMMATORY RESPONSES BY FACTOR XI

(75) Inventors: Brett P. Monia, Encinitas, CA (US); Jeffrey R. Crosby, Carlsbad, CA (US); Robert A. MacLeod, San Diego, CA (US); Susan M. Freier, San Diego, CA (US)

(73) Assignee: Isis Pharmaceuitcals, inc., Carlsbad, CA (US)

(21) Appl. No.: 13/262,904

(22) PCT Filed: Apr. 15, 2010
(86) PCT No.: PCT/US10/31311

§ 371 Date: Dec. 15, 2011

Related U.S. Application Data
(60) Provisional application No. 61/169,701, filed on Apr. 15, 2009.

(30) Foreign Application Priority Data
Oct. 15, 2009 (US) PCT/US2009/006092

(51) Int. Cl. A61K 31/7125; A61K 31/7115; A61P 29/00; A61P 19/02; A61P 1/00; A61P 19/06; A61P 37/00; A61P 37/08; A61P 11/06; A61P 35/00; A61P 3/00; A61P 37/06; A61K 31/712; A61P 3/10

(52) U.S. Cl. 514/44 R

(57) ABSTRACT

Disclosed herein are antisense compounds and methods for modulating Factor XI and modulating an inflammatory disease, disorder or condition in an individual in need thereof. Inflammatory diseases in an individual such as arthritis and colitis can be ameliorated or prevented with the administration of antisense compounds targeted to Factor XI.

### SEQUENCE LISTING

The present application is being filed along with a Sequence Listing in electronic format. The Sequence Listing is provided as a file entitled BIOL0109WOSEQ.TXT created Apr. 14, 2010, which is 84 Kb in size. The information in the electronic format of the sequence listing is incorporated herein by reference in its entirety.

### FIELD OF THE INVENTION

The present invention provides methods, compounds, and compositions for modulating an inflammatory response by administering a Factor XI modulator to an animal.

### BACKGROUND OF THE INVENTION

### Factor XI

Factor XI, synthesized in the liver, is a member of the coagulation cascade “intrinsic pathway” which ultimately activates thrombin to prevent blood loss. The intrinsic pathway is triggered by activation of Factor XII to XIIa. Factor XIIa converts Factor XI to Factor XIa, and Factor XIa converts Factor IX to Factor IXa. Factor IXa associates with its cofactor Factor VIIIa to convert Factor X to Factor Xa. Factor Xa associates Factor Va to convert prothrombin (Factor II) to thrombin (Factor IIa).

Factor XI deficiency (also known as plasma thromboplastin antecedent (PTA) deficiency, Rosenthal syndrome and hemophilia C) is an autosomal recessive disease associated with a tendency to bleed. Most patients with Factor XI deficiency do not bleed spontaneously but can bleed seriously after trauma. Low levels of Factor XI can also occur in other disease states, including Noonan syndrome.

### Inflammation

Inflammation is a complex biological process of the body in response to an injury or abnormal stimulation caused by a physical, chemical or biological stimulus. Inflammation is a protective process by which the body attempts to remove the injury or stimulus and begins to heal affected tissue in the body.

The inflammatory response to injury or stimulus is characterized by clinical signs of increased redness (rubor), temperature (calor), swelling (tumor), pain (dolor) and/or loss of function (functio laesa) in a tissue. Increased redness and temperature is caused by vasodilation leading to increased blood supply at core body temperature to the inflamed tissue site. Swelling is caused by vascular permeability and accumulation of protein and fluid at the inflamed tissue site. Pain is due to the release of chemicals (e.g. bradykinin) at the inflamed tissue site that stimulate nerve endings. Loss of function may be due to several causes.

Inflammation is now recognized as a type of non-specific immune response to an injury or stimulus. The inflammatory response has a cellular component and an exudative component. In the cellular component, resident macrophages at the site of injury or stimulus initiate the inflammatory response by releasing inflammatory mediators such as TNFalpha, IFNalpha, IL-1, IL-6, IL12, IL-18 and others. Leukocytes are then recruited to move into the inflamed tissue area and perform various functions such as release of additional cellular mediators, phagocytosis, release of enzymatic granules and other functions. The exudative component involves the passage of plasma fluid containing proteins from blood vessels to the inflamed tissue site. Inflammatory mediators such as bradykinin, nitric oxide, and histamine cause blood vessels to become dilated, slow the blood flow in the vessels and increase the blood vessel permeability, allowing the movement of fluid and protein into the tissue. Biochemical cascades are activated in order to propagate the inflammatory response (e.g., complement system in response to infection, fibrinolysis and coagulation systems in response to necrosis due to a burn or trauma, kinin system to sustain inflammation) (Robbins Pathologic Basis of Disease, Philadelphia, W.B Saunders Company).

Inflammation can be acute or chronic. Acute inflammation has a fairly rapid onset, quickly becomes severe and quickly and distinctly clears after a few days to a few weeks. Chronic inflammation can begin rapidly or slowly and tends to persist for weeks, months or years with a vague and indefinite termination. Chronic inflammation can result when an injury or stimulus, or products resulting from its presence, persists at the site of injury or stimulation and the body's immune response is not sufficient to overcome its effects.

Inflammatory responses, although generally helpful to the body to clear an injury or stimulus, can sometimes cause injury to the body. In some cases, a body's immune response inappropriately triggers an inflammatory response where there is no known injury or stimulus to the body. In these cases, categorized as autoimmune diseases, the body attacks its own tissues causing injury to its own tissues.

Treatment to decrease inflammation includes non-steroidal anti-inflammatory drugs (NSAIDS) as well as disease modifying drugs. Many of these drugs have unwanted side effects. For example, with NSAIDS, the most common side effects are nausea, vomiting, diarrhea, constipation, decreased appetite, rash, dizziness, headache, and drowsiness. NSAIDs may also cause fluid retention, leading to edema. The most serious side effects are kidney failure, liver failure, ulcers and prolonged bleeding after an injury or surgery.

Accordingly, there is a need to find alternative treatments for inflammation with more attractive clinical profiles. Little is known about the role of Factor XI in inflammation making it an attractive target for investigation. Antisense technology is emerging as an effective means for reducing the expression of certain gene products and may therefore prove to be uniquely useful in a number of therapeutic, diagnostic, and research applications for the modulation of Factor XI.

### SUMMARY OF THE INVENTION

Provided herein are methods, compounds, and compositions for modulating levels of Factor XI mRNA and/or protein in an animal. Provided herein are methods, compounds, and compositions for modulating levels of Factor XI mRNA and/or protein in an animal in order to modulate an inflammatory response in the animal. Also provided herein are methods, compounds, and compositions for administering a therapeutically effective amount of a compound targeting Factor XI to an animal for ameliorating an inflammatory disease in an animal; treating an animal at risk for an inflammatory disease; inhibiting Factor XI expression in an animal suffering from an inflammatory disease; and reducing the risk of inflammatory disease in an animal.

In certain embodiments, Factor XI specific inhibitors modulate (i.e., decrease) levels of Factor XI mRNA and/or protein. In certain embodiments, Factor XI specific inhibitors are nucleic acids, proteins, or small molecules.

In certain embodiments, an animal at risk for an inflammatory disease is treated by administering to the animal a therapeutically effective amount of a compound comprising a modified oligonucleotide consisting of 12 to 30 linked nucleosides, wherein the modified oligonucleotide is complementary to a Factor XI nucleic acid as shown in SEQ ID NO: 1, SEQ ID NO: 2, SEQ ID NO: 6 or SEQ ID NO: 274 or a therapeutically effective amount of a compound comprising a modified oligonucleotide consisting of 12 to 30 linked nucleosides and having a nucleobase sequence comprising at least 8 contiguous nucleobases of a nucleobase sequence selected from any one of nucleobase sequences recited in SEQ ID NOs: 15 to 269.

In certain embodiments, an animal having an inflammatory disease is treated by administering to the animal a therapeutically effective amount of a compound comprising a modified oligonucleotide consisting of 12 to 30 linked nucleosides, wherein the modified oligonucleotide is complementary to a Factor XI nucleic acid as shown in SEQ ID NO: 1, SEQ ID NO: 2, SEQ ID NO: 6 or SEQ ID NO: 274 or a therapeutically effective amount of a compound comprising a modified oligonucleotide consisting of 12 to 30 linked nucleosides and having a nucleobase sequence comprising at least 8 contiguous nucleobases of a nucleobase sequence selected from any one of nucleobase sequences recited in SEQ ID NOs: 15 to 269 or comprises at least 8 contiguous nucleobases complementary to a target segment or target region as described herein. In certain embodiments the modified oligonucleotide has a nucleobase sequence comprising a contiguous nucleobase portion of a nucleobase sequence selected from any one of nucleobase sequences recited in SEQ ID NOs: 15 to 269 or comprises a contiguous nucleobase portion complementary to a target segment or target region as described herein.

In certain embodiments, modulation can occur in a cell, tissue, organ or organism. In certain embodiments, the cell, tissue or organ is in an animal. In certain embodiments, the animal is a human. In certain embodiments, Factor XI mRNA levels are reduced. In certain embodiments, Factor XI protein levels are reduced. Such reduction can occur in a time-dependent manner or in a dose-dependent manner.

Also provided are methods, compounds, and compositions useful for preventing, treating, and ameliorating diseases, disorders, and conditions related to inflammation. In certain embodiments, such diseases, disorders, and conditions are inflammatory diseases, disorders or conditions.

In certain embodiments, methods of treatment include administering a Factor XI specific inhibitor to an individual in need thereof.

In certain embodiments, the inflammation is not sepsis related. In certain embodiments, the inflammation is not related to infection.

Also provided are compounds and compositions that include a modified oligonucleotide consisting of 12 to 30 linked nucleosides, wherein the modified oligonucleotide is complementary to a Factor XI nucleic acid as shown in SEQ ID NO: 1, SEQ ID NO: 2, SEQ ID NO: 6 or SEQ ID NO: 274. In certain embodiments the modified oligonucleotide has a nucleobase sequence comprising a contiguous nucleobase portion of a nucleobase sequence selected from any one of nucleobase sequences recited in SEQ ID NOs: 15 to 269 or comprises a contiguous nucleobase portion complementary to a target segment or target region as described herein. In certain embodiments, the modified oligonucleotide has a nucleobase sequence comprising at least 8 contiguous nucleobases of a nucleobase sequence selected from among the nucleobase sequences recited in SEQ ID NOs: 15-269 or comprises at least 8 contiguous nucleobases complementary to a target segment or target region as described herein.

### BRIEF DESCRIPTION OF THE DRAWINGS

FIG. 1 displays charts illustrating the effects of antisense oligonucleotide (ASO) inhibition on collagen-induced arthritis (CIA) in mice, as described in Example 11, infra. FIG. 1A displays the effect of ASO treatment on the percentage of mice developing CIA. Factor XI ASO treated mice developed a lower incidence of CIA compared to the untreated and Factor VII treated mice. FIG. 1B displays the effect of ASO treatment on the percentage of arthritis affected paws in mice. Factor XI ASO treated mice developed a lower incidence of affected paws compared to the untreated and Factor VII treated mice.

FIG. 2 displays charts illustrating the effects of antisense oligonucleotide (ASO) inhibition on collagen-induced arthritis (CIA) in mice, as described in Example 11, infra. FIG. 2A displays the effect of ASO treatment on the average number of affected paws in mice. Factor XI ASO treated mice had fewer affected paws on average. FIG. 2B displays the effect of ASO treatment on the arthritis severity in the mice. Factor XI ASO treated mice developed less severe arthritis than the control mice.

FIG. 3 displays a timeline of the effect of CIA incidence in mice with or without ASO treatment, as described in Example 11, infra. Factor XI ASO treated mice developed CIA at a later stage with fewer affected mice.

FIG. 4 displays charts showing the arthritis severity and liver quantities of Factor XI mRNA in collagen induced arthritic mice with or without Factor XI antisense treatment, as described in Example 11, infra. FIG. 4A shows the arthritis severity in the collagen induced arthritic mice after 10 weeks of treatment with Factor XI antisense oligonucleotide ISIS 404071 (F11#1), ISIS 404057 (F11#2) or control oligonucleotide (F11 MM). FIG. 4B shows the effect of the same oligonucleotides on Factor XI mRNA in the liver of the treated mice

FIG. 5 displays charts showing the effect of Factor XI antisense oligonucleotide treatment on organ weight, as described in Example 11, infra. FIG. 5A shows the liver weight of the treated mice as a percent of the body weight of the mice. FIG. 5B shows the spleen weight of the treated mice as a percent of the body weight of the mice.

FIG. 6 displays charts showing the effect of Factor XI antisense oligonucleotide treatment on liver enzymes, as described in Example 11, infra. FIG. 6A shows the ALT level. FIG. 6B shows the AST level.

FIG. 7 displays a timeline and charts illustrating the effect of antisense oligonucleotide (ASO) inhibition on colitis in mice, as described in Example 12, infra. FIG. 7A displays a timeline of the effect of DSS-induced colitis incidence with or without ASO treatment on the body weight of mice. The timeline is a measure of the body weights at different time points as a percentage of the body weight at the start of the study. Factor XI ASO treated mice did not have any significant change in body weight during the study period. FIG. 7B displays the final body weight change compared to the DSS control on day 6. The PBS control mice and Factor VII ASO treated mice had a decrease in body weight. The Factor XI ASO treated mice had no significant change in body weight. FIG. 7C displays the colon length measurements after the treatment period. The PBS control mice and Factor VII ASO treated mice had a decrease in colon length. The Factor XI ASO treated mice had no significant change in colon length.

FIG. 8 displays histological slides of mouse colon tissue stained with hematoxylin and eosin as described in Example 12, infra. FIG. 8A displays mouse colon tissue from control mice injected subcutaneously with PBS only and has normal colon histology appearance. FIG. 8B displays mouse colon tissue from mice treated with DSS to induce colitis. The tissue shows lesions of ulcerative colitis consisting of mucosa ulcers (2-4/animal), diffused neutrophil infiltration throughout the entire colon, submucosa edema and muscularis propria thickening. FIG. 8C displays mouse colon tissue from mice treated with Factor VII ASO and subsequently with DSS to induce colitis and shows the same histology as the DSS control. FIG. 8D displays mouse colon tissue from mice treated with Factor XI and subsequently with DSS to induce colitis and shows significantly milder ulcerative colitis lesions with less mucosa ulcers (>1/animal) compared to the DSS control.

FIG. 9 displays a timeline and chart illustrating the effect of antisense oligonucleotide (ASO) inhibition on colitis in mice, as described in Example 12, infra. FIG. 9A displays the timeline of the effect of treatment with PBS, Factor XI ASO (ISIS 404071), Factor XI ASO (ISIS 404057), or a control ASO (ISIS 421208) on the body weight of DSS-induced colitis mice. The timeline is a measure of the body weights at different time points as a percentage of the body weight at the start of the study. ISIS 404071 treated mice and ISIS 404057 treated mice did not have any significant change in body weight during the study period. FIG. 9B displays the final body weight change compared to the DSS control at the end of the study period on day 7. ISIS 404071 treated mice and ISIS 404057 treated mice did not have any significant change in body weight. Astericks in the chart denote statistically significant changes from the DSS only treated mice.

FIG. 10 displays charts illustrating the effect of antisense oligonucleotide (ASO) inhibition on colitis in mice, as described in Example 12, infra. FIG. 10 displays Factor XI mRNA levels in the liver of mice treated with the following: 1) PBS only as a control; 2) PBS and subsequently with DSS to induce colitis; 3) ISIS 404071 and subsequently with DSS to induce colitis; 4) ISIS 404057 and subsequently with DSS to induce colitis; and 5) with control ASO ISIS 421208 and subsequently with DSS to induce colitis.

FIG. 11 displays a timeline and charts illustrating the dose response effect of Factor XI antisense oligonucleotide (ASO) inhibition on colitis in mice, as described in Example 12, infra. Doses of 10 mg/kg Factor XI ASO, 20 mg/kg Factor XI ASO, 40 mg/kg Factor XI ASO or 40 mg/kg of a control non-Factor XI ASO were administered to DSS-induced colitis mice. FIG. 11A displays the timeline of the effect of treatment with the various doses of Factor XI ASO on body weight in DSS-induced colitis mice. FIG. 11B displays the effect of the various doses of Factor XI ASO on stool softness/diarrhea in the DSS-induced colitis mice. FIG. 11C displays the effect of the various doses of Factor XI ASO on colon lengths in the DSS-induced colitis mice.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“Active pharmaceutical agent” means the substance or substances in a pharmaceutical composition that provide a therapeutic benefit when administered to an individual. For example, in certain embodiments an antisense oligonucleotide targeted to Factor XI is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Amelioration” refers to a lessening of at least one indicator, sign, or symptom of an associated disease, disorder, or condition. In certain embodiments, amelioration includes a delay or slowing in the progression of one or more indicators of a condition or disease. The severity of indicators may be determined by subjective or objective measures, which are known to those skilled in the art. For example, amelioration of arthritis in collagen-induced arthritic mice can be determined by clinically scoring the amount of arthritis in the mice as described by Marty et al. (J. Clin. Invest 107:631-640 (2001)).

“Animal” refers to a human or non-human animal, including, but not limited to, mice, rats, rabbits, dogs, cats, pigs, and non-human primates, including, but not limited to, monkeys and chimpanzees.

“Antidote compound” refers to a compound capable decreasing the intensity or duration of any antisense activity.

“Antidote oligonucleotide” means an antidote compound comprising an oligonucleotide that is complementary to and capable of hybridizing with an antisense compound.

“Antidote protein” means an antidote compound comprising a peptide.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses concomitant, parallel or sequential administration.

“Coagulation factor” means any of factors I, II, III, IV, V, VII, VIII, IX, X, XI, XII, or XIII in the blood coagulation cascade. “Coagulation factor nucleic acid” means any nucleic acid encoding a coagulation factor. For example, in certain embodiments, a coagulation factor nucleic acid includes, without limitation, a DNA sequence encoding a coagulation factor (including genomic DNA comprising introns and exons), an RNA sequence transcribed from DNA encoding a coagulation factor, and an mRNA sequence encoding a coagulation factor. “Coagulation factor mRNA” means an mRNA encoding a coagulation factor protein.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, the diluent in an injected composition may be a liquid, e.g. saline solution.

“Disease modifying drug” refers to any agent that modifies the symptoms and/or progression associated with an inflammatory disease, disorder or condition, including autoimmune diseases (e.g. arthritis, colitis or diabetes), trauma or surgery-related disorders, sepsis, allergic inflammation and asthma. DMARDs modify one or more of the symptoms and/or disease progression associated with these diseases, disorders or conditions.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Factor XI”, “FXI”, “Factor 11” and “F11” is used interchangeably herein.

“Factor XI nucleic acid” or “Factor XI nucleic acid” means any nucleic acid encoding Factor XI. For example, in certain embodiments, a Factor XI nucleic acid includes a DNA sequence encoding Factor XI, an RNA sequence transcribed from DNA encoding Factor XI (including genomic DNA comprising introns and exons), and an mRNA sequence encoding Factor XI. “Factor XI mRNA” means an mRNA encoding a Factor XI protein.

“Factor XI specific inhibitor” refers to any agent capable of specifically inhibiting the expression of Factor XI mRNA and/or Factor XI protein at the molecular level. For example, Factor XI specific inhibitors include nucleic acids (including antisense compounds), peptides, antibodies, small molecules, and other agents capable of inhibiting the expression of Factor XI mRNA and/or Factor XI protein. In certain embodiments, by specifically modulating Factor XI mRNA level and/or Factor XI protein expression, Factor XI specific inhibitors may affect components of the inflammatory pathway. Similarly, in certain embodiments, Factor XI specific inhibitors may affect other molecular processes in an animal.

“Factor XI specific inhibitor antidote” means a compound capable of decreasing the effect of a Factor XI specific inhibitor. In certain embodiments, a Factor XI specific inhibitor antidote is selected from a Factor XI peptide; a Factor XI antidote oligonucleotide, including a Factor XI antidote compound complementary to a Factor XI antisense compound; and any compound or protein that affects the intrinsic or extrinsic coagulation pathway.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap segment” and the external regions may be referred to as “wing segments.”

“Gap-widened” means a chimeric antisense compound having a gap segment of 12 or more contiguous 2′-deoxynucleosides positioned between and immediately adjacent to 5′ and 3′ wing segments having from one to six nucleosides.

“Hybridization” means the annealing of complementary nucleic acid molecules. In certain embodiments, complementary nucleic acid molecules include an antisense compound and a target nucleic acid.

“Identifying an animal at risk for an inflammatory disease, disorder or condition” means identifying an animal having been diagnosed with an inflammatory disease, disorder or condition or identifying an animal predisposed to develop an inflammatory disease, disorder or condition. Individuals predisposed to develop an inflammatory disease, disorder or condition, for example in individuals with a familial history of colitis or arthritis. Such identification may be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Inflammatory response” refers to any disease, disorder or condition related to inflammation in an animal. Examples of inflammatory responses include an immune response by the body of the animal to clear the injury or stimulus responsible for initiating the inflammatory response. Alternatively, an inflammatory response can be initiated in the body even when no known injury or stimulus is found such as in autoimmune diseases. Inflammation can be mediated by a Th1 or a Th2 response. Th1 and Th2 responses include production of selective cytokines and cellular migration or recruitment to the inflammatory site. Cell types that can migrate to an inflammatory site include, but are not limited to, eosinophils and macrophages. Th1 cytokines include, but are not limited to IL-1, IL-6, TNFα, INFγ and keratinocyte chemoattractanct (KC). Th2 cytokines include, but are not limited to, IL-4 and IL-5. A decrease in cytokine(s) level or cellular migration can be an indication of decreased inflammation. Accordingly, cytokine level or cellular migration can be a marker for certain types of inflammation such as Th1 or Th2 mediated inflammation.

“Inflammatory disease”, “inflammatory disorder” or “inflammatory condition” means a disease, disorder or condition related to an inflammatory response to injury or stimulus characterized by clinical signs of increased redness (rubor), temperature (calor), swelling (tumor), pain (dolor) and/or loss of function (functio laesa) in a tissue.

“Internucleoside linkage” refers to the chemical bond between nucleosides.

“Linked nucleosides” means adjacent nucleosides which are bonded together.

“Mismatch” or “non-complementary nucleobase” or “MM” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising a modified internucleoside linkage, a modified sugar, or a modified nucleobase.

“Modified sugar” refers to a substitution or change from a natural sugar.

“Modulating” refers to changing or adjusting a feature in a cell, tissue, organ or organism. For example, modulating Factor XI mRNA can mean to increase or decrease the level of Factor XI mRNA and/or Factor XI protein in a cell, tissue, organ or organism. Modulating Factor XI mRNA and/or protein can lead to an increase or decrease in an inflammatory response in a cell, tissue, organ or organism. A “modulator” effects the change in the cell, tissue, organ or organism. For example, a Factor XI antisense oligonucleotide can be a modulator that increases or decreases the amount of Factor XI mRNA and/or Factor XI protein in a cell, tissue, organ or organism. “Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“NSAID” refers to a Non-Steroidal Anti-Inflammatory Drug. NSAIDs reduce inflammatory reactions in a subject but in general do not ameliorate or prevent a disease from occurring or progressing.

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleotide” means a nucleoside having a phosphate group covalently linked to the sugar portion of the nucleoside.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition may comprise one or more active pharmaceutical agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset, development or progression of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment.

“3′ target site” refers to the 3′-most nucleotide of a target segment.

“Th1 related disease, disorder or condition” means an inflammatory disease, disorder or condition mediated by a Th1 immune response. Examples of Th1 diseases include, but is not limited to, allergic diseases (e.g., allergic rhinitis), autimmune diseases (e.g, multiple sclerosis, arthritis, scleroderma, psoriasis, celiac disease), cardiovascular diseases, colitis, diabetes (e.g., type 1 insulin-dependent diabetes mellitus), hypersensitivities (e.g., Type 4 hypersensitivity), infectious diseases (e.g., viral infection, mycobacterial infection) and posterior uveitis.

“Th2 related disease, disorder or condition” means an inflammatory disease, disorder or condition mediated by a Th2 immune response. Examples of Th2 diseases include, but is not limited to, allergic diseases (e.g, chronic rhinosinusitis), airway hyperresponsiveness, asthma, atopic dermatitis, colitis, endometriosis, infectious diseases (e.g., helminth infection), thyroid disease (e.g., Graves' disease), hypersensitivities (e.g, Types 1, 2 or 3 hypersensitivity) and pancreatitis.

“Th1” or “Th2” responses include production of selective cytokines and cellular migration or recruitment to an inflammatory site. Cell types that can migrate to an inflammatory site include, but are not limited to, eosinophils and macrophages. Accordingly, cytokine level or cellular migration can be a marker for certain types of inflammation such as Th1 or Th2 mediated inflammation. Th1 markers include, but are not limited to cytokines IL-1, IL-6, TNFα, INFγ and keratinocyte chemoattractanct (KC). Th2 markers include, but are not limited to, eosinophil infiltration, mucus production and cytokines IL-4 and IL-5. A decrease in cytokine(s) level or cellular migration can be an indication of decreased inflammation.

“Therapeutically effective amount” means an amount of a pharmaceutical agent that provides a therapeutic benefit to an individual.

“Treat” refers to administering a pharmaceutical composition to an animal in order to effect an alteration or improvement of a disease, disorder, or condition in the animal. In certain embodiments, one or more pharmaceutical compositions can be administered to the animal.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleotide) or a DNA nucleotide (i.e. β-D-deoxyribonucleotide).

### CERTAIN EMBODIMENTS

In certain embodiments, provided are methods, compounds, and compositions for modulating an inflammatory response by administering the compound to an animal, wherein the compound comprises a Factor XI modulator. Modulation of Factor XI can lead to an increase or decrease of Factor XI mRNA and protein expression in order to increase or decrease an inflammatory response as needed. In certain embodiments, Factor XI inhibition in an animal is reversed by administering a modulator targeting Factor XI. In certain embodiments of the invention, Factor XI is inhibited by the modulator. The Factor XI modulator can be a modified oligonucleotide targeting Factor XI.

In certain embodiments, provided are methods, compounds, and compositions for the treatment, prevention, or amelioration of inflammatory diseases, disorders and conditions associated with Factor XI in an animal in need thereof. In an embodiment, the method for ameliorating an inflammatory disease in an animal comprises administering to the animal a compound targeting Factor XI.

In certain embodiments provided are methods, compounds and compositions for treating an animal at risk for an inflammatory disease, disorder or condition, comprising administering a therapeutically effective amount of a compound targeting Factor XI to the animal at risk.

In certain embodiments, provided are methods, compounds and compositions for inhibiting Factor XI expression in an animal suffering from an inflammatory disease, disorder or condition, comprising administering a compound targeting Factor XI to the animal.

In certain embodiments, provided are methods, compounds and compositions for reducing the risk of inflammatory disease, disorder or condition, in an animal comprising administering a compound targeting Factor XI to the animal.

In certain embodiments, provided is a Factor XI modulator, wherein the Factor XI modulator is a Factor XI specific inhibitor, for use in treating, preventing, or ameliorating an inflammatory response, disease, disorder or condition. In certain embodiments, Factor XI specific inhibitors are nucleic acids (including antisense compounds), peptides, antibodies, small molecules, and other agents capable of inhibiting the expression of Factor XI mRNA and/or Factor XI protein.

In certain embodiments, the inflammatory disease, disorder or condition is a fibrin related inflammatory disease, disorder or condition.

In certain embodiments, the inflammatory disease, disorder or condition is not sepsis or infection related.

In certain embodiments, the inflammatory disease, disorder or condition is Th1 mediated. In certain embodiments, a marker for the Th1 mediated inflammatory disease, disorder or condition is decreased. Markers for Th1 include, but are not limited to cytokines such as IL-1, IL-6, TNF-α or KC. In certain embodiments, the compounds of the invention prevent or ameliorate a Th1 mediated disease. Th1 mediated diseases include, but is not limited to, allergic diseases (e.g., allergic rhinitis), autimmune diseases (e.g, multiple sclerosis, arthritis, scleroderma, psoriasis, celiac disease), cardiovascular diseases, colitis, diabetes (e.g., type 1 insulin-dependent diabetes mellitus), hypersensitivities (e.g., Type 4 hypersensitivity), infectious diseases (e.g., viral infection, mycobacterial infection) and posterior uveitis.

In certain embodiments, the inflammatory disease, disorder or condition is Th2 mediated. In certain embodiments, a marker for the Th2 mediated inflammatory disease, disorder or condition is decreased. Markers for Th2 include, but are not limited to, eosinophil infiltration to the site of inflammation, mucus production and cytokines such as IL-4, IL-5. In certain embodiments, the compounds of the invention prevent or ameliorate a Th2 mediated disease. Th2 mediated diseases include, but is not limited to, allergic diseases (e.g, chronic rhinosinusitis), airway hyperresponsiveness, asthma, atopic dermatitis, colitis, endometriosis, infectious diseases (e.g., helminth infection), thyroid disease (e.g., Graves' disease), hypersensitivities (e.g, Types 1, 2 or 3 hypersensitivity) and pancreatitis.

In certain embodiments, provided are compounds targeted to a Factor XI nucleic acid. In certain embodiments, the Factor XI nucleic acid is any of the sequences set forth in GENBANK Accession No. NM_{—}000128.3 (incorporated herein as SEQ ID NO: 1), nucleotides 19598000 to 19624000 of GENBANK Accession No. NT_{—}022792.17 (incorporated herein as SEQ ID NO: 2), and GENBANK Accession No. NM_{—}028066.1 (incorporated herein as SEQ ID NO: 6), exons 1-15 GENBANK Accession No. NW_{—}001118167.1 (incorporated herein as SEQ ID NO: 274).

In certain embodiments, the invention provides a compound comprising a modified oligonucleotide. In certain embodiments, the compound of the invention comprises a modified oligonucleotide consisting of 12 to 30 linked nucleosides.

In certain embodiments, the compound of the invention may comprise a modified oligonucleotide comprising a nucleobase sequence at least 80%, at least 85%, at least 90%, at least 95%, at least 96%, at least 97%, at least 98%, or at least 99% complementary to an equal length portion of SEQ ID NO: 1. In certain embodiments, the compound of the invention may comprise a modified oligonucleotide comprising a nucleobase sequence 100% complementary to an equal length portion of SEQ ID NO: 1.

In certain embodiments, the invention provides a compound comprising a modified oligonucleotide comprising a nucleobase sequence complementary to at least a portion of a target region as set out below as nucleobase ranges on the target RNA sequence.

In certain embodiments, the invention provides a compound comprising a modified oligonucleotide comprising a nucleobase sequence complementary to at least a portion of nucleobases 656 to 676 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 656 to 676 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 665 to 687 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 665 to 687 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 50% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 675 to 704 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 675 to 704 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 50% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 677 to 704 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 677 to 704 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 60% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 678 to 697 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 678 to 697 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 70% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 680 to 703 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 680 to 703 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 3 and Example 14).

In certain embodiments, the invention provides a compound comprising a modified oligonucleotide comprising a nucleobase sequence complementary to at least a portion of nucleobases 683 to 702 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 683 to 702 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 90% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 738 to 759 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 738 to 759 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 3 and Example 14).

In certain embodiments, the invention provides a compound comprising a modified oligonucleotide comprising a nucleobase sequence complementary to at least a portion of nucleobases 738 to 760 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 738 to 760 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 60% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 738 to 762 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 738 to 762 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 45% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1018 to 1042 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1018 to 1042 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1062 to 1089 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1062 to 1089 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 70% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1062 to 1090 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1062 to 1090 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 60% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1062 to 1091 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1062 to 1091 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 20% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1275 to 1301 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1062 to 1091 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1276 to 1301 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1062 to 1091 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 14).

In certain embodiments, the invention provides a compound comprising a modified oligonucleotide comprising a nucleobase sequence complementary to at least a portion of nucleobases 1284 to 1308 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1062 to 1091 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1291 to 1317 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1062 to 1091 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 80% inhibition of human mRNA levels as determined using an RT-PCR assay method, optionally in HepG2 cells (e.g. as described in Example 3).

In certain embodiments, the invention provides a compound comprising a modified oligonucleotide comprising a nucleobase sequence complementary to at least a portion of nucleobases 1275 to 1318 of SEQ ID NO: 1. Said modified oligonucleotide may comprise at least 8, at least 9, at least 10, at least 11, at least 12, at least 13, at least 14, at least 15, at least 16, at least 17, at least 18, at least 19 or 20 contiguous nucleobases complementary to an equal length portion of nucleobases 1275 to 1318 of SEQ ID NO: 1. Said modified oligonucleotide may comprise a nucleobase sequence at least 80%, at least 85%, at least 90%, at least 95%, at least 96%, at least 97%, at least 98%, or at least 99% complementary to an equal length portion of SEQ ID NO: 1. Said modified oligonucleotide may comprise a nucleobase sequence 100% complementary to an equal length portion of SEQ ID NO: 1. Said modified oligonucleotide may achieve at least 70% inhibition of human mRNA levels as determined using an RT-PCR assay method, optionally in HepG2 cells (e.g. as described in Example 3).

Embodiments of the present invention provide compounds comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19 or 20 contiguous nucleobases of a nucleobase sequence selected from among the nucleobase sequences recited in SEQ ID NOs: 15 to 241.

Embodiments of the present invention provide compounds comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19 or 20 contiguous nucleobases of a nucleobase sequence selected from among the nucleobase sequences recited in SEQ ID NOs: 15 to 269.

Embodiments of the present invention provide compounds comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19 or 20 contiguous nucleobases of a nucleobase sequence selected from among the nucleobase sequences recited in SEQ ID NOs: 242 to 269.

Certain embodiments of the present invention provide compounds comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19 or 20 contiguous nucleobases of a nucleobase sequence selected from among the nucleobase sequences recited in SEQ ID NOs: 15 to 269.

Certain embodiments of the present invention provide compounds comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19 or 20 contiguous nucleobases of a nucleobase sequence selected from among the nucleobase sequences recited in SEQ ID NOs: 242 to 269.

In certain embodiments, the modified oligonucleotide comprises at least 8, at least 9, at least 10, at least 11, at least 12, at least 13, at least 14, at least 15, at least 16, at least 17, at least 18, at least 19 or 20 nucleobases of a nucleobase sequence selected from ISIS Nos: 22, 31, 32, 34, 36 to 38, 40, 41, 43, 51 to 53, 55, 56, 59, 60, 64, 66, 71, 73, 75, 96, 98 to 103, 105 to 109, 113 to 117, 119, 124, 127, 129, 171, 172, 174, 176, 178, 179, 181 to 197, 199 to 211, and 213 to 232. In certain embodiments, the modified oligonucleotide comprises a nucleobase sequence selected from SEQ ID NOs: 22, 31, 32, 34, 36 to 38, 40, 41, 43, 51 to 53, 55, 56, 59, 60, 64, 66, 71, 73, 75, 96, 98 to 103, 105 to 109, 113 to 117, 119, 124, 127, 129, 171, 172, 174, 176, 178, 179, 181 to 197, 199 to 211, and 213 to 232. In certain embodiments, the modified oligonucleotide consists of a nucleobase sequence selected from SEQ ID NOs: 22, 31, 32, 34, 36 to 38, 40, 41, 43, 51 to 53, 55, 56, 59, 60, 64, 66, 71, 73, 75, 96, 98 to 103, 105 to 109, 113 to 117, 119, 124, 127, 129, 171, 172, 174, 176, 178, 179, 181 to 197, 199 to 211, and 213 to 232. Said modified oligonucleotide may achieve at least 70% inhibition of human mRNA levels as determined using an RT-PCR assay method, optionally in HepG2 cells (e.g. as described in Example 3).

In certain embodiments, the modified oligonucleotide comprises at least 8, at least 9, at least 10, at least 11, at least 12, at least 13, at least 14, at least 15, at least 16, at least 17, at least 18, at least 19 or 20 nucleobases of a nucleobase sequence selected from ISIS Nos: 22, 31, 34, 37, 40, 43, 51 to 53, 60, 98, 100 to 102, 105 to 109, 114, 115, 119, 171, 174, 176, 179, 181, 186, 188 to 193, 195, 196, 199 to 210, and 213 to 232. In certain embodiments, the modified oligonucleotide comprises a nucleobase sequence selected from SEQ ID NOs: 22, 31, 34, 37, 40, 43, 51 to 53, 60, 98, 100 to 102, 105 to 109, 114, 115, 119, 171, 174, 176, 179, 181, 186, 188 to 193, 195, 196, 199 to 210, and 213 to 232. In certain embodiments, the modified oligonucleotide consists of a nucleobase sequence selected from SEQ ID NOs: 22, 31, 34, 37, 40, 43, 51 to 53, 60, 98, 100 to 102, 105 to 109, 114, 115, 119, 171, 174, 176, 179, 181, 186, 188 to 193, 195, 196, 199 to 210, and 213 to 232. Said modified oligonucleotide may achieve at least 80% inhibition of human mRNA levels as determined using an RT-PCR assay method, optionally in HepG2 cells (e.g. as described in Example 3).

In certain embodiments, the modified oligonucleotide comprises at least 8, at least 9, at least 10, at least 11, at least 12, at least 13, at least 14, at least 15, at least 16, at least 17, at least 18, at least 19 or 20 nucleobases of a nucleobase sequence selected from ISIS Nos: 31, 37, 100, 105, 179, 190 to 193, 196, 202 to 207, 209, 210, 214 to 219, 221 to 224, 226, 227, 229 and 231. In certain embodiments, the modified oligonucleotide comprises a nucleobase sequence selected from SEQ ID NOs: 31, 37, 100, 105, 179, 190 to 193, 196, 202 to 207, 209, 210, 214 to 219, 221 to 224, 226, 227, 229 and 231. In certain embodiments, the modified oligonucleotide consists of a nucleobase sequence selected from SEQ ID NOs: 31, 37, 100, 105, 179, 190 to 193, 196, 202 to 207, 209, 210, 214 to 219, 221 to 224, 226, 227, 229 and 231. Said modified oligonucleotide may achieve at least 90% inhibition of human mRNA levels as determined using an RT-PCR assay method, optionally in HepG2 cells (e.g. as described in Example 3).

In certain embodiments, the modified oligonucleotide comprises at least 8, at least 9, at least 10, at least 11, at least 12, at least 13, at least 14, at least 15, at least 16, at least 17, at least 18, at least 19 or 20 nucleobases of a nucleobase sequence selected from SEQ ID NOs: 34, 52, 53, 114, 115, 190, 213 to 232, 242 to 260, and 262 to 266. In certain embodiments, the modified oligonucleotide comprises a nucleobase sequence selected from SEQ ID NOs: 34, 52, 53, 114, 115, 190, 213 to 232, 242 to 260, and 262 to 266. In certain embodiments, the modified oligonucleotide consists of a nucleobase sequence selected from SEQ ID NOs: 34, 52, 53, 114, 115, 190, 213 to 232, 242 to 260, and 262 to 266. Said modified oligonucleotides may achieve at least 70% inhibition of human mRNA levels as determined using an RT-PCR assay method, optionally in HepG2 cells (e.g. as described in Example 14).

In certain embodiments, the modified oligonucleotide comprises at least 8, at least 9, at least 10, at least 11, at least 12, at least 13, at least 14, at least 15, at least 16, at least 17, at least 18, at least 19 or 20 nucleobases of a nucleobase sequence selected from SEQ ID NOs: 34, 52, 53, 114, 115, 190, 213 to 216, 218 to 226, 243 to 246, 248, 249, 252 to 259, 264 and 265. In certain embodiments, the modified oligonucleotide comprises a nucleobase sequence selected from SEQ ID NOs: 34, 52, 53, 114, 115, 190, 213 to 216, 218 to 226, 243 to 246, 248, 249, 252 to 259, 264 and 265. In certain embodiments, the modified oligonucleotide consists of a nucleobase sequence selected from SEQ ID NOs: 34, 52, 53, 114, 115, 190, 213 to 216, 218 to 226, 243 to 246, 248, 249, 252 to 259, 264 and 265. Said modified oligonucleotides may achieve at least 80% inhibition of human mRNA levels as determined using an RT-PCR assay method, optionally in HepG2 cells (e.g. as described in Example 14).

In certain embodiments, the modified oligonucleotide comprises at least 8, at least 9, at least 10, at least 11, at least 12, at least 13, at least 14, at least 15, at least 16, at least 17, at least 18, at least 19 or 20 nucleobases of a nucleobase sequence selected from SEQ ID NOs: 34, 190, 215, 222, 223, 226, 246 and 254. In certain embodiments, the modified oligonucleotide comprises a nucleobase sequence selected from SEQ ID NOs: 34, 190, 215, 222, 223, 226, 246 and 254. In certain embodiments, the modified oligonucleotide consists of a nucleobase sequence selected from SEQ ID NOs: 34, 190, 215, 222, 223, 226, 246 and 254. Said modified oligonucleotides may achieve at least 90% inhibition of human mRNA levels as determined using an RT-PCR assay method, optionally in HepG2 cells (e.g. as described in Example 14).

In certain embodiments, the compound of the invention consists of a single-stranded modified oligonucleotide.

In certain embodiments, the modified oligonucleotide consists of 12 to 30 linked nucleosides or 20 linked nucleosides.

In certain embodiments, the nucleobase sequence of the modified oligonucleotide is 100% complementary to a nucleobase sequence of SEQ ID NO: 1, SEQ ID NO: 2, SEQ ID NO: 6 or SEQ ID NO: 274.

In certain embodiments, the compound has at least one modified internucleoside linkage. In certain embodiments, the modified internucleoside linkage is a phosphorothioate internucleoside linkage. In certain embodiments, each modified internucleoside linkage is a phosphorothioate internucleoside linkage.

In certain embodiments, the compound has at least one nucleoside comprising a modified sugar. In certain embodiments, at least one modified sugar is a bicyclic sugar. In certain embodiments, at least one modified sugar comprises a 2′-O-methoxyethyl (2′MOE).

In certain embodiments, the compound has at least one nucleoside comprising a modified nucleobase. In certain embodiments, the modified nucleobase is a 5-methylcytosine.

In certain embodiments, the modified oligonucleotide of the compound comprises:

(i) a gap segment consisting of linked deoxynucleosides;
(ii) a 5′ wing segment consisting of linked nucleosides;
(iii) a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In certain embodiments, the modified oligonucleotide of the compound comprises:

(i) a gap segment consisting of ten linked deoxynucleosides;
(ii) a 5′ wing segment consisting of linked nucleosides;
(iii) a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In certain embodiments, the modified oligonucleotide of the compound comprises:

(i) a gap segment consisting of ten linked deoxynucleosides;
(ii) a 5′ wing segment consisting of five linked nucleosides;
(iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the modified oligonucleotide of the compound comprises:

(i) a gap segment consisting of fourteen linked deoxynucleosides;
(ii) a 5′ wing segment consisting of three linked nucleosides;
(iii) a 3′ wing segment consisting of three linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the modified oligonucleotide of the compound comprises:

(i) a gap segment consisting of thirteen linked deoxynucleosides;
(ii) a 5′ wing segment consisting of two linked nucleosides;
(iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, provided are methods, compounds and compositions for treating an animal at risk for an inflammatory disease, disorder or condition or an animal having an inflammatory disease, disorder or condition comprising administering to the animal a therapeutically effective amount of a compound comprising a modified oligonucleotide consisting of 12 to 30 linked nucleosides, wherein the modified oligonucleotide is complementary to a Factor XI nucleic acid as shown in SEQ ID NO: 1 or SEQ ID NO: 2.

In certain embodiments, provided are methods, compounds and compositions for treating an animal at risk for an inflammatory disease, disorder or condition or an animal having an inflammatory disease, disorder or condition comprising administering to the animal a therapeutically effective amount of a compound comprising a modified oligonucleotide consisting of 12 to 30 linked nucleosides and having a nucleobase sequence comprising at least 8 contiguous nucleobases of a nucleobase sequence selected from any one of nucleobase sequences recited in SEQ ID NOs: 15 to 269.

In certain embodiments, administration of a Factor XI modulator to an animal does not cause injurious bleeding in the animal or exacerbate a bleeding condition.

In certain embodiments, the animal is pre-treated with one or more Factor XI modulators.

In certain embodiments, the animal is a human.

In certain embodiments, the compounds of the invention treats, prevents or ameliorates an inflammatory response, disease, disorder or condition in an animal. In certain embodiments, the response, disease, disorder or condition is associated with Factor XI. In certain embodiments the inflammatory response, disease, disorder, or condition may include, but is not limited to, or may be due to or associated with arthritis, colitis, fibrosis, allergic inflammation and asthma, cardiovascular disease, diabetes, sepsis, immunoproliferative disease, antiphospholipid syndrome, graft-related diseases and autoimmune diseases, or any combination thereof.

Examples of arthritis include, but are not limited to, rheumatoid arthritis, juvenile rheumatoid arthritis, arthritis uratica, gout, chronic polyarthritis, periarthritis humeroscapularis, cervical arthritis, lumbosacral arthritis, osteoarthritis, psoriatic arthritis, enteropathic arthritis and ankylosing spondylitis.

Examples of colitis include, but are not limited to, ulcerative colitis, Inflammatory Bowel Disease (IBD) and Crohn's Disease.

Examples of graft-related disorders include, but are not limited to, graft versus host disease (GVHD), disorders associated with graft transplantation rejection, chronic rejection, and tissue or cell allografts or xenografts.

Examples of immunoproliferative diseases include, but are not limited to, cancers (e.g., lung cancers) and benign hyperplasias.

Examples of autoimmune diseases include, but are not limited to, lupus (e.g., lupus erythematosus, lupus nephritis), Hashimoto's thyroiditis, primary myxedema, Graves' disease, pernicious anemia, autoimmune atrophic gastritis, Addison's disease, diabetes (e.g. insulin dependent diabetes mellitus, type I diabetes mellitus, type II diabetes mellitus), good pasture's syndrome, myasthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic action hepatitis, ulcerative colitis, Sjogren's syndrome, rheumatic diseases (e.g., rheumatoid arthritis), polymyositis, scleroderma, psoriasis, and mixed connective tissue disease.

In certain embodiments, the compounds and compositions are administered to an animal to treat, prevent or ameliorate an inflammatory disease. In certain embodiments, administration to an animal is by a parenteral route. In certain embodiments, the parenteral administration is any of subcutaneous or intravenous administration.

In certain embodiments, the compound is co-administered with one or more second agent(s). In certain embodiments the second agent is a NSAID or a disease modifying drug.

NSAIDS include, but are not limited to, acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, meloxicam and tramadol. The compound of the invention and one or more NSAIDS can be administered concomitantly or sequentially.

Examples of disease modifying drugs include, but are not limited to, methotrexate, abatacept, infliximab, cyclophosphamide, azathioprine, corticosteroids, cyclosporin A, aminosalicylates, sulfasalazine, hydroxychloroquine, leflunomide, etanercept, efalizumab, 6-mercapto-purine (6-MP), and tumor necrosis factor-alpha (TNFalpha) or other cytokine blockers or antagonists. The compound of the invention and one or more disease modifying drug can be administered concomitantly or sequentially.

In certain embodiments, a compound or oligonucleotide is in salt form.

In certain embodiments, the compounds or compositions are formulated with a pharmaceutically acceptable carrier or diluent.

In certain embodiments, provided are methods and compounds useful for the treatment, prevention, or amelioration of an inflammatory response or inflammatory disease, disorder, or condition. Factor XI has a sequence as shown in SEQ ID NO: 1, SEQ ID NO: 2, SEQ ID NO: 6 or SEQ ID NO: 274. In certain embodiments, a modified oligonucleotide is used for treating an inflammatory response or inflammatory disease, disorder, or condition. In certain embodiments, the modified oligonucleotide has a nucleobase sequence comprising at least 8 contiguous nucleobases of a nucleobase sequence selected from among the nucleobase sequences recited in SEQ ID NOs: 15-269.

In certain embodiments, provided are methods and compounds useful in the manufacture of a medicament for the treatment, prevention, or amelioration of an inflammatory response or inflammatory disease, disorder, or condition. Factor XI has a sequence as shown in SEQ ID NO: 1, SEQ ID NO: 2, SEQ ID NO: 6 or SEQ ID NO: 274. In certain embodiments, a modified oligonucleotide is used in the manufacture of a medicament for treating an inflammatory response or inflammatory disease, disorder, or condition. In certain embodiments, the modified oligonucleotide has a nucleobase sequence comprising a portion of contiguous nucleobases of a nucleobase sequence selected from among the nucleobase sequences recited in SEQ ID NOs: 15-269 or comprises a portion of nucleobases complementary to a target segment or target region as described herein. In certain embodiments, the modified oligonucleotide has a nucleobase sequence comprising at least 8 contiguous nucleobases of a nucleobase sequence selected from among the nucleobase sequences recited in SEQ ID NOs: 15-269 or comprises at least 8 contiguous nucleobases complementary to a target segment or target region as described herein.

In certain embodiments, provided is the use of a Factor XI modulator as described herein in the manufacture of a medicament for treating, ameliorating, or preventing inflammatory diseases, disorders, and conditions associated with Factor XI.

In certain embodiments, provided is a Factor XI modulator as described herein for use in treating, preventing, or ameliorating an inflammatory response or inflammatory disease, disorder, or condition as described herein. The Factor XI modulator can be used in combination therapy with one or more additional agent or therapy as described herein. Agents or therapies can be administered concomitantly or sequentially to an animal.

In certain embodiments, provided is the use of a Factor XI modulator as described herein in the manufacture of a medicament for treating, preventing, or ameliorating an inflammatory disease, disorder or condition as described herein. The Factor XI modulator can be used in combination therapy with one or more additional agent or therapy as described herein. Agents or therapies can be administered concomitantly or sequentially to an animal.

In certain embodiments, provided is a kit for treating, preventing, or ameliorating an inflammatory response, disease, disorder or condition as described herein wherein the kit comprises:

(i) a Factor XI specific inhibitor as described herein; and optionally
(ii) an additional agent or therapy as described herein.

A kit of the present invention may further include instructions for using the kit to treat, prevent, or ameliorate an inflammatory disease, disorder or condition as described herein by combination therapy as described herein.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a Factor XI nucleic acid is 12 to 30 subunits in length. In other words, antisense compounds are from 12 to 30 linked subunits. In other embodiments, the antisense compound is 8 to 80, 12 to 50, 15 to 30, 18 to 24, 19 to 22, or 20 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleotides.

In certain embodiments, a shortened or truncated antisense compound targeted to a Factor XI nucleic acid has a single subunit deleted from the 5′ end (5′ truncation), or alternatively from the 3′ end (3′ truncation). A shortened or truncated antisense compound targeted to a Factor XI nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a Factor XI nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers of the present invention include, but are not limited to, for example 5-10-5, 4-8-4, 4-12-3, 4-12-4, 3-14-3, 2-13-5, 2-16-2, 1-18-1, 3-10-3, 2-10-2, 1-10-1 or 2-8-2.

In certain embodiments, the antisense compound as a “wingmer” motif, having a wing-gap or gap-wing configuration, i.e. an X-Y or Y-Z configuration as described above for the gapmer configuration. Thus, wingmer configurations of the present invention include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to a Factor XI nucleic acid possess a 5-10-5 gapmer motif.

In certain embodiments, antisense compounds targeted to a Factor XI nucleic acid possess a 3-14-3 gapmer motif.

In certain embodiments, antisense compounds targeted to a Factor XI nucleic acid possess a 2-13-5 gapmer motif.

In certain embodiments, an antisense compound targeted to a Factor XI nucleic acid has a gap-widened motif.

In certain embodiments, a gap-widened antisense oligonucleotide targeted to a Factor XI nucleic acid has a gap segment of fourteen 2′-deoxyribonucleosides positioned immediately adjacent to and between wing segments of three chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to a Factor XI nucleic acid has a gap segment of thirteen 2′-deoxyribonucleosides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

Nucleotide sequences that encode Factor XI include, without limitation, the following: GENBANK® Accession No. NM_{—}000128.3, first deposited with GENBANK® on Mar. 24, 1999 incorporated herein as SEQ ID NO: 1; GENBANK® Accession No. NT_{—}022792.17, truncated from 19598000 to 19624000, first deposited with GENBANK® on Nov. 29, 2000, and incorporated herein as SEQ ID NO: 2; GENBANK® Accession No. NM_{—}028066.1, first deposited with GENBANK® on Jun. 2, 2002, incorporated herein as SEQ ID NO: 6; and exons 1-15 of GENBANK Accession No. NW_{—}001118167.1 (incorporated herein as SEQ ID NO: 274).

It is understood that the sequence set forth in each SEQ ID NO in the examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for Factor XI can be obtained by accession number from sequence databases such as NCBI and such information is incorporated herein by reference. In certain embodiments, a target region may encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in Factor XI mRNA levels are indicative of inhibition of Factor XI expression. Reductions in levels of a Factor XI protein are also indicative of inhibition of target mRNA levels. Further, phenotypic changes are indicative of inhibition of Factor XI expression. For example, a prolonged aPTT time can be indicative of inhibition of Factor XI expression. In another example, prolonged aPTT time in conjunction with a normal PT time can be indicative of inhibition of Factor XI expression. In another example, a decreased quantity of Platelet Factor 4 (PF-4) can be indicative of inhibition of Factor XI expression. In another example, reduced formation of inflammation (e.g., in thrombus, asthma, arthritis or colitis formation) can be indicative of inhibition of Factor XI expression. Alternatively, increased time for inflammation formation (e.g, in thrombus, asthma, arthritis or colitis formation) can be indicative of inhibition of Factor XI expression.

### Hybridization

In some embodiments, hybridization occurs between an antisense compound disclosed herein and a Factor XI nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a Factor XI nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a Factor XI nucleic acid).

Non-complementary nucleobases between an antisense compound and a Factor XI nucleic acid may be tolerated provided that the antisense compound remains able to specifically hybridize to a target nucleic acid. Moreover, an antisense compound may hybridize over one or more segments of a Factor XI nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a Factor XI nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound may be fully complementary to a Factor XI nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a Factor XI nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a Factor XI nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a Factor XI nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity or some other beneficial biological property to the antisense compounds. In certain embodiments, nucleosides comprise a chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R1)(R)2 (R═H, C1-C12 alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH3 and 2′-O(CH2)2OCH3 substituent groups. The substituent at the 2′ position can also be selected from allyl, amino, azido, thio, O-allyl, O—C1-C10 alkyl, OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl, O-alkaryl or O-aralkyl, substituted alkyl, alkenyl, alkynyl, alkaryl, aralkyl, SH, SCH₃, OCN, Cl, Br, CN, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties

Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH2)-O-2′ (LNA); 4′-(CH2)-S-2; 4′-(CH2)2-O-2′ (ENA); 4′-C(CH3)2-O-2′ (see PCT/US2008/068922); 4′-CH(CH3)-O-2′ and 4′-C—H(CH2OCH3)-O-2′ (see U.S. Pat. No. 7,399,845, issued on Jul. 15, 2008); 4′-CH2-N(OCH3)-2′ (see PCT/US2008/064591); 4′-CH2-O—N(CH3)-2′ (see published U.S. Patent Application US2004-0171570, published Sep. 2, 2004); 4′-CH2-N(R)—O-2′ (see U.S. Pat. No. 7,427,672, issued on Sep. 23, 2008); 4′-CH2-CH(CH3)-2′ (see Chattopadhyaya et al., J. Org. Chem., 2009, 74, 118-134) and 4′-CH2-C—(═CH2)-2′ (see PCT/US2008/066154); and wherein R is, independently, H, C1-C12 alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226). Previously, α-L-methyleneoxy (4′-CH₂—O-2′) BNA's have also been incorporated into antisense oligonucleotides that showed antisense activity (Frieden et al., Nucleic Acids Research, 2003, 21, 6365-6372).

Further reports related to bicyclic nucleosides can be found in published literature (see for example: Srivastava et al., J. Am. Chem. Soc., 2007, 129, 8362-8379; U.S. Pat. Nos. 7,053,207; 6,268,490; 6,770,748; 6,794,499; 7,034,133; and 6,525,191; Elayadi et al., Curr. Opinion Invens. Drugs, 2001, 2, 558-561; Braasch et al., Chem. Biol., 2001, 8, 1-7; and Orum et al., Curr. Opinion Mol. Ther., 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. No. 7,399,845; U.S. patent Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′,4′-(CH₂)₂-2′,4′-(CH₂)₃-2′,4′-CH₂—O-2′,4′-(CH₂)₂—O-2′,4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, and (F) Methyl(methyleneoxy) (4′-CH(CH₃)—O-2′) BNA, (G) Methylene-thio (4′-CH₂—S-2′) BNA, (H) Methylene-amino (4′-CH₂—N(R)-2′) BNA, (I) Methyl carbocyclic (4′-CH₂—CH(CH₃)-2′) BNA, and (J) Propylene carbocyclic (4′-(CH₂)₃-2′) BNA as depicted below.

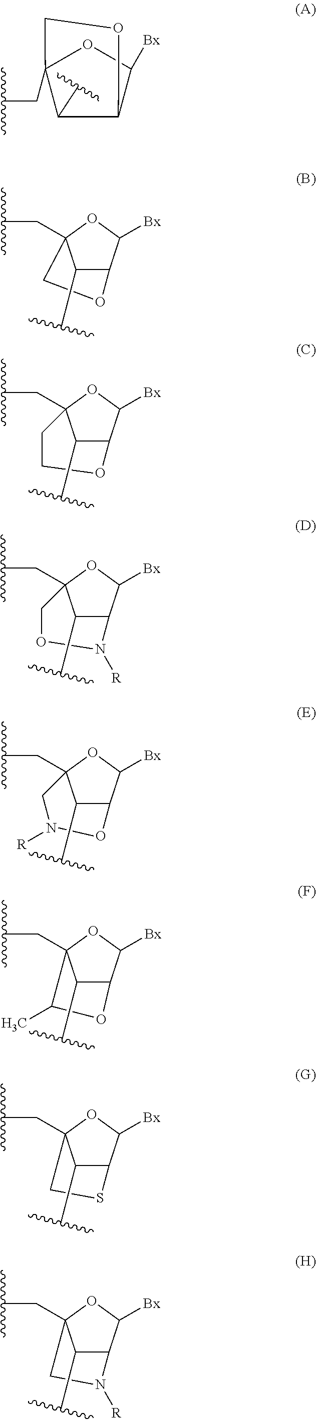

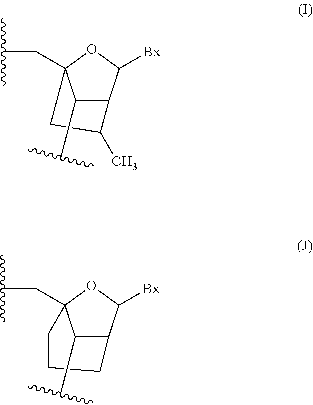

wherein Bx is the base moiety and R is independently H, a protecting group or C₁-C₁₂ alkyl.

In certain embodiments, bicyclic nucleoside having Formula I:

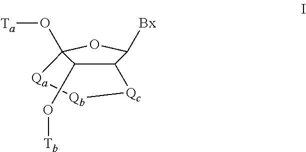

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleoside having Formula II:

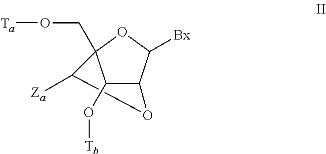

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleoside having Formula III:

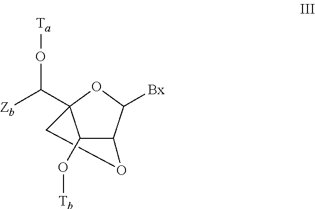

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleoside having Formula IV:

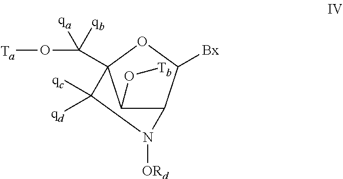

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleoside having Formula V:

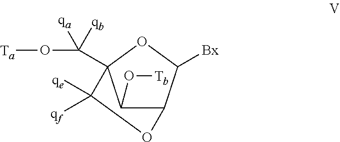

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleoside having Formula VI:

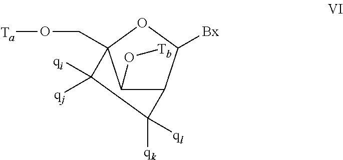

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂₋₇

C₁₋₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qᵢ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

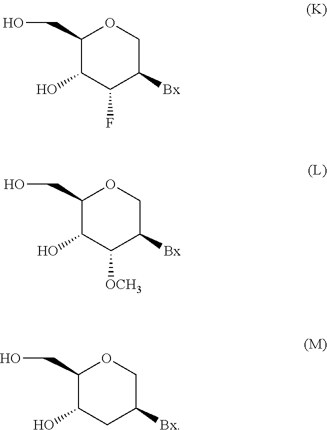

Many other bicyclo and tricyclo sugar surrogate ring systems are also know in the art that can be used to modify nucleosides for incorporation into antisense compounds (see for example review article: Leumann, Christian J., Bioorg. Med. Chem., 2002, 10, 841-854)). Such ring systems can undergo various additional substitutions to enhance activity. See for example compounds having Formula VII:

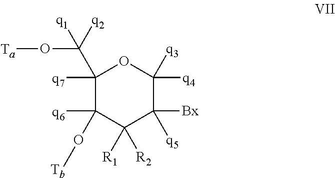

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H (M). In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro (K). In certain embodiments, THP nucleosides of Formula VII are provided wherein one of R₁ and R₂ is methoxyethoxy. In certain embodiments, R₁ is fluoro and R₂ is H; R₁ is H and R₂ is fluoro; R₁ is methoxy and R₂ is H, and R₁ is H and R₂ is methoxyethoxy. Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds targeted to a Factor XI nucleic acid comprise one or more nucleotides having modified sugar moieties. In certain embodiments, the modified sugar moiety is 2′-MOE. In certain embodiments, the 2′-MOE modified nucleotides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleotides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a Factor XI nucleic acid comprise one or more modified nucleobases. In certain embodiments, gap-widened antisense oligonucleotides targeted to a Factor XI nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides may be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

In certain embodiments, one or more modified oligonucleotides of the present invention can be formulated as a prodrug. A prodrug can be produced by modifying a pharmaceutically active compound such that the active compound will be regenerated upon in vivo administration. For example, a prodrug can include the incorporation of additional nucleosides at one or both ends of an antisense compound which are cleaved by endogenous nucleases within the body, to form the active antisense compound. The prodrug can be designed to alter the metabolic stability or the transport characteristics of a drug, to mask side effects or toxicity, to improve the flavor of a drug or to alter other characteristics or properties of a drug. In certain embodiments, upon in vivo administration, a prodrug is chemically converted to the biologically, pharmaceutically or therapeutically more active form of a modified oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a pharmaceutical composition of the present invention is administered in the form of a dosage unit (e.g., tablet, capsule, bolus, etc.). In certain embodiments, such pharmaceutical compositions comprise a modified oligonucleotide in a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In certain such embodiments, a pharmaceutical composition of the present invention comprises a dose of modified oligonucleotide selected from 25 mg, 50 mg, 75 mg, 100 mg, 150 mg, 200 mg, 250 mg, 300 mg, 350 mg, 400 mg, 500 mg, 600 mg, 700 mg, and 800 mg.

In certain embodiments, a pharmaceutical composition comprises a sterile lyophilized modified oligonucleotide that is reconstituted with a suitable diluent, e.g., sterile water for injection or sterile saline for injection. The reconstituted product is administered as a subcutaneous injection or as an intravenous infusion after dilution into saline. The lyophilized drug product consists of a modified oligonucleotide which has been prepared in water for injection, or in saline for injection, adjusted to pH 7.0-9.0 with acid or base during preparation, and then lyophilized. The lyophilized modified oligonucleotide may be 25-800 mg, or any dose between 25-800 mg as described above, of a modified oligonucleotide. The lyophilized drug product may be packaged in a 2 mL Type I, clear glass vial (ammonium sulfate-treated), stoppered with a bromobutyl rubber closure and sealed with an aluminum FLIP-OFF® overseal.

In certain embodiments, the compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. Such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

In certain embodiments, pharmaceutical compositions of the present invention comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition of the present invention is prepared using known techniques, including, but not limited to mixing, dissolving, granulating, dragee-making, levigating, emulsifying, encapsulating, entrapping or tabletting processes.

In certain embodiments, the compounds of the invention targeted to a Factor XI nucleic acid can be utilized in pharmaceutical compositions by combining the antisense compound with a suitable pharmaceutically acceptable diluent or carrier. A pharmaceutically acceptable diluent includes, but is not limited to, water, oils, alcohols, or phosphate-buffered saline (PBS). PBS is a diluent suitable for use in compositions to be delivered parenterally. Accordingly, in one embodiment, employed in the methods described herein is a pharmaceutical composition comprising an compound targeted to a Factor XI nucleic acid and a pharmaceutically acceptable diluent. In certain embodiments, the pharmaceutically acceptable diluent is PBS. In certain embodiments, the compound is an antisense oligonucleotide.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, buffered saline, glycols, oils, alcohols, flavoring agents, preservatives, and coloring agents.

In certain embodiments, a pharmaceutical composition of the present invention is a solid (e.g., a powder, tablet, and/or capsule). In certain of such embodiments, a solid pharmaceutical composition comprising one or more oligonucleotides is prepared using ingredients known in the art, including, but not limited to, starches, sugars, diluents, granulating agents, lubricants, binders, and disintegrating agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, a pharmaceutical composition of the present invention is prepared for oral administration. In certain of such embodiments, a pharmaceutical composition is formulated by combining one or more compounds comprising a modified oligonucleotide with one or more pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. In certain embodiments, pharmaceutical compositions for oral use are obtained by mixing oligonucleotide and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer (e.g., PBS). In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In certain embodiments, a pharmaceutical composition is prepared for rectal administration, such as a suppositories or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, a pharmaceutical composition of the present invention comprises a modified oligonucleotide in a therapeutically effective amount. In certain embodiments, the therapeutically effective amount is sufficient to prevent, alleviate or ameliorate symptoms of a disease or to prolong the survival of the subject being treated.

### Routes of Administration

In certain embodiments, administering to a subject comprises parenteral administration. In certain embodiments, administering to a subject comprises intravenous administration. In certain embodiments, administering to a subject comprises subcutaneous administration.

In certain embodiments, administration includes pulmonary administration. In certain embodiments, pulmonary administration comprises delivery of aerosolized oligonucleotide to the lung of a subject by inhalation. Following inhalation by a subject of aerosolized oligonucleotide, oligonucleotide distributes to cells of both normal and inflamed lung tissue, including alveolar macrophages, eosinophils, epithelium, blood vessel endothelium, and bronchiolar epithelium. A suitable device for the delivery of a pharmaceutical composition comprising a modified oligonucleotide includes, but is not limited to, a standard nebulizer device. Additional suitable devices include dry powder inhalers or metered dose inhalers.

In certain embodiments, pharmaceutical compositions are administered to achieve local rather than systemic exposures. For example, pulmonary administration delivers a pharmaceutical composition to the lung, with minimal systemic exposure.

Additional suitable administration routes include, but are not limited to, oral, rectal, transmucosal, intestinal, enteral, topical, suppository, intrathecal, intraventricular, intraperitoneal, intranasal, intraocular, intramuscular, intramedullary, and intratumoral.

### Conjugated Antisense Compounds

In certain embodiments, the compounds of the invention can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

In certain embodiments, antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of Factor XI nucleic acids can be tested in vitro in a variety of cell types. Cell types used for such analyses are available from commercial vendors (e.g. American Type Culture Collection, Manassus, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE® (Invitrogen, Carlsbad, Calif.). Antisense oligonucleotide is mixed with LIPOFECTAMINE® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE®. Antisense oligonucleotides are used at higher concentrations ranging from 625 to 20,000 nM when transfected using electroporation.

RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of a Factor XI nucleic acid can be assayed in a variety of ways known in the art. For example, target nucleic acid levels can be quantitated by, e.g., Northern blot analysis, competitive polymerase chain reaction (PCR), or quantitaive real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM® 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT, real-time-PCR reactions are carried out by methods well known to those skilled in the art.

Gene (or RNA) target quantities obtained by real time PCR are normalized using either the expression level of a gene whose expression is constant, such as cyclophilin A or GAPDH, or by quantifying total RNA using RIBOGREEN® (Invitrogen, Inc. Carlsbad, Calif.). Cyclophilin A or GAPDH expression is quantified by real time PCR, by being run simultaneously with the target, multiplexing, or separately. Total RNA is quantified using RIBOGREEN® RNA quantification reagent (Invitrogen, Carlsbad, Calif.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a Factor XI nucleic acid. Methods for designing real-time PCR probes and primers are well known in the art, and may include the use of software such as PRIMER EXPRESS® Software (Applied Biosystems, Foster City, Calif.).

The PCR probes have JOE or FAM covalently linked to the 5′ end and TAMRA or MGB covalently linked to the 3′ end, where JOE or FAM is the fluorescent reporter dye and TAMRA or MGB is the quencher dye. In some cell types, primers and probe designed to a sequence from a different species are used to measure expression. For example, a human GAPDH primer and probe set can be used to measure GAPDH expression in monkey-derived cells and cell lines.

### Analysis of Protein Levels

Antisense inhibition of Factor XI nucleic acids can be assessed by measuring Factor XI protein levels. Protein levels of Factor XI can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of human and rat Factor XI are commercially available.

### In Vivo Testing of Antisense Compounds

Antisense compounds, for example, antisense oligonucleotides, are tested in animals to assess their ability to inhibit expression of Factor XI and produce phenotypic changes, such as, prolonged aPTT, prolonged aPTT time in conjunction with a normal PT, decreased quantity of Platelet Factor 4 (PF-4), reduced induction of asthma, reduced formation of arthritis, reduced formation of colitis, increased time for asthma formation, arthritis formation and increased time for colitis formation. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. In one embodiment, following a period of treatment with antisense oligonucleotides, RNA is isolated from liver tissue and changes in Factor XI nucleic acid expression are measured. Changes in Factor XI protein levels can be measured by determining clot times, e.g. PT and aPTT, using plasma from treated animals, or by measuring the level of inflammation, inflammatory conditions (e.g., asthma, arthritis, colitis) or inflammatory markers (inflammatory cytokines) present in the animal.

### Certain Indications

In certain embodiments, the invention provides methods of treating an individual comprising administering one or more pharmaceutical compositions of the present invention. In certain embodiments, the individual has or is at risk for an inflammatory disease, disorder or condition. In certain embodiments, the individual is at risk for an inflammatory disease, disorder or condition as described supra. In certain embodiments the invention provides methods for prophylactically reducing Factor XI expression in an individual. Certain embodiments include treating an individual in need thereof by administering to an individual a therapeutically effective amount of an antisense compound targeted to a Factor XI nucleic acid.

In certain embodiments, administration of a therapeutically effective amount of an antisense compound targeted to a Factor XI nucleic acid is accompanied by monitoring of Factor XI levels in the serum of an individual, to determine an individual's response to administration of the antisense compound. An individual's response to administration of the antisense compound is used by a physician to determine the amount and duration of therapeutic intervention.

In certain embodiments, administration of an antisense compound targeted to a Factor XI nucleic acid results in reduction of Factor XI expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a Factor XI nucleic acid results in a change in a measure of blood clotting as measured by a standard test, for example, but not limited to, activated partial thromboplastin time (aPTT) test, prothrombin time (PT) test, thrombin time (TCT), bleeding time, or D-dimer. Alternatively, a change in inflammation (e.g., asthma, arthritis or colitis levels) can be determined in animal models with inflammation (e.g., induced asthma, arthritis or colitis). In certain embodiments, administration of a Factor XI antisense compound increases the measure by at least 15, 20, 25, 30, 35, 40, 45, 50, 55, 60, 65, 70, 75, 80, 85, 90, 95 or 99%, or a range defined by any two of these values. In some embodiments, administration of a Factor XI antisense compound decreases the measure by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to Factor XI are used for the preparation of a medicament for treating a patient suffering or susceptible to an inflammatory disease, disorder or condition.

### Certain Combination Therapies

In certain embodiments, one or more pharmaceutical compositions of the present invention are co-administered with one or more other pharmaceutical agents. In certain embodiments, such one or more other pharmaceutical agents are designed to treat the same disease, disorder, or condition as the one or more pharmaceutical compositions of the present invention. In certain embodiments, such one or more other pharmaceutical agents are designed to treat a different disease, disorder, or condition as the one or more pharmaceutical compositions of the present invention. In certain embodiments, such one or more other pharmaceutical agents are designed to treat an undesired side effect of one or more pharmaceutical compositions of the present invention. In certain embodiments, one or more pharmaceutical compositions of the present invention are co-administered with another pharmaceutical agent to treat an undesired effect of that other pharmaceutical agent. In certain embodiments, one or more pharmaceutical compositions of the present invention are co-administered with another pharmaceutical agent to produce a combinational effect. In certain embodiments, one or more pharmaceutical compositions of the present invention are co-administered with another pharmaceutical agent to produce a synergistic effect.

In certain embodiments, one or more pharmaceutical compositions of the present invention and one or more other pharmaceutical agents are administered at the same time. In certain embodiments, one or more pharmaceutical compositions of the present invention and one or more other pharmaceutical agents are administered at different times. In certain embodiments, one or more pharmaceutical compositions of the present invention and one or more other pharmaceutical agents are prepared together in a single formulation. In certain embodiments, one or more pharmaceutical compositions of the present invention and one or more other pharmaceutical agents are prepared separately.

In certain embodiments, pharmaceutical agents that may be co-administered with a pharmaceutical composition of the present invention include NSAIDS and/or disease modifying drugs as described supra. In certain embodiments, the disease modifying drug is administered prior to administration of a pharmaceutical composition of the present invention. In certain embodiments, the disease modifying drug is administered following administration of a pharmaceutical composition of the present invention. In certain embodiments the disease modifying drugs is administered at the same time as a pharmaceutical composition of the present invention. In certain embodiments the dose of a co-administered disease modifying drugs is the same as the dose that would be administered if the disease modifying drug was administered alone. In certain embodiments the dose of a co-administered disease modifying drug is lower than the dose that would be administered if the disease modifying drugs was administered alone. In certain embodiments the dose of a co-administered disease modifying drug is greater than the dose that would be administered if the disease modifying drugs was administered alone.

In certain embodiments, the co-administration of a second compound enhances the effect of a first compound, such that co-administration of the compounds results in an effect that is greater than the effect of administering the first compound alone. In other embodiments, the co-administration results in effects that are additive of the effects of the compounds when administered alone. In certain embodiments, the co-administration results in effects that are supra-additive of the effects of the compounds when administered alone. In certain embodiments, the first compound is an antisense compound. In certain embodiments, the second compound is an antisense compound.

In certain embodiments, an antidote is administered anytime after the administration of a Factor XI specific inhibitor. In certain embodiments, an antidote is administered anytime after the administration of an antisense oligonucleotide targeting Factor XI. In certain embodiments, the antidote is administered minutes, hours, days, weeks, or months after the administration of an antisense compound targeting Factor XI. In certain embodiments, the antidote is a complementary (e.g. the sense strand) to the antisense compound targeting Factor XI. In certain embodiments, the antidote is a Factor 7, Factor 7a, Factor XI, or Factor XIa protein. In certain embodiments, the Factor 7, Factor 7a, Factor XI, or Factor XIa protein is a human Factor 7, human Factor 7a, human Factor XI, or human Factor XIa protein. In certain embodiments, the Factor 7 protein is NovoSeven.

### ADVANTAGES OF THE INVENTION

Provided herein, for the first time, are methods and compositions for the modulation of Factor XI that can treat, prevent and/or ameliorate an inflammatory response. In a particular embodiment, provided are Factor XI oligonucleotides (oligonucleotides targeting a nucleic acid encoding Factor XI protein) to ameliorate an inflammatory condition such as arthritis or colitis.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of Human Factor XI in HepG2 Cells

Antisense oligonucleotides targeted to a Factor XI nucleic acid were tested for their effects on Factor XI mRNA in vitro. Cultured HepG2 cells at a density of 10,000 cells per well were transfected using lipofectin reagent with 75 nM antisense oligonucleotide. After a treatment period of approximately 24 hours, RNA was isolated from the cells and Factor XI mRNA levels were measured by quantitative real-time PCR. Factor XI mRNA levels were adjusted according to total RNA content, as measured by RIBOGREEN®. Results are presented as percent inhibition of Factor XI, relative to untreated control cells.

The chimeric antisense oligonucleotides in Tables 1 and 2 were designed as 5-10-5 MOE gapmers. The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 1 is targeted to SEQ ID NO: 1 (GENBANK Accession No. NM_{—}000128.3) and each gapmer listed in Table 2 is targeted to SEQ ID NO: 2 (GENBANK Accession No. NT_{—}022792.17, truncated from 19598000 to 19624000).

TABLE 1
Inhibiton of human Factor XI mRNA levels by chimeric antisense
oligonucleotides having 5-10-5 MOE wings and deoxy
gap targeted to SEQ ID NO: 1

| | Target | Target | | % | SEQ |
|---|---|---|---|---|---|
| Oligo ID | Start Site | Stop Site | Sequence | inhibition | ID NO |
| 412187 | 38 | 57 | TTCAAACAAGTGACATACAC | 21 | 15 |
| 412188 | 96 | 115 | TGAGAGAATTGCTTGCTTTC | 21 | 16 |
| 412189 | 106 | 125 | AAATATACCTTGAGAGAATT | 8 | 17 |
| 412190 | 116 | 135 | AGTATGTCAGAAATATACCT | 24 | 18 |
| 412191 | 126 | 145 | TTAAAATCTTAGTATGTCAG | 14 | 19 |
| 412192 | 146 | 165 | CAGCATATTTGTGAAAGTCG | 44 | 20 |
| 412193 | 222 | 241 | TGTGTAGGAAATGGTCACTT | 38 | 21 |
| 412194 | 286 | 305 | TGCAATTCTTAATAAGGGTG | 80 | 22 |
| 412195 | 321 | 340 | AAATCATCCTGAAAAGACCT | 22 | 23 |
| 412196 | 331 | 350 | TGATATAAGAAAATCATCCT | 25 | 24 |
| 412197 | 376 | 395 | ACACATTCACCAGAAACTGA | 45 | 25 |
| 412198 | 550 | 569 | TTCAGGACACAAGTAAACCA | 21 | 26 |
| 412199 | 583 | 602 | TTCACTCTTGGCAGTGTTTC | 66 | 27 |
| 412200 | 612 | 631 | AAGAATACCCAGAAATCGCT | 59 | 28 |
| 412201 | 622 | 641 | CATTGCTTGAAAGAATACCC | 66 | 29 |
| 412202 | 632 | 651 | TTGGTGTGAGCATTGCTTGA | 65 | 30 |
| 412203 | 656 | 675 | AATGTCTTTGTTGCAAGCGC | 91 | 31 |
| 412204 | 676 | 695 | TTCATGTCTAGGTCCACATA | 74 | 32 |
| 412205 | 686 | 705 | GTTTATGCCCTTCATGTCTA | 69 | 33 |
| 412206 | 738 | 757 | CCGTGCATCTTTCTTGGCAT | 87 | 34 |
| 412207 | 764 | 783 | CGTGAAAAAGTGGCAGTGGA | 64 | 35 |
| 412208 | 811 | 830 | AGACAAATGTTACGATGCTC | 73 | 36 |
| 412209 | 821 | 840 | GTGCTTCAGTAGACAAATGT | 91 | 37 |
| 412210 | 896 | 915 | TGCACAGGATTTCAGTGAAA | 73 | 38 |
| 412211 | 906 | 925 | GATTAGAAAGTGCACAGGAT | 64 | 39 |
| 412212 | 1018 | 1037 | CCGGGATGATGAGTGCAGAT | 88 | 40 |
| 412213 | 1028 | 1047 | AAACAAGCAACCGGGATGAT | 71 | 41 |
| 412214 | 1048 | 1067 | TCCTGGGAAAAGAAGGTAAA | 58 | 42 |
| 412215 | 1062 | 1081 | ATTCTTTGGGCCATTCCTGG | 81 | 43 |
| 412216 | 1077 | 1096 | AAAGATTTCTTTGAGATTCT | 43 | 44 |
| 412217 | 1105 | 1124 | AATCCACTCTCAGATGTTTT | 47 | 45 |
| 412218 | 1146 | 1165 | AACCAGAAAGAGCTTTGCTC | 27 | 46 |
| 412219 | 1188 | 1207 | GGCAGAACACTGGGATGCTG | 56 | 47 |
| 412220 | 1204 | 1223 | TGGTAAAATGAAGAATGGCA | 58 | 48 |
| 412221 | 1214 | 1233 | ATCAGTGTCATGGTAAAATG | 48 | 49 |
| 412222 | 1241 | 1263 | AACAATATCCAGTTCTTCTC | 5 | 50 |
| 412223 | 1275 | 1294 | ACAGTTTCTGGCAGGCCTCG | 84 | 51 |
| 412224 | 1285 | 1304 | GCATTGGTGCACAGTTTCTG | 87 | 52 |
| 412225 | 1295 | 1314 | GCAGCGGACGGCATTGGTGC | 86 | 53 |
| 412226 | 1371 | 1390 | TTGAAGAAAGCTTTAAGTAA | 17 | 54 |
| 412227 | 1391 | 1410 | AGTATTTTAGTTGGAGATCC | 75 | 55 |
| 412228 | 1425 | 1444 | ATGTGTATCCAGAGATGCCT | 71 | 56 |
| 412229 | 1456 | 1475 | GTACACTCATTATCCATTTT | 64 | 57 |
| 412230 | 1466 | 1485 | GATTTTGGTGGTACACTCAT | 52 | 58 |
| 412231 | 1476 | 1495 | TCCTGGGCTTGATTTTGGTG | 74 | 59 |
| 412232 | 1513 | 1532 | GGCCACTCACCACGAACAGA | 80 | 60 |
| 412233 | 1555 | 1574 | TGTCTCTGAGTGGGTGAGGT | 64 | 61 |
| 412234 | 1583 | 1602 | GTTTCCAATGATGGAGCCTC | 60 | 62 |
| 412235 | 1593 | 1612 | ATATCCACTGGTTTCCAATG | 57 | 63 |
| 412236 | 1618 | 1637 | CCATAGAAACAGTGAGCGGC | 72 | 64 |
| 412237 | 1628 | 1647 | TGACTCTACCCCATAGAAAC | 48 | 65 |
| 412238 | 1642 | 1661 | CGCAAAATCTTAGGTGACTC | 71 | 66 |
| 412239 | 1673 | 1692 | TTCAGATTGATTTAAAATGC | 43 | 67 |
| 412240 | 1705 | 1724 | TGAACCCCAAAGAAAGATGT | 32 | 68 |
| 412241 | 1715 | 1734 | TATTATTTCTTGAACCCCAA | 41 | 69 |
| 412242 | 1765 | 1784 | AACAAGGCAATATCATACCC | 49 | 70 |
| 412243 | 1775 | 1794 | TTCCAGTTTCAACAAGGCAA | 70 | 71 |
| 412244 | 1822 | 1841 | GAAGGCAGGCATATGGGTCG | 53 | 72 |
| 412245 | 1936 | 1955 | GTCACTAAGGGTATCTTGGC | 75 | 73 |
| 412246 | 1992 | 2011 | AGATCATCTTATGGGTTATT | 68 | 74 |
| 412247 | 2002 | 2021 | TAGCCGGCACAGATCATCTT | 75 | 75 |
| 412248 | 2082 | 2101 | CCAGATGCCAGACCTCATTG | 53 | 76 |
| 412249 | 2195 | 2214 | CATTCACACTGCTTGAGTTT | 55 | 77 |
| 412250 | 2268 | 2287 | TGGCACAGTGAACTCAACAC | 63 | 78 |
| 412251 | 2326 | 2345 | CTAGCATTTTCTTACAAACA | 58 | 79 |
| 412252 | 2450 | 2469 | TTATGGTAATTCTTGGACTC | 39 | 80 |
| 412253 | 2460 | 2479 | AAATATTGCCTTATGGTAAT | 20 | 81 |
| 412254 | 2485 | 2504 | TATCTGCCTATATAGTAATC | 16 | 82 |
| 412255 | 2510 | 2529 | GCCACTACTTGGTTATTTTC | 38 | 83 |
| 412256 | 2564 | 2583 | AACAAATCTATTTATGGTGG | 39 | 84 |
| 412257 | 2622 | 2641 | CTGCAAAATGGTGAAGACTG | 57 | 85 |
| 412258 | 2632 | 2651 | GTGTAGATTCCTGCAAAATG | 44 | 86 |
| 412259 | 2882 | 2901 | TTTTCAGGAAAGTGTATCTT | 37 | 87 |
| 412260 | 2892 | 2911 | CACAAATCATTTTTCAGGAA | 27 | 88 |
| 412261 | 2925 | 2944 | TCCCAAGATATTTTAAATAA | 3 | 89 |
| 412262 | 3168 | 3187 | AATGAGATAAATATTTGCAC | 34 | 90 |
| 412263 | 3224 | 3243 | TGAAAGCTATGTGGTGACAA | 33 | 91 |
| 412264 | 3259 | 3278 | CACACTTGATGAATTGTATA | 27 | 92 |
| 413460 | 101 | 120 | TACCTTGAGAGAATTGCTTG | 40 | 93 |
| 413461 | 111 | 130 | GTCAGAAATATACCTTGAGA | 39 | 94 |
| 413462 | 121 | 140 | ATCTTAGTATGTCAGAAATA | 12 | 95 |
| 413463 | 381 | 400 | GAGTCACACATTCACCAGAA | 74 | 96 |
| 413464 | 627 | 646 | GTGAGCATTGCTTGAAAGAA | 42 | 97 |
| 413465 | 637 | 656 | CTTATTTGGTGTGAGCATTG | 80 | 98 |
| 413466 | 661 | 680 | ACATAAATGTCTTTGTTGCA | 79 | 99 |
| 413467 | 666 | 685 | GGTCCACATAAATGTCTTTG | 91 | 100 |
| 413468 | 671 | 690 | GTCTAGGTCCACATAAATGT | 84 | 101 |
| 413469 | 681 | 700 | TGCCCTTCATGTCTAGGTCC | 84 | 102 |
| 413470 | 692 | 711 | GTTATAGTTTATGCCCTTCA | 72 | 103 |
| 413471 | 816 | 835 | TCAGTAGACAAATGTTACGA | 67 | 104 |
| 413472 | 826 | 845 | TGGGTGTGCTTCAGTAGACA | 99 | 105 |
| 413473 | 911 | 930 | AGCCAGATTAGAAAGTGCAC | 80 | 106 |
| 413474 | 1023 | 1042 | AGCAACCGGGATGATGAGTG | 84 | 107 |
| 413475 | 1053 | 1072 | GCCATTCCTGGGAAAAGAAG | 80 | 108 |
| 413476 | 1067 | 1086 | TTGAGATTCTTTGGGCCATT | 88 | 109 |
| 413477 | 1151 | 1170 | ACTGAAACCAGAAAGAGCTT | 54 | 110 |
| 413478 | 1193 | 1212 | AGAATGGCAGAACACTGGGA | 53 | 111 |
| 413479 | 1209 | 1228 | TGTCATGGTAAAATGAAGAA | 40 | 112 |
| 413480 | 1219 | 1238 | AAGAAATCAGTGTCATGGTA | 71 | 113 |
| 413481 | 1280 | 1299 | GGTGCACAGTTTCTGGCAGG | 86 | 114 |
| 413482 | 1290 | 1309 | GGACGGCATTGGTGCACAGT | 85 | 115 |
| 413483 | 1300 | 1319 | AACTGGCAGCGGACGGCATT | 78 | 116 |
| 413484 | 1430 | 1449 | CCTTAATGTGTATCCAGAGA | 74 | 117 |
| 413485 | 1461 | 1480 | TGGTGGTACACTCATTATCC | 68 | 118 |
| 413486 | 1471 | 1490 | GGCTTGATTTTGGTGGTACA | 83 | 119 |
| 413487 | 1481 | 1500 | AACGATCCTGGGCTTGATTT | 57 | 120 |
| 413488 | 1560 | 1579 | ACAGGTGTCTCTGAGTGGGT | 49 | 121 |
| 413489 | 1588 | 1607 | CACTGGTTTCCAATGATGGA | 68 | 122 |
| 413490 | 1623 | 1642 | CTACCCCATAGAAACAGTGA | 57 | 123 |
| 413491 | 1633 | 1652 | TTAGGTGACTCTACCCCATA | 73 | 124 |
| 413492 | 1647 | 1666 | AGACACGCAAAATCTTAGGT | 68 | 125 |
| 413493 | 1710 | 1729 | TTTCTTGAACCCCAAAGAAA | 65 | 126 |
| 413494 | 1780 | 1799 | GTGGTTTCCAGTTTCAACAA | 70 | 127 |
| 413495 | 1921 | 1940 | TTGGCTTTCTGGAGAGTATT | 58 | 128 |
| 413496 | 1997 | 2016 | GGCACAGATCATCTTATGGG | 72 | 129 |
| 413497 | 2627 | 2646 | GATTCCTGCAAAATGGTGAA | 39 | 130 |
| 413498 | 2637 | 2656 | GCAGAGTGTAGATTCCTGCA | 60 | 131 |
| 413499 | 2887 | 2906 | ATCATTTTTCAGGAAAGTGT | 52 | 132 |

TABLE 2
Inhibition of human Factor XI mRNA levels by chimeric antisense
oligonucleotides having 5-10-5 MOW wings and deoxy
gap targeted to SEQ ID NO: 2

| | Target | Target | | % | SEQ ID |
|---|---|---|---|---|---|
| Oligo ID | Start Site | Stop Site | Sequence | inhibition | NO |
| 413500 | 1658 | 1677 | GTGAGACAAATCAAGACTTC | 15 | 133 |
| 413501 | 2159 | 2178 | TTAGTTTACTGACACTAAGA | 23 | 134 |
| 413502 | 2593 | 2612 | CTGCTTTATGAAAAACCAAC | 22 | 135 |
| 413503 | 3325 | 3344 | ATACCTAGTACAATGTAAAT | 29 | 136 |
| 413504 | 3548 | 3567 | GGCTTGTGTGTGGTCAATAT | 54 | 137 |
| 413505 | 5054 | 5073 | TGGGAAAGCTTTCAATATTC | 57 | 138 |
| 413506 | 6474 | 6493 | ATGGAATTGTGCTTATGAGT | 57 | 139 |
| 413507 | 7590 | 7609 | TTTCAAGCTCAGGATGGGAA | 55 | 140 |
| 413508 | 7905 | 7924 | GTTGGTAAAATGCAACCAAA | 64 | 141 |
| 413509 | 8163 | 8182 | TCAGGACACAAGTAAACCTG | 66 | 142 |
| 413510 | 9197 | 9216 | TGCAAGCTGGAAATAAAAGC | 17 | 143 |
| 413511 | 9621 | 9640 | TGCCAATTTAAAAGTGTAGC | 43 | 144 |
| 413512 | 9800 | 9819 | ATATTTCAAAATCCAGTATG | 39 | 145 |
| 413513 | 9919 | 9938 | TTCTGAATATACAAATTAAT | 27 | 146 |
| 413514 | 9951 | 9970 | TTTACTATGAAAATCTAAAT | 5 | 147 |
| 413515 | 11049 | 11068 | GGTATCCTGAGTGAGATCTA | 36 | 148 |
| 413516 | 11269 | 11288 | CCAGCTATCAGGAAAATTCC | 50 | 149 |
| 413517 | 12165 | 12184 | AAAGCTATTGGAGACTCAGA | 51 | 150 |
| 413518 | 12584 | 12603 | ATGGAATCTCTTCATTTCAT | 49 | 151 |
| 413519 | 12728 | 12747 | ATGGAGACATTCATTTCCAC | 59 | 152 |
| 413520 | 13284 | 13303 | GCTCTGAGAGTTCCAATTCA | 52 | 153 |
| 413521 | 14504 | 14523 | CTGGGAAGGTGAATTTTTAG | 62 | 154 |
| 413522 | 14771 | 14790 | TCAAGAGTCTTCATGCTACC | 42 | 155 |
| 413523 | 15206 | 15225 | TCAGTTTACCTGGGATGCTG | 61 | 156 |
| 413524 | 15670 | 15689 | GACATTATACTCACCATTAT | 7 | 157 |
| 413525 | 15905 | 15924 | GTATAAATGTGTCAAATTAA | 43 | 158 |
| 413526 | 16482 | 16501 | GTAAAGTTTTACCTTAACCT | 47 | 159 |
| 413527 | 17298 | 17317 | CCATAATGAAGAAGGAAGGG | 52 | 160 |
| 413528 | 17757 | 17776 | TTAAGTTACATTGTAGACCA | 48 | 161 |
| 413529 | 18204 | 18223 | TGTGTGGGTCCTGAAATTCT | 52 | 162 |
| 413530 | 18981 | 19000 | ATCTTGTAATTACACACCCC | 27 | 163 |
| 413531 | 19174 | 19193 | GTACACTCTGCAACAGAAGC | 47 | 164 |
| 413532 | 19604 | 19623 | AGGGAATAACATGAAGGCCC | 32 | 165 |
| 413533 | 20936 | 20955 | ATCCAGTTCACCATTGGAGA | 48 | 166 |
| 413534 | 21441 | 21460 | TTTTCCAGAAGAGACTCTTC | 31 | 167 |
| 413535 | 21785 | 21804 | GTCACATTTAAAATTTCCAA | 41 | 168 |
| 413536 | 23422 | 23441 | TTAATATACTGCAGAGAACC | 37 | 169 |
| 413537 | 25893 | 25912 | AGAAATATCCCCAGACAGAG | 16 | 170 |

### Example 2

### Dose-Dependent Antisense Inhibition of Human Factor XI in HepG2 Cells

Twelve gapmers, exhibiting over 84 percent or greater in vitro inhibition of human Factor XI, were tested at various doses in HepG2 cells. Cells were plated at a density of 10,000 cells per well and transfected using lipofectin reagent with 9.375 nM, 18.75 nM, 37.5 nM, 75 nM, and 150 nM concentrations of antisense oligonucleotide, as specified in Table 3. After a treatment period of approximately 16 hours, RNA was isolated from the cells and Factor XI mRNA levels were measured by quantitative real-time PCR. Human Factor XI primer probe set RTS 2966 (forward sequence: CAGCCTGGAGCATCGTAACA, incorporated herein as SEQ ID NO: 3; reverse sequence: TTTATCGAGCTTCGTTATTCTGGTT, incorporated herein as SEQ ID NO: 4; probe sequence: TTGTCTACTGAAGCACACCCAAACAGGGAX, wherein X is the fluorophore, incorporated herein as SEQ ID NO: 5) was used to measure mRNA levels. Factor XI mRNA levels were adjusted according to total RNA content, as measured by RIBOGREEN®. Results are presented as percent inhibition of Factor XI, relative to untreated control cells. As illustrated in Table 3, Factor XI mRNA levels were reduced in a dose-dependent manner in antisense oligonucleotide treated cells.

TABLE 3
Dose-dependent antisense inhibition
of human Factor XI in HepG2 cells

| | 9.375 | 18.75 | 37.5 | 75 | 150 | SEQ |
|---|---|---|---|---|---|---|
| | nM | nM | nM | nM | nM | ID No. |
| 412203 | 29 | 15 | 61 | 77 | 82 | 31 |
| 412206 | 28 | 44 | 68 | 80 | 89 | 34 |
| 412212 | 28 | 45 | 59 | 73 | 88 | 40 |
| 412223 | 33 | 48 | 62 | 76 | 81 | 51 |
| 412224 | 24 | 45 | 57 | 70 | 81 | 52 |
| 412225 | 32 | 42 | 65 | 78 | 73 | 53 |
| 413467 | 2 | 35 | 49 | 61 | 47 | 100 |
| 413468 | 14 | 34 | 56 | 78 | 75 | 101 |
| 413469 | 24 | 33 | 53 | 70 | 84 | 102 |
| 413476 | 26 | 44 | 64 | 73 | 82 | 109 |
| 413481 | 22 | 38 | 56 | 67 | 83 | 114 |
| 413482 | 26 | 39 | 59 | 74 | 82 | 115 |

### Example 3

### Antisense Inhibition of Human Factor XI in HepG2 Cells by Oligonucleotides Designed by Microwalk

Additional gapmers were designed based on the gapmers presented in Table 3. These gapmers were designed by creating gapmers shifted slightly upstream and downstream (i.e. “microwalk”) of the original gapmers from Table 3. Gapmers were also created with various motifs, e.g. 5-10-5 MOE, 3-14-3 MOE, and 2-13-5 MOE. These gapmers were tested in vitro. Cultured HepG2 cells at a density of 10,000 cells per well were transfected using lipofectin reagent with 75 nM antisense oligonucleotide. After a treatment period of approximately 24 hours, RNA was isolated from the cells and Factor XI mRNA levels were measured by quantitative real-time PCR. Factor XI mRNA levels were adjusted according to total RNA content, as measured by RIBOGREEN®. Results are presented as percent inhibition of Factor XI, relative to untreated control cells.

The in vitro inhibition data for the gapmers designed by microwalk were then compared with the in vitro inhibition data for the gapmers from Table 3, as indicated in Tables 4, 5, 6, 7, and 8. The oligonucleotides are displayed according to the region on the human mRNA (GENBANK Accession No. NM_{—}000128.3) to which they map.

The chimeric antisense oligonucleotides in Table 4 were designed as 5-10-5 MOE, 3-14-3 MOE, and 2-13-5 MOE gapmers. The first listed gapmers in Table 4 are the original gapmers (see Table 3) from which the remaining gapmers were designed via microwalk and are designated by an asterisk. The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 4 is targeted to SEQ ID NO: 1 (GENBANK Accession No. NM_{—}000128.3).

As shown in Table 4, all of the 5-10-5 MOE gapmers, 3-14-3 MOE gapmers, and 2-13-5 MOE gapmers targeted to the target region beginning at target start site 656 and ending at the target stop site 675 (i.e. nucleobases 656-675) of SEQ ID NO: 1 exhibit at least 20% inhibition of Factor XI mRNA. Many of the gapmers exhibit at least 60% inhibition. Several of the gapmers exhibit at least 80% inhibition, including ISIS numbers: 416806, 416809, 416811, 416814, 416821, 416825, 416826, 416827, 416828, 416868, 416869, 416878, 416879, 416881, 416883, 416890, 416891, 416892, 416893, 416894, 416895, 416896, 416945, 416946, 416969, 416970, 416971, 416972, 416973, 412203, 413467, 413468, and 413469. The following ISIS numbers exhibited at least 90% inhibition: 412203, 413467, 416825, 416826, 416827, 416868, 416878, 416879, 416892, 416893, 416895, 416896, 416945, 416972, and 416973. The following ISIS numbers exhibited at least 95% inhibition: 416878, 416892, 416895, and 416896.

TABLE 4
Inhibition of human Factor XI mRNA levels by chimeric antisense
oligonucleotides targeted to nucleobases 656 to 704 of SEQ ID NO: 1
(GENBANK Accession No. NM_000128.3.3)

| | Target | Target | | % | | SEQ |
|---|---|---|---|---|---|---|
| ISIS No. | Start Site | Stop Site | Sequence (5′ to 3′) | inhibition | Motif | ID No. |
| *412203 | 656 | 675 | AATGTCTTTGTTGCAAGCGC | 91 | 5-10-5 | 31 |
| *413467 | 666 | 685 | GGTCCACATAAATGTCTTTG | 92 | 5-10-5 | 100 |
| *413468 | 671 | 690 | GTCTAGGTCCACATAAATGT | 83 | 5-10-5 | 101 |
| *413469 | 681 | 700 | TGCCCTTCATGTCTAGGTCC | 86 | 5-10-5 | 102 |
| 416868 | 656 | 675 | AATGTCTTTGTTGCAAGCGC | 93 | 3-14-3 | 31 |
| 416945 | 656 | 675 | AATGTCTTTGTTGCAAGCGC | 94 | 2-13-5 | 31 |
| 416806 | 657 | 676 | AAATGTCTTTGTTGCAAGCG | 86 | 5-10-5 | 171 |
| 416869 | 657 | 676 | AAATGTCTTTGTTGCAAGCG | 81 | 3-14-3 | 171 |
| 416946 | 657 | 676 | AAATGTCTTTGTTGCAAGCG | 86 | 2-13-5 | 171 |
| 416807 | 658 | 677 | TAAATGTCTTTGTTGCAAGC | 51 | 5-10-5 | 172 |
| 416870 | 658 | 677 | TAAATGTCTTTGTTGCAAGC | 76 | 3-14-3 | 172 |
| 416947 | 658 | 677 | TAAATGTCTTTGTTGCAAGC | 62 | 2-13-5 | 172 |
| 416808 | 659 | 678 | ATAAATGTCTTTGTTGCAAG | 55 | 5-10-5 | 173 |
| 416871 | 659 | 678 | ATAAATGTCTTTGTTGCAAG | 28 | 3-14-3 | 173 |
| 416948 | 659 | 678 | ATAAATGTCTTTGTTGCAAG | 62 | 2-13-5 | 173 |
| 416809 | 660 | 679 | CATAAATGTCTTTGTTGCAA | 86 | 5-10-5 | 174 |
| 416872 | 660 | 679 | CATAAATGTCTTTGTTGCAA | 20 | 3-14-3 | 174 |
| 416949 | 660 | 679 | CATAAATGTCTTTGTTGCAA | 64 | 2-13-5 | 174 |
| 416873 | 661 | 680 | ACATAAATGTCTTTGTTGCA | 51 | 3-14-3 | 99 |
| 416950 | 661 | 680 | ACATAAATGTCTTTGTTGCA | 71 | 2-13-5 | 99 |
| 416810 | 662 | 681 | CACATAAATGTCTTTGTTGC | 68 | 5-10-5 | 175 |
| 416874 | 662 | 681 | CACATAAATGTCTTTGTTGC | 49 | 3-14-3 | 175 |
| 416951 | 662 | 681 | CACATAAATGTCTTTGTTGC | 48 | 2-13-5 | 175 |
| 416811 | 663 | 682 | CCACATAAATGTCTTTGTTG | 84 | 5-10-5 | 176 |
| 416875 | 663 | 682 | CCACATAAATGTCTTTGTTG | 75 | 3-14-3 | 176 |
| 416952 | 663 | 682 | CCACATAAATGTCTTTGTTG | 51 | 2-13-5 | 176 |
| 416812 | 664 | 68 | TCCACATAAATGTCTTTGTT | 59 | 5-10-5 | 177 |
| 416876 | 664 | 683 | TCCACATAAATGTCTTTGTT | 37 | 3-14-3 | 177 |
| 416953 | 664 | 683 | TCCACATAAATGTCTTTGTT | 45 | 2-13-5 | 177 |
| 416813 | 665 | 684 | GTCCACATAAATGTCTTTGT | 70 | 5-10-5 | 178 |
| 416877 | 665 | 684 | GTCCACATAAATGTCTTTGT | 51 | 3-14-3 | 178 |
| 416954 | 665 | 684 | GTCCACATAAATGTCTTTGT | 61 | 2-13-5 | 178 |
| 416878 | 666 | 685 | GGTCCACATAAATGTCTTTG | 95 | 3-14-3 | 100 |
| 416955 | 666 | 685 | GGTCCACATAAATGTCTTTG | 75 | 2-13-5 | 100 |
| 416814 | 667 | 686 | AGGTCCACATAAATGTCTTT | 83 | 5-10-5 | 179 |
| 416879 | 667 | 686 | AGGTCCACATAAATGTCTTT | 92 | 3-14-3 | 179 |
| 416956 | 667 | 686 | AGGTCCACATAAATGTCTTT | 61 | 2-13-5 | 179 |
| 416815 | 668 | 687 | TAGGTCCACATAAATGTCTT | 63 | 5-10-5 | 180 |
| 416880 | 668 | 687 | TAGGTCCACATAAATGTCTT | 66 | 3-14-3 | 180 |
| 416957 | 668 | 687 | TAGGTCCACATAAATGTCTT | 59 | 2-13-5 | 180 |
| 416816 | 669 | 688 | CTAGGTCCACATAAATGTCT | 79 | 5-10-5 | 181 |
| 416881 | 669 | 688 | CTAGGTCCACATAAATGTCT | 81 | 3-14-3 | 181 |
| 416958 | 669 | 688 | CTAGGTCCACATAAATGTCT | 43 | 2-13-5 | 181 |
| 416817 | 670 | 689 | TCTAGGTCCACATAAATGTC | 74 | 5-10-5 | 182 |
| 416882 | 670 | 689 | TCTAGGTCCACATAAATGTC | 60 | 3-14-3 | 182 |
| 416959 | 670 | 689 | TCTAGGTCCACATAAATGTC | 25 | 2-13-5 | 182 |
| 416883 | 671 | 690 | GTCTAGGTCCACATAAATGT | 82 | 3-14-3 | 101 |
| 416960 | 671 | 690 | GTCTAGGTCCACATAAATGT | 60 | 2-13-5 | 101 |
| 416818 | 672 | 691 | TGTCTAGGTCCACATAAATG | 76 | 5-10-5 | 183 |
| 416884 | 672 | 691 | TGTCTAGGTCCACATAAATG | 69 | 3-14-3 | 183 |
| 416961 | 672 | 691 | TGTCTAGGTCCACATAAATG | 40 | 2-13-5 | 183 |
| 416819 | 673 | 692 | ATGTCTAGGTCCACATAAAT | 56 | 5-10-5 | 184 |
| 416885 | 673 | 692 | ATGTCTAGGTCCACATAAAT | 67 | 3-14-3 | 184 |
| 416962 | 673 | 692 | ATGTCTAGGTCCACATAAAT | 77 | 2-13-5 | 184 |
| 416820 | 674 | 693 | CATGTCTAGGTCCACATAAA | 77 | 5-10-5 | 185 |
| 416886 | 674 | 693 | CATGTCTAGGTCCACATAAA | 74 | 3-14-3 | 185 |
| 416963 | 674 | 693 | CATGTCTAGGTCCACATAAA | 48 | 2-13-5 | 185 |
| 416821 | 675 | 694 | TCATGTCTAGGTCCACATAA | 84 | 5-10-5 | 186 |
| 416964 | 675 | 694 | TCATGTCTAGGTCCACATAA | 69 | 2-13-5 | 186 |
| 412204 | 676 | 695 | TTCATGTCTAGGTCCACATA | 76 | 5-10-5 | 32 |
| 416888 | 676 | 695 | TTCATGTCTAGGTCCACATA | 76 | 3-14-3 | 32 |
| 416965 | 676 | 695 | TTCATGTCTAGGTCCACATA | 53 | 2-13-5 | 32 |
| 416822 | 677 | 696 | CTTCATGTCTAGGTCCACAT | 76 | 5-10-5 | 187 |
| 416889 | 677 | 696 | CTTCATGTCTAGGTCCACAT | 60 | 3-14-3 | 187 |
| 416966 | 677 | 696 | CTTCATGTCTAGGTCCACAT | 64 | 2-13-5 | 187 |
| 116823 | 678 | 697 | CCTTCATGTCTAGGTCCACA | 77 | 5-10-5 | 188 |
| 416890 | 678 | 697 | CCTTCATGTCTAGGTCCACA | 87 | 3-14-3 | 188 |
| 416967 | 678 | 697 | CCTTCATGTCTAGGTCCACA | 75 | 2-13-5 | 188 |
| 416824 | 679 | 698 | CCCTTCATGTCTAGGTCCAC | 64 | 5-10-5 | 189 |
| 416891 | 679 | 698 | CCCTTCATGTCTAGGTCCAC | 81 | 3-14-3 | 189 |
| 416968 | 679 | 698 | CCCTTCATGTCTAGGTCCAC | 73 | 2-13-5 | 189 |
| 416825 | 680 | 699 | GCCCTTCATGTCTAGGTCCA | 92 | 5-10-5 | 190 |
| 416892 | 680 | 699 | GCCCTTCATGTCTAGGTCCA | 100 | 3-14-3 | 190 |
| 416969 | 680 | 699 | GCCCTTCATGTCTAGGTCCA | 80 | 2-13-5 | 190 |
| 416893 | 681 | 700 | TGCCCTTCATGTCTAGGTCC | 90 | 3-14-3 | 102 |
| 416970 | 681 | 700 | TGCCCTTCATGTCTAGGTCC | 88 | 2-13-5 | 102 |
| 416826 | 682 | 701 | ATGCCCTTCATGTCTAGGTC | 94 | 5-10-5 | 191 |
| 416894 | 682 | 701 | ATGCCCTTCATGTCTAGGTC | 84 | 3-14-3 | 191 |
| 416971 | 682 | 701 | ATGCCCTTCATGTCTAGGTC | 83 | 2-13-5 | 191 |
| 416827 | 683 | 702 | TATGCCCTTCATGTCTAGGT | 93 | 5-10-5 | 192 |
| 416895 | 683 | 702 | TATGCCCTTCATGTCTAGGT | 95 | 3-14-3 | 192 |
| 416972 | 683 | 702 | TATGCCCTTCATGTCTAGGT | 90 | 2-13-5 | 192 |
| 416828 | 684 | 703 | TTATGCCCTTCATGTCTAGG | 87 | 5-10-5 | 193 |
| 416896 | 684 | 703 | TTATGCCCTTCATGTCTAGG | 95 | 3-14-3 | 193 |
| 416973 | 684 | 703 | TTATGCCCTTCATGTCTAGG | 92 | 2-13-5 | 193 |
| 416829 | 685 | 704 | TTTATGCCCTTCATGTCTAG | 72 | 5-10-5 | 194 |
| 416897 | 685 | 704 | TTTATGCCCTTCATGTCTAG | 66 | 3-14-3 | 194 |
| 416974 | 685 | 704 | TTTATGCCCTTCATGTCTAG | 73 | 2-13-5 | 194 |

The chimeric antisense oligonucleotides in Table 5 were designed as 5-10-5 MOE, 3-14-3 MOE, and 2-13-5 MOE gapmers. The first listed gapmer in Table 5 is the original gapmer (see Table 3) from which the remaining gapmers were designed via microwalk and is designated by an asterisk. The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 5 is targeted to SEQ ID NO: 1 (GENBANK Accession No. NM_{—}000128.3).

As shown in Table 5, all of the 5-10-5 MOE gapmers, 3-14-3 MOE gapmers, and 2-13-5 MOE gapmers targeted to the target region beginning at target start site 738 and ending at the target stop site 762 (i.e. nucleobases 738-762) of SEQ ID NO: 1 exhibit at least 45% inhibition of Factor XI mRNA. Most of the gapmers exhibit at least 60% inhibition. Several of the gapmers exhibit at least 80% inhibition, including ISIS numbers: 412206, 416830, 416831, 416898, 416899, 416900, 416903, 416975, 416976, 416977, and 416980. The following ISIS numbers exhibited at least 90% inhibition: 412206, 416831, and 416900.

TABLE 5
Inhibition of human Factor XI mRNA levels by chimeric antisense
oligonucleotides targeted to nucleobases 738 to 762 SEQ ID NO: 1
(GENBANK Accession No. NM_000128.3.3)

| | Target | Target | | % | | SEQ |
|---|---|---|---|---|---|---|
| ISIS No. | Start Site | Stop Site | Sequence (5′ to 3′) | inhibition | Motif | ID No. |
| *412206 | 738 | 757 | CCGTGCATCTTTCTTGGCAT | 93 | 5-10-5 | 34 |
| 416898 | 738 | 757 | CCGTGCATCTTTCTTGGCAT | 88 | 3-14-3 | 34 |
| 416975 | 738 | 757 | CCGTGCATCTTTCTTGGCAT | 87 | 2-13-5 | 34 |
| 416830 | 739 | 758 | TCCGTGCATCTTTCTTGGCA | 81 | 5-10-5 | 195 |
| 416899 | 739 | 758 | TCCGTGCATCTTTCTTGGCA | 86 | 3-14-3 | 195 |
| 416976 | 739 | 758 | TCCGTGCATCTTTCTTGGCA | 83 | 2-13-5 | 195 |
| 416831 | 740 | 759 | ATCCGTGCATCTTTCTTGGC | 91 | 5-10-5 | 196 |
| 416900 | 740 | 759 | ATCCGTGCATCTTTCTTGGC | 90 | 3-14-3 | 196 |
| 416977 | 740 | 759 | ATCCGTGCATCTTTCTTGGC | 82 | 2-13-5 | 196 |
| 416832 | 741 | 760 | CATCCGTGCATCTTTCTTGG | 79 | 5-10-5 | 197 |
| 416901 | 741 | 760 | CATCCGTGCATCTTTCTTGG | 65 | 3-14-3 | 197 |
| 416978 | 741 | 760 | CATCCGTGCATCTTTCTTGG | 76 | 2-13-5 | 197 |
| 416833 | 742 | 761 | TCATCCGTGCATCTTTCTTG | 65 | 5-10-5 | 198 |
| 416902 | 742 | 761 | TCATCCGTGCATCTTTCTTG | 46 | 3-14-3 | 198 |
| 416979 | 742 | 761 | TCATCCGTGCATCTTTCTTG | 63 | 2-13-5 | 198 |
| 416834 | 743 | 762 | GTCATCCGTGCATCTTTCTT | 58 | 5-10-5 | 199 |
| 416903 | 743 | 762 | GTCATCCGTGCATCTTTCTT | 88 | 3-14-3 | 199 |
| 416980 | 743 | 762 | GTCATCCGTGCATCTTTCTT | 87 | 2-13-5 | 199 |

The chimeric antisense oligonucleotides in Table 6 were designed as 5-10-5 MOE, 3-14-3 MOE, and 2-13-5 MOE gapmers. The first listed gapmers in Table 6 are the original gapmers (see Table 3) from which the remaining gapmers were designed via microwalk and are designated by an asterisk. The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 6 is targeted to SEQ ID NO: 1 (GENBANK Accession No. NM_{—}000128.3).

As shown in Table 6, all of the 5-10-5 MOE gapmers, 3-14-3 MOE gapmers, and 2-13-5 MOE gapmers targeted to the target region beginning at target start site 1018 and ending at the target stop site 1042 (i.e. nucleobases 1018-1042) of SEQ ID NO: 1 exhibit at least 80% inhibition of Factor XI mRNA. The following ISIS numbers exhibited at least 90% inhibition: 413474, 416837, 416838, 416904, 416907, and 416908.

TABLE 6
Inhibition of human Factor XI mRNA levels by chimeric antisense
oligonucleotides targeted to nucleobases 1018 to 1042 of SEQ ID NO: 1
(GENBANK Accession No. NM_000128.3.3)

| | Target | Target | | % | | SEQ |
|---|---|---|---|---|---|---|
| ISIS No. | Start Site | Stop Site | Sequence (5′ to 3′) | inhibition | Motif | ID No. |
| *412212 | 1018 | 1037 | CCGGGATGATGAGTGCAGAT | 89 | 5-10-5 | 40 |
| 416904 | 1018 | 1037 | CCGGGATGATGAGTGCAGAT | 90 | 3-14-3 | 40 |
| 416981 | 1018 | 1037 | CCGGGATGATGAGTGCAGAT | 87 | 2-13-5 | 40 |
| 416835 | 1019 | 1038 | ACCGGGATGATGAGTGCAGA | 83 | 5-10-5 | 200 |
| 416905 | 1019 | 1038 | ACCGGGATGATGAGTGCAGA | 85 | 3-14-3 | 200 |
| 416982 | 1019 | 1038 | ACCGGGATGATGAGTGCAGA | 84 | 2-13-5 | 200 |
| 416836 | 1020 | 1039 | AACCGGGATGATGAGTGCAG | 89 | 5-10-5 | 201 |
| 416906 | 1020 | 1039 | AACCGGGATGATGAGTGCAG | 88 | 3-14-3 | 201 |
| 416983 | 1020 | 1039 | AACCGGGATGATGAGTGCAG | 86 | 2-13-5 | 201 |
| 416837 | 1021 | 1040 | CAACCGGGATGATGAGTGCA | 90 | 5-10-5 | 202 |
| 416907 | 1021 | 1040 | CAACCGGGATGATGAGTGCA | 90 | 3-14-3 | 202 |
| 416984 | 1021 | 1040 | CAACCGGGATGATGAGTGCA | 89 | 2-13-5 | 202 |
| 416838 | 1022 | 1041 | GCAACCGGGATGATGAGTGC | 94 | 5-10-5 | 203 |
| 416908 | 1022 | 1041 | GCAACCGGGATGATGAGTGC | 98 | 3-14-3 | 203 |
| 416985 | 1022 | 1041 | GCAACCGGGATGATGAGTGC | 88 | 2-13-5 | 203 |
| 413474 | 1023 | 1042 | AGCAACCGGGATGATGAGTG | 93 | 5-10-5 | 107 |

The chimeric antisense oligonucleotides in Table 7 were designed as 5-10-5 MOE, 3-14-3 MOE, and 2-13-5 MOE gapmers. The first listed gapmer in Table 7 is the original gapmer (see Table 3) from which the remaining gapmers were designed via microwalk and is designated by an asterisk. The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 7 is targeted to SEQ ID NO: 1 (GENBANK Accession No. NM_{—}000128.3).

As shown in Table 7, all of the 5-10-5 MOE gapmers, 3-14-3 MOE gapmers, and 2-13-5 MOE gapmers targeted to the target region beginning at target start site 1062 and ending at the target stop site 1091 (i.e. nucleobases 1062-1091) of SEQ ID NO: 1 exhibit at least 20% inhibition of Factor XI mRNA. Many of the gapmers exhibit at least 50% inhibition, including: 412215, 413476, 413476, 416839, 416840, 416841, 416842, 416843, 416844, 416845, 416846, 416847, 416909, 416910, 416911, 416912, 416913, 416914, 416915, 416916, 416917, 416918, 416986, 416987, 416988, 416989, 416990, 416991, 416992, 416993, 416994, 416995. The following ISIS numbers exhibited at least 80% inhibition: 412215, 413476, 413476, 416839, 416840, 416841, 416842, 416843, 416844, 416845, 416910, 416911, 416912, 416913, 416914, 416916, 416917, 416986, 416987, 416989, 416991, 416992, 416993, and 416994. The following ISIS numbers exhibited at least 90% inhibition: 413476, 413476, 416842, 416844, 416910, 416911, 416912, 416913, 416916, 416917, and 416993.

TABLE 7
Inhibition of human Factor XI mRNA levels by chimeric antisense
oligonucleotides targeted to nucleobases 1062 to 1091 of SEQ ID NO: 1
(GENBANK Accession No. NM_000128.3.3)

| | Target | Target | | % | | SEQ |
|---|---|---|---|---|---|---|
| ISIS No. | Start Site | Stop Site | Sequence (5′ to 3′) | inhibition | Motif | ID No. |
| *413476 | 1067 | 1086 | TTGAGATTCTTTGGGCCATT | 93 | 5-10-5 | 109 |
| 412215 | 1062 | 1081 | ATTCTTTGGGCCATTCCTGG | 82 | 5-10-5 | 43 |
| 416909 | 1062 | 1081 | ATTCTTTGGGCCATTCCTGG | 78 | 3-14-3 | 43 |
| 416986 | 1062 | 1081 | ATTCTTTGGGCCATTCCTGG | 88 | 2-13-5 | 43 |
| 416839 | 1063 | 1082 | GATTCTTTGGGCCATTCCTG | 89 | 5-10-5 | 204 |
| 416910 | 1063 | 1082 | GATTCTTTGGGCCATTCCTG | 90 | 3-14-3 | 204 |
| 416987 | 1063 | 1082 | GATTCTTTGGGCCATTCCTG | 80 | 2-13-5 | 204 |
| 416840 | 1064 | 1083 | AGATTCTTTGGGCCATTCCT | 85 | 5-10-5 | 205 |
| 416911 | 1064 | 1083 | AGATTCTTTGGGCCATTCCT | 90 | 3-14-3 | 205 |
| 416988 | 1064 | 1083 | AGATTCTTTGGGCCATTCCT | 76 | 2-13-5 | 205 |
| 416841 | 1065 | 1084 | GAGATTCTTTGGGCCATTCC | 87 | 5-10-5 | 206 |
| 416912 | 1065 | 1084 | GAGATTCTTTGGGCCATTCC | 92 | 3-14-3 | 206 |
| 416989 | 1065 | 1084 | GAGATTCTTTGGGCCATTCC | 88 | 2-13-5 | 206 |
| 416842 | 1066 | 1085 | TGAGATTCTTTGGGCCATTC | 94 | 5-10-5 | 207 |
| 416913 | 1066 | 1085 | TGAGATTCTTTGGGCCATTC | 93 | 3-14-3 | 207 |
| 416990 | 1066 | 1085 | TGAGATTCTTTGGGCCATTC | 76 | 2-13-5 | 207 |
| 413476 | 1067 | 1086 | TTGAGATTCTTTGGGCCATT | 93 | 5-10-5 | 109 |
| 416914 | 1067 | 1086 | TTGAGATTCTTTGGGCCATT | 87 | 3-14-3 | 109 |
| 416991 | 1067 | 1086 | TTGAGATTCTTTGGGCCATT | 87 | 2-13-5 | 109 |
| 416843 | 1068 | 1087 | TTTGAGATTCTTTGGGCCAT | 89 | 5-10-5 | 208 |
| 416915 | 1068 | 1087 | TTTGAGATTCTTTGGGCCAT | 79 | 3-14-3 | 208 |
| 416992 | 1068 | 1087 | TTTGAGATTCTTTGGGCCAT | 84 | 2-13-5 | 208 |
| 416844 | 1069 | 1088 | CTTTGAGATTCTTTGGGCCA | 90 | 5-10-5 | 209 |
| 416916 | 1069 | 1088 | CTTTGAGATTCTTTGGGCCA | 91 | 3-14-3 | 209 |
| 416993 | 1069 | 1088 | CTTTGAGATTCTTTGGGCCA | 91 | 2-13-5 | 209 |
| 416845 | 1070 | 1089 | TCTTTGAGATTCTTTGGGCC | 86 | 5-10-5 | 210 |
| 416917 | 1070 | 1089 | TCTTTGAGATTCTTTGGGCC | 92 | 3-14-3 | 210 |
| 416994 | 1070 | 1089 | TCTTTGAGATTCTTTGGGCC | 83 | 2-13-5 | 210 |
| 416846 | 1071 | 1090 | TTCTTTGAGATTCTTTGGGC | 72 | 5-10-5 | 211 |
| 416918 | 1071 | 1090 | TTCTTTGAGATTCTTTGGGC | 63 | 3-14-3 | 211 |
| 416995 | 1071 | 1090 | TTCTTTGAGATTCTTTGGGC | 64 | 2-13-5 | 211 |
| 416847 | 1072 | 1091 | TTTCTTTGAGATTCTTTGGG | 50 | 5-10-5 | 212 |
| 416919 | 1072 | 1091 | TTTCTTTGAGATTCTTTGGG | 27 | 3-14-3 | 212 |
| 416996 | 1072 | 1091 | TTTCTTTGAGATTCTTTGGG | 22 | 2-13-5 | 212 |

The chimeric antisense oligonucleotides in Table 8 were designed as 5-10-5 MOE, 3-14-3 MOE, and 2-13-5 MOE gapmers. The first listed gapmers in Table 8 are the original gapmers (see Table 3) from which the remaining gapmers were designed via microwalk and are designated by an asterisk. The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 8 is targeted to SEQ ID NO: 1 (GENBANK Accession No. NM_{—}000128.3).

As shown in Table 8, all of the 5-10-5 MOE gapmers, 3-14-3 MOE gapmers, and 2-13-5 MOE gapmers targeted to the target region beginning at target start site 1275 and ending at the target stop site 1318 (i.e. nucleobases 1275-1318) of SEQ ID NO: 1 exhibit at least 70% inhibition of Factor XI mRNA. Many of the gapmers exhibit at least 80% inhibition, including: 412223, 412224, 412225, 413482, 416848, 416849, 416850, 416851, 416852, 416853, 416854, 416855, 416856, 416857, 416858, 416859, 416860, 416861, 416862, 416863, 416864, 416865, 416866, 416867, 416920, 416921, 416922, 416923, 416924, 416925, 416926, 416927, 416928, 416929, 416930, 416931, 416932, 416933, 416934, 416935, 416936, 416937, 416938, 416939, 416940, 416941, 416942, 416943, 416944, 416997, 416998, 416999, 417000, 417001, 417002, 417003, 417004, 417006, 417007, 417008, 417009, 417010, 417011, 417013, 417014, 417015, 417016, 417017, 417018, 417019, and 417020. The following ISIS numbers exhibited at least 90% inhibition: 412224, 416850, 416853, 416856, 416857, 416858, 416861, 416862, 416864, 416922, 416923, 416924, 416925, 416926, 416928, 416931, 416932, 416933, 416934, 416935, 416937, 416938, 416940, 416941, 416943, 416999, and 417002.

TABLE 8
Inhibition of human Factor XI mRNA levels by chimeric antisense
oligonucleotides targeted to nucleobases 1275 to 1318 of SEQ ID NO: 1
(GENBANK Accession No. NM_000128.3.3)

| | Target | Target | | % | | SEQ |
|---|---|---|---|---|---|---|
| ISIS No. | Start Site | Stop Site | Sequence (5′ to 3′) | inhibition | Motif | ID No. |
| *412223 | 1275 | 1294 | ACAGTTTCTGGCAGGCCTCG | 85 | 5-10-5 | 51 |
| *412224 | 1285 | 1304 | GCATTGGTGCACAGTTTCTG | 93 | 5-10-5 | 52 |
| *413482 | 1290 | 1309 | GGACGGCATTGGTGCACAGT | 89 | 5-10-5 | 115 |
| *412225 | 1295 | 1314 | GCAGCGGACGGCATTGGTGC | 86 | 5-10-5 | 53 |
| 416920 | 1275 | 1294 | ACAGTTTCTGGCAGGCCTCG | 88 | 3-14-3 | 51 |
| 416997 | 1275 | 1295 | ACAGTTTCTGGCAGGCCTCG | 84 | 2-13-5 | 51 |
| 416848 | 1276 | 1295 | CACAGTTTCTGGCAGGCCTC | 86 | 5-10-5 | 213 |
| 416921 | 1276 | 1295 | CACAGTTTCTGGCAGGCCTC | 88 | 3-14-3 | 213 |
| 416998 | 1276 | 1295 | CACAGTTTCTGGCAGGCCTC | 88 | 2-13-5 | 213 |
| 416849 | 1277 | 1296 | GCACAGTTTCTGGCAGGCCT | 88 | 5-10-5 | 214 |
| 416922 | 1277 | 1294 | GCACAGTTTCTGGCAGGCCT | 94 | 3-14-3 | 214 |
| 416999 | 1277 | 1296 | GCACAGTTTCTGGCAGGCCT | 92 | 2-13-5 | 214 |
| 416850 | 1278 | 1297 | TGCACAGTTTCTGGCAGGCC | 93 | 5-10-5 | 215 |
| 416923 | 1278 | 1297 | TGCACAGTTTCTGGCAGGCC | 96 | 3-14-3 | 215 |
| 417000 | 1278 | 1297 | TGCACAGTTTCTGGCAGGCC | 89 | 2-13-5 | 215 |
| 416851 | 1279 | 1298 | GTGCACAGTTTCTGGCAGGC | 88 | 5-10-5 | 216 |
| 416924 | 1279 | 1298 | GTGCACAGTTTCTGGCAGGC | 97 | 3-14-3 | 216 |
| 417001 | 1279 | 1298 | GTGCACAGTTTCTGGCAGGC | 83 | 2-13-5 | 216 |
| 416925 | 1280 | 1299 | GGTGCACAGTTTCTGGCAGG | 98 | 3-14-3 | 114 |
| 417002 | 1280 | 1299 | GGTGCACAGTTTCTGGCAGG | 92 | 2-13-5 | 114 |
| 416852 | 1281 | 1300 | TGGTGCACAGTTTCTGGCAG | 84 | 5-10-5 | 217 |
| 416926 | 1281 | 1300 | TGGTGCACAGTTTCTGGCAG | 93 | 3-14-3 | 217 |
| 417003 | 1281 | 1300 | TGGTGCACAGTTTCTGGCAG | 89 | 2-13-5 | 217 |
| 416853 | 1282 | 1301 | TTGGTGCACAGTTTCTGGCA | 91 | 5-10-5 | 218 |
| 416927 | 1282 | 1301 | TTGGTGCACAGTTTCTGGCA | 87 | 3-14-3 | 218 |
| 417004 | 1282 | 1301 | TTGGTGCACAGTTTCTGGCA | 86 | 2-13-5 | 218 |
| 416854 | 1283 | 1302 | ATTGGTGCACAGTTTCTGGC | 90 | 5-10-5 | 219 |
| 416928 | 1283 | 1302 | ATTGGTGCACAGTTTCTGGC | 91 | 3-14-3 | 219 |
| 417005 | 1283 | 1302 | ATTGGTGCACAGTTTCTGGC | 79 | 2-13-5 | 219 |
| 416855 | 1284 | 1303 | CATTGGTGCACAGTTTCTGG | 87 | 5-10-5 | 220 |
| 416929 | 1284 | 1303 | CATTGGTGCACAGTTTCTGG | 83 | 3-14-3 | 220 |
| 417006 | 1284 | 1303 | CATTGGTGCACAGTTTCTGG | 81 | 2-13-5 | 220 |
| 416930 | 1285 | 1304 | GCATTGGTGCACAGTTTCTG | 87 | 3-14-3 | 52 |
| 417007 | 1285 | 1304 | GCATTGGTGCACAGTTTCTG | 82 | 2-13-5 | 52 |
| 416856 | 1286 | 1305 | GGCATTGGTGCACAGTTTCT | 95 | 5-10-5 | 221 |
| 416931 | 1286 | 1305 | GGCATTGGTGCACAGTTTCT | 96 | 3-14-3 | 221 |
| 417008 | 1286 | 1305 | GGCATTGGTGCACAGTTTCT | 82 | 2-13-5 | 221 |
| 416857 | 1287 | 1306 | CGGCATTGGTGCACAGTTTC | 92 | 5-10-5 | 222 |
| 416932 | 1287 | 1306 | CGGCATTGGTGCACAGTTTC | 92 | 3-14-3 | 222 |
| 417009 | 1287 | 1306 | CGGCATTGGTGCACAGTTTC | 85 | 2-13-5 | 222 |
| 416858 | 1288 | 1307 | ACGGCATTGGTGCACAGTTT | 93 | 5-10-5 | 223 |
| 416933 | 1288 | 1307 | ACGGCATTGGTGCACAGTTT | 92 | 3-14-3 | 223 |
| 417010 | 1288 | 1307 | ACGGCATTGGTGCACAGTTT | 81 | 2-13-5 | 223 |
| 416859 | 1289 | 1308 | GACGGCATTGGTGCACAGTT | 90 | 5-10-5 | 224 |
| 416934 | 1289 | 1308 | GACGGCATTGGTGCACAGTT | 90 | 3-14-3 | 224 |
| 417011 | 1289 | 1308 | GACGGCATTGGTGCACAGTT | 86 | 2-13-5 | 224 |
| 416935 | 1290 | 1309 | GGACGGCATTGGTGCACAGT | 92 | 3-14-3 | 115 |
| 417012 | 1290 | 1309 | GGACGGCATTGGTGCACAGT | 72 | 2-13-5 | 115 |
| 416860 | 1291 | 1310 | CGGACGGCATTGGTGCACAG | 88 | 5-10-5 | 225 |
| 416936 | 1291 | 1310 | CGGACGGCATTGGTGCACAG | 89 | 3-14-3 | 225 |
| 417013 | 1291 | 1310 | CGGACGGCATTGGTGCACAG | 86 | 2-13-5 | 225 |
| 416861 | 1292 | 1311 | GCGGACGGCATTGGTGCACA | 92 | 5-10-5 | 226 |
| 416937 | 1292 | 1311 | GCGGACGGCATTGGTGCACA | 93 | 3-14-3 | 226 |
| 417014 | 1292 | 1311 | GCGGACGGCATTGGTGCACA | 87 | 2-13-5 | 226 |
| 416862 | 1293 | 1312 | AGCGGACGGCATTGGTGCAC | 90 | 5-10-5 | 227 |
| 416938 | 1293 | 1312 | AGCGGACGGCATTGGTGCAC | 90 | 3-14-3 | 227 |
| 417015 | 1293 | 1312 | AGCGGACGGCATTGGTGCAC | 87 | 2-13-5 | 227 |
| 416863 | 1294 | 1313 | CAGCGGACGGCATTGGTGCA | 83 | 5-10-5 | 228 |
| 416939 | 1294 | 1313 | CAGCGGACGGCATTGGTGCA | 88 | 3-14-3 | 228 |
| 417016 | 1294 | 1313 | CAGCGGACGGCATTGGTGCA | 85 | 2-13-5 | 228 |
| 416940 | 1295 | 1314 | GCAGCGGACGGCATTGGTGC | 92 | 3-14-3 | 53 |
| 417017 | 1295 | 1314 | GCAGCGGACGGCATTGGTGC | 82 | 2-13-5 | 53 |
| 416864 | 1296 | 1315 | GGCAGCGGACGGCATTGGTG | 93 | 5-10-5 | 229 |
| 416941 | 1296 | 1315 | GGCAGCGGACGGCATTGGTG | 95 | 3-14-3 | 229 |
| 417018 | 1296 | 1315 | GGCAGCGGACGGCATTGGTG | 82 | 2-13-5 | 229 |
| 416865 | 1297 | 1316 | TGGCAGCGGACGGCATTGGT | 88 | 5-10-5 | 230 |
| 416942 | 1297 | 1316 | TGGCAGCGGACGGCATTGGT | 85 | 3-14-3 | 230 |
| 417019 | 1297 | 1316 | TGGCAGCGGACGGCATTGGT | 84 | 2-13-5 | 230 |
| 416866 | 1298 | 1317 | CTGGCAGCGGACGGCATTGG | 88 | 5-10-5 | 231 |
| 416943 | 1298 | 1317 | CTGGCAGCGGACGGCATTGG | 92 | 3-14-3 | 231 |
| 417020 | 1298 | 1317 | CTGGCAGCGGACGGCATTGG | 84 | 2-13-5 | 231 |
| 416867 | 1299 | 1318 | ACTGGCAGCGGACGGCATTG | 83 | 5-10-5 | 232 |
| 416944 | 1299 | 1318 | ACTGGCAGCGGACGGCATTG | 83 | 3-14-3 | 232 |
| 417021 | 1299 | 1318 | ACTGGCAGCGGACGGCATTG | 74 | 2-13-5 | 232 |

### Example 4

### Dose-Dependent Antisense Inhibition of Human Factor XI in HepG2 Cells

Gapmers from Example 3 (see Tables 4, 5, 6, 7, and 8), exhibiting in vitro inhibition of human Factor XI, were tested at various doses in HepG2 cells. Cells were plated at a density of 10,000 cells per well and transfected using lipofectin reagent with 9.375 nM, 18.75 nM, 37.5 nM and 75 nM concentrations of antisense oligonucleotide, as specified in Table 9. After a treatment period of approximately 16 hours, RNA was isolated from the cells and Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN®. Results are presented as percent inhibition of Factor XI, relative to untreated control cells. As illustrated in Table 9, Factor XI mRNA levels were reduced in a dose-dependent manner in antisense oligonucleotide treated cells.

TABLE 9
Dose-dependent antisense inhibition of human Factor XI in HepG2
cells via transfection of oligonucleotides with lipofectin

| | 9.375 | 18.75 | 37.5 | 75 | | SEQ |
|---|---|---|---|---|---|---|
| | nM | nM | nM | nM | Motif | ID No. |
| 412203 | 33 | 40 | 62 | 74 | 5-10-5 | 31 |
| 412206 | 24 | 47 | 69 | 86 | 5-10-5 | 34 |
| 413467 | 35 | 51 | 62 | 69 | 5-10-5 | 100 |
| 413474 | 29 | 44 | 57 | 67 | 5-10-5 | 107 |
| 413476 | 24 | 58 | 62 | 77 | 5-10-5 | 109 |
| 416825 | 23 | 52 | 73 | 92 | 5-10-5 | 190 |
| 416826 | 8 | 36 | 58 | 84 | 5-10-5 | 191 |
| 416827 | 31 | 42 | 62 | 77 | 5-10-5 | 192 |
| 416838 | 31 | 51 | 64 | 86 | 5-10-5 | 203 |
| 416842 | 18 | 33 | 62 | 71 | 5-10-5 | 207 |
| 416850 | 4 | 30 | 67 | 84 | 5-10-5 | 215 |
| 416856 | 21 | 45 | 58 | 74 | 5-10-5 | 221 |
| 416858 | 0 | 28 | 54 | 82 | 5-10-5 | 223 |
| 416864 | 18 | 43 | 62 | 78 | 5-10-5 | 229 |
| 416878 | 22 | 34 | 60 | 82 | 5-10-5 | 100 |
| 416892 | 16 | 50 | 70 | 85 | 3-14-3 | 190 |
| 416895 | 39 | 57 | 66 | 71 | 3-14-3 | 192 |
| 416896 | 22 | 39 | 57 | 81 | 3-14-3 | 193 |
| 416908 | 36 | 57 | 67 | 76 | 3-14-3 | 203 |
| 416922 | 14 | 25 | 49 | 75 | 3-14-3 | 214 |
| 416923 | 36 | 47 | 60 | 67 | 3-14-3 | 215 |
| 416924 | 25 | 38 | 56 | 59 | 3-14-3 | 216 |
| 416925 | 13 | 38 | 59 | 75 | 3-14-3 | 114 |
| 416926 | 31 | 43 | 63 | 82 | 3-14-3 | 217 |
| 416931 | 44 | 39 | 57 | 71 | 3-14-3 | 221 |
| 416941 | 33 | 54 | 63 | 78 | 3-14-3 | 229 |
| 416945 | 34 | 45 | 62 | 65 | 2-13-5 | 31 |
| 416969 | 17 | 39 | 61 | 76 | 2-13-5 | 190 |
| 416972 | 32 | 40 | 60 | 69 | 2-13-5 | 192 |
| 416973 | 60 | 75 | 85 | 87 | 2-13-5 | 193 |
| 416984 | 26 | 50 | 62 | 81 | 2-13-5 | 202 |
| 416985 | 17 | 30 | 47 | 57 | 2-13-5 | 203 |
| 416989 | 18 | 41 | 62 | 83 | 2-13-5 | 206 |
| 416993 | 15 | 37 | 50 | 68 | 2-13-5 | 209 |
| 416999 | 24 | 37 | 55 | 73 | 2-13-5 | 214 |
| 417000 | 35 | 47 | 58 | 70 | 2-13-5 | 215 |
| 417002 | 35 | 52 | 67 | 70 | 2-13-5 | 114 |
| 417003 | 26 | 44 | 60 | 56 | 2-13-5 | 217 |

The gapmers were also transfected via electroporation and their dose dependent inhibition of human Factor XI mRNA was measured. Cells were plated at a density of 20,000 cells per well and transfected via electroporation with 0.7 μM, 2.2 μM, 6.7 μM, and 20 μM concentrations of antisense oligonucleotide, as specified in Table 10. After a treatment period of approximately 16 hours, RNA was isolated from the cells and Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN®. Results are presented as percent inhibition of Factor XI, relative to untreated control cells. As illustrated in Table 10, Factor XI mRNA levels were reduced in a dose-dependent manner in antisense oligonucleotide treated cells.

TABLE 10
Dose-dependent antisense inhibition of human Factor XI in HepG2
cells via transfection of oligonucleotides with electroporation

| | 0.7 | | | | SEQ ID |
|---|---|---|---|---|---|
| | μM | 2.2 μM | 6.7 μM | 20 μM | No. |
| 412203 | 11 | 60 | 70 | 91 | 31 |
| 412206 | 22 | 39 | 81 | 94 | 34 |
| 413467 | 5 | 31 | 65 | 89 | 100 |
| 413474 | 0 | 5 | 52 | 81 | 107 |
| 413476 | 40 | 69 | 88 | 93 | 109 |
| 416825 | 27 | 74 | 92 | 98 | 190 |
| 416826 | 2 | 47 | 86 | 82 | 191 |
| 416827 | 37 | 68 | 87 | 92 | 192 |
| 416838 | 5 | 30 | 55 | 83 | 203 |
| 416842 | 0 | 10 | 66 | 92 | 207 |
| 416850 | 14 | 25 | 81 | 91 | 215 |
| 416856 | 0 | 29 | 47 | 93 | 221 |
| 416858 | 5 | 20 | 56 | 86 | 223 |
| 416864 | 32 | 65 | 78 | 90 | 229 |
| 416878 | 1 | 26 | 75 | 85 | 100 |
| 416892 | 14 | 52 | 82 | 92 | 190 |
| 416895 | 0 | 62 | 70 | 91 | 192 |
| 416896 | 12 | 35 | 81 | 89 | 193 |
| 416908 | 7 | 58 | 74 | 89 | 203 |
| 416922 | 35 | 51 | 77 | 91 | 214 |
| 416923 | 15 | 30 | 60 | 90 | 215 |
| 416924 | 22 | 40 | 63 | 70 | 216 |
| 416925 | 0 | 40 | 76 | 80 | 114 |
| 416926 | 47 | 71 | 91 | 94 | 217 |
| 416931 | 7 | 24 | 60 | 82 | 221 |
| 416941 | 16 | 38 | 79 | 89 | 229 |
| 416945 | 48 | 70 | 81 | 88 | 31 |
| 416969 | 25 | 34 | 86 | 92 | 190 |
| 416972 | 25 | 30 | 48 | 88 | 192 |
| 416973 | 20 | 48 | 86 | 93 | 193 |
| 416984 | 43 | 54 | 88 | 90 | 202 |
| 416985 | 12 | 48 | 45 | 69 | 203 |
| 416989 | 32 | 65 | 88 | 94 | 206 |
| 416993 | 22 | 48 | 87 | 92 | 209 |
| 416999 | 20 | 42 | 77 | 88 | 214 |
| 417000 | 46 | 73 | 76 | 89 | 215 |
| 417002 | 32 | 38 | 82 | 91 | 114 |
| 417003 | 0 | 34 | 75 | 89 | 217 |

### Example 5

### Selection and Confirmation of Effective Dose-Dependent Antisense Inhibition of Human Factor XI in HepG2 Cells

Gapmers exhibiting significant dose-dependent inhibition of human Factor XI in Example 4 were selected and tested at various doses in HepG2 cells. Cells were plated at a density of 10,000 cells per well and transfected using lipofectin reagent with 2.34 nM, 4.69 nM, 9.375 nM, 18.75 nM, 37.5 nM, and 75 nM concentrations of antisense oligonucleotide, as specified in Table 11. After a treatment period of approximately 16 hours, RNA was isolated from the cells and human Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN®. Results are presented as percent inhibition of human Factor XI, relative to untreated control cells. As illustrated in Table 11, Factor XI mRNA levels were reduced in a dose-dependent manner in antisense oligonucleotide treated cells compared to the control.

TABLE 11
Dose-dependent antisense inhibition of human Factor XI in HepG2
cells via transfection of oligonucleotides with lipofectin

| | 2.34 | 4.69 | 9.375 | 18.75 | 37.5 | 75 | | SEQ |
|---|---|---|---|---|---|---|---|---|
| | nM | nM | nM | nM | nM | nM | Motif | ID No. |
| 416825 | 4 | 22 | 39 | 57 | 79 | 89 | 5-10-5 | 190 |
| 416826 | 15 | 22 | 32 | 54 | 76 | 90 | 5-10-5 | 191 |
| 416838 | 21 | 37 | 50 | 63 | 74 | 83 | 5-10-5 | 203 |
| 416850 | 24 | 31 | 49 | 55 | 70 | 77 | 5-10-5 | 215 |
| 416858 | 11 | 35 | 46 | 61 | 75 | 77 | 5-10-5 | 223 |
| 416864 | 13 | 34 | 42 | 65 | 68 | 80 | 5-10-5 | 229 |
| 416892 | 14 | 34 | 49 | 70 | 84 | 93 | 3-14-3 | 190 |
| 416925 | 24 | 34 | 45 | 56 | 67 | 72 | 3-14-3 | 114 |
| 416999 | 10 | 26 | 42 | 62 | 72 | 80 | 2-13-5 | 214 |
| 417002 | 17 | 26 | 49 | 61 | 81 | 84 | 2-13-5 | 114 |
| 417003 | 6 | 29 | 48 | 64 | 73 | 82 | 2-13-5 | 217 |

The gapmers were also transfected via electroporation and their dose dependent inhibition of human Factor XI mRNA was measured. Cells were plated at a density of 20,000 cells per well and transfected via electroporation with 625 nM, 1250 nM, 2500 nM, 5,000 nM, 10,000 nM, and 20,000 nM concentrations of antisense oligonucleotide, as specified in Table 12. After a treatment period of approximately 16 hours, RNA was isolated from the cells and human Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN®. Results are presented as percent inhibition of human Factor XI, relative to untreated control cells. As illustrated in Table 12, Factor XI mRNA levels were reduced in a dose-dependent manner in antisense oligonucleotide treated cells compared to the control.

TABLE 12
Dose-dependent antisense inhibition of human Factor XI in HepG2
cells via transfection of oligonucleotides with electroporation

| | 625 | 1250 | 2500 | 5000 | 10000 | 20000 | SEQ |
|---|---|---|---|---|---|---|---|
| | nM | nM | nM | nM | nM | nM | ID No. |
| 416825 | 69 | 84 | 91 | 94 | 96 | 97 | 190 |
| 416826 | 67 | 82 | 89 | 92 | 95 | 97 | 191 |
| 416838 | 66 | 79 | 87 | 90 | 93 | 96 | 203 |
| 416850 | 69 | 80 | 87 | 90 | 93 | 96 | 215 |
| 416858 | 65 | 77 | 87 | 89 | 93 | 93 | 223 |
| 416864 | 45 | 74 | 84 | 87 | 92 | 94 | 229 |
| 416892 | 66 | 86 | 96 | 97 | 100 | 100 | 190 |
| 416925 | 64 | 80 | 88 | 91 | 95 | 96 | 114 |
| 416999 | 61 | 82 | 89 | 94 | 94 | 97 | 214 |
| 417002 | 59 | 72 | 86 | 90 | 94 | 96 | 114 |
| 417003 | 60 | 74 | 86 | 90 | 95 | 95 | 217 |

### Example 6

### Selection and Confirmation of Effective Dose-Dependent Antisense Inhibition of Human Factor XI in Cyno Primary Hepatocytes

Gapmers from Example 4 exhibiting significant dose dependent in vitro inhibition of human Factor XI were also tested at various doses in cynomolgus monkey (cyno) primary hepatocytes. Cells were plated at a density of 35,000 cells per well and transfected via electroporation with 0.74 nM, 2.2 nM, 6.7 nM, 20 nM, 60 nM, and 180 nM concentrations of antisense oligonucleotide, as specified in Table 13. After a treatment period of approximately 16 hours, RNA was isolated from the cells and human Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN®. Results are presented as percent inhibition of human Factor XI, relative to untreated control cells. As illustrated in Table 13, Factor XI mRNA levels were reduced in a dose-dependent manner in antisense oligonucleotide treated cells compared to the control.

TABLE 13
Dose-dependent antisense inhibition of human
Factor XI in cyno primary hepatocytes

| | 0.74 | 2.2 | 6.7 | 20 | 60 | 180 | SEQ |
|---|---|---|---|---|---|---|---|
| | nM | nM | nM | nM | nM | nM | ID No. |
| 416825 | 5 | 22 | 51 | 61 | 77 | 84 | 190 |
| 416826 | 13 | 24 | 34 | 67 | 69 | 71 | 191 |
| 416838 | 0 | 0 | 21 | 34 | 48 | 62 | 203 |
| 416850 | 2 | 20 | 24 | 65 | 69 | 67 | 215 |
| 416858 | 2 | 13 | 22 | 44 | 63 | 68 | 223 |
| 416864 | 0 | 1 | 15 | 23 | 47 | 64 | 229 |
| 416892 | 20 | 20 | 43 | 62 | 88 | 92 | 190 |
| 416925 | 0 | 9 | 1 | 48 | 55 | 76 | 114 |
| 416999 | 3 | 40 | 36 | 62 | 67 | 82 | 214 |
| 417002 | 32 | 16 | 28 | 38 | 55 | 71 | 114 |
| 417003 | 12 | 18 | 19 | 39 | 58 | 74 | 217 |

### Example 7

### Selection and Confirmation of Effective Dose-Dependent Antisense Inhibition of Human Factor XI in HepB3 Cells by Gapmers

Gapmers exhibiting in vitro inhibition of human Factor XI in Example 4 were tested at various doses in human HepB3 cells. Cells were plated at a density of 4,000 cells per well and transfected using lipofectin reagent with 2.3 nM, 4.7 nM, 9.4 nM, 18.75 nM, 37.5 nM, and 75 nM concentrations of antisense oligonucleotide, as specified in Table 14. After a treatment period of approximately 16 hours, RNA was isolated from the cells and human Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN®. Results are presented as percent inhibition of Factor XI, relative to untreated control cells. As illustrated in Table 14, Factor XI mRNA levels were reduced in a dose-dependent manner in antisense oligonucleotide treated cells compared to the control.

TABLE 14
Dose-dependent antisense inhibition
of human Factor XI in HepB3 cells

| ISIS | 2.3 | 4.7 | 9.4 | 18.75 | 37.5 | 75 | SEQ |
|---|---|---|---|---|---|---|---|
| No. | nM | nM | nM | nM | nM | nM | ID No. |
| 416825 | 0 | 15 | 34 | 36 | 53 | 59 | 190 |
| 416826 | 16 | 28 | 38 | 55 | 64 | 66 | 191 |
| 416838 | 23 | 34 | 43 | 59 | 71 | 56 | 203 |
| 416850 | 22 | 32 | 43 | 56 | 75 | 60 | 215 |
| 416858 | 17 | 34 | 43 | 57 | 74 | 62 | 223 |
| 416864 | 24 | 37 | 42 | 66 | 76 | 63 | 229 |
| 416892 | 28 | 34 | 50 | 68 | 82 | 72 | 190 |
| 416925 | 26 | 33 | 45 | 59 | 72 | 60 | 114 |
| 416999 | 19 | 33 | 42 | 60 | 71 | 59 | 214 |
| 417002 | 24 | 30 | 46 | 57 | 71 | 65 | 114 |
| 417003 | 11 | 28 | 40 | 40 | 63 | 58 | 217 |

The gapmers were also transfected via electroporation and their dose dependent inhibition of human Factor XI mRNA was measured. Cells were plated at a density of 20,000 cells per well and transfected via electroporation with 41.15 nM, 123.457 nM, 370.37 nM, 1111.11 nM, 3333.33 nM, and 10,000 nM concentrations of antisense oligonucleotide, as specified in Table 15. After a treatment period of approximately 16 hours, RNA was isolated from the cells and human Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN®. Results are presented as percent inhibition of human Factor XI, relative to untreated control cells. As illustrated in Table 15, Factor XI mRNA levels were reduced in a dose-dependent manner in antisense oligonucleotide treated cells compared to the control.

TABLE 15
Dose-dependent antisense inhibition
of human Factor XI in HepB3 cells

| | 41.15 | 123.457 | 370.37 | 1111.11 | 3333.33 | 10000 | SEQ |
|---|---|---|---|---|---|---|---|
| | nM | nM | nM | nM | nM | nM | ID No. |
| 416825 | 32 | 40 | 48 | 75 | 90 | 92 | 190 |
| 416826 | 0 | 0 | 34 | 61 | 87 | 92 | 191 |
| 416838 | 12 | 9 | 28 | 40 | 77 | 88 | 203 |
| 416850 | 26 | 38 | 51 | 73 | 90 | 95 | 215 |
| 416858 | 23 | 45 | 52 | 64 | 87 | 92 | 223 |
| 416864 | 4 | 3 | 6 | 35 | 75 | 87 | 229 |
| 416892 | 9 | 12 | 28 | 65 | 89 | 98 | 190 |
| 416925 | 27 | 39 | 50 | 73 | 88 | 96 | 114 |
| 416999 | 31 | 45 | 62 | 78 | 94 | 97 | 214 |
| 417002 | 19 | 0 | 31 | 47 | 86 | 93 | 114 |
| 417003 | 31 | 0 | 15 | 43 | 84 | 92 | 217 |

### Example 8

### Antisense Inhibition of Murine Factor XI in Primary Mouse Hepatocytes

Chimeric antisense oligonucleotides targeting murine Factor XI were designed as 5-10-5 MOE gapmers targeting murine Factor XI (GENBANK Accession No. NM_{—}028066.1, incorporated herein as SEQ ID NO: 6). The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in each wing segment has a 2′-MOE modification. The internucleoside linkages throughout each gaper are phosphorothioate (P═S) linkages. All cytidine residues throughout each gapmer are 5-methylcytidines. The antisense oligonucleotides were evaluated for their ability to reduce murine Factor XI mRNA in primary mouse hepatocytes.

Primary mouse hepatocytes were treated with 6.25 nM, 12.5 nM, 25 nM, 50 nM, 100 nM, and 200 nM of antisense oligonucleotides for a period of approximately 24 hours. RNA was isolated from the cells and murine Factor XI mRNA levels were measured by quantitative real-time PCR. Murine Factor XI primer probe set RTS 2898 (forward sequence ACATGACAGGCGCGATCTCT, incorporated herein as SEQ ID NO: 7; reverse sequence TCTAGGTTCACGTACACATCTTTGC, incorporated herein as SEQ ID NO: 8; probe sequence TTCCTTCAAGCAATGCCCTCAGCAATX, incorporated herein as SEQ ID NO: 9) was used to measure mRNA levels. Factor XI mRNA levels were adjusted according to total RNA content as measured by RIBOGREEN®. Several of the murine antisense oligonucleotides reduced Factor XI mRNA levels in a dose-dependent manner.

### Example 9

### Cross-Reactive Antisense Inhibition of Murine Factor XI in Primary Mouse Hepatocytes

Antisense oligonucleotides targeted to a murine Factor XI nucleic acid were tested for their effects on Factor XI mRNA in vitro. Cultured primary mouse hepatocytes at a density of 10,000 cells per well were treated with 100 nM antisense oligonucleotide. After a treatment period of approximately 24 hours, RNA was isolated from the cells and mouse Factor XI mRNA levels were measured by quantitative real-time PCR. Factor XI mRNA levels were adjusted according to total RNA content, as measured by RIBOGREEN®. Results are presented as percent inhibition of Factor XI, relative to untreated control cells.

The chimeric antisense oligonucleotides in Tables 16 were designed as 5-10-5 MOE gapmers. The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Mouse target start site” indicates the 5′-most nucleotide to which the gapmer is targeted. “Mouse target stop site” indicates the 3′-most nucleotide to which the gapmer is targeted. All the mouse oligonucleotides listed show cross-reactivity between the mouse Factor XI mRNA (GENBANK Accession No. NM_{—}028066.1), incorporated herein as SEQ ID NO: 6 and the human Factor XI mRNA (GENBANK Accession No. NM_{—}000128.3), incorporated herein as SEQ ID NO: 1. “Human Target Start Site” indicates the 5′-most nucleotide in the human mRNA (GENBANK Accession No. NM_{—}000128.3) to which the antisense oligonucleotide is targeted. “Human Target Stop Site” indicates the 3′-most nucleotide in the human mRNA (GENBANK Accession No. NM_{—}000128.3) to which the antisense oligonucleotide is targeted. “Number of mismatches” indicates the mismatches between the mouse oligonucleotide and the human mRNA sequence.

TABLE 16
Inhibition of mouse Factor XI mRNA levels by chimeric antisense oligonucleotides
having 5-10-5 MOE wings and deoxy gap targeted to SEQ ID NO: 1 and SEQ ID NO: 6

| | Mouse | Mouse | | | | Human | Human | |
|---|---|---|---|---|---|---|---|---|
| | Target | Target | | SEQ | SEQ | Target | Target | No. of |
| ISIS No | Start Site | Stop Site | Sequence (5′ to 3′) | ID No. | ID No. | Start Site | Stop Site | Mismatches |
| 404050 | 379 | 398 | TGCTTGAAGGAATATCCAGA | 82 | 233 | 619 | 638 | 2 |
| 404054 | 448 | 467 | TAGTTCATGCCCTTCATGTC | 45 | 234 | 688 | 707 | 1 |
| 404055 | 453 | 472 | TGTTATAGTTCATGCCCTTC | 27 | 235 | 693 | 712 | 1 |
| 404066 | 686 | 705 | AATGTCCCTGATACAAGCCA | 37 | 236 | 926 | 945 | 1 |
| 404067 | 691 | 710 | GGGAAAATGTCCCTGATACA | 39 | 237 | 931 | 950 | 1 |
| 404083 | 1299 | 1318 | TGTGCAGAGTCACCTGCCAT | 47 | 238 | 1533 | 1552 | 2 |
| 404087 | 1466 | 1485 | TTCTTGAACCCTGAAGAAAG | 29 | 239 | 1709 | 1728 | 2 |
| 404089 | 1477 | 1496 | TGAATTATCATTTCTTGAAC | 6 | 240 | 1720 | 1739 | 2 |
| 404090 | 1483 | 1502 | TGATCATGAATTATCATTTC | 42 | 241 | 1726 | 1745 | 2 |

### Example 10

### In Vivo Antisense Inhibition of Murine Factor XI

Several antisense oligonucleotides targeted to murine Factor XI mRNA (GENBANK Accession No. NM_{—}028066.1, incorporated herein as SEQ ID NO: 6) showing statistically significant dose-dependent inhibition from the in vitro study were evaluated in vivo. BALB/c mice were treated with ISIS 404057 (TCCTGGCATTCTCGAGCATT, target start site 487, incorporated herein as SEQ ID NO: 10) and ISIS 404071 (TGGTAATCCACTTTCAGAGG, target start site 869, incorporated herein as SEQ ID NO: 11).

### Treatment

BALB/c mice were injected with 5 mg/kg, 10 mg/kg, 25 mg/kg, or 50 mg/kg of ISIS 404057 or ISIS 404071 twice a week for 3 weeks. A control group of mice was injected with phosphate buffered saline (PBS) twice a week for 3 weeks. Mice were sacrificed 5 days after receiving the last dose. Whole liver was harvested for RNA analysis and plasma was collected for protein analysis.

### RNA Analysis

RNA was extracted from liver tissue for real-time PCR analysis of Factor XI. As shown in Table 17, the antisense oligonucleotides achieved dose-dependent reduction of murine Factor XI over the PBS control. Results are presented as percent inhibition of Factor XI, relative to control.

TABLE 17
Dose-dependent antisense inhibition of murine Factor XI mRNA
in BALB/c mice

| | | % |
|---|---|---|
| | mg/kg | inhibition |
| 404057 | 5 | 40 |
| | 10 | 64 |
| | 25 | 85 |
| | 50 | 95 |
| 404071 | 5 | 72 |
| | 10 | 82 |
| | 25 | 93 |
| | 50 | 96 |

### Protein Analysis

As shown in Table 18, treatment with ISIS 404071 resulted in a significant dose-dependent reduction of Factor XI protein. Results are presented as percent inhibition of Factor XI, relative to PBS control.

TABLE 18
Dose-dependent inhibition of murine Factor XI protein by ISIS 404071
in BALB/c mice

| Dose in | % |
|---|---|
| mg/kg | Inhibition |
| 5 | 39 |
| 10 | 67 |
| 25 | 89 |
| 50 | 96 |

### Example 11

### In Vivo Effect of Antisense Inhibition of Murine Factor XI on Collagen-Induced Arthritis

The effect of inhibition of Factor XI and its role in fibrin accumulation in the joints leading to joint inflammation and rheumatoid arthritis was evaluated in a murine collagen-induced arthritis (CIA) model. Administration of collagen to animals is a well known method to induce arthritis and has been previously described by Trentham et al. (J Exp Med, 146:857-68, 1977), Courtenay et al. (Nature, 283:666-668, 1980), Cathcart et al. (Lab Invest, 54:26-31, 1986), Wooley (Methods Enzymol 162:361-373, 1988) and Holmdahl et al. (Arthritis Rheum 29:106, 1986). Arthritis induced in mice administered collagen can be visually assessed and clinically scored as described by Marty et al. (J Clin Invest, 107:631-640, 2001) where 1 point is given for each swollen digit except the thumb (maximum, 4), 1 point is given for the tarsal or carpal joint, and 1 point is given for the metatarsal or metacarpal joint with a maximum score of 6 for a hindpaw and 5 for a forepaw. Each paw was graded individually, the cumulative clinical arthritic score per mouse reaching a maximum of 22 points

ISIS 404071 (TGGTAATCCACTTTCAGAGG, incorporated herein as SEQ ID NO: 11) is a chimeric antisense oligonucleotide designed as a 5-10-5 MOE gapmer targeting murine Factor XI (GENBANK Accession No. NM_{—}028066.1, incorporated herein as SEQ ID NO: 6; oligonucleotide target site starting at position 869). The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytidine residues throughout the gapmer are 5′ methylcytidines.

ISIS 403102 (CCATAGAACAGCTTCACAGG, incorporated herein as SEQ ID NO: 275) is a chimeric antisense oligonucleotide designed as a 5-10-5 MOE gapmer targeting murine Factor VII. The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytidine residues throughout the gapmer are 5′ methylcytidines.

ISIS 421208 (TCGGAAGC GACTCTTATATG, incorporated herein AS SEQ ID NO: 14), a control oligonucleotide for ISIS 404071 with 8 mismatches (MM), was used as a control. ISIS 421208 is a chimeric antisense oligonucleotide designed as a 5-10-5 MOE gapmer targeting murine Factor XI (GENBANK Accession No. NM_{—}028066.1, incorporated herein as SEQ ID NO: 6; oligonucleotide target site starting at position 869). The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytidine residues throughout the gapmer are 5′ methylcytidines.

ISIS 404057 (TCCTGGCATT CTCGAGCATT, incorporated herein as SEQ ID NO: 10) is a chimeric antisense oligonucleotide designed as a 5-10-5 MOE gapmer targeting murine Factor XI (GENBANK Accession No. NM_{—}028066.1, incorporated herein as SEQ ID NO: 6; oligonucleotide target site starting at position 487). The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytidine residues throughout the gapmer are 5′ methylcytidines.

Male DBA/1J mice were obtained from The Jackson Laboratory (Bar Harbor, Me.).

### Arthritis Study A: Effect of Factor XI Inhibition (ISIS 404071) on Arthritis

The effect of inhibiting Factor XI with ISIS 404071 and its role in ameliorating arthritis was evaluated in a collagen-induced arthritis (CIA) model.

Male DBA/1J mice were separated in groups and treated as shown in Table 19.

TABLE 19
Summary of Mice Study Groups

| Group (N) | ASO | CIA |
|---|---|---|
| 1. (15) | None | No |
| 2. (15) | None | Yes |
| 3. (15) | F7 (403102) 20 mpk | Yes |
| 4. (15) | F11 (404071) 20 mpk | Yes |

In a group of 15 DBA/1J mice, 20 mg/kg of ISIS 404071 was injected subcutaneously twice a week for 12 weeks. One control group of 15 mice was injected with 20 mg/kg of ISIS 403102 twice a week for 12 weeks. Two control groups of 15 mice each were injected with PBS twice a week for 12 weeks. Two weeks after the first oligonucleotide dose, type II bovine collagen (Chondrex Inc, Redmond, Wash.) was mixed with complete Freund's adjuvant, homogenized on ice and the emulsion, containing 100 μg of collagen, was injected subcutaneously at the base of the tail in the Factor XI group, the Factor VII group and one of the PBS control groups. A booster injection containing 100 μg collagen type II in incomplete Freund's adjuvant was injected subcutaneously at the base of the tail at a different injection site on day 21 after the first collagen injection in these groups.

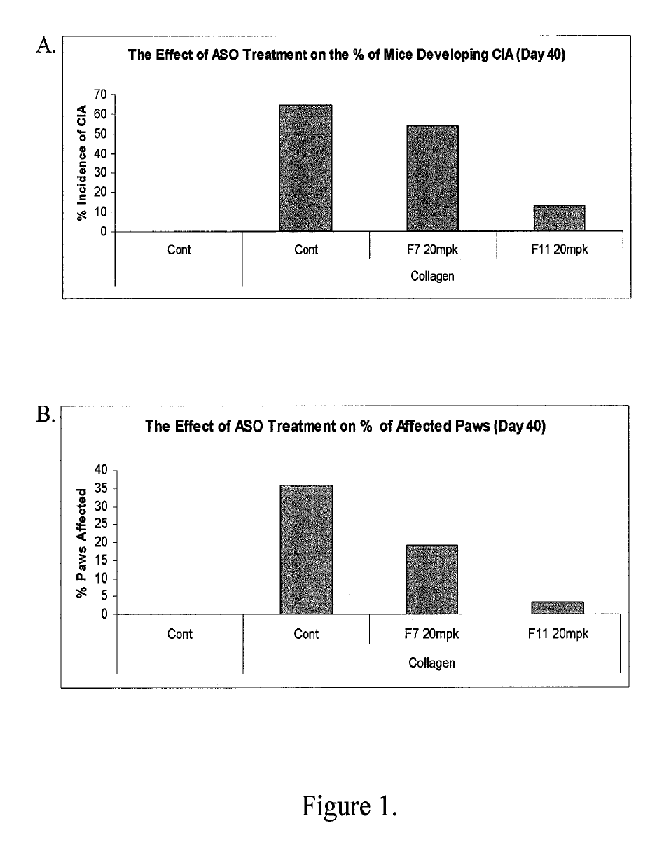

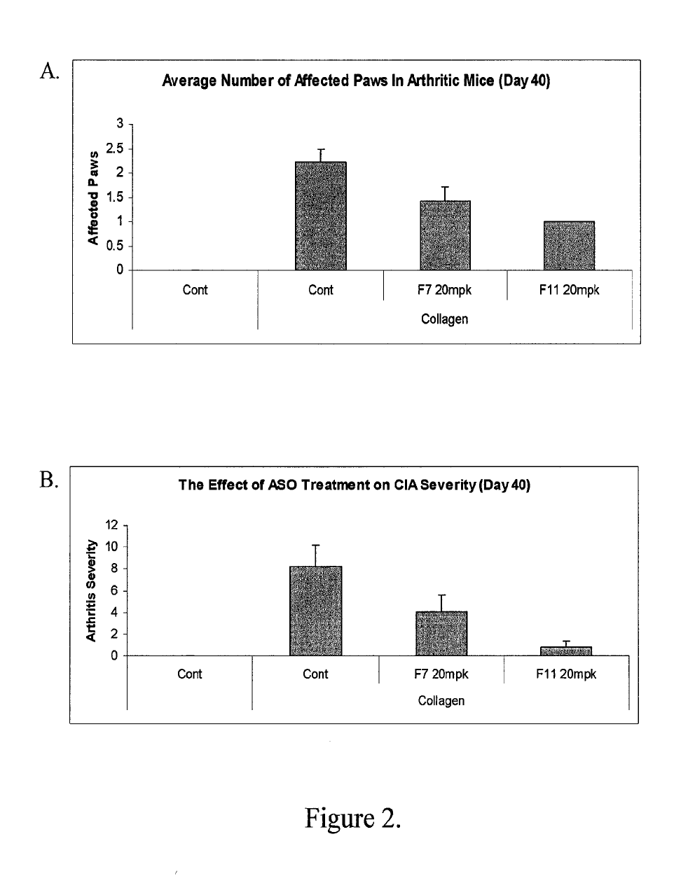

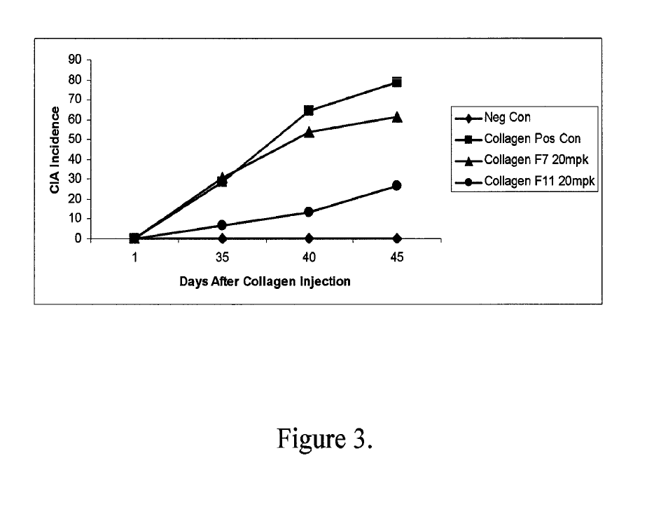

Starting 35 days from the first collagen injection, mice in all groups were examined daily for the visual appearance of arthritis, such as swelling and stiffness, in peripheral joints. The results are presented in Table 20 (expressed as a percentage of the PBS control) and in FIGS. 1-3.

TABLE 20
Clinical Effects of Antisense Oligonucleotide Treatment on CIA

| | Collagen- | | |
|---|---|---|---|
| | treated | ISIS 404071- | ISIS 403102- |
| | control | treated | treated |
| % of CIA | 64 | 13 | 54 |
| % of paws | 36 | 3 | 19 |
| affected | | | |
| Average no. of | 2 | 1 | 1 |
| paws affected | | | |
| Clinical severity | 8 | 1 | 4 |
| of CIA | | | |

‘Incidence of CIA’ refers to the percentage of mice in each group that had CIA at day 40. The ‘percentage of paws affected’ refers to the percentage of paws out of a total of 60 paws in each group of mice that were affected with arthritis. ‘Average number of affected paws’ refers to the number of affected paws in mice that were diagnosed to have arthritis. The ‘clinical severity of CIA’ was scored as described by Marty et al. (J Clin Invest, 107:631-640, 2001) and as follows: 1 point for each swollen digit except the thumb (maximum, 4), 1 point for the tarsal or carpal joint, and 1 point for the metatarsal or metacarpal joint with a maximum score of 6 for a hindpaw and 5 for a forepaw. Each paw was graded individually, the cumulative clinical arthritic score per mouse reaching a maximum of 22 points.

As shown in Table 20 and FIGS. 1-3, treatment with ISIS 404071 was observed to significantly inhibit the incidence of CIA.

### Arthritis Study B: Effect of Factor XI Inhibition (ISIS 404071 and ISIS 404057) on Arthritis

The effect of inhibiting Factor XI with ISIS 404071 or ISIS 404057 and their role in ameliorating arthritis was evaluated in a collagen-induced arthritis model.

Male DBA/1J mice were separated in groups and treated as shown in Table 21.

TABLE 21
Summary of Mice Study Groups

| Group (N) | ASO | CIA |
|---|---|---|
| 1. (20) | None | No |
| 2. (20) | None | Yes |
| 3. (20) | F11 (404071) 20 mpk | Yes |
| 4. (20) | F11 (404057) 20 mpk | Yes |
| 5. (20) | F11 MM (421208) 20 mpk | Yes |

In a group of 20 DBA/1J mice, 20 mg/kg of ISIS 404071 were injected subcutaneously twice a week for ten weeks. In a second group of 20 DBA/1J mice, 20 mg/kg of ISIS 404057 were injected subcutaneously twice a week for ten weeks. In a third control group of 20 DBA/1J mice, 20 mg/kg of ISIS 421208 were injected subcutaneously twice a week for ten weeks. Two control groups of 20 mice each were injected with PBS twice a week for ten weeks.

Two and a half weeks after the first oligonucleotide dose, type II bovine collagen in complete Freund's adjuvant was injected subcutaneously at the base of the tail in all the experimental groups and one PBS control group. A booster injection containing 100 μg collagen type II in incomplete Freund's adjuvant was injected subcutaneously at the base of the tail at a different injection site on day 21 after the first collagen injection in these groups.

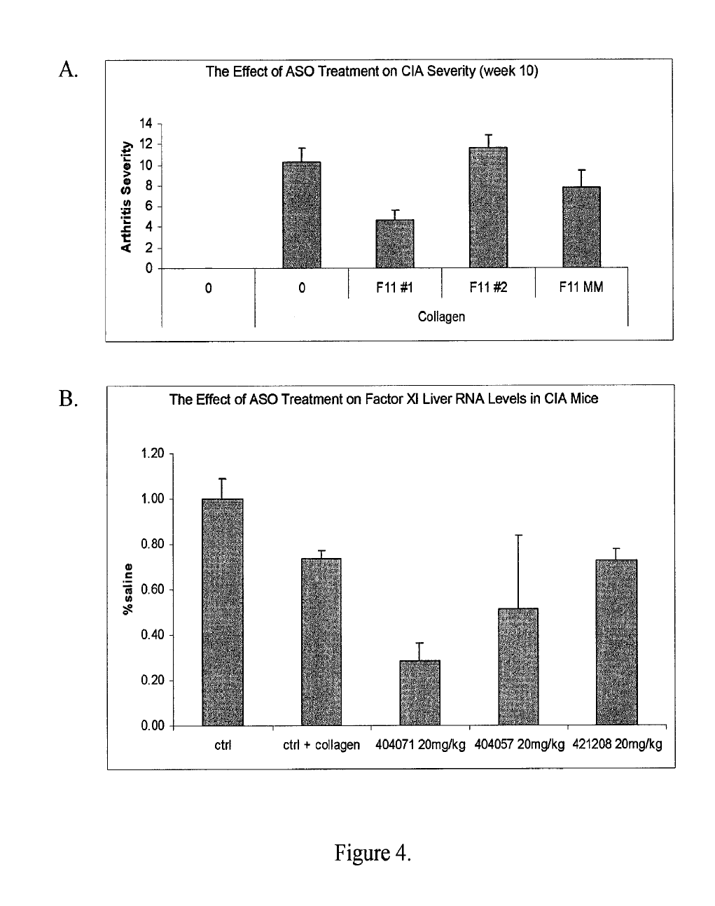

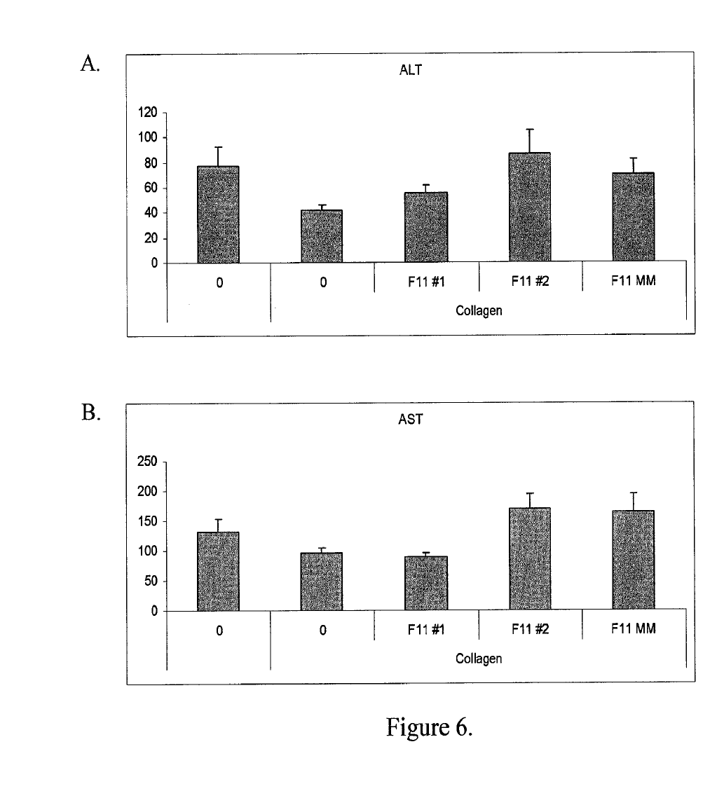

Mice in all groups were examined daily after day 30 after the first collagen injection for the visual appearance of arthritis in peripheral joints. The effects of Factor XI antisense oligonucleotide treatment at the end of the study are shown in Table 22 and FIGS. 4-6. The results in Table 22 are expressed as percent change compared to the PBS control except for the liver and spleen weight.

TABLE 22
Effects of Antisense Oligonucleotide Treatment on CIA

| | Collagen-treated | ISIS 404071- | ISIS 421208- |
|---|---|---|---|
| | control | treated | treated |
| % of CIA | 100% | 64% | 81% |
| % of paws affected | 67% | 20% | 45% |
| Average no. of paws | 2.5 | 0.82 | 1.7 |
| affected | | | |
| Clinical severity of CIA | 10.3 | 4.6 | 7.75 |
| Liver Weight | 5.56% | 6.089% | 6.14% |
| as a % of Body Weight | | | |
| Spleen Weight | 0.608 | 0.759 | 0.718 |
| as a % of Body Weight | | | |
| Liver ALT | 42.25 | 56.6 | 70.06 |
| Liver AST | 97.3 | 86.75 | 165.12 |

Treatment of collagen-induced arthritic mice with Factor XI antisense oligonucleotide ISIS 404071 significantly decreased the amount of arthritis assessed in the mice compared to untreated mice (Table 22 and FIG. 4A). Treatment of collagen-induced arthritic mice with Factor XI antisense oligonucleotide ISIS 404057 did not affect the amount of arthritis assessed in the mice compared to untreated mice. The difference between the effective of the two Factor XI antisense oligonucleotides may be related to the relative effectiveness of each oligonucleotide in inhibiting Factor XI mRNA as shown in FIG. 4B. The lack of Factor XI inhibition by ISIS 404057 correlated with the lack of arthritis inhibition in the mice.

In summary, this example shows for the first time known to the inventors that Factor XI plays a role in arthritis and that treatment of an animal with a Factor XI inhibitor will ameliorate arthritis in the animal. Treatment with a Factor XI inhibitor is also shown to reduce the risk and progression of arthritis in an animal.

### Example 12

### In Vivo Effect of Antisense Inhibition of Murine Factor XI on DSS-Induced Colitis

The effect of antisense oligonucleotide inhibition of Factor XI and its role in ameliorating colitis was evaluated in a dextran sulfate sodium (DSS)-induced colitis model. Administration of DSS to animals is a well known method to induce colitis and has been previously described by Okayasu et al. (Gastroenterology, 1990, 98:694-702), Cooper et al. (Lab Invest, 1993, 69:238-249) and Dieleman et al. (Clin Exp Immunol, 1998, 114:385-391).

Colitis in humans has symptoms that can include persistent diarrhea (loose, watery, or frequent bowel movements), crampy abdominal pain, fever, rectal bleeding, loss of appetite and weight loss. Pathological changes in colitis can include changes to the colon such as colon shortening (Gore, 1992, AJR, 158:59-61), formation of inflammatory lesions, diffused neutrophil infiltration, submucosa edema and muscularis propria thickening.

Antisense oligonucleotides targeting Factor XI were described in Example 11, supra. Swiss Webb mice were from Charles River Laboratories (Wilmington, Mass.).

### Colitis Study A: Effect of Factor XI Inhibition (ISIS 404071) on Colitis

The effect of inhibiting Factor XI with ISIS 404071 and its role in ameliorating colitis was evaluated in a dextran sulfate sodium (DSS)-induced colitis model.

Female Swiss Webb mice were separated in groups and treated as shown in Table 23.

TABLE 23
Summary of Mice Study Groups

| Group (N) | ASO | DSS |
|---|---|---|
| 1. (8) | None | No |
| 2. (8) | None | Yes |
| 3. (8) | F7 (403102) 20 mpk | Yes |
| 4. (8) | F11 (404071) 20 mpk | Yes |

In groups of 8 Swiss Webb mice, 20 mg/kg of ISIS 404071 or ISIS 403102 were injected subcutaneously twice a week for 3 weeks. Two control groups of 8 mice each were injected with PBS twice a week for 3 weeks. After the oligonucleotide treatments, 4% DSS in water was administered ad libitum for 6 days to the experimental groups and one PBS control group.

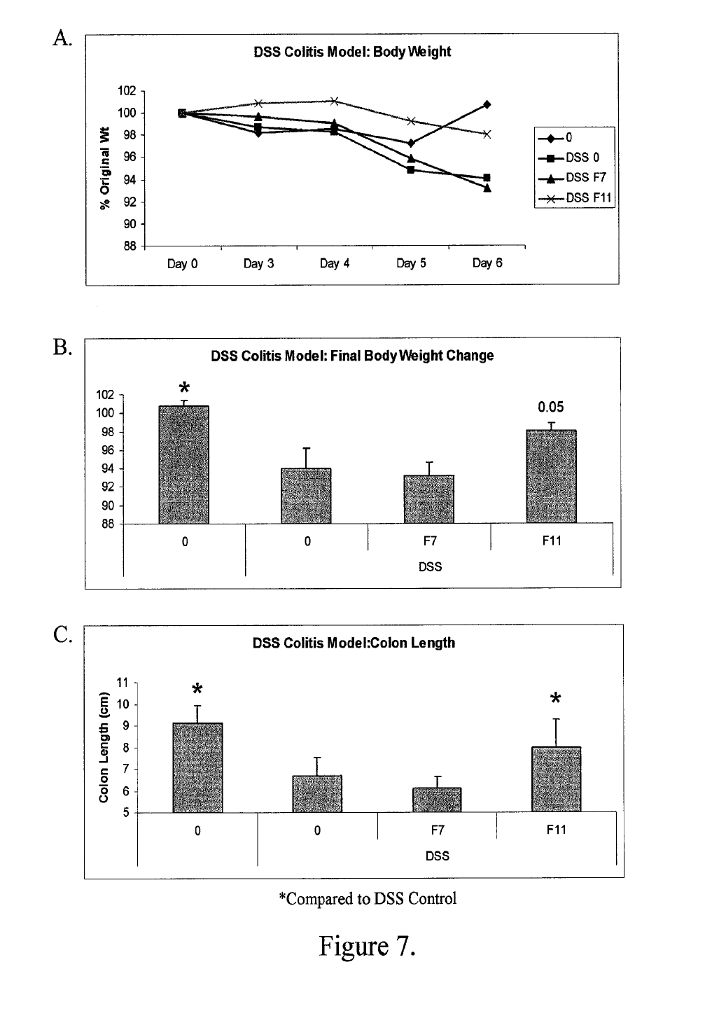

Mice in all groups were weighed at day 0. Mice were sacrificed at the end of the study on day 7 after DSS was administered and their body weights and colon lengths were measured. Results are presented in Table 24 (expressed as a percentage of the PBS control) and FIG. 7.

TABLE 24
Effect of Factor XI inhibition on the DSS-induced colitis model on day 7

| | DSS-treated | ISIS 404071 | ISIS 403102 |
|---|---|---|---|
| | control | treated | treated |
| Body weight | −6 | −2 | −7 |
| change | | | |
| Colon length | −27 | −13 | −34 |
| change | | | |

Colon length was assessed in the DSS-induced colitis mice. Treatment with Factor XI oligonucleotide decreased the amount of colon shortening symptomatic of colitis.

Mouse colon tissue from a mouse (DSS induced) ulcer colitis model treated with Factor VII or XI oligonucleotide was studied to determine the amount of inflammation present.

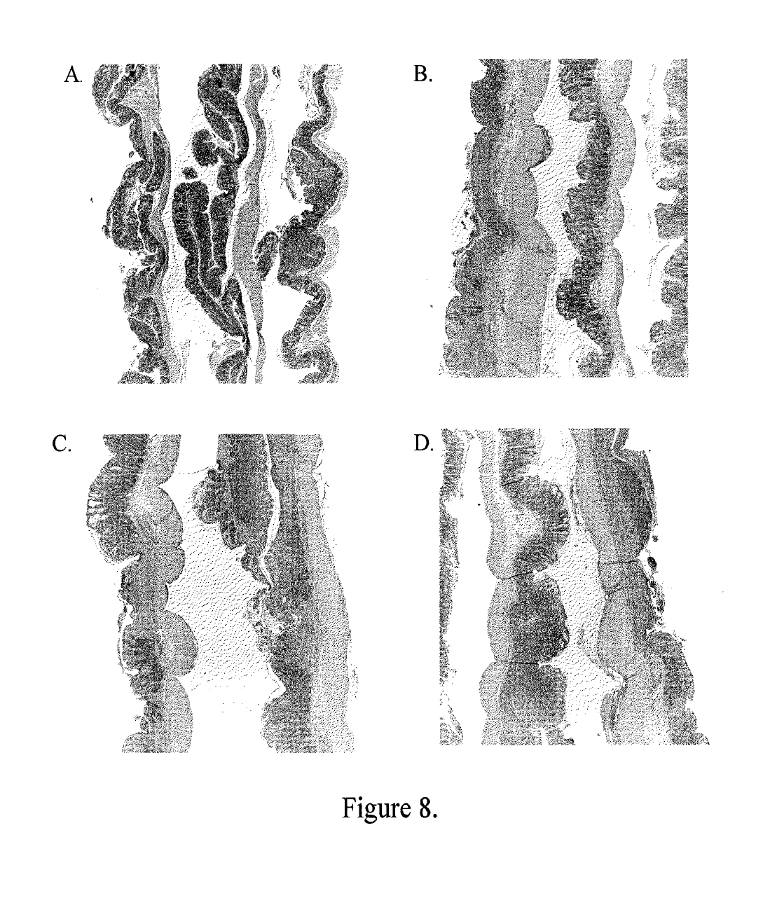

The mice were sacrificed using sodium pentobarbital (160 mg/kg). Colon sections, divided into three equal segments, cut lengthwise, and fixed in 10% neutral-buffered formalin, paraffin-embedded, sectioned at 4 μm, and stained with hematoxylin and eosin for light microscopic examination. The slides were reviewed microscopically by a pathologist and assigned a histological severity score for intestinal inflammation as shown in FIG. 8 and Table 25. The amount of inflammation in 8C (Factor VII treated) and 8D (Factor XI treated) were compared to the negative control in 8A (no inflammation) and the positive control in 8B (maximal inflammation) to determine the severity of inflammation.

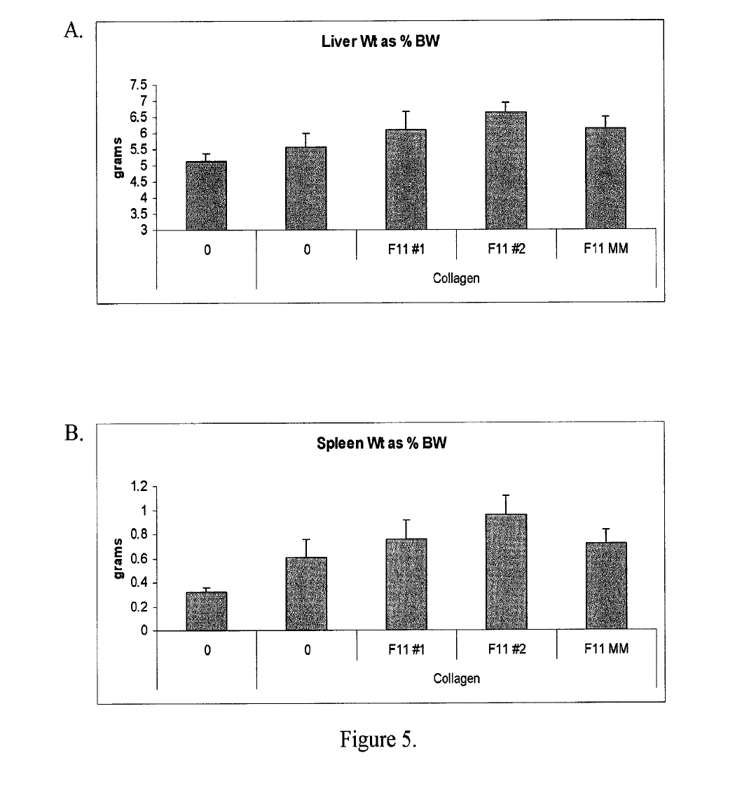

TABLE 25
Colon tissue histopathology severity scores

| | | | Severity |
|---|---|---|---|
| Group | Treatment | ASO/Target | Score |
| 1 (FIG. 5A) | None | None | |
| 2 (FIG. 5B) | DSS | None | ++++ |
| 3 (FIG. 5C) | DSS | FVII | ++++ |
| 4 (FIG. 5D) | DSS | FXI | + |

Multi-lesion colitis was observed in DSS treated colons (FIG. 8B) compared to colons not treated with DSS (FIG. 8A). The colon in FIG. 8C was treated with the control oligonucleotide ISIS 403102 targeting Factor VII and exhibits lesions similar in appearance to the DSS treated colon in FIG. 8B. The colon in FIG. 8D was treated with ISIS 404071 and exhibits significantly fewer mucosa ulcerative lesions than the colon in FIG. 8B or 8C.

### Colitis Study B: Effect of Factor XI Inhibition (ISIS 404071 and ISIS 404057) on Colitis

The effect of inhibiting Factor XI with ISIS 404071 and ISIS 404057 and their roles in ameliorating colitis was evaluated in a dextran sulfate sodium (DSS)-induced colitis model.

Swiss Webb mice were separated in groups and treated as shown in Table 26.

TABLE 26
Summary of Mice Study Groups

| Group (N) | ASO | DSS |
|---|---|---|
| 1. (8) | None | No |
| 2. (8) | None | Yes |
| 3. (8) | F11 (404071) 20 mpk | Yes |
| 4. (8) | F11 (404057) 20 mpk | Yes |
| 5. (8) | F11 MM (421208) 20 mpk | Yes |

In a first group of 8 Swiss Webb mice, 20 mg/kg of ISIS 404071 was injected subcutaneously twice a week for 3 weeks. In a second group of 8 Swiss Webb mice, 20 mg/kg of ISIS 404057 was subcutaneously injected twice a week for 3 weeks. In a third control group of 8 Swiss Webb mice, 20 mg/kg of ISISI 421208 was injected subcutaneously twice a week for 3 weeks. Two control groups of 8 mice each were injected with PBS twice a week for 3 weeks. After the oligonucleotide treatment, 4% DSS in water was administered ad libitum for 6 days to all the experimental groups and one PBS control group.

Mice in all groups were weighed at day 0 and daily after administration of DSS. Mice were sacrificed at the end of the study on day 7 after DSS was administered, their livers and colons were harvested for RNA analysis, and their body weights and colon lengths were measured.

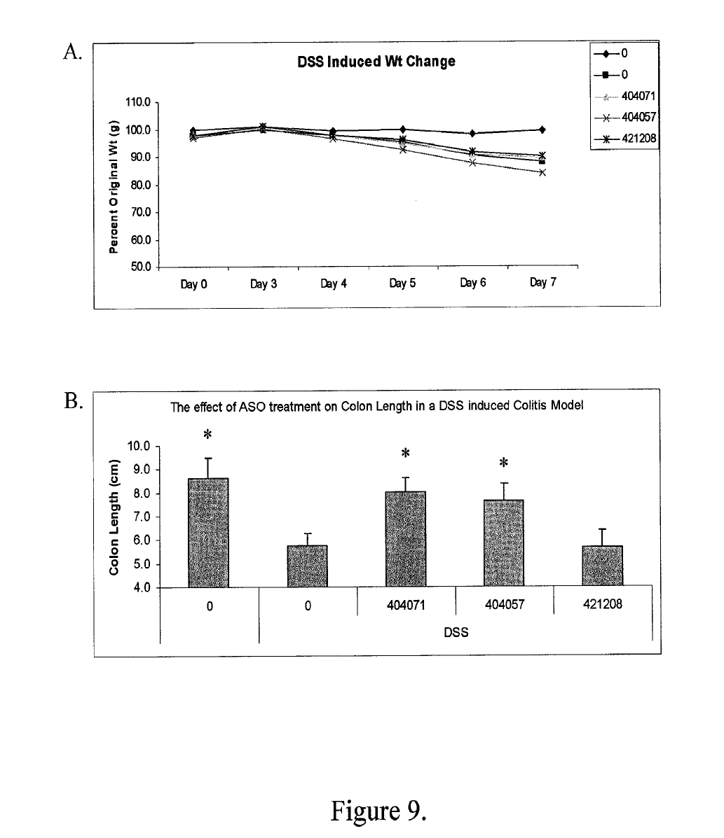

The effect of the antisense oligonucleotides on weight change and body colon length is shown in Table 27 and FIG. 9.

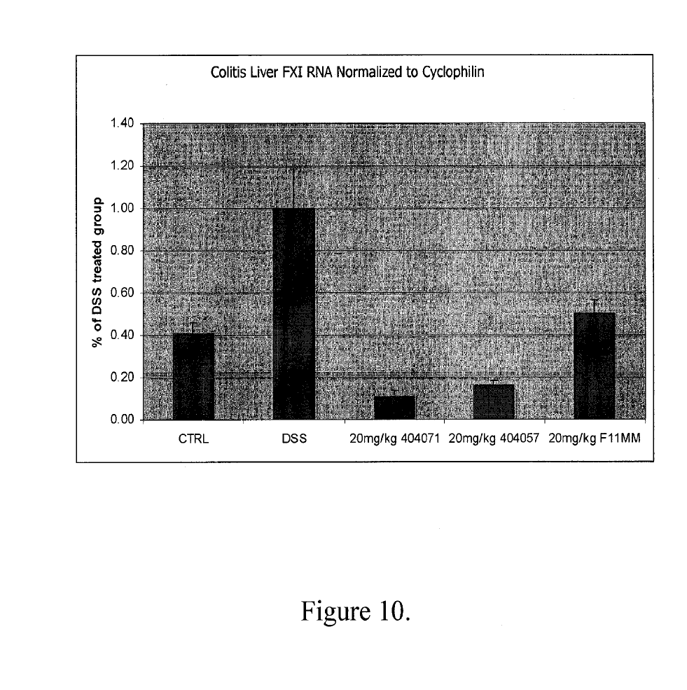

RT-PCR analysis of Factor XI mRNA was performed. As presented in Table 27 and FIG. 10, antisense oligonucleotides targeting Factor XI achieved statistically significant dose-dependent reduction of murine Factor XI over the PBS control in the liver. All the measurements are normalized to cyclophilin.

TABLE 27
Effects of Factor XI Inhibition on DSS-Induced Colitis on Day 7

| | DSS control | ISIS 404071 | ISIS 404057 | ISIS 421208 |
|---|---|---|---|---|
| Liver Factor | +150 | −75 | −50 | +25 |
| XI mRNA | | | | |
| Colon length | −33 | −11 | −11 | −33 |
| Body weight | −12 | −10 | −16 | −10 |

All the results in Table 27 are expressed as percent change compared to the PBS control.

### Colitis Study C: Effect of Factor XI Inhibition (ISIS 404071) on Colitis

A third study on colitis using Factor XI oligonucleotide (ISIS 404071, SEQ ID NO: 11) was conducted. The study was performed essentially as described earlier in this example. A stool softness/diarrhea study was conducted. After seven days, control mice not administered DSS did not have diarrhea, mice administered DSS produced diarrhea and mice administered Factor XI oligonucleotide produced normal to soft stool but no diarrhea.

P-selectin has been implicated in exacerbating colitis and ablation of P-selectin has been found to ameriolate colitis (Gironella, M. et al., J. Leukoc. Biol. 2002. 72: 56-64). The effect of antisense inhibition of Factor XI on serum P-selectin levels was evaluated in this study. DSS administration led to increase in P-selectin levels which was reduced by treatment with ISIS 404071. The results are presented in Table 28.

TABLE 28
Effects of Factor XI Inhibition on P-selectin levels in the serum

| | P-selectin (ng/mL) |
|---|---|
| Control | 86 |
| DSS control | 106 |
| ISIS 404071 | 83 |

### Colitis Study D: Effect of Various Doses of Factor XI Inhibition (ISIS 404071) on Colitis

The effect of inhibiting Factor XI with various doses of ISIS 404071 in ameliorating colitis was evaluated in a dextran sulfate sodium (DSS)-induced colitis model.

Female Swiss Webb mice were separated in groups and treated as shown in Table 29.

TABLE 29
Summary of Mice Study Groups

| Group (N) | ASO | DSS |
|---|---|---|
| 1. (8) | None | No |
| 2. (8) | None | Yes |
| 3. (8) | F11 (404071) 40 mpk | Yes |
| 4. (8) | F11 (404071) 20 mpk | Yes |
| 5. (8) | F11 (404071) 10 mpk | Yes |

In a first group of 8 Swiss Webb mice, 10 mg/kg of ISIS 404071 was injected subcutaneously twice a week for 3 weeks. In a second group of 8 Swiss Webb mice, 20 mg/kg of ISIS 404057 was subcutaneously injected twice a week for 3 weeks. In a third control group of 8 Swiss Webb mice, 40 mg/kg of ISISI 404057 was injected subcutaneously twice a week for 3 weeks. Two control groups of 8 mice each were injected with PBS twice a week for 3 weeks. After the oligonucleotide treatment, 4% DSS in water was administered ad libitum for 7 days to all the experimental groups and one PBS control group.

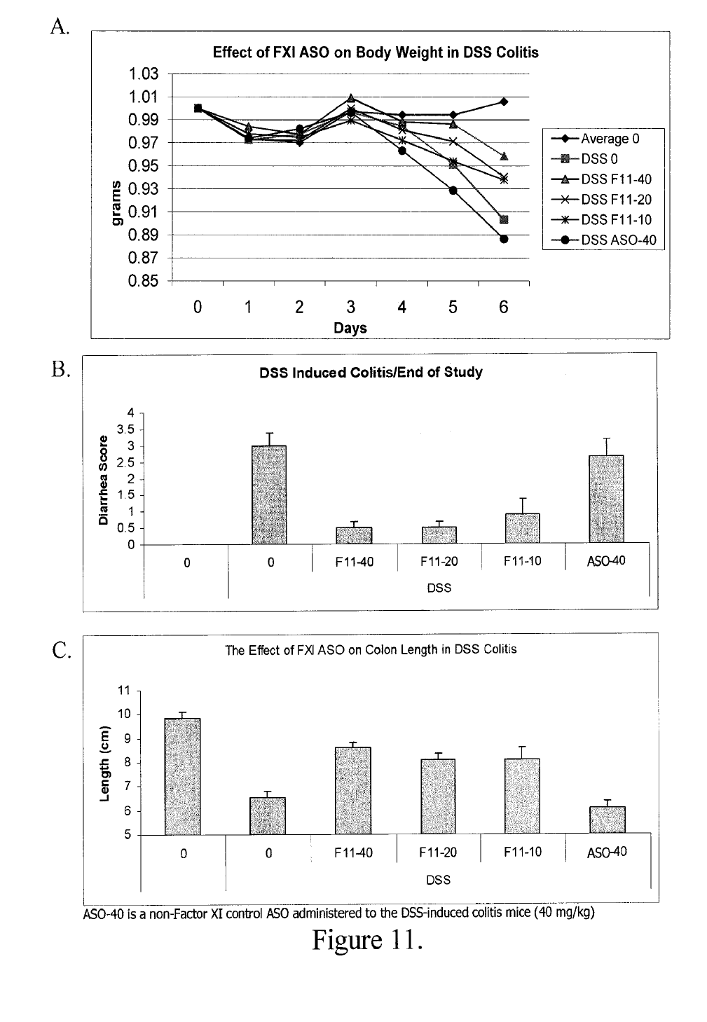

Mice in all groups were weighed at day 0 and daily starting on day 3 after administration of DSS as shown in FIG. 11A. The stool softness/diarrhea of the mice was analyzed on day 7 after DSS was administered as shown in FIG. 11B. Mice were sacrificed at the end of the study on day 7 after DSS was administered and their colon lengths were measured as shown in FIG. 11C.

The antisense oligonucleotide showed statistically significant dose effects on body weights and diarrhea scores (FIGS. 11A and 11B). Although the various doses of oligonucleotide did not show a statistical significant effect between the various doses on colon length, administration of any of the three doses significantly improved the colon length when compared to placebo as shown in FIG. 11C.

Mice with dextran sodium sulfate (DSS)-induced colitis have elevated level of thrombin-antithrombin (TAT) complexes in blood (Anthoni, C. et al., J. Exp. Med. 204: 1595-1601, 2007) that is also observed in patients with ulcerative colitis (Kume, K. et al., Intern Med. 2007. 46: 1323-9). The effect of antisense inhibition of Factor XI on TAT levels in the colon was evaluated (Thrombi-Anti-Thrombin III complex, Siemens Healthcare Diagnostics, Deerfield, Ill.) and the results are presented in Table 30. As demonstrated, DSS administration increased TAT levels, which were decreased in a dose-dependent manner by treatment with ISIS 404071.

Plasma levels of soluble CD40 ligand (CD40L) are known to be elevated in cases of inflammatory bowel disease (Ludwiczek, O. et al., Int. J. Colorectal Disease. 2003. 18: 142-147) and may be considered a marker of intestinal inflammation. The effect of antisense inhibition of Factor XI on CD40L levels in the plasma was evaluated (Bender Medsystems, Vienna, Austria; eBioscience, San Diego, Calif.) and the results are presented in Table 31. As demonstrated, DSS administration increased CD40L levels, which were decreased by treatment with ISIS 404071.

Observations on experimental models and humans with ulcerative colitis suggest a pathogenetic role of the kallikrein-kinin system in inflammatory bowel diseases (Devani, M. et al., Digestive and Liver Disease. 2005. 37: 665-673). The effect of antisense inhibition of Factor XI on kinin levels in the colon was evaluated (Phoenix Pharmaceuticals, Burlingame, Calif.) and the results are presented in Table 32. As demonstrated, DSS administration increased bradykinin levels, which were decreased by treatment with ISIS 404071.

TABLE 30
TAT levels in colon of mice groups

| Group | | | TAT levels |
|---|---|---|---|
| (N) | ASO | DSS | (ng/mg protein) |
| 1. (8) | None | No | 0.03 |
| 2. (8) | None | Yes | 2.57 |
| 3. (8) | F11 (404071) 40 mpk | Yes | 0.75 |
| 4. (8) | F11 (404071) 20 mpk | Yes | 0.65 |
| 5. (8) | F11 (404071) 10 mpk | Yes | 1.79 |

TABLE 31
CD40L levels in colon of mice groups

| | | | CD40L levels |
|---|---|---|---|
| Group (N) | ASO | DSS | (ng/mg protein) |
| 1. (8) | None | No | 0.67 |
| 2. (8) | None | Yes | 0.39 |
| 3. (8) | F11 (404071) 40 mpk | Yes | 0.39 |
| 4. (8) | F11 (404071) 20 mpk | Yes | 0.26 |
| 5. (8) | F11 (404071) 10 mpk | Yes | 0.19 |

TABLE 32
Bradykinin levels in colon of mice groups

| | | | Bradykinin levels |
|---|---|---|---|
| Group (N) | ASO | DSS | (ng/mg protein) |
| 1. (8) | None | No | 0.00 |
| 2. (8) | None | Yes | 0.09 |
| 3. (8) | F11 (404071) 40 mpk | Yes | 0.02 |
| 4. (8) | F11 (404071) 20 mpk | Yes | 0.00 |
| 5. (8) | F11 (404071) 10 mpk | Yes | 0.03 |

In summary, this example shows that Factor XI oligonucleotide treatment significantly ameliorated DSS induced ulcerative colitis in an animal. Treatment with a Factor XI inhibitor is also shown to reduce the risk and progression of colitis in an animal.

### Colitis Study E: to Determine if Native Human Factor XI Protein can Reverse the Effect of Factor XI Inhibition (ISIS 404071) in a Colitis Model.

The efficacy of human factor XI protein in reversing the effect of inhibiting Factor XI with ISIS 404071 was evaluated in a dextran sulfate sodium (DSS)-induced colitis model.

Female Swiss Webb mice were separated in groups and treated as shown in Table 33.

TABLE 33
Summary of Mice Study Groups

| | | | Human |
|---|---|---|---|
| Group (N) | ASO | DSS | recombinant FXI |
| 1. (8) | None | No | 0 |
| 2. (8) | None | Yes | 0 |
| 3. (8) | F11 (404071) 40 mpk | Yes | 0 |
| 4. (8) | F11 (404071) 40 mpk | Yes | 20 ug/mouse/day |

Two groups 8 Swiss Webb mice were dosed with 40 mg/kg of ISIS 404071 injected subcutaneously twice a week for 3 weeks. Two control groups of 8 Swiss Webb mice were dosed with PBS subcutaneously twice a week for 3 weeks. Both the ASO treated groups and one of the control groups were then given 4% DSS in distilled water for 7 days ad libitum. One of the ASO and DSS treated groups was also given intravenous injections of 20 μg of recombinant human Factor XI protein (Haematologic Technologies Inc.) for 7 consecutive days, starting the day before DSS treatment. Mice were sacrificed at the end of the study on day 7 after DSS was administered.

The effect of antisense inhibition by ISIS 404071 on Factor XI mRNA levels is presented in Table 34. Factor XI levels are significantly decreased in mice treated with ISIS 404071 alone compared to the untreated PBS control.

Mice in all groups were weighed at day 0 and at the end of the study. The results are presented in Table 35 and demonstrate the weight change in the different groups over the time of the study. Mice were sacrificed at the end of the study on day 7 after DSS was administered and their colon lengths were measured. The results are presented in Table 36 and demonstrate that the increase in colon length due to treatment with ISIS 404071 is eliminated by administration of the recombinant Factor XI protein. The stool softness/diarrhea of the mice was analyzed on day 7 after DSS was administered and the score is presented in Table 37. The amelioration of diarrhea in mice treated with ISIS 404071 is eliminated on addition of recombinant Factor XI protein.

Mice with dextran sodium sulfate (DSS)-induced colitis have elevated level of thrombin-antithrombin (TAT) complexes in blood (Anthoni, C. et al., J. Exp. Med. 204: 1595-1601, 2007) and is also observed in patients with ulcerative colitis (Kume, K. et al., Intern Med. 2007. 46: 1323-9). The effect of antisense inhibition of Factor XI on TAT levels in the colon was evaluated (Siemens Healthcare Diagnostics, Deerfield, Ill.) at the end of the study on day 7 after DSS was administered and the results are presented in Table 38. As demonstrated, DSS administration increased TAT levels, which were decreased by treatment with ISIS 404071. Administration of recombinant Factor XI protein caused a near restoration of TAT levels to that of the DSS treated mice.

Plasma levels of soluble CD40 ligand (CD40L) are known to be elevated in cases of inflammatory bowel disease (Ludwiczek, O. et al., Int. J. Colorectal Disease. 2003. 18: 142-147) and may be considered a marker of intestinal inflammation. The effect of antisense inhibition of Factor XI on CD40L levels in the plasma was evaluated at the end of the study on day 7 after DSS was administered and the results are presented in Table 39. CD40L levels in plasma were measured by commercially available ELISA kits (Bender MedSystems, Vienna, Austria, eBioscience, San Diego, Calif.) according to the manufacture's protocols. As demonstrated, DSS administration increased CD40L levels, which were decreased by treatment with ISIS 404071. Administration of recombinant Factor XI protein caused a restoration of CD40L levels to that of the DSS treated mice.

Observations on experimental models and humans with ulcerative colitis suggest a pathogenetic role of the kallikrein-kinin system in inflammatory bowel diseases (Devani, M. et al., Digestive and Liver Disease. 2005. 37: 665-673). The effect of antisense inhibition of Factor XI on kinin levels in the colon was evaluated (Phoenix Pharmaceuticals, Burlingame, Calif.) at the end of the study on day 7 after DSS was administered and the results are presented in Table 40. As demonstrated, DSS administration increased bradykinin levels, which were decreased by treatment with ISIS 404071. Administration of recombinant Factor XI protein caused a restoration of bradykinin levels to that of the DSS treated mice.

The cytokine levels of IFN-γ, IL-10, IL-12, IL-1β, IL-2, IL-4, IL-5, TNF-α, and keratinocyte chemoattractant (KC) were measured in the colon of the mice groups at the end of the study on day 7 after DSS was administered. Colons were homogenized on ice in PBS supplemented with protease inhibitors (Sigma, St. Louis, Mo.) and extracted with rotation at 4° C. for 1 hour. After removal of insoluble material by centrifugation, colon homogenates were used for the cytokine analysis by multiplex ELISA (Mouse TH1/TH2 9-Plex Ultra-Sensitive Kit, Meso Scale Discovery, Gaithersburg, Md.) according to the manufacture's protocol. Cytokine levels in colon extracts were normalized to the protein concentration measured by protein assay kit (BioRad, Hercules, Calif.). The cytokine level results are presented in Table 41. The levels of pro-inflammatory cytokines, IFN-γ, IL-1β, IL-10, IL-2, IL-5, TNF-α, and KC were elevated by DSS administration, and were decreased by treatment with ISIS 404071. Administration of recombinant Factor XI protein caused a restoration of cytokine levels to that of the DSS treated mice.

In summary, this example shows that antisense treatment of DSS induced ulcerative colitis in an animal ameliorated the ulcerative colitis and decreased certain proinflammatory cytokines such as Th1 cytokines INF-γ, IL-10, TNF-α and KC. Additionally, proinflammatory cytokines such as Th2 cytokines IL-4 and IL-5 were decreased compared to untreated mice. This example also shows that human Factor XI protein treatment can successfully reverse the effect of antisense treatment of DSS induced ulcerative colitis in an animal. Therefore, recombinant human Factor XI protein may serve as an antidote for ISIS 404071 treatment.

TABLE 34
Antisense inhibition by ISIS 404071 and recovery of Factor XI levels by
recombinant Factor XI protein compared to untreated PBS control

| | | | Human | |
|---|---|---|---|---|
| | | | recombinant | % |
| Group (N) | ASO | DSS | FXI | inhibition |
| 2. (8) | None | Yes | 0 | 0 |
| 3. (8) | F11 (404071) 40 mpk | Yes | 0 | 82 |
| 4. (8) | F11 (404071) 40 mpk | Yes | 20 ug/mouse/day | 83 |

TABLE 35
Weight change (%) in mice groups

| | | | Human | |
|---|---|---|---|---|
| | | | recombinant | Weight |
| Group (N) | ASO | DSS | FXI | change |
| 1. (8) | None | No | 0 | +1.1 |
| 2. (8) | None | Yes | 0 | −0.4 |
| 3. (8) | F11 (404071) 40 mpk | Yes | 0 | +1.8 |
| 4. (8) | F11 (404071) 40 mpk | Yes | 20 ug/mouse/day | +0.3 |

TABLE 36
Colon length in mice groups

| | | | Human | Colon |
|---|---|---|---|---|
| | | | recombinant | length |
| Group (N) | ASO | DSS | FXI | (cm) |
| 1. (8) | None | No | 0 | 11.3 |
| 2. (8) | None | Yes | 0 | 7.9 |
| 3. (8) | F11 (404071) 40 mpk | Yes | 0 | 9.9 |
| 4. (8) | F11 (404071) 40 mpk | Yes | 20 ug/mouse/day | 7.3 |

TABLE 37
Stool analysis in mice groups

| | | | Human | |
|---|---|---|---|---|
| | | | recombinant | Diarrhea |
| Group (N) | ASO | DSS | FXI | score |
| 1. (8) | None | No | 0 | 0.0 |
| 2. (8) | None | Yes | 0 | 2.7 |
| 3. (8) | F11 (404071) 40 mpk | Yes | 0 | 0.5 |
| 4. (8) | F11 (404071) 40 mpk | Yes | 20 ug/mouse/day | 2.0 |

TABLE 38
Thrombin-antithrombin (TAT) levels in mice groups

| | | | Human | TAT levels |
|---|---|---|---|---|
| Group | | | recombinant | (ng/mg |
| (N) | ASO | DSS | FXI | protein) |
| 1. (8) | None | No | 0 | 0.2 |
| 2. (8) | None | Yes | 0 | 1.5 |
| 3. (8) | F11 (404071) 40 mpk | Yes | 0 | 0.1 |
| 4. (8) | F11 (404071) 40 mpk | Yes | 20 ug/mouse/day | 0.8 |

TABLE 39
CD40L plasma levels in mice groups

| | | | Human | CD40L |
|---|---|---|---|---|
| | | | recombinant | levels |
| Group (N) | ASO | DSS | FXI | (ng/mL) |
| 1. (8) | None | No | 0 | 1.2 |
| 2. (8) | None | Yes | 0 | 1.8 |
| 3. (8) | F11 (404071) 40 mpk | Yes | 0 | 1.5 |
| 4. (8) | F11 (404071) 40 mpk | Yes | 20 ug/mouse/day | 1.6 |

TABLE 40
Bradykinin levels in the colons of mice groups

| | | | Human | Bradykinin |
|---|---|---|---|---|
| Group | | | recombinant | levels |
| (N) | ASO | DSS | FXI | (ng/mg) |
| 1. (8) | None | No | 0 | 0.0 |
| 2. (8) | None | Yes | 0 | 0.4 |
| 3. (8) | F11 (404071) 40 mpk | Yes | 0 | 0.1 |
| 4. (8) | F11 (404071) 40 mpk | Yes | 20 ug/mouse/day | 0.3 |

TABLE 41
Cytokine levels (pg/mg) in the colons of mice groups

| Group | | | | | | | | | | | | |
|---|---|---|---|---|---|---|---|---|---|---|---|---|
| (N) | ASO | DSS | rFXI | IFN-γ | IL-10 | IL-12 | IL-1β | IL-2 | IL-4 | IL-5 | TNF-α | KC |
| 1. (8) | None | No | 0 | 0.08 | 2.3 | 47 | 5 | 0.08 | 0.004 | 0.04 | 0.09 | 9 |
| 2. (8) | None | Yes | 0 | 6.65 | 7.1 | 63 | 150 | 0.32 | 0.020 | 0.13 | 0.77 | 56 |
| 3. (8) | 404071 | Yes | 0 | 1.10 | 4.7 | 64 | 50 | 0.11 | 0.012 | 0.04 | 0.24 | 27 |
| 4. (8) | 404071 | Yes | 20 ug/day | 4.51 | 5.8 | 96 | 103 | 0.28 | 0.004 | 0.17 | 0.91 | 62 |

### Colitis Study F: Effect of ISIS 404071 on Colitis

The effect of inhibiting Factor XI with ISIS 404071 in ameliorating colitis was evaluated in a dextran sulfate sodium (DSS)-induced colitis model.

Female Swiss Webb mice were separated in groups and treated as shown in Table 42.

TABLE 42
Summary of Mice Study Groups

| Group (N) | ASO | DSS |
|---|---|---|
| 1. (8) | None | No |
| 2. (8) | None | Yes |
| 3. (8) | F11 (404071) 50 mpk | Yes |

In a first group of 8 Swiss Webb mice, 50 mg/kg of ISIS 404071 was injected subcutaneously twice a week for 3 weeks. Two control groups of 8 mice each were injected with PBS twice a week for 3 weeks. After the oligonucleotide treatment, 4% DSS in water was administered ad libitum for 7 days to the experimental group and one PBS control group. Mice were sacrificed at the end of the study on day 7 after DSS was administered.

Mice in all groups were weighed at day 0 and daily for 7 days after administration of DSS. The weight of the mice at day 0 and 7 are shown in Table 43. The stool softness/diarrhea of the mice was analyzed for seven days after DSS was administered as shown in Table 44. Mice were sacrificed at the end of the study on day 7 after DSS was administered and their colon lengths and weight were measured as shown in Table 45.

The antisense oligonucleotide showed statistically significant dose effects on diarrhea scores and colon length (Tables 44 and 45). ISIS 404071 did not significantly affect body weight when compared to placebo as shown in Table 43.

The mRNA levels of cytokines IL-1, IL-6, IL-10, IL-12, IL-17 and TNF-α were measured in the colon of the mice groups at the end of the study on day 7 after DSS was administered. Colons were homogenized on ice in PBS supplemented with protease inhibitors (Sigma, St. Louis, Mo.) and extracted with rotation at 4° C. for 1 hour. After removal of insoluble material by centrifugation, colon homogenates were used for the cytokine analysis by multiplex ELISA (Meso Scale Discovery, Gaithersburg, Md.) according to the manufacture's protocol. Cytokine levels in colon extracts were normalized to the protein concentration measured by protein assay kit (BioRad, Hercules, Calif.). The results are presented in Table 46. The levels of pro-inflammatory cytokines, including Th1 cytokines IL-1 and IL-6, elevated by DSS administration were decreased by treatment with ISIS 404071. GATA-3 is a transcription factor and has been shown to promote secretion of cytokines IL-4, IL-5 and IL-13 from Th2 cells. The effect of antisense oligonucleotide on GATA-3 is presented in Table 46.

Plasma levels of aminotransferases (ALT and AST), blood urea nitrogen (BUN), creatinine (CREAT), cholesterol (CHOL) and total bilirubin (TBIL) were measured after treatment and are shown in Table 47.

The effect of antisense inhibition in the liver and colon levels of Factor XI mRNA after treatment by ISIS 404071 is presented in Table 48 (as a percent of the PBS treated control).

In summary, this example shows that Factor XI oligonucleotide treatment significantly ameliorated DSS induced ulcerative colitis in an animal. Treatment with a Factor XI inhibitor is also shown to reduce the risk and progression of colitis in an animal. Treatment with a Factor XI inhibitor also decreased Th1 cytokines IL-1 and IL-6

TABLE 43
Body weight (grams) change after treatment

| | Weight | Weight |
|---|---|---|
| | (day 0) | (day 7) |
| PBS | 28.3 | 29.6 |
| DSS control | 26.8 | 23.1 |
| ISIS 404071 | 28 | 24.7 |

TABLE 44
Stool scores after treatment with ISIS 404071

| | Day 3 | Day 4 | Day 5 | Day 6 | Day 7 | Day 8 |
|---|---|---|---|---|---|---|
| PBS | 0 | 0 | 0 | 0 | 0 | 0 |
| DSS | 0 | 0 | 1 | 1 | 1.63 | 2.5 |
| ISIS | 0 | 0.25 | 1 | 1 | 1 | 2 |
| 404071 | | | | | | |

TABLE 45
Colon length and weight after treatment

| | Length | Weight |
|---|---|---|
| | (cm) | (mg) |
| PBS | 10.8 | 293.8 |
| DSS control | 5.9 | 276.4 |
| ISIS 404071 | 7.8 | 339.3 |

TABLE 46
Cytokine mRNA levels (as a % of PBS treated animals)
in the colon after treatment with ISIS 404071

| | IL-1 | IL-6 | IL-10 | IL-12 | IL-17 | TNF-α | GATA-3 |
|---|---|---|---|---|---|---|---|
| PBS | 100 | 100 | 100 | 100 | 100 | 100 | 100 |
| DSS | 3312 | 1515 | 103 | 244 | 1478 | 520 | 445 |
| ISIS | 1189 | 1056 | 202 | 234 | 787 | 1127 | 359 |
| 404071 | | | | | | | |

TABLE 47
Plasma levels of ALT, AST, BUN, CREAT, CHOL
and TBIL after treatment with ISIS 404071

| | ALT | AST | BUN | CREAT | CHOL | TBIL |
|---|---|---|---|---|---|---|
| | (U/L) | (U/L) | (mg/dl) | (mg/dl) | (mg/dl) | (mg/dl) |
| PBS | 166 | 207.6 | 35.2 | 0.048 | 105.6 | 0.25 |
| DSS control | 56.8 | 192.9 | 31.6 | 0.089 | 175.8 | 0.29 |
| ISIS 404071 | 133.6 | 339.3 | 29.5 | −0.003 | 170.3 | 0.7 |

TABLE 48
Factor XI mRNA levels in the liver and colon after treatment with
ISIS 404071 compared to PBS treatment

| | Liver | Colon |
|---|---|---|
| | (% of normal) | (% of normal) |
| PBS | 100 | 100 |
| DSS control | 256 | 166 |
| ISIS 404071 | 24 | 148 |

### Example 13

### In Vivo Effect of Antisense Inhibition of Murine Factor XI on an Ova-Induced Asthma Model

The effect of antisense oligonucleotide inhibition of Factor XI and its role in ameliorating asthma was evaluated in an OVA/alum-induced asthma model. Administration of ovalbumin to animals is a well known method to induce colitis and has been previously described by Henderson et al. (J. Exp. Med., 1996, 184: 1483-1494).

Asthma in humans has symptoms that can include wheezing, dyspnea, non-productive coughing, chest tightness and pain, rapid heart rate and sweating. During asthma attacks or exacerbation of asthma, there is inflammation in the lung tissue, constriction of the smooth muscle cells of the bronchi, blockade of airways and difficulty in breathing (Fanta, C. H. N. Engl. J. Med. 2009. 360: 1002-1014).

Antisense oligonucleotides targeting Factor XI were described in Example 11, supra.

### Treatment

BALB/c mice (available from Charles River Laboratories, Wilmington, Mass.) were maintained on a 12-hour light/dark cycle and fed ad libitum Teklad lab chow (Harlan Laboratories, Indianapolis, Ind.). Animals were acclimated for at least 7 days in the research facility before initiation of the experiment. Antisense oligonucleotides (ASOs) were prepared in PBS and sterilized by filtering through a 0.2 micron filter. Oligonucleotides were dissolved in 0.9% PBS for injection.

The mice were divided into four treatment groups of 5 mice each. One group received subcutaneous injections of ISIS 404071 at a dose of 50 mg/kg twice a week for 4 weeks. One group of mice received subcutaneous injections of control oligonucleotide, ISIS 421208, which is a mismatch oligonucleotide sequence of ISIS 404071, at a dose of 50 mg/kg twice a week for 4 weeks. Two groups of mice received subcutaneous injections of PBS twice a week for 4 weeks. One PBS group remained untreated and served as the control group. The second PBS group and both the oligonucleotide treated groups were injected with OVA/alum on days 0 and 14 and nebulized with OVA in PBS on days 24, 25, and 26. The first OVA application served to sensitize the mice against OVA while the second was a challenge application to provoke an asthmatic reaction. Two days following the final dose, the mice were euthanized, bronchial lavage (BAL) was collected and analyses done.

Bronchial asthma, even in its mild form, is characterized by local infiltration and activation of inflammatory and immunoeffector cells, including T lymphocytes, macrophages, eosinophils, and mast cells (Smith D. L. et al., Am. Rev. Respir. Dis. 1993. 148: 523-532). The effect of treatment with ISIS 404071 on bronchoalveolar lavage (BAL) eosinophil recruitment was assessed. BAL cells were stained with hemotoxylin and eosin (H&E). The results are presented in Table 49 as a percentage of total cells in BAL. The data demonstrates that treatment with ISIS 404071 decreased the eosinophil recruitment.

Lung sections were stained with periodic acid shift (PAS) base stain that stains mucus, which is produced during asthma attacks (Rogers, D. F. Curr. Opin Pharmacol. 2004. 4: 241-250). The number of airways containing mucus as a percentage of the total airways in the lungs was evaluated. The data is presented in Table 50 and indicates that treatment with ISIS 404071 decreased mucus production in the lungs

TABLE 49
BAL eosinophil recruitment after treatment

| | Eosinophils |
|---|---|
| | (%) |
| PBS | 0 |
| OVA control | 48 |
| ISIS 404071 | 32 |
| ISIS 421208 | 51 |

TABLE 50
Mucus production after treatment

| | % airways |
|---|---|
| PBS | 0 |
| OVA control | 58 |
| ISIS 404071 | 34 |
| ISIS 421208 | 49 |

In summary, this example shows that Factor XI oligonucleotide treatment significantly ameliorated OVA-induced asthma in an animal. Treatment with a Factor XI inhibitor is also shown to reduce the risk and progression of asthma in an animal.

### Example 14

### Antisense Inhibition of Human Factor XI in HepG2 Cells by Oligonucleotides Designed by Microwalk

Additional gapmers were designed based on ISIS 416850 and ISIS 416858 (see Table 8 above). These gapmers were shifted slightly upstream and downstream (i.e. “microwalk”) of ISIS 416850 and ISIS 416858. The microwalk gapmers were designed with either 5-8-5 MOE or 6-8-6 MOE motifs.

These microwalk gapmers were tested in vitro. Cultured HepG2 cells at a density of 20,000 cells per well were transfected using electroporation with 8,000 nM antisense oligonucleotide. After a treatment period of approximately 24 hours, RNA was isolated from the cells and Factor XI mRNA levels were measured by quantitative real-time PCR. Factor XI mRNA levels were adjusted according to total RNA content, as measured by RIBOGREEN. Results are presented as percent inhibition of Factor XI, relative to untreated control cells.

ISIS 416850 and ISIS 416858, as well as selected gapmers from Tables 1 and 8 (i.e., ISIS 412206, ISIS 412223, ISIS 412224, ISIS 412225, ISIS 413481, ISIS 413482, ISIS 416825, ISIS 416848, ISIS 416849, ISIS 416850, ISIS 416851, ISIS 416852, ISIS 416853, ISIS 416854, ISIS 416855, ISIS 416856, ISIS 416857, ISIS 416858, ISIS 416859, ISIS 416860, ISIS 416861, ISIS 416862, ISIS 416863, ISIS 416864, ISIS 416865, ISIS 416866, and ISIS 416867) were retested in vitro along with the microwalk gapmers under the same condition as described above.

The chimeric antisense oligonucleotides in Table 51 were designed as 5-10-5 MOE, 5-8-5 and 6-8-6 MOE gapmers. The first two listed gapmers in Table 51 are the original gapmers (ISIS 416850 and ISIS 416858) from which ISIS 445493-445543 were designed via microwalk, and are designated by an asterisk. The 5-10-5 gapmers are 20 nucleotides in length, wherein the central gap segment is comprised of ten 2′-deoxynucleotides and is flanked on both sides (in the 5′ and 3′ directions) by wings comprising five nucleotides each. The 5-8-5 gapmers are 18 nucleotides in length, wherein the central gap segment is comprised of eight 2′-deoxynucleotides and is flanked on both sides (in the 5′ and 3′ directions) by wings comprising five nucleotides each. The 6-8-6 gapmers are 20 nucleotides in length, wherein the central gap segment is comprised of eight 2′-deoxynucleotides and is flanked on both sides (in the 5′ and 3′ directions) by wings comprising six nucleotides each. For each of the motifs (5-10-5, 5-8-5 and 6-8-6), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Human Target start site” indicates the 5′-most nucleotide to which the gapmer is targeted in the human sequence. “Human Target stop site” indicates the 3′-most nucleotide to which the gapmer is targeted in the human sequence. Each gapmer listed in Table 51 is targeted to SEQ ID NO: 1 (GENBANK Accession No. NM_{—}000128.3). Each gapmer is Table 51 is also fully cross-reactive with the rhesus monkey Factor XI gene sequence, designated herein as SEQ ID NO: 274 (exons 1-15 GENBANK Accession No. NW_{—}001118167.1). ‘Rhesus monkey start site’ indicates the 5′-most nucleotide to which the gapmer is targeted in the rhesus monkey sequence. ‘Rhesus monkey stop site’ indicates the 3′-most nucleotide to which the gapmer is targeted to the rhesus monkey sequence.

As shown in Table 51, all of the microwalk designed gapmers targeted to the target region beginning at the target start site 1275 and ending at the target stop site 1317 (i.e. nucleobases 1275-1317) of SEQ ID NO: 1 exhibited at least 60% inhibition of Factor XI mRNA. Similarly, all of the re-tested gapmers from Tables 1 and 8 exhibited at least 60% inhibition.

Several of the gapmers exhibited at least 70% inhibition, including ISIS numbers: ISIS 412206, 412224, 412225, 413481, 413482, 416825, 416848, 416849, 416850, 416851, 416852, 416853, 416854, 416855, 416856, 416857, 416858, 416859, 416860, 416861, 416862, 416863, 416864, 416865, 416866, 416867, 445494, 445495, 445496, 445497, 445498, 445499, 445500, 445501, 445502, 445503, 445504, 445505, 445506, 445507, 445508, 445509, 445510, 445511, 445512, 445513, 445514, 445515, 445516, 445517, 445518, 445519, 445520, 445521, 445522, 445523, 445524, 445525, 445526, 445527, 445528, 445529, 445530, 445531, 445532, 445533, 445534, 445535, 445536, 445537, 455538, 445539, 445540, 445541, 445542, and 445543.

Several of the gapmers exhibited at least 80% inhibition, including ISIS numbers: ISIS 412206, 412224, 412225, 413481, 413482, 416825, 416848, 416849, 416850, 416851, 416852, 416853, 416854, 416855, 416856, 416857, 416858, 416859, 416860, 416861, 416862, 416863, 416864, 416865, 416866, 416867, 445494, 445495, 445496, 445497, 445498, 445500, 445501, 445502, 445503, 445504, 445505, 445506, 445507, 445508, 445509, 445510, 445513, 445514, 445519, 445520, 445521, 445522, 445525, 445526, 445529, 445530, 445531, 445532, 445533, 445534, 445535, 445536, 455538, 445541, and 445542.

Several of the gapmers exhibited at least 90% inhibition, including ISIS numbers: ISIS 412206, 416825, 416850, 416857, 416858, 416861, 445522, and 445531.

TABLE 51
Inhibition of human Factor XI mRNA levels by chimeric antisense
oligonucleotides targeted to SEQ ID NO: 1
(GENBANK Accession No. NM_000128.3)

| | Human | Human | | | | SEQ | rhesus | Rhesus |
|---|---|---|---|---|---|---|---|---|
| | Start | Stop | | Percent | | ID | monkey | monkey |
| ISIS No. | Site | Site | Sequence (5′ to 3′) | inhibition | Motif | No. | Start Site | Stop Site |
| *416850 | 1278 | 1297 | TGCACAGTTTCTGGCAG | 91 | 5/10/2005 | 215 | 1277 | 1296 |
| | | | GCC | | | | | |
| *416858 | 1288 | 1307 | ACGGCATTGGTGCACAG | 90 | 5/10/2005 | 223 | 1287 | 1306 |
| | | | TTT | | | | | |
| 416825 | 680 | 699 | GCCCTTCATGTCTAGGT | 90 | 5/10/2005 | 190 | 679 | 698 |
| | | | CCA | | | | | |
| 412206 | 738 | 757 | CCGTGCATCTTTCTTGG | 91 | 5/10/2005 | 34 | 737 | 756 |
| | | | CAT | | | | | |
| 412223 | 1275 | 1294 | ACAGTTTCTGGCAGGCC | 62 | 5/10/2005 | 51 | 1274 | 1293 |
| | | | TCG | | | | | |
| 445493 | 1275 | 1294 | ACAGTTTCTGGCAGGCC | 69 | 6/8/2006 | 51 | 1274 | 1293 |
| | | | TCG | | | | | |
| 445518 | 1275 | 1292 | AGTTTCTGGCAGGCCTC | 75 | 5/8/2005 | 242 | 1274 | 1291 |
| | | | G | | | | | |
| 416848 | 1276 | 1295 | CACAGTTTCTGGCAGGC | 87 | 5/10/2005 | 213 | 1275 | 1294 |
| | | | CTC | | | | | |
| 445494 | 1276 | 1295 | CACAGTTTCTGGCAGGC | 85 | 6/8/2006 | 213 | 1275 | 1294 |
| | | | CTC | | | | | |
| 445519 | 1276 | 1293 | CAGTTTCTGGCAGGCCT | 81 | 5/8/2005 | 243 | 1275 | 1292 |
| | | | C | | | | | |
| 416849 | 1277 | 1296 | GCACAGTTTCTGGCAGG | 88 | 5/10/2005 | 214 | 1276 | 1295 |
| | | | CCT | | | | | |
| 445495 | 1277 | 1296 | GCACAGTTTCTGGCAGG | 89 | 6/8/2006 | 214 | 1276 | 1295 |
| | | | CCT | | | | | |
| 445520 | 1277 | 1294 | ACAGTTTCTGGCAGGCC | 82 | 5/8/2005 | 244 | 1276 | 1293 |
| | | | T | | | | | |
| 445496 | 1278 | 1297 | TGCACAGTTTCTGGCAG | 87 | 6/8/2006 | 215 | 1277 | 1296 |
| | | | GCC | | | | | |
| 445521 | 1278 | 1295 | CACAGTTTCTGGCAGGC | 87 | 5/8/2005 | 245 | 1277 | 1294 |
| | | | C | | | | | |
| 416851 | 1279 | 1298 | GTGCACAGTTTCTGGCA | 89 | 5/10/2005 | 216 | 1278 | 1297 |
| | | | GGC | | | | | |
| 445497 | 1279 | 1298 | GTGCACAGTTTCTGGCA | 81 | 6/8/2006 | 216 | 1278 | 1297 |
| | | | GGC | | | | | |
| 445522 | 1279 | 1296 | GCACAGTTTCTGGCAGG | 91 | 5/8/2005 | 246 | 1278 | 1295 |
| | | | C | | | | | |
| 413481 | 1280 | 1299 | GGTGCACAGTTTCTGGC | 82 | 5/10/2005 | 114 | 1279 | 1298 |
| | | | AGG | | | | | |
| 445498 | 1280 | 1299 | GGTGCACAGTTTCTGGC | 83 | 6/8/2006 | 114 | 1279 | 1298 |
| | | | AGG | | | | | |
| 445523 | 1280 | 1297 | TGCACAGTTTCTGGCAG | 73 | 5/8/2005 | 267 | 1279 | 1296 |
| | | | G | | | | | |
| 416852 | 1281 | 1300 | TGGTGCACAGTTTCTGG | 87 | 5/10/2005 | 217 | 1280 | 1299 |
| | | | CAG | | | | | |
| 445499 | 1281 | 1300 | TGGTGCACAGTTTCTGG | 75 | 6/8/2006 | 217 | 1280 | 1299 |
| | | | CAG | | | | | |
| 445524 | 1281 | 1298 | GTGCACAGTTTCTGGCA | 75 | 5/8/2005 | 247 | 1280 | 1297 |
| | | | G | | | | | |
| 416853 | 1282 | 1301 | TTGGTGCACAGTTTCTG | 84 | 5/10/2005 | 218 | 1281 | 1300 |
| | | | GCA | | | | | |
| 445500 | 1282 | 1301 | TTGGTGCACAGTTTCTG | 81 | 6/8/2006 | 218 | 1281 | 1300 |
| | | | GCA | | | | | |
| 445525 | 1282 | 1299 | GGTGCACAGTTTCTGGC | 85 | 5/8/2005 | 248 | 1281 | 1298 |
| | | | A | | | | | |
| 416854 | 1283 | 1302 | ATTGGTGCACAGTTTCT | 86 | 5/10/2005 | 219 | 1282 | 1301 |
| | | | GGC | | | | | |
| 445501 | 1283 | 1302 | ATTGGTGCACAGTTTCT | 83 | 6/8/2006 | 219 | 1282 | 1301 |
| | | | GGC | | | | | |
| 445526 | 1283 | 1300 | TGGTGCACAGTTTCTGG | 81 | 5/8/2005 | 249 | 1282 | 1299 |
| | | | C | | | | | |
| 416855 | 1284 | 1303 | CATTGGTGCACAGTTTC | 85 | 5/10/2005 | 220 | 1283 | 1302 |
| | | | TGG | | | | | |
| 445502 | 1284 | 1303 | CATTGGTGCACAGTTTC | 83 | 6/8/2006 | 220 | 1283 | 1302 |
| | | | TGG | | | | | |
| 445527 | 1284 | 1301 | TTGGTGCACAGTTTCTG | 70 | 5/8/2005 | 250 | 1283 | 1300 |
| | | | G | | | | | |
| 412224 | 1285 | 1304 | GCATTGGTGCACAGTTT | 84 | 5/10/200 | 552 | 1284 | 1303 |
| | | | CTG | | | | | |
| 445503 | 1285 | 1304 | GCATTGGTGCACAGTTT | 89 | 6/8/200 | 652 | 1284 | 1303 |
| | | | CTG | | | | | |
| 445528 | 1285 | 1302 | ATTGGTGCACAGTTTCT | 73 | 5/8/2005 | 251 | 1284 | 1301 |
| | | | G | | | | | |
| 416856 | 1286 | 1305 | GGCATTGGTGCACAGTT | 84 | 5/10/2005 | 221 | 1285 | 1304 |
| | | | TCT | | | | | |
| 445504 | 1286 | 1305 | GGCATTGGTGCACAGTT | 87 | 6/8/2006 | 221 | 1285 | 1304 |
| | | | TCT | | | | | |
| 445529 | 1286 | 1303 | CATTGGTGCACAGTTTC | 85 | 5/8/2005 | 252 | 1285 | 1302 |
| | | | T | | | | | |
| 416857 | 1287 | 1306 | CGGCATTGGTGCACAGT | 91 | 5/10/2005 | 222 | 1286 | 1305 |
| | | | TTC | | | | | |
| 445505 | 1287 | 1306 | CGGCATTGGTGCACAGT | 89 | 6/8/2006 | 222 | 1286 | 1305 |
| | | | TTC | | | | | |
| 445530 | 1287 | 1304 | GCATTGGTGCACAGTTT | 83 | 5/8/2005 | 253 | 1286 | 1303 |
| | | | C | | | | | |
| 445506 | 1288 | 1307 | ACGGCATTGGTGCACAG | 86 | 6/8/2006 | 223 | 1287 | 1306 |
| | | | TTT | | | | | |
| 445531 | 1288 | 1305 | GGCATTGGTGCACAGTT | 90 | 5/8/2005 | 254 | 1287 | 1304 |
| | | | T | | | | | |
| 416859 | 1289 | 1308 | GACGGCATTGGTGCACA | 85 | 5/10/2005 | 224 | 1288 | 1307 |
| | | | GTT | | | | | |
| 445507 | 1289 | 1308 | GACGGCATTGGTGCACA | 85 | 6/8/2006 | 224 | 1288 | 1307 |
| | | | GTT | | | | | |
| 445532 | 1289 | 1306 | CGGCATTGGTGCACAGT | 89 | 5/8/2005 | 255 | 1288 | 1305 |
| | | | T | | | | | |
| 413482 | 1290 | 1309 | GGACGGCATTGGTGCAC | 88 | 5/10/2005 | 115 | 1289 | 1308 |
| | | | AGT | | | | | |
| 445508 | 1290 | 1309 | GGACGGCATTGGTGCAC | 81 | 6/8/2006 | 115 | 1289 | 1308 |
| | | | AGT | | | | | |
| 445533 | 1290 | 1307 | ACGGCATTGGTGCACAG | 87 | 5/8/2005 | 256 | 1289 | 1306 |
| | | | T | | | | | |
| 416860 | 1291 | 1310 | CGGACGGCATTGGTGCA | 89 | 5/10/2005 | 225 | 1290 | 1309 |
| | | | CAG | | | | | |
| 445509 | 1291 | 1310 | CGGACGGCATTGGTGCA | 84 | 6/8/2006 | 225 | 1290 | 1309 |
| | | | CAG | | | | | |
| 445534 | 1291 | 1308 | GACGGCATTGGTGCACA | 82 | 5/8/2005 | 257 | 1290 | 1307 |
| | | | G | | | | | |
| 416861 | 1292 | 1311 | GCGGACGGCATTGGTGC | 90 | 5/10/2005 | 226 | 1291 | 1310 |
| | | | ACA | | | | | |
| 445510 | 1292 | 1311 | GCGGACGGCATTGGTGC | 88 | 6/8/2006 | 226 | 1291 | 1310 |
| | | | ACA | | | | | |
| 445535 | 1292 | 1309 | GGACGGCATTGGTGCAC | 83 | 5/8/2005 | 258 | 1291 | 1308 |
| | | | A | | | | | |
| 416862 | 1293 | 1312 | AGCGGACGGCATTGGTG | 89 | 5/10/2005 | 227 | 1292 | 1311 |
| | | | CAC | | | | | |
| 445511 | 1293 | 1312 | AGCGGACGGCATTGGTG | 77 | 6/8/2006 | 227 | 1292 | 1311 |
| | | | CAC | | | | | |
| 445536 | 1293 | 1310 | CGGACGGCATTGGTGCA | 82 | 5/8/2005 | 259 | 1292 | 1309 |
| | | | C | | | | | |
| 416863 | 1294 | 1313 | CAGCGGACGGCATTGGT | 86 | 5/10/2005 | 228 | 1293 | 1312 |
| | | | GCA | | | | | |
| 445512 | 1294 | 1313 | CAGCGGACGGCATTGGT | 79 | 6/8/2006 | 228 | 1293 | 1312 |
| | | | GCA | | | | | |
| 445537 | 1294 | 1311 | GCGGACGGCATTGGTGC | 78 | 5/8/2005 | 260 | 1293 | 1310 |
| | | | A | | | | | |
| 412225 | 1295 | 1314 | GCAGCGGACGGCATTGG | 86 | 5/10/2005 | 53 | 1294 | 1313 |
| | | | TGC | | | | | |
| 445513 | 1295 | 1314 | GCAGCGGACGGCATTGG | 85 | 6/8/2006 | 53 | 1294 | 1313 |
| | | | TGC | | | | | |
| 445538 | 1295 | 1312 | AGCGGACGGCATTGGTG | 80 | 5/8/2005 | 261 | 1294 | 1311 |
| | | | C | | | | | |
| 416864 | 1296 | 1315 | GGCAGCGGACGGCATTG | 88 | 5/10/2005 | 229 | 1295 | 1314 |
| | | | GTG | | | | | |
| 445514 | 1296 | 1315 | GGCAGCGGACGGCATTG | 81 | 6/8/2006 | 229 | 1295 | 1314 |
| | | | GTG | | | | | |
| 445539 | 1296 | 1313 | CAGCGGACGGCATTGGT | 79 | 5/8/2005 | 262 | 1295 | 1312 |
| | | | G | | | | | |
| 416865 | 1297 | 1316 | TGGCAGCGGACGGCATT | 86 | 5/10/2005 | 230 | 1296 | 1315 |
| | | | GGT | | | | | |
| 445515 | 1297 | 1316 | TGGCAGCGGACGGCATT | 75 | 6/8/2006 | 230 | 1296 | 1315 |
| | | | GGT | | | | | |
| 445540 | 1297 | 1314 | GCAGCGGACGGCATTGG | 74 | 5/8/2005 | 263 | 1296 | 1313 |
| | | | T | | | | | |
| 416866 | 1298 | 1317 | CTGGCAGCGGACGGCAT | 84 | 5/10/2005 | 231 | 1297 | 1316 |
| | | | TGG | | | | | |
| 445516 | 1298 | 1317 | CTGGCAGCGGACGGCAT | 79 | 6/8/2006 | 231 | 1297 | 1316 |
| | | | TGG | | | | | |
| 445541 | 1298 | 1315 | GGCAGCGGACGGCATTG | 80 | 5/8/2005 | 264 | 1297 | 1314 |
| | | | G | | | | | |
| 416867 | 1299 | 1318 | ACTGGCAGCGGACGGCA | 85 | 5/10/2005 | 232 | 1298 | 1317 |
| | | | TTG | | | | | |
| 445517 | 1299 | 1318 | ACTGGCAGCGGACGGCA | 74 | 6/8/2006 | 232 | 1298 | 1317 |
| | | | TTG | | | | | |
| 445542 | 1299 | 1316 | TGGCAGCGGACGGCATT | 83 | 5/8/2005 | 265 | 1298 | 1315 |
| | | | G | | | | | |
| 445543 | 1300 | 1317 | CTGGCAGCGGACGGCAT | 74 | 5/8/2005 | 266 | 1299 | 1316 |
| | | | T | | | | | |

### Example 15

### Dose-Dependent Antisense Inhibition of Human Factor XI in HepG2 Cells

Gapmers from Example 14 exhibiting in vitro inhibition of human Factor XI were tested at various doses in HepG2 cells. Cells were plated at a density of 20,000 cells per well and transfected using electroporation with 123.46 nM, 370.37 nM, 1,111.11 nM, 3,333.33 nM and 10,000 nM concentrations of antisense oligonucleotide, as specified in Table 52. After a treatment period of approximately 16 hours, RNA was isolated from the cells and Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN. Results are presented as percent inhibition of Factor XI, relative to untreated control cells. As illustrated in Table 52, Factor XI mRNA levels were reduced in a dose-dependent manner in antisense oligonucleotide treated cells.

The half maximal inhibitory concentration (IC₅₀) of each oligonucleotide was calculated by plotting the concentrations of antisense oligonucleotides used versus the percent inhibition of Factor XI mRNA expression achieved at each concentration, and noting the concentration of antisense oligonucleotide at which 50% inhibition of Factor XI mRNA expression was achieved compared to the PBS control. IC₅₀ values are presented in Table 52.

TABLE 52
Dose-dependent antisense inhibition of human Factor XI in HepG2
cells via transfection of oligonucleotides using electroporation

| ISIS | 123.47 | 370.37 | 1,111.11 | 3,333.33 | 10,000.0 | IC₅₀ |
|---|---|---|---|---|---|---|
| No. | nM | nM | nM | nM | nM | (μM) |
| 416849 | 5 | 5 | 26 | 57 | 68 | 2.7 |
| 416850 | 0 | 12 | 36 | 74 | 73 | 2.8 |
| 416851 | 13 | 35 | 36 | 64 | 72 | 1.5 |
| 416856 | 12 | 23 | 35 | 59 | 83 | 1.6 |
| 416857 | 2 | 20 | 35 | 62 | 72 | 2.3 |
| 416858 | 0 | 27 | 36 | 64 | 70 | 2.2 |
| 416860 | 0 | 28 | 39 | 41 | 40 | n.d. |
| 416861 | 0 | 15 | 27 | 66 | 80 | 2.0 |
| 445498 | 3 | 1 | 27 | 50 | 58 | 4.8 |
| 445503 | 0 | 0 | 22 | 36 | 60 | 5.9 |
| 445504 | 8 | 20 | 38 | 53 | 68 | 2.7 |
| 445505 | 12 | 30 | 39 | 59 | 77 | 1.8 |
| 445522 | 0 | 0 | 44 | 63 | 74 | 2.9 |
| 445531 | 8 | 16 | 52 | 61 | 77 | 1.8 |
| 445532 | 5 | 12 | 39 | 60 | 70 | 2.0 |

n.d. = no data

### Example 16

### Dose-Dependent Antisense Inhibition of Human Factor XI in HepG2 Cells by Oligonucleotides Designed by Microwalk

Additional gapmers were designed based on ISIS 416850 and ISIS 416858 (see Table 8 above). These gapmers are shifted slightly upstream and downstream (i.e. microwalk) of ISIS 416850 and ISIS 416858. Gapmers designed by microwalk have 3-8-3 MOE, 4-8-4 MOE, 2-10-2 MOE, 3-10-3 MOE, or 4-10-4 MOE motifs.

These gapmers were tested at various doses in HepG2 cells. Cells were plated at a density of 20,000 cells per well and transfected using electroporation with 375 nM, 750 nM, 1,500 nM, 3,000 nM, 6,000 nM and 12,000 nM concentrations of antisense oligonucleotide, as specified in Table 54. After a treatment period of approximately 16 hours, RNA was isolated from the cells and Factor XI mRNA levels were measured by quantitative real-time PCR. Human Factor XI primer probe set RTS 2966 was used to measure mRNA levels. Factor XI mRNA levels were adjusted according to total RNA content, as measured by RIBOGREEN. Results are presented as percent inhibition of Factor XI, relative to untreated control cells.

ISIS 416850, ISIS 416858, ISIS 445522, and ISIS 445531 (see Table 52 above) were re-tested in vitro along with the microwalk gapmers under the same conditions described above.

The chimeric antisense oligonucleotides in Table 53 were designed as 3-8-3 MOE, 4-8-4 MOE, 2-10-2 MOE, 3-10-3 MOE, or 4-10-4 MOE gapmers. The 3-8-3 gapmer is 14 nucleotides in length, wherein the central gap segment is comprised of eight 2′-deoxynucleotides and is flanked on both sides (in the 5′ and 3′ directions) by wings comprising three nucleotides each. The 4-8-4 gapmer is 16 nucleotides in length, wherein the central gap segment is comprised of eight 2′-deoxynucleotides and is flanked on both sides (in the 5′ and 3′ directions) by wings comprising four nucleotides each. The 2-10-2 gapmer is 14 nucleotides in length, wherein the central gap segment is comprised of ten 2′-deoxynucleotides and is flanked on both sides (in the 5′ and 3′ directions) by wings comprising two nucleotides each. The 3-10-3 gapmer is 16 nucleotides in length, wherein the central gap segment is comprised of ten 2′-deoxynucleotides and is flanked on both sides (in the 5′ and 3′ directions) by wings comprising three nucleotides each. The 4-10-4 gapmer is 18 nucleotides in length, wherein the central gap segment is comprised of ten 2′-deoxynucleotides and is flanked on both sides (in the 5′ and 3′ directions) by wings comprising four nucleotides each. For each of the motifs (3-8-3, 4-8-4, 2-10-2, 3-10-3, and 4-10-4), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Human Target start site” indicates the 5′-most nucleotide to which the gapmer is targeted in the human sequence. “Human Target stop site” indicates the 3′-most nucleotide to which the gapmer is targeted in the human sequence. Each gapmer listed in Table 53 is targeted to SEQ ID NO: 1 (GENBANK Accession No. NM_{—}000128.3). Each gapmer is Table 53 is also fully cross-reactive with the rhesus monkey Factor XI gene sequence, designated herein as SEQ ID NO: 274 (exons 1-15 GENBANK Accession No. NW_{—}001118167.1). ‘Rhesus monkey start site’ indicates the 5′-most nucleotide to which the gapmer is targeted in the rhesus monkey sequence. ‘Rhesus monkey stop site’ indicates the 3′-most nucleotide to which the gapmer is targeted to the rhesus monkey sequence.

TABLE 53
Chimeric antisense oligonucleotides targeted to SEQ ID NO: 1
(GENBANK Accession No. NM_000128.3)
and designed by microwalk of ISIS 416850 and ISIS 416858

| | Human | Human | | | | Rhesus | Rhesus |
|---|---|---|---|---|---|---|---|
| | Target | Target | | | SEQ | monkey | monkey |
| ISIS No. | Start Site | Stop Site | Sequence (5′ to 3′) | Motif | ID No. | Start Site | Stop Site |
| 449707 | 1280 | 1295 | CACAGTTTCTGGCAGG | 4-8-4 | 268 | 1279 | 1294 |
| 449708 | 1281 | 1294 | ACAGTTTCTGGCAG | 3-8-3 | 269 | 1280 | 1293 |
| 449709 | 1279 | 1296 | GCACAGTTTCTGGCAGGC | 4-10-4 | 246 | 1278 | 1295 |
| 449710 | 1280 | 1295 | CACAGTTTCTGGCAGG | 3-10-3 | 268 | 1279 | 1294 |
| 449711 | 1281 | 1294 | ACAGTTTCTGGCAG | 2-10-2 | 269 | 1280 | 1293 |

Dose-response inhibition data is given in Table 54. As illustrated in Table 54, Factor XI mRNA levels were reduced in a dose-dependent manner in antisense oligonucleotide treated cells. The IC₅₀ of each antisense oligonucleotide was also calculated and presented in Table 54. The first two listed gapmers in Table 54 are the original gapmers (ISIS 416850 and ISIS 416858) from which the remaining gapmers were designed via microwalk and are designated by an asterisk.

TABLE 54
Dose-dependent antisense inhibition of human Factor XI in HepG2
cells via transfection of oligonucleotides using electroporation

| ISIS | 375 | 750 | 1,500 | 3,000 | 6,000 | 12,000 | IC₅₀ |
|---|---|---|---|---|---|---|---|
| No. | nM | nM | nM | nM | nM | nM | (μM) |
| *416850 | 40 | 59 | 69 | 87 | 90 | 95 | 0.56 |
| *416858 | 31 | 35 | 78 | 85 | 90 | 93 | 0.83 |
| 445522 | 59 | 71 | 83 | 82 | 81 | 92 | n.d. |
| 445531 | 44 | 64 | 78 | 86 | 91 | 93 | 0.44 |
| 449707 | 7 | 35 | 63 | 73 | 85 | 91 | 1.26 |
| 449708 | 0 | 0 | 22 | 33 | 61 | 85 | 4.46 |
| 449709 | 52 | 71 | 80 | 87 | 92 | 95 | 0.38 |
| 449710 | 2 | 21 | 52 | 70 | 82 | 87 | 1.59 |
| 449711 | 6 | 14 | 1 | 7 | 32 | 52 | 11.04 |

n.d. = no data

### Example 17

### Tolerability of Antisense Oligonucleotides Targeting Human Factor XI in CD1 Mice

CD1 mice were treated with ISIS antisense oligonucleotides targeting human Factor XI and evaluated for changes in the levels of various metabolic markers.

### Treatment

Groups of five CD1 mice each were injected subcutaneously twice a week for 2, 4, or 6 weeks with 50 mg/kg of ISIS 416825, ISIS 416826, ISIS 416838, ISIS 416850, ISIS 416858, ISIS 416864, ISIS 416892, ISIS 416925, ISIS 416999, ISIS 417002, or ISIS 417003. A control group of five mice was injected subcutaneously with PBS for 2 weeks. All experimental groups (i.e. ASO treated mice at 2, 4, 6 weeks) were compared to the control group (i.e. PBS, 2 weeks).

Three days after the last dose was administered to all groups, the mice were sacrificed. Organ weights were measured and blood was collected for further analysis.

### Organ Weight

Liver, spleen, and kidney weights were measured at the end of the study, and are presented in Tables 55, 56, and 57 as a percent of the PBS control, normalized to body weight. Those antisense oligonucleotides which did not affect more than six-fold increases in liver and spleen weight above the PBS controls were selected for further studies.

TABLE 55
Percent change in liver weight of CD1 mice after antisense
oligonucleotide treatment

| ISIS | | | |
|---|---|---|---|
| No. | 2 weeks | 4 weeks | 6 weeks |
| 416825 | +5 | +22 | +13 |
| 416826 | +10 | +32 | +33 |
| 416838 | +8 | −6 | 0 |
| 416850 | +5 | +3 | +6 |
| 416858 | +7 | +1 | +10 |
| 416864 | −2 | +2 | −5 |
| 416925 | +14 | +14 | +33 |
| 416999 | +13 | +30 | +47 |
| 417002 | +14 | +8 | +35 |
| 416892 | +35 | +88 | +95 |
| 417003 | +8 | +42 | +32 |

TABLE 56
Percent change in spleen weight of CD1 mice after antisense
oligonucleotide treatment

| ISIS | | | |
|---|---|---|---|
| No. | 2 weeks | 4 weeks | 6 weeks |
| 416825 | −12 | +19 | +21 |
| 416826 | −12 | −5 | +22 |
| 416838 | +21 | −8 | +9 |
| 416850 | −4 | +6 | +48 |
| 416858 | −2 | +8 | +28 |
| 416864 | −10 | −2 | −6 |
| 416925 | −7 | +33 | +78 |
| 416999 | +7 | +22 | +38 |
| 417002 | +29 | +26 | +108 |
| 416892 | +24 | +30 | +65 |
| 417003 | +12 | +101 | +98 |

TABLE 57
Percent change in kidney weight of CD1 mice after antisense
oligonucleotide treatment

| ISIS | | | |
|---|---|---|---|
| No. | 2 weeks | 4 weeks | 6 weeks |
| 416825 | −12 | −12 | −11 |
| 416826 | −13 | −7 | −22 |
| 416838 | −2 | −12 | −8 |
| 416850 | −10 | −12 | −11 |
| 416858 | +1 | −18 | −10 |
| 416864 | −4 | −9 | −15 |
| 416925 | −4 | −14 | −2 |
| 416999 | −9 | −6 | −7 |
| 417002 | +3 | −5 | −2 |
| 416892 | +2 | −3 | +19 |
| 417003 | −9 | −2 | −1 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Measurements of alanine transaminase (ALT) and aspartate transaminase (AST) are expressed in IU/L and the results are presented in Tables 58 and 59. Plasma levels of bilirubin and albumin were also measured using the same clinical chemistry analyzer and expressed in mg/dL. The results are presented in Tables 60 and 61. Those antisense oligonucleotides which did not affect an increase in ALT/AST levels above seven-fold of control levels were selected for further studies. Those antisense oligonucleotides which did not increase levels of bilirubin more than two-fold of the control levels were selected for further studies.

TABLE 58
Effect of antisense oligonucleotide treatment on ALT (IU/L) in CD1 mice

| | 2 weeks | 4 weeks | 6 weeks |
|---|---|---|---|
| PBS | 36 | n.d. | n.d. |
| ISIS 416825 | 64 | 314 | 507 |
| ISIS 416826 | 182 | 126 | 1954 |
| ISIS 416838 | 61 | 41 | 141 |
| ISIS 416850 | 67 | 58 | 102 |
| ISIS 416858 | 190 | 57 | 216 |
| ISIS 416864 | 44 | 33 | 92 |
| ISIS 416925 | 160 | 284 | 1284 |
| ISIS 416999 | 61 | 160 | 1302 |
| ISIS 417002 | 71 | 138 | 2579 |
| ISIS 416892 | 66 | 1526 | 1939 |
| ISIS 417003 | 192 | 362 | 2214 |

n.d. = no data

TABLE 59
Effect of antisense oligonucleotide treatment on AST (IU/L) in CD1 mice

| | 2 weeks | 4 weeks | 6 weeks |
|---|---|---|---|
| PBS | 68 | n.d. | n.d. |
| ISIS 416825 | 82 | 239 | 301 |
| ISIS 416826 | 274 | 156 | 1411 |
| ISIS 416838 | 106 | 73 | 107 |
| ISIS 416850 | 72 | 88 | 97 |
| ISIS 416858 | 236 | 108 | 178 |
| ISIS 416864 | 58 | 46 | 101 |
| ISIS 416925 | 144 | 206 | 712 |
| ISIS 416999 | 113 | 130 | 671 |
| ISIS 417002 | 96 | 87 | 1166 |
| ISIS 416892 | 121 | 1347 | 1443 |
| ISIS 417003 | 152 | 249 | 839 |

n.d. = no data

TABLE 60
Effect of antisense oligonucleotide treatment on
bilirubin (mg/dL) in CD1 mice

| | 2 weeks | 4 weeks | 6 weeks |
|---|---|---|---|
| PBS | 0.28 | n.d. | n.d. |
| ISIS 416825 | 0.41 | 0.69 | 0.29 |
| ISIS 416826 | 0.39 | 0.20 | 0.37 |
| ISIS 416838 | 0.57 | 0.24 | 0.20 |
| ISIS 416850 | 0.46 | 0.23 | 0.22 |
| ISIS 416858 | 0.57 | 0.24 | 0.16 |
| ISIS 416864 | 0.40 | 0.26 | 0.22 |
| ISIS 416925 | 0.45 | 0.25 | 0.25 |
| ISIS 416999 | 0.48 | 0.18 | 0.28 |
| ISIS 417002 | 0.50 | 0.25 | 0.29 |
| ISIS 416892 | 0.38 | 2.99 | 0.50 |
| ISIS 417003 | 0.33 | 0.15 | 0.24 |

n.d. = no data

TABLE 61
Effect of antisense oligonucleotide treatment on
albumin (mg/dL) in CD1 mice

| | 2 weeks | 4 weeks | 6 weeks |
|---|---|---|---|
| PBS | 3.7 | n.d. | n.d. |
| ISIS 416825 | 3.6 | 3.4 | 3.5 |
| ISIS 416826 | 3.3 | 3.4 | 3.4 |
| ISIS 416838 | 3.5 | 3.8 | 3.6 |
| ISIS 416850 | 3.6 | 3.5 | 3.1 |
| ISIS 416858 | 3.4 | 3.5 | 2.8 |
| ISIS 416864 | 3.5 | 3.6 | 3.5 |
| ISIS 416925 | 3.5 | 3.5 | 3.2 |
| ISIS 416999 | 3.4 | 3.3 | 3.2 |
| ISIS 417002 | 3.2 | 3.4 | 3.4 |
| ISIS 416892 | 3.2 | 4.0 | 4.4 |
| ISIS 417003 | 3.4 | 3.4 | 3.2 |

n.d. = no data

### Kidney Function

To evaluate the effect of ISIS oligonucleotides on kidney function, plasma concentrations of blood urea nitrogen (BUN) and creatinine were measured using an automated clinical chemistry analyzer (Hitachi Olympus AU400e, Melville, N.Y.). Results are presented in Tables 62 and 63, expressed in mg/dL. Those antisense oligonucleotides which did not affect more than a two-fold increase in BUN levels compared to the PBS control were selected for further studies.

TABLE 62
Effect of antisense oligonucleotide treatment on
BUN (mg/dL) in CD1 mice

| | 2 weeks | 4 weeks | 6 weeks |
|---|---|---|---|
| PBS | 30 | n.d. | n.d. |
| ISIS 416825 | 29 | 35 | 31 |
| ISIS 416826 | 24 | 34 | 27 |
| ISIS 416838 | 25 | 38 | 30 |
| ISIS 416850 | 25 | 30 | 23 |
| ISIS 416858 | 21 | 29 | 19 |
| ISIS 416864 | 22 | 31 | 28 |
| ISIS 416925 | 21 | 30 | 17 |
| ISIS 416999 | 22 | 27 | 22 |
| ISIS 417002 | 19 | 23 | 19 |
| ISIS 416892 | 19 | 28 | 23 |
| ISIS 417003 | 23 | 26 | 24 |

n.d. = no data

TABLE 63
Effect of antisense oligonucleotide treatment on
creatinine (mg/dL) in CD1 mice

| | 2 weeks | 4 weeks | 6 weeks |
|---|---|---|---|
| PBS | 0.14 | n.d. | n.d. |
| ISIS 416825 | 0.14 | 0.21 | 0.17 |
| ISIS 416826 | 0.15 | 0.20 | 0.15 |
| ISIS 416838 | 0.09 | 0.27 | 0.14 |
| ISIS 416850 | 0.13 | 0.22 | 0.19 |
| ISIS 416858 | 0.13 | 0.23 | 0.10 |
| ISIS 416864 | 0.11 | 0.22 | 0.16 |
| ISIS 416925 | 0.12 | 0.25 | 0.13 |
| ISIS 416999 | 0.07 | 0.18 | 0.13 |
| ISIS 417002 | 0.06 | 0.16 | 0.10 |
| ISIS 416892 | 0.11 | 0.20 | 0.17 |
| ISIS 417003 | 0.17 | 0.24 | 0.18 |

n.d. = no data

### Hematology Assays

Blood obtained from all mice groups were sent to Antech Diagnostics for hematocrit (HCT), mean corpuscular volume (MCV), mean corpuscular hemoglobin (MCH), and mean corpuscular hemoglobin concentration (MCHC) measurements and analyses, as well as measurements of the various blood cells, such as WBC (neutrophils, lymphocytes, and monocytes), RBC, and platelets, and total hemoglobin content. The results are presented in Tables 64-74. Percentages given in the tables indicate the percent of total blood cell count. Those antisense oligonucleotides which did not affect a decrease in platelet count of more than 50% and/or an increase in monocyte count of more than three-fold were selected for further studies.

TABLE 64
Effect of antisense oligonucleotide treatment on HCT (%) in CD1 mice

| | 2 weeks | 4 weeks | 6 weeks |
|---|---|---|---|
| PBS | 50 | n.d. | n.d. |
| ISIS 416825 | 49 | 46 | 40 |
| ISIS 416826 | 47 | 41 | 37 |
| ISIS 416838 | 42 | 44 | 39 |
| ISIS 416850 | 44 | 44 | 38 |
| ISIS 416858 | 50 | 45 | 46 |
| ISIS 416864 | 50 | 45 | 42 |
| ISIS 416925 | 51 | 47 | 47 |
| ISIS 416999 | 51 | 42 | 40 |
| ISIS 417002 | 44 | 44 | 51 |
| ISIS 416892 | 48 | 42 | 45 |
| ISIS 417003 | 48 | 41 | 43 |

n.d. = no data

TABLE 65
Effect of antisense oligonucleotide treatment on MCV (fL) in CD1 mice

| | 2 weeks | 4 weeks | 6 weeks |
|---|---|---|---|
| PBS | 61 | n.d. | n.d. |
| ISIS 416825 | 58 | 53 | 51 |
| ISIS 416826 | 56 | 52 | 53 |
| ISIS 416838 | 56 | 54 | 48 |
| ISIS 416850 | 57 | 51 | 50 |
| ISIS 416858 | 59 | 51 | 50 |
| ISIS 416864 | 57 | 52 | 51 |
| ISIS 416925 | 61 | 52 | 47 |
| ISIS 416999 | 60 | 49 | 48 |
| ISIS 417002 | 61 | 50 | 52 |
| ISIS 416892 | 59 | 49 | 53 |
| ISIS 417003 | 60 | 48 | 45 |

n.d. = no data

TABLE 66
Effect of antisense oligonucleotide treatment on MCH (pg) in CD1 mice

| ISIS No. | 2 weeks | 4 weeks | 6 weeks |
|---|---|---|---|
| PBS | 18 | n.d. | n.d. |
| ISIS 416825 | 17 | 16 | 15 |
| ISIS 416826 | 17 | 16 | 16 |
| ISIS 416838 | 17 | 17 | 15 |
| ISIS 416850 | 17 | 16 | 15 |
| ISIS 416858 | 17 | 16 | 15 |
| ISIS 416864 | 18 | 16 | 16 |
| ISIS 416925 | 17 | 16 | 15 |
| ISIS 416999 | 17 | 16 | 15 |
| ISIS 417002 | 17 | 16 | 16 |
| ISIS 416892 | 18 | 16 | 16 |
| ISIS 417003 | 17 | 16 | 16 |

n.d. = no data

TABLE 67
Effect of antisense oligonucleotide treatment on MCHC (%) in CD1 mice

| | 2 weeks | 4 weeks | 6 weeks |
|---|---|---|---|
| PBS | 30 | n.d. | n.d. |
| ISIS 416825 | 29 | 31 | 31 |
| ISIS 416826 | 29 | 31 | 30 |
| ISIS 416838 | 30 | 31 | 32 |
| ISIS 416850 | 30 | 31 | 31 |
| ISIS 416858 | 30 | 32 | 31 |
| ISIS 416864 | 31 | 31 | 31 |
| ISIS 416925 | 30 | 32 | 32 |
| ISIS 416999 | 27 | 32 | 31 |
| ISIS 417002 | 29 | 32 | 31 |
| ISIS 416892 | 30 | 32 | 30 |
| ISIS 417003 | 29 | 32 | 33 |

n.d. = no data

TABLE 68
Effect of antisense oligonucleotide treatment on WBC
count (cells/nL) in CD1 mice

| | 2 weeks | 4 weeks | 6 weeks |
|---|---|---|---|
| PBS | 6 | n.d. | n.d. |
| ISIS 416825 | 8 | 8 | 6 |
| ISIS 416826 | 5 | 6 | 8 |
| ISIS 416838 | 4 | 6 | 5 |
| ISIS 416850 | 4 | 5 | 5 |
| ISIS 416858 | 6 | 7 | 4 |
| ISIS 416864 | 7 | 6 | 5 |
| ISIS 416925 | 6 | 6 | 11 |
| ISIS 416999 | 4 | 9 | 7 |
| ISIS 417002 | 8 | 8 | 16 |
| ISIS 416892 | 5 | 8 | 9 |
| ISIS 417003 | 7 | 9 | 10 |

n.d. = no data

TABLE 69
Effect of antisense oligonucleotide treatment on RBC
count (cells/pL) in CD1 mice

| | 2 weeks | 4 weeks | 6 weeks |
|---|---|---|---|
| PBS | 8 | n.d. | n.d. |
| ISIS 416825 | 9 | 9 | 8 |
| ISIS 416826 | 8 | 8 | 7 |
| ISIS 416838 | 8 | 8 | 8 |
| ISIS 416850 | 8 | 9 | 8 |
| ISIS 416858 | 9 | 9 | 9 |
| ISIS 416864 | 9 | 9 | 8 |
| ISIS 416925 | 9 | 9 | 10 |
| ISIS 416999 | 9 | 9 | 8 |
| ISIS 417002 | 9 | 9 | 10 |
| ISIS 416892 | 7 | 9 | 9 |
| ISIS 417003 | 8 | 9 | 10 |

n.d. = no data

TABLE 70
Effect of antisense oligonucleotide treatment on neutrophil
count (%) in CD1 mice

| | 2 weeks | 4 weeks | 6 weeks |
|---|---|---|---|
| PBS | 16 | n.d. | n.d. |
| ISIS 416825 | 15 | 43 | 23 |
| ISIS 416826 | 26 | 33 | 23 |
| ISIS 416838 | 19 | 33 | 31 |
| ISIS 416850 | 15 | 21 | 16 |
| ISIS 416858 | 14 | 24 | 27 |
| ISIS 416864 | 13 | 27 | 20 |
| ISIS 416925 | 12 | 39 | 33 |
| ISIS 416999 | 12 | 25 | 22 |
| ISIS 417002 | 14 | 31 | 36 |
| ISIS 416892 | 19 | 43 | 28 |
| ISIS 417003 | 10 | 39 | 24 |

n.d. = no data

TABLE 71
Effect of antisense oligonucleotide treatment on lymphocyte
count (%) in CD1 mice

| | 2 weeks | 4 weeks | 6 weeks |
|---|---|---|---|
| PBS | 81 | n.d. | n.d. |
| ISIS 416825 | 82 | 53 | 71 |
| ISIS 416826 | 70 | 61 | 67 |
| ISIS 416838 | 76 | 64 | 60 |
| ISIS 416850 | 82 | 73 | 76 |
| ISIS 416858 | 83 | 73 | 65 |
| ISIS 416864 | 84 | 71 | 74 |
| ISIS 416925 | 86 | 58 | 57 |
| ISIS 416999 | 86 | 72 | 69 |
| ISIS 417002 | 83 | 64 | 51 |
| ISIS 416892 | 79 | 52 | 64 |
| ISIS 417003 | 86 | 54 | 66 |

n.d. = no data

TABLE 72
Effect of antisense oligonucleotide treatment on monocyte
count (%) in CD1 mice

| | 2 weeks | 4 weeks | 6 weeks |
|---|---|---|---|
| PBS | 3 | n.d. | n.d. |
| ISIS 416825 | 2 | 5 | 4 |
| ISIS 416826 | 3 | 5 | 8 |
| ISIS 416838 | 2 | 2 | 6 |
| ISIS 416850 | 3 | 6 | 6 |
| ISIS 416858 | 2 | 3 | 7 |
| ISIS 416864 | 2 | 2 | 5 |
| ISIS 416925 | 2 | 4 | 8 |
| ISIS 416999 | 2 | 4 | 8 |
| ISIS 417002 | 3 | 4 | 12 |
| ISIS 416892 | 3 | 6 | 7 |
| ISIS 417003 | 2 | 6 | 8 |

n.d. = no data

TABLE 73
Effect of antisense oligonucleotide treatment on platelet
count (cells/nL) in CD1 mice

| | 2 weeks | 4 weeks | 6 weeks |
|---|---|---|---|
| PBS | 2126 | n.d. | n.d. |
| ISIS 416825 | 1689 | 1229 | 942 |
| ISIS 416826 | 1498 | 970 | 645 |
| ISIS 416838 | 1376 | 1547 | 1229 |
| ISIS 416850 | 1264 | 1302 | 1211 |
| ISIS 416858 | 2480 | 1364 | 1371 |
| ISIS 416864 | 1924 | 1556 | 933 |
| ISIS 416925 | 1509 | 1359 | 1211 |
| ISIS 416999 | 1621 | 1219 | 1057 |
| ISIS 417002 | 1864 | 1245 | 1211 |
| ISIS 416892 | 1687 | 636 | 1004 |
| ISIS 417003 | 1309 | 773 | 922 |

n.d. = no data

TABLE 74
Effect of antisense oligonucleotide treatment on hemoglobin
content (g/dL) in CD1 mice

| | 2 weeks | 4 weeks | 6 weeks |
|---|---|---|---|
| PBS | 15.1 | n.d. | n.d. |
| ISIS 416825 | 14.5 | 14.1 | 12.1 |
| ISIS 416826 | 13.4 | 12.8 | 11.0 |
| ISIS 416838 | 12.4 | 13.6 | 12.6 |
| ISIS 416850 | 13.1 | 13.5 | 11.6 |
| ISIS 416858 | 14.8 | 14.2 | 14.1 |
| ISIS 416864 | 15.2 | 13.9 | 13.0 |
| ISIS 416925 | 14.9 | 14.8 | 15.3 |
| ISIS 416999 | 14.2 | 13.3 | 12.8 |
| ISIS 417002 | 14.7 | 14.0 | 15.7 |
| ISIS 416892 | 13.0 | 13.5 | 13.1 |
| ISIS 417003 | 13.7 | 13.4 | 14.0 |

n.d. = no data

### Example 18

### Measurement of Half-Life of Antisense Oligonucleotide in CD1 Mice Liver

CD1 mice were treated with ISIS antisense oligonucleotides targeting human Factor XI and the oligonucleotide half-life as well as the elapsed time for oligonucleotide degradation and elimination from the liver was evaluated.

### Treatment

Groups of fifteen CD1 mice each were injected subcutaneously twice per week for 2 weeks with 50 mg/kg of ISIS 416825, ISIS 416826, ISIS 416838, ISIS 416850, ISIS 416858, ISIS 416864, ISIS 416892, ISIS 416925, ISIS 416999, ISIS 417002, or ISIS 417003. Five mice from each group were sacrificed 3 days, 28 days and 56 days following the final dose. Livers were harvested for analysis.

### Measurement of Oligonucleotide Concentration

The concentration of the full-length oligonucleotide as well as the total oligonucleotide concentration (including the degraded form) was measured. The method used is a modification of previously published methods (Leeds et al., 1996; Geary et al., 1999) which consist of a phenol-chloroform (liquid-liquid) extraction followed by a solid phase extraction. An internal standard (ISIS 355868, a 27-mer 2′-O-methoxyethyl modified phosphorothioate oligonucleotide, GCGTTTGCTCTTCTTCTTGCGTTTTTT, designated herein as SEQ ID NO: 270) was added prior to extraction. Tissue sample concentrations were calculated using calibration curves, with a lower limit of quantitation (LLOQ) of approximately 1.14 μg/g. Half-lives were then calculated using WinNonlin software (PHARSIGHT).

The results are presented in Tables 75 and 76, expressed as μg liver tissue. The half-life of each oligonucleotide is presented in Table 77.

TABLE 75
Full-length oligonucleotide concentration (μg/g) in the liver of CD1 mice

| ISIS No. | Motif | day 3 | day 28 | day 56 |
|---|---|---|---|---|
| 416825 | 5-10-5 | 151 | 52 | 7 |
| 416826 | 5-10-5 | 186 | 48 | 8 |
| 416838 | 5-10-5 | 170 | 46 | 10 |
| 416850 | 5-10-5 | 238 | 93 | 51 |
| 416858 | 5-10-5 | 199 | 102 | 18 |
| 416864 | 5-10-5 | 146 | 38 | 25 |
| 416999 | 2-13-5 | 175 | 26 | 0 |
| 417002 | 2-13-5 | 119 | 24 | 1 |
| 417003 | 2-13-5 | 245 | 42 | 4 |
| 416925 | 3-14-3 | 167 | 39 | 5 |
| 416892 | 3-14-3 | 135 | 31 | 6 |

TABLE 76
Total oligonucleotide concentration (μg/g) in the liver of CD1 mice

| ISIS No. | Motif | day 3 | day 28 | day 56 |
|---|---|---|---|---|
| 416825 | 5-10-5 | 187 | 90 | 39 |
| 416826 | 5-10-5 | 212 | 61 | 12 |
| 416838 | 5-10-5 | 216 | 98 | 56 |
| 416850 | 5-10-5 | 295 | 157 | 143 |
| 416858 | 5-10-5 | 273 | 185 | 56 |
| 416864 | 5-10-5 | 216 | 86 | 112 |
| 416999 | 2-13-5 | 232 | 51 | 0 |
| 417002 | 2-13-5 | 206 | 36 | 1 |
| 417003 | 2-13-5 | 353 | 74 | 4 |
| 416925 | 3-14-3 | 280 | 72 | 8 |
| 416892 | 3-14-3 | 195 | 54 | 6 |

TABLE 77
Half-life of antisense oligonucleotides in the liver of CD1 mice

| | | Half-life |
|---|---|---|
| ISIS No. | Motif | (days) |
| 416825 | 5-10-5 | 16 |
| 416826 | 5-10-5 | 13 |
| 416838 | 5-10-5 | 13 |
| 416850 | 5-10-5 | 18 |
| 416858 | 5-10-5 | 26 |
| 416864 | 5-10-5 | 13 |
| 416999 | 2-13-5 | 9 |
| 417002 | 2-13-5 | 11 |
| 417003 | 2-13-5 | 10 |
| 416925 | 3-14-3 | 12 |
| 416892 | 3-14-3 | 12 |

### Example 19

### Tolerability of Antisense Oligonucleotides Targeting Human Factor XI in Sprague-Dawley Rats

Sprague-Dawley rats were treated with ISIS antisense oligonucleotides targeting human Factor XI and evaluated for changes in the levels of various metabolic markers.

### Treatment

Groups of four Sprague Dawley rats each were injected subcutaneously twice per week for 6 weeks with 50 mg/kg of ISIS 416825, ISIS 416826, ISIS 416838, ISIS 416850, ISIS 416858, ISIS 416848, ISIS 416864, ISIS 416892, ISIS 416925, ISIS 416999, ISIS 417002, or ISIS 417003. A control group of four Sprague Dawley rats was injected subcutaneously with PBS twice per week for 6 weeks. Body weight measurements were taken before and throughout the treatment period. Urine samples were taken before the start of treatment. Three days after the last dose, urine samples were taken and the rats were sacrificed. Organ weights were measured and blood was collected for further analysis.

### Body Weight and Organ Weight

Body weights of the rats were measured at the onset of the study and subsequently twice per week. The body weights are presented in Table 78 and are expressed as a percent change over the weights taken at the start of the study. Liver, spleen, and kidney weights were measured at the end of the study and are presented in Table 78 as a percent of the saline control normalized to body weight. Those antisense oligonucleotides which did not affect more than a six-fold increase in liver and spleen weight above the PBS control were selected for further studies.

TABLE 78
Percent change in organ weight of Sprague Dawley rats after
antisense oligonucleotide treatment

| ISIS | | | | Body |
|---|---|---|---|---|
| No. | Liver | Spleen | Kidney | weight |
| 416825 | +20 | +245 | +25 | −18 |
| 416826 | +81 | +537 | +44 | −40 |
| 416838 | +8 | +212 | −0.5 | −23 |
| 416850 | +23 | +354 | +47 | −33 |
| 416858 | +8 | +187 | +5 | −21 |
| 416864 | +16 | +204 | +16 | −24 |
| 416925 | +44 | +371 | +48 | −32 |
| 416999 | +51 | +405 | +71 | −37 |
| 417002 | +27 | +446 | +63 | −29 |
| 416892 | +38 | +151 | +32 | −39 |
| 417003 | +51 | +522 | +25 | −40 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Measurements of alanine transaminase (ALT) and aspartate transaminase (AST) are expressed in IU/L and the results are presented in Table 79. Those antisense oligonucleotides which did not affect an increase in ALT/AST levels above seven-fold of control levels were selected for further studies. Plasma levels of bilirubin and albumin were also measured with the same clinical analyzer and the results are also presented in Table 79, expressed in mg/dL. Those antisense oligonucleotides which did not affect an increase in levels of bilirubin more than two-fold of the control levels by antisense oligonucleotide treatment were selected for further studies.

TABLE 79
Effect of antisense oligonucleotide treatment on metabolic markers
in the liver of Sprague-Dawley rats

| | ALT | AST | Bilirubin | Albumin |
|---|---|---|---|---|
| | (IU/L) | (IU/L) | (mg/dL) | (mg/dL) |
| PBS | 9 | 5 | 20 | 2 |
| ISIS 416825 | 89 | 17 | 4 | 2 |
| ISIS 416826 | 611 | 104 | 115 | 6 |
| ISIS 416838 | 5 | 2 | 4 | 2 |
| ISIS 416850 | 80 | 5 | 1 | 4 |
| ISIS 416858 | 13 | 4 | 4 | 2 |
| ISIS 416864 | 471 | 68 | 3 | 4 |
| ISIS 416925 | 102 | 20 | 13 | 5 |
| ISIS 416999 | 92 | 28 | 54 | 5 |
| ISIS 417002 | 44 | 11 | 12 | 3 |
| ISIS 416892 | 113 | 183 | 1 | 8 |
| ISIS 417003 | 138 | 23 | 50 | 6 |

### Kidney Function

To evaluate the effect of kidney function, plasma concentrations of blood urea nitrogen (BUN) and creatinine were measured using an automated clinical chemistry analyzer (Hitachi Olympus AU400e, Melville, N.Y.). Results are presented in Table 80, expressed in mg/dL. Those antisense oligonucleotides which did not affect more than a two-fold increase in BUN levels compared to the PBS control were selected for further studies. The ratio of urine protein to creatinine in total urine samples was also calculated before and after antisense oligonucleotide treatment and is presented in Table 81. Those antisense oligonucleotides which did not affect more than a five-fold increase in urine protein/creatinine ratios compared to the PBS control were selected for further studies.

TABLE 80
Effect of antisense oligonucleotide treatment on metabolic markers
in the kidney of Sprague-Dawley rats

| | BUN | Creatinine |
|---|---|---|
| PBS | 4 | 8 |
| ISIS 416825 | 7 | 17 |
| ISIS 416826 | 25 | 6 |
| ISIS 416838 | 4 | 5 |
| ISIS 416850 | 5 | 7 |
| ISIS 416858 | 8 | 4 |
| ISIS 416864 | 5 | 6 |
| ISIS 416925 | 7 | 5 |
| ISIS 416999 | 2 | 4 |
| ISIS 417002 | 11 | 1 |
| ISIS 416892 | 188 | 1 |
| ISIS 417003 | 9 | 9 |

TABLE 81
Effect of antisense oligonucleotide treatment on urine protein/creatinine
ratio in Sprague Dawley rats

| | Before | After |
|---|---|---|
| PBS | 1.2 | 1.3 |
| 416825 | 1.1 | 5.4 |
| 416826 | 1.0 | 11.4 |
| 416838 | 1.2 | 3.7 |
| 416850 | 1.0 | 4.0 |
| 416858 | 0.9 | 4.4 |
| 416864 | 1.2 | 4.0 |
| 416925 | 1.0 | 4.3 |
| 416999 | 1.3 | 9.1 |
| 417002 | 1.0 | 2.4 |
| 416892 | 0.8 | 21.3 |
| 417003 | 0.9 | 4.8 |

### Hematology Assays

Blood obtained from all rat groups were sent to Antech Diagnostics for hematocrit (HCT), mean corpuscular volume (MCV), mean corpuscular hemoglobin (MCV), and mean corpuscular hemoglobin concentration (MCHC) measurements and analyses, as well as measurements of various blood cells, such as WBC (neutrophils, lymphocytes and monocytes), RBC, and platelets as well as hemoglobin content. The results are presented in Tables 82 and 83. Those antisense oligonucleotides which did not affect a decrease in platelet count of more than 50% and an increase in monocyte count of more than three-fold were selected for further studies.

TABLE 82
Effect of antisense oligonucleotide treatment
on blood cell count in Sprague-Dawley rats

| | | | Neutro- | Lym- | Mono- | |
|---|---|---|---|---|---|---|
| | WBC | RBC | phils | phocytes | cytes | Platelets |
| | (/nL) | (/pL) | (%) | (%) | (%) | (10³/μL) |
| PBS | 21 | 6 | 37 | 7 | 26 | 18 |
| ISIS 416825 | 22 | 2 | 25 | 3 | 15 | 6 |
| ISIS 416826 | 7 | 5 | 30 | 5 | 7 | 11 |
| ISIS 416838 | 13 | 4 | 17 | 3 | 6 | 27 |
| ISIS 416850 | 16 | 7 | 48 | 8 | 11 | 26 |
| ISIS 416858 | 28 | 2 | 20 | 3 | 10 | 19 |
| ISIS 416864 | 15 | 4 | 26 | 2 | 29 | 12 |
| ISIS 416925 | 24 | 6 | 20 | 4 | 23 | 8 |
| ISIS 416999 | 12 | 5 | 23 | 3 | 20 | 12 |
| ISIS 417002 | 23 | 5 | 22 | 4 | 25 | 7 |
| ISIS 416892 | 68 | 12 | 92 | 18 | 58 | 66 |
| ISIS 417003 | 83 | 11 | 17 | 3 | 6 | 19 |

TABLE 83
Effect of antisense oligonucleotide treatment on hematologic
factors (% control) in Sprague-Dawley rats

| | Hemoglobin | HCT | MCV | MCH | MCHC |
|---|---|---|---|---|---|
| | (g/dL) | (%) | (fL) | (pg) | (%) |
| PBS | 6 | 4 | 6 | 2 | 4 |
| ISIS 416825 | 2 | 2 | 4 | 2 | 4 |
| ISIS 416826 | 7 | 7 | 6 | 3 | 4 |
| ISIS 416838 | 2 | 5 | 4 | 2 | 5 |
| ISIS 416850 | 4 | 5 | 3 | 4 | 2 |
| ISIS 416858 | 2 | 3 | 2 | 2 | 1 |
| ISIS 416864 | 4 | 2 | 4 | 2 | 4 |
| ISIS 416925 | 6 | 8 | 5 | 2 | 4 |
| ISIS 416999 | 6 | 5 | 2 | 3 | 1 |
| ISIS 417002 | 5 | 7 | 7 | 3 | 5 |
| ISIS 416892 | 14 | 13 | 1 | 2 | 0 |
| ISIS 417003 | 11 | 8 | 6 | 4 | 4 |

### Example 20

### Measurement of Half-Life of Antisense Oligonucleotide in Sprague-Dawley Rat Liver and Kidney

Sprague Dawley rats were treated with ISIS antisense oligonucleotides targeting human Factor XI and the oligonucleotide half-life as well as the elapsed time for oligonucleotide degradation and elimination from the liver and kidney was evaluated.

### Treatment

Groups of four Sprague Dawley rats each were injected subcutaneously twice a week for 2 weeks with 20 mg/kg of ISIS416825, ISIS 416826, ISIS 416838, ISIS 416850, ISIS 416858, ISIS 416864, ISIS 416892, ISIS 416925, ISIS 416999, ISIS 417002, or ISIS 417003. Three days after the last dose, the rats were sacrificed and livers and kidneys were collected for analysis.

### Measurement of Oligonucleotide Concentration

The concentration of the full-length oligonucleotide as well as the total oligonucleotide concentration (including the degraded form) was measured. The method used is a modification of previously published methods (Leeds et al., 1996; Geary et al., 1999) which consist of a phenol-chloroform (liquid-liquid) extraction followed by a solid phase extraction. An internal standard (ISIS 355868, a 27-mer 2′-O-methoxyethyl modified phosphorothioate oligonucleotide, GCGTTTGCTCTTCTTCTTGCGTTTTTT, designated herein as SEQ ID NO: 270) was added prior to extraction. Tissue sample concentrations were calculated using calibration curves, with a lower limit of quantitation (LLOQ) of approximately 1.14 μg/g. The results are presented in Tables 84 and 85, expressed as μg/g liver or kidney tissue. Half-lives were then calculated using WinNonlin software (PHARSIGHT) and presented in Table 86.

TABLE 84
Full-length oligonucleotide concentration (μg/g) in the liver and
kidney of Sprague-Dawley rats

| ISIS No. | Motif | Kidney | Liver |
|---|---|---|---|
| 416825 | 5-10-5 | 632 | 236 |
| 416826 | 5-10-5 | 641 | 178 |
| 416838 | 5-10-5 | 439 | 171 |
| 416850 | 5-10-5 | 259 | 292 |
| 416858 | 5-10-5 | 575 | 255 |
| 416864 | 5-10-5 | 317 | 130 |
| 416999 | 2-13-5 | 358 | 267 |
| 417002 | 2-13-5 | 291 | 118 |
| 417003 | 2-13-5 | 355 | 199 |
| 416925 | 3-14-3 | 318 | 165 |
| 416892 | 3-14-3 | 351 | 215 |

TABLE 85
Total oligonucleotide concentration (μg/g) in the liver and
kidney of Sprague-Dawley rats

| ISIS No. | Motif | Kidney | Liver |
|---|---|---|---|
| 416825 | 5-10-5 | 845 | 278 |
| 416826 | 5-10-5 | 775 | 214 |
| 416838 | 5-10-5 | 623 | 207 |
| 416850 | 5-10-5 | 352 | 346 |
| 416858 | 5-10-5 | 818 | 308 |
| 416864 | 5-10-5 | 516 | 209 |
| 416999 | 2-13-5 | 524 | 329 |
| 417002 | 2-13-5 | 490 | 183 |
| 417003 | 2-13-5 | 504 | 248 |
| 416925 | 3-14-3 | 642 | 267 |
| 416892 | 3-14-3 | 608 | 316 |

TABLE 86
Half-life (days) of ISIS oligonucleotides in the liver and
kidney of Sprague-Dawley rats

| ISIS No. | Motif | Half-life |
|---|---|---|
| 416825 | 5-10-5 | 16 |
| 416826 | 5-10-5 | 13 |
| 416838 | 5-10-5 | 13 |
| 416850 | 5-10-5 | 18 |
| 416858 | 5-10-5 | 26 |
| 416864 | 5-10-5 | 13 |
| 416999 | 2-13-5 | 9 |
| 417002 | 2-13-5 | 11 |
| 417003 | 2-13-5 | 10 |
| 416925 | 3-14-3 | 12 |
| 416892 | 3-14-3 | 12 |

### Example 21

### Tolerability of Antisense Oligonucleotides Targeting Human Factor XI in CD1 Mice

CD1 mice were treated with ISIS antisense oligonucleotides targeting human Factor XI and evaluated for changes in the levels of various metabolic markers.

### Treatment

Groups of five CD1 mice each were injected subcutaneously twice per week for 6 weeks with 50 mg/kg of ISIS 412223, ISIS 412224, ISIS 412225, ISIS 413481, ISIS 413482, ISIS 416848, ISIS 416849, ISIS 416850, ISIS 416851, ISIS 416852, ISIS 416853, ISIS 416854, ISIS 416855, ISIS 416856, ISIS 416857, ISIS 416858, ISIS 416859, ISIS 416860, ISIS 416861, ISIS 416862, ISIS 416863, ISIS 416864, ISIS 416865, ISIS 416866, or ISIS 416867. A control group of ten CD1 mice was injected subcutaneously with PBS twice per week for 6 weeks. Body weight measurements were taken before and throughout the treatment period. Three days after the last dose, the mice were sacrificed, organ weights were measured, and blood was collected for further analysis.

### Body Weight and Organ Weights

Body weight was measured at the onset of the study and subsequently twice per week. The body weights of the mice are presented in Table 87 and are expressed increase in grams over the PBS control weight taken before the start of treatment. Liver, spleen, and kidney weights were measured at the end of the study, and are also presented in Table 87 as percentage of the body weight. Those antisense oligonucleotides which did not affect more than six-fold increases in liver and spleen weight above the PBS control were selected for further studies.

TABLE 87
Change in body and organ weights of CD1 mice after antisense
oligonucleotide treatment

| | | | | body |
|---|---|---|---|---|
| | Liver | Kidney | Spleen | weight |
| | (%) | (%) | (%) | (g) |
| PBS | 5 | 1.5 | 0.3 | 7 |
| ISIS 416850 | 6 | 1.6 | 0.4 | 12 |
| ISIS 416858 | 7 | 1.6 | 0.6 | 12 |
| ISIS 416864 | 5 | 1.6 | 0.3 | 12 |
| ISIS 412223 | 6 | 1.5 | 0.4 | 12 |
| ISIS 412224 | 6 | 1.6 | 0.5 | 10 |
| ISIS 412225 | 6 | 1.5 | 0.4 | 10 |
| ISIS 413481 | 6 | 1.5 | 0.5 | 9 |
| ISIS 413482 | 6 | 1.6 | 0.5 | 11 |
| ISIS 416848 | 6 | 1.5 | 0.4 | 11 |
| ISIS 416849 | 8 | 1.5 | 0.4 | 8 |
| ISIS 416851 | 7 | 1.5 | 0.5 | 11 |
| ISIS 416852 | 6 | 1.5 | 0.4 | 10 |
| ISIS 416853 | 8 | 1.5 | 0.7 | 13 |
| ISIS 416854 | 7 | 1.2 | 0.4 | 13 |
| ISIS 416855 | 8 | 1.4 | 0.6 | 12 |
| ISIS 416856 | 6 | 1.4 | 0.4 | 10 |
| ISIS 416857 | 7 | 1.6 | 0.5 | 10 |
| ISIS 416859 | 6 | 1.5 | 0.4 | 10 |
| ISIS 416860 | 6 | 1.4 | 0.4 | 10 |
| ISIS 416861 | 5 | 1.3 | 0.4 | 9 |
| ISIS 416862 | 6 | 1.5 | 0.4 | 10 |
| ISIS 416863 | 5 | 1.5 | 0.4 | 9 |
| ISIS 416865 | 6 | 1.5 | 0.4 | 8 |
| ISIS 416866 | 5 | 1.6 | 0.4 | 10 |
| ISIS 416867 | 5 | 1.4 | 0.4 | 9 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Measurements of alanine transaminase (ALT) and aspartate transaminase (AST) are expressed in IU/L and the results are presented in Table 88. Those antisense oligonucleotides which did not affect an increase in ALT/AST levels above seven-fold of control levels were selected for further studies. Plasma levels of bilirubin, cholesterol and albumin were also measured using the same clinical chemistry analyzer and are presented in Table 88 expressed in mg/dL. Those antisense oligonucleotides which did not affect an increase in levels of bilirubin more than two-fold of the control levels by antisense oligonucleotide treatment were selected for further studies.

TABLE 88
Effect of antisense oligonucleotide treatment on metabolic markers
in the liver of CD1 mice

| | ALT | AST | Bilirubin | Albumin | Cholesterol |
|---|---|---|---|---|---|
| | (IU/L) | (IU/L) | (mg/dL) | (mg/dL) | (mg/dL) |
| PBS | 32 | 68 | 0.25 | 3.7 | 135 |
| ISIS 416850 | 75 | 99 | 0.21 | 3.5 | 142 |
| ISIS 416858 | 640 | 547 | 0.28 | 4.4 | 181 |
| ISIS 416864 | 36 | 67 | 0.19 | 2.6 | 152 |
| ISIS 412223 | 60 | 125 | 0.20 | 3.0 | 117 |
| ISIS 412224 | 214 | 183 | 0.19 | 3.4 | 114 |
| ISIS 412225 | 40 | 69 | 0.23 | 3.3 | 128 |
| ISIS 413481 | 85 | 143 | 0.18 | 3.2 | 153 |
| ISIS 413482 | 54 | 77 | 0.24 | 3.0 | 138 |
| ISIS 416848 | 153 | 153 | 0.19 | 3.1 | 151 |
| ISIS 416849 | 1056 | 582 | 0.22 | 2.5 | 109 |
| ISIS 416851 | 47 | 76 | 0.19 | 3.1 | 106 |
| ISIS 416852 | 49 | 91 | 0.16 | 4.9 | 125 |
| ISIS 416853 | 1023 | 1087 | 0.25 | 3.1 | 164 |
| ISIS 416854 | 1613 | 1140 | 0.21 | 5.5 | 199 |
| ISIS 416855 | 786 | 580 | 0.25 | 4.2 | 162 |
| ISIS 416856 | 130 | 129 | 0.23 | 5.2 | 109 |
| ISIS 416857 | 370 | 269 | 0.22 | 3.7 | 94 |
| ISIS 416859 | 214 | 293 | 0.20 | 4.2 | 160 |
| ISIS 416860 | 189 | 160 | 0.23 | 3.5 | 152 |
| ISIS 416861 | 38 | 85 | 0.27 | 4.3 | 133 |
| ISIS 416862 | 225 | 172 | 0.36 | 3.9 | 103 |
| ISIS 416863 | 41 | 101 | 0.24 | 3.6 | 118 |
| ISIS 416865 | 383 | 262 | 0.27 | 4.1 | 95 |
| ISIS 416866 | 36 | 120 | 0.29 | 4.3 | 113 |
| ISIS 416867 | 45 | 82 | 0.21 | 3.3 | 144 |

### Kidney Function

To evaluate the effect of ISIS oligonucleotides on kidney function, plasma concentrations of blood urea nitrogen (BUN) were measured using an automated clinical chemistry analyzer and results are presented in Table 89 expressed in mg/dL. Those antisense oligonucleotides which did not affect more than a two-fold increase in BUN levels compared to the PBS control were selected for further studies.

TABLE 89
Effect of antisense oligonucleotide treatment on BUN levels (mg/dL)
in the kidney of CD1 mice

| | BUN |
|---|---|
| PBS | 22 |
| ISIS 416850 | 24 |
| ISIS 416858 | 23 |
| ISIS 416864 | 24 |
| ISIS 412223 | 28 |
| ISIS 412224 | 29 |
| ISIS 412225 | 23 |
| ISIS 413481 | 23 |
| ISIS 413482 | 27 |
| ISIS 416848 | 23 |
| ISIS 416849 | 23 |
| ISIS 416851 | 21 |
| ISIS 416852 | 21 |
| ISIS 416853 | 22 |
| ISIS 416854 | 27 |
| ISIS 416855 | 23 |
| ISIS 416856 | 21 |
| ISIS 416857 | 17 |
| ISIS 416859 | 18 |
| ISIS 416860 | 25 |
| ISIS 416861 | 23 |
| ISIS 416862 | 21 |
| ISIS 416863 | 22 |
| ISIS 416865 | 20 |
| ISIS 416866 | 22 |
| ISIS 416867 | 20 |

### Hematology Assays

Blood obtained from all the mice groups were sent to Antech Diagnostics for hematocrit (HCT) measurements, as well as measurements of various blood cells, such as WBC (neutrophils, lymphocytes, and monocytes), RBC, and platelets, as well as total hemoglobin content analysis. The results are presented in Tables 90 and 91. Those antisense oligonucleotides which did not affect a decrease in platelet count of more than 50% and an increase in monocyte count of more than three-fold were selected for further studies.

TABLE 90
Effect of antisense oligonucleotide treatment on hematologic
factors in CD1 mice

| | RBC | Hemoglobin | HCT | WBC |
|---|---|---|---|---|
| | (10⁶/μL) | (g/dL) | (%) | (10³/μL) |
| PBS | 10 | 15 | 51 | 7 |
| ISIS 416850 | 10 | 15 | 49 | 5 |
| ISIS 416858 | 9 | 14 | 50 | 8 |
| ISIS 416864 | 10 | 15 | 52 | 5 |
| ISIS 412223 | 9 | 15 | 48 | 7 |
| ISIS 412224 | 10 | 15 | 50 | 9 |
| ISIS 412225 | 9 | 15 | 50 | 7 |
| ISIS 413481 | 9 | 13 | 45 | 7 |
| ISIS 413482 | 10 | 15 | 50 | 8 |
| ISIS 416848 | 9 | 14 | 47 | 7 |
| ISIS 416849 | 9 | 14 | 48 | 9 |
| ISIS 416851 | 9 | 14 | 47 | 6 |
| ISIS 416852 | 9 | 14 | 49 | 5 |
| ISIS 416853 | 11 | 17 | 56 | 8 |
| ISIS 416854 | 9 | 13 | 43 | 12 |
| ISIS 416855 | 9 | 14 | 50 | 6 |
| ISIS 416856 | 9 | 14 | 47 | 5 |
| ISIS 416857 | 10 | 15 | 53 | 6 |
| ISIS 416859 | 10 | 15 | 49 | 6 |
| ISIS 416860 | 10 | 15 | 51 | 7 |
| ISIS 416861 | 9 | 14 | 48 | 7 |
| ISIS 416862 | 9 | 14 | 49 | 6 |
| ISIS 416863 | 9 | 14 | 48 | 7 |
| ISIS 416865 | 9 | 14 | 50 | 7 |
| ISIS 416866 | 9 | 15 | 51 | 6 |
| ISIS 416867 | 10 | 14 | 47 | 8 |

TABLE 91
Effect of antisense oligonucleotide treatment on blood cell count in
CD1 mice

| | Neutrophil | Lymphocyte | Monocytes | Platelets |
|---|---|---|---|---|
| | (cells/μL) | (cells/μL) | (cells/μL) | (10³/μL) |
| PBS | 1023 | 6082 | 205 | 940 |
| ISIS 416850 | 1144 | 4004 | 156 | 916 |
| ISIS 416858 | 2229 | 5480 | 248 | 782 |
| ISIS 416864 | 973 | 3921 | 141 | 750 |
| ISIS 412223 | 1756 | 4599 | 200 | 862 |
| ISIS 412224 | 2107 | 6284 | 195 | 647 |
| ISIS 412225 | 1547 | 4969 | 293 | 574 |
| ISIS 413481 | 1904 | 4329 | 204 | 841 |
| ISIS 413482 | 1958 | 5584 | 275 | 818 |
| ISIS 416848 | 1264 | 5268 | 180 | 953 |
| ISIS 416849 | 1522 | 6967 | 253 | 744 |
| ISIS 416851 | 1619 | 4162 | 194 | 984 |
| ISIS 416852 | 1241 | 3646 | 189 | 903 |
| ISIS 416853 | 2040 | 5184 | 225 | 801 |
| ISIS 416854 | 2082 | 9375 | 455 | 1060 |
| ISIS 416855 | 1443 | 4236 | 263 | 784 |
| ISIS 416856 | 1292 | 3622 | 151 | 753 |
| ISIS 416857 | 1334 | 3697 | 215 | 603 |
| ISIS 416859 | 1561 | 4363 | 229 | 826 |
| ISIS 416860 | 1291 | 4889 | 161 | 937 |
| ISIS 416861 | 1122 | 5119 | 219 | 836 |
| ISIS 416862 | 1118 | 4445 | 174 | 1007 |
| ISIS 416863 | 1330 | 5617 | 226 | 1131 |
| ISIS 416865 | 1227 | 5148 | 315 | 872 |
| ISIS 416866 | 1201 | 4621 | 211 | 1045 |
| ISIS 416867 | 1404 | 6078 | 188 | 1006 |

### Example 22

### Measurement of Half-Life of Antisense Oligonucleotide in CD1 Mouse Liver

Fifteen antisense oligonucleotides which had been evaluated in CD1 mice (Example 21) were further evaluated. CD1 mice were treated with ISIS antisense oligonucleotides and the oligonucleotide half-life as well the elapsed time for oligonucleotide degradation and elimination in the liver was evaluated.

### Treatment

Groups of fifteen CD1 mice each were injected subcutaneously twice per week for 2 weeks with 50 mg/kg of ISIS 412223, ISIS 412225, ISIS 413481, ISIS 413482, ISIS 416851, ISIS 416852, ISIS 416856, ISIS 416860, ISIS 416861, ISIS 416863, ISIS 416866, ISIS 416867, ISIS 412224, ISIS 416848 or ISIS 416859. Five mice from each group were sacrificed 3 days, 28 days, and 56 days after the last dose, livers were collected for analysis.

### Measurement of Oligonucleotide Concentration

The concentration of the full-length oligonucleotide was measured. The method used is a modification of previously published methods (Leeds et al., 1996; Geary et al., 1999) which consist of a phenol-chloroform (liquid-liquid) extraction followed by a solid phase extraction. An internal standard (ISIS 355868, a 27-mer 2′-O-methoxyethyl modified phosphorothioate oligonucleotide, GCGTTTGCTCTTCTTCTTGCGTTTTTT, designated herein as SEQ ID NO: 270) was added prior to extraction. Tissue sample concentrations were calculated using calibration curves, with a lower limit of quantitation (LLOQ) of approximately 1.14 μg/g. The results are presented in Table 92 expressed as μg/g liver tissue. The half-life of each oligonucleotide was also presented in Table 92.

TABLE 92
Full-length oligonucleotide concentration and half-life in the liver of
CD1 mice

| | | | | | Half-Life |
|---|---|---|---|---|---|
| ISIS No | Motif | day 3 | day 28 | day 56 | (days) |
| 412223 | 5-10-5 | 276 | 127 | 52 | 21.9 |
| 412224 | 5-10-5 | 287 | 111 | 31 | 16.6 |
| 412225 | 5-10-5 | 279 | 91 | 47 | 20.7 |
| 413481 | 5-10-5 | 185 | 94 | 31 | 20.6 |
| 413482 | 5-10-5 | 262 | 95 | 40 | 19.5 |
| 416848 | 5-10-5 | 326 | 147 | 68 | 23.5 |
| 416851 | 5-10-5 | 319 | 147 | 68 | 23.8 |
| 416852 | 5-10-5 | 306 | 145 | 83 | 28.4 |
| 416856 | 5-10-5 | 313 | 115 | 46 | 19.2 |
| 416859 | 5-10-5 | 380 | 156 | 55 | 19.0 |
| 416860 | 5-10-5 | 216 | 96 | 36 | 20.6 |
| 416861 | 5-10-5 | 175 | 59 | 39 | 24.5 |
| 416863 | 5-10-5 | 311 | 101 | 48 | 19.8 |
| 416866 | 5-10-5 | 246 | 87 | 25 | 16.0 |
| 416867 | 5-10-5 | 246 | 87 | 35 | 18.9 |

### Example 23

### Tolerability of Antisense Oligonucleotides Targeting Human Factor XI in Sprague-Dawley Rats

Fifteen antisense oligonucleotides which had been evaluated in CD1 mice (Example 21) were further evaluated in Sprague-Dawley rats for changes in the levels of various metabolic markers.

### Treatment

Groups of four Sprague Dawley rats each were injected subcutaneously twice per week for 6 weeks with 50 mg/kg of ISIS 412223, ISIS 412224, ISIS 412225, ISIS 413481, ISIS 413482, ISIS 416848, ISIS 416851, ISIS 416852, ISIS 416856, ISIS 416859, ISIS 416860, ISIS 416861, ISIS 416863, ISIS 416866, or ISIS 416867. A control group of four Sprague Dawley rats was injected subcutaneously with PBS twice per week for 6 weeks. Body weight measurements were taken before and throughout the treatment period. Three days after the last dose, urine samples were collected and the rats were then sacrificed, organ weights were measured, and blood was collected for further analysis.

### Body Weight and Organ Weights

The body weights of the rats were measured at the onset of the study and subsequently twice per week. The body weights are presented in Table 93 and are expressed as increase in grams over the PBS control weight taken before the start of treatment. Liver, spleen and kidney weights were measured at the end of the study, and are also presented in Table 93 as a percentage of the body weight. Those antisense oligonucleotides which did not affect more than six-fold increases in liver and spleen weight above the PBS control were selected for further studies.

TABLE 93
Change in body and organ weights of Sprague Dawley rats after antisense
oligonucleotide treatment

| | Body | Liver | Kidney | Spleen |
|---|---|---|---|---|
| | weight (g) | (%) | (%) | (%) |
| PBS | 179 | 4 | 0.9 | 0.2 |
| ISIS 412223 | 126 | 5 | 1.0 | 0.5 |
| ISIS 412224 | 165 | 5 | 1.0 | 0.5 |
| ISIS 412225 | 184 | 4 | 1.0 | 0.5 |
| ISIS 413481 | 147 | 5 | 0.9 | 0.3 |
| ISIS 413482 | 158 | 5 | 1.0 | 0.6 |
| ISIS 416848 | 117 | 5 | 1.1 | 0.8 |
| ISIS 416851 | 169 | 5 | 0.9 | 0.3 |
| ISIS 416852 | 152 | 5 | 1.0 | 0.4 |
| ISIS 416856 | 156 | 5 | 1.0 | 0.4 |
| ISIS 416859 | 128 | 4 | 1.0 | 0.4 |
| ISIS 416860 | 123 | 5 | 1.0 | 0.5 |
| ISIS 416861 | 182 | 5 | 0.9 | 0.3 |
| ISIS 416863 | 197 | 5 | 1.0 | 0.4 |
| ISIS 416866 | 171 | 5 | 1.0 | 0.5 |
| ISIS 416867 | 129 | 5 | 1.0 | 0.5 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Measurements of alanine transaminase (ALT) and aspartate transaminase (AST) are expressed in IU/L and the results are presented in Table 94. Those antisense oligonucleotides which did not affect an increase in ALT/AST levels above seven-fold of control levels were selected for further studies. Plasma levels of bilirubin and albumin were also measured using the same clinical chemistry analyzer and results are presented in Table 94 and expressed in mg/dL. Those antisense oligonucleotides which did not affect an increase in levels of bilirubin more than two-fold of the control levels by antisense oligonucleotide treatment were selected for further studies.

TABLE 94
Effect of antisense oligonucleotide treatment on metabolic markers in
the liver of Sprague-Dawley rats

| | ALT | AST | Bilirubin | Albumin |
|---|---|---|---|---|
| | (IU/L) | (IU/L) | (mg/dL) | (mg/dL) |
| PBS | 42 | 71 | 0.13 | 4 |
| ISIS 412223 | 85 | 180 | 0.14 | 5 |
| ISIS 412224 | 84 | 132 | 0.12 | 4 |
| ISIS 412225 | 48 | 108 | 0.15 | 5 |
| ISIS 413481 | 54 | 80 | 0.22 | 4 |
| ISIS 413482 | 59 | 157 | 0.14 | 4 |
| ISIS 416848 | 89 | 236 | 0.14 | 3 |
| ISIS 416851 | 64 | 91 | 0.14 | 4 |
| ISIS 416852 | 49 | 87 | 0.15 | 4 |
| ISIS 416856 | 123 | 222 | 0.13 | 4 |
| ISIS 416859 | 114 | 206 | 0.21 | 5 |
| ISIS 416860 | 70 | 157 | 0.15 | 4 |
| ISIS 416861 | 89 | 154 | 0.15 | 5 |
| ISIS 416863 | 47 | 78 | 0.13 | 4 |
| ISIS 416866 | 41 | 78 | 0.16 | 4 |
| ISIS 416867 | 47 | 126 | 0.17 | 4 |

### Kidney Function

To evaluate the effect of ISIS oligonucleotides on the kidney function, plasma concentrations of blood urea nitrogen (BUN) and creatinine were measured using an automated clinical chemistry analyzer (Hitachi Olympus AU400e, Melville, N.Y.). Results are presented in Table 95, expressed in mg/dL. Those antisense oligonucleotides which did not affect more than a two-fold increase in BUN levels compared to the PBS control were selected for further studies. The total urine protein and ratio of urine protein to creatinine in total urine samples after antisense oligonucleotide treatment was calculated and is also presented in Table 95. Those antisense oligonucleotides which did not affect more than a five-fold increase in urine protein/creatinine ratios compared to the PBS control were selected for further studies.

TABLE 95
Effect of antisense oligonucleotide treatment on metabolic markers in the
kidney of Sprague-Dawley rats

| | | | Total | |
|---|---|---|---|---|
| | | | urine | Urine |
| | BUN | Creatinine | protein | protein/creatinine |
| | (mg/dL) | (mg/dL) | (mg/dL) | ratio |
| PBS | 19 | 38 | 60 | 1.7 |
| ISIS 412223 | 24 | 46 | 224 | 4.6 |
| ISIS 412224 | 24 | 44 | 171 | 3.8 |
| ISIS 412225 | 23 | 58 | 209 | 4.0 |
| ISIS 413481 | 26 | 45 | 148 | 3.6 |
| ISIS 413482 | 23 | 34 | 157 | 4.8 |
| ISIS 416848 | 26 | 64 | 231 | 3.9 |
| ISIS 416851 | 24 | 70 | 286 | 4.0 |
| ISIS 416852 | 25 | 60 | 189 | 3.0 |
| ISIS 416856 | 23 | 48 | 128 | 2.7 |
| ISIS 416859 | 24 | 44 | 144 | 3.3 |
| ISIS 416860 | 23 | 58 | 242 | 4.6 |
| ISIS 416861 | 22 | 39 | 205 | 5.1 |
| ISIS 416863 | 29 | 73 | 269 | 3.8 |
| ISIS 416866 | 22 | 85 | 486 | 6.2 |
| ISIS 416867 | 22 | 70 | 217 | 3.1 |

### Hematology Assays

Blood obtained from all rat groups were sent to Antech Diagnostics for hematocrit (HCT) measurements, as well as measurements of the various blood cells, such as WBC (neutrophils and lymphocytes), RBC, and platelets, and total hemoglobin content. The results are presented in Tables 96 and 97. Those antisense oligonucleotides which did not affect a decrease in platelet count of more than 50% and an increase in monocyte count of more than three-fold were selected for further studies.

TABLE 96
Effect of antisense oligonucleotide treatment on hematologic factors in
Sprague-Dawley rats

| | RBC | Hemoglobin | | WBC |
|---|---|---|---|---|
| | (10⁶/mL) | (g/dL) | HCT (%) | (10³/mL) |
| PBS | 6.9 | 13.2 | 42 | 9 |
| ISIS 412223 | 7.2 | 13.1 | 41 | 20 |
| ISIS 412224 | 7.4 | 13.4 | 42 | 20 |
| ISIS 412225 | 7.4 | 13.4 | 42 | 15 |
| ISIS 413481 | 7.5 | 14.2 | 43 | 14 |
| ISIS 413482 | 7.1 | 13.2 | 40 | 13 |
| ISIS 416848 | 6.0 | 11.1 | 35 | 17 |
| ISIS 416851 | 7.4 | 13.7 | 42 | 11 |
| ISIS 416852 | 7.2 | 13.4 | 42 | 13 |
| ISIS 416856 | 7.7 | 14.1 | 43 | 19 |
| ISIS 416859 | 7.8 | 14.0 | 45 | 16 |
| ISIS 416860 | 7.8 | 14.1 | 45 | 17 |
| ISIS 416861 | 7.7 | 14.6 | 45 | 15 |
| ISIS 416863 | 7.6 | 14.1 | 45 | 17 |
| ISIS 416866 | 7.8 | 14.0 | 44 | 20 |
| ISIS 416867 | 7.8 | 14.0 | 45 | 14 |

TABLE 97
Effect of antisense oligonucleotide treatment on blood cell count in
Sprague-Dawley rats

| | Neutrophil | Lymphocyte | Platelets |
|---|---|---|---|
| | (/mL) | (/mL) | (10³/mL) |
| PBS | 988 | 7307 | 485 |
| ISIS 412223 | 1826 | 16990 | 567 |
| ISIS 412224 | 1865 | 16807 | 685 |
| ISIS 412225 | 1499 | 13204 | 673 |
| ISIS 413481 | 1046 | 12707 | 552 |
| ISIS 413482 | 1125 | 11430 | 641 |
| ISIS 416848 | 1874 | 14316 | 384 |
| ISIS 416851 | 1001 | 9911 | 734 |
| ISIS 416852 | 836 | 11956 | 632 |
| ISIS 416856 | 3280 | 14328 | 740 |
| ISIS 416859 | 1414 | 14323 | 853 |
| ISIS 416860 | 1841 | 13986 | 669 |
| ISIS 416861 | 1813 | 12865 | 1008 |
| ISIS 416863 | 1720 | 14669 | 674 |
| ISIS 416866 | 1916 | 16834 | 900 |
| ISIS 416867 | 3044 | 10405 | 705 |

### Example 24

### Measurement of Half-Life of Antisense Oligonucleotide in the Liver and Kidney of Sprague-Dawley Rats

Sprague Dawley rats were treated with ISIS antisense oligonucleotides targeting human Factor XI and the oligonucleotide half-life as well as the elapsed time for oligonucleotide degradation and elimination from the liver and kidney was evaluated.

### Treatment

Groups of four Sprague Dawley rats each were injected subcutaneously twice per week for 2 weeks with 20 mg/kg of ISIS 412223, ISIS 412224, ISIS 412225, ISIS 413481, ISIS 413482, ISIS 416848, ISIS 416851, ISIS 416852, ISIS 416856, ISIS 416859, ISIS 416860, ISIS 416861, ISIS 416863, ISIS 416866, or ISIS 416867. Three days after the last dose, the rats were sacrificed, and livers and kidneys were harvested.

### Measurement of Oligonucleotide Concentration

The concentration of the full-length oligonucleotide as well as the total oligonucleotide concentration (including the degraded form) was measured. The method used is a modification of previously published methods (Leeds et al., 1996; Geary et al., 1999) which consist of a phenol-chloroform (liquid-liquid) extraction followed by a solid phase extraction. An internal standard (ISIS 355868, a 27-mer 2′-O-methoxyethyl modified phosphorothioate oligonucleotide, GCGTTTGCTCTTCTTCTTGCGTTTTTT, designated herein as SEQ ID NO: 270) was added prior to extraction. Tissue sample concentrations were calculated using calibration curves, with a lower limit of quantitation (LLOQ) of approximately 1.14 μg/g. The results are presented in Tables 98 and 99, expressed as μg/g liver or kidney tissue. Half-lives were then calculated using WinNonlin software (PHARSIGHT) and presented in Table 100.

TABLE 98
Full-length oligonucleotide concentration (μg/g) in the liver and kidney
of Sprague-Dawley rats

| ISIS No | Motif | Kidney | Liver |
|---|---|---|---|
| 412223 | 5-10-5 | 551 | 97 |
| 412224 | 5-10-5 | 487 | 107 |
| 412225 | 5-10-5 | 202 | 119 |
| 413481 | 5-10-5 | 594 | 135 |
| 413482 | 5-10-5 | 241 | 95 |
| 416848 | 5-10-5 | 488 | 130 |
| 416851 | 5-10-5 | 264 | 193 |
| 416852 | 5-10-5 | 399 | 108 |
| 416856 | 5-10-5 | 378 | 84 |
| 416859 | 5-10-5 | 253 | 117 |
| 416860 | 5-10-5 | 247 | 94 |
| 416861 | 5-10-5 | 187 | 159 |
| 416863 | 5-10-5 | 239 | 82 |
| 416866 | 5-10-5 | 210 | 98 |
| 416867 | 5-10-5 | 201 | 112 |

TABLE 99
Total oligonucleotide concentration (μg/g) in the liver and kidney of
Sprague-Dawley rats

| ISIS No | Motif | Kidney | Liver |
|---|---|---|---|
| 412223 | 5-10-5 | 395 | 86 |
| 412224 | 5-10-5 | 292 | 78 |
| 412225 | 5-10-5 | 189 | 117 |
| 413481 | 5-10-5 | 366 | 96 |
| 413482 | 5-10-5 | 217 | 91 |
| 416848 | 5-10-5 | 414 | 115 |
| 416851 | 5-10-5 | 204 | 178 |
| 416852 | 5-10-5 | 304 | 87 |
| 416856 | 5-10-5 | 313 | 80 |
| 416859 | 5-10-5 | 209 | 112 |
| 416860 | 5-10-5 | 151 | 76 |
| 416861 | 5-10-5 | 165 | 144 |
| 416863 | 5-10-5 | 203 | 79 |
| 416866 | 5-10-5 | 145 | 85 |
| 416867 | 5-10-5 | 157 | 98 |

TABLE 100
Half-life (days) of ISIS oligonucleotides in the liver and kidney of
Sprague-Dawley rats

| ISIS No | Motif | Half-life |
|---|---|---|
| 412223 | 5-10-5 | 22 |
| 412224 | 5-10-5 | 17 |
| 412225 | 5-10-5 | 21 |
| 413481 | 5-10-5 | 21 |
| 413482 | 5-10-5 | 20 |
| 416848 | 5-10-5 | 24 |
| 416851 | 5-10-5 | 24 |
| 416852 | 5-10-5 | 28 |
| 416856 | 5-10-5 | 19 |
| 416859 | 5-10-5 | 19 |
| 416860 | 5-10-5 | 21 |
| 416861 | 5-10-5 | 25 |
| 416863 | 5-10-5 | 20 |
| 416866 | 5-10-5 | 16 |
| 416867 | 5-10-5 | 19 |

### Example 25

### Tolerability of Antisense Oligonucleotides Targeting Human Factor XI in CD1 Mice

ISIS oligonucleotides with 6-8-6 MOE and 5-8-5 MOE motifs targeting human Factor XI were administered in CD1 mice evaluated for changes in the levels of various metabolic markers.

### Treatment

Groups of five CD1 mice each were injected subcutaneously twice per week for 6 weeks with 50 mg/kg of ISIS 416850, ISIS 445498, ISIS 445503, ISIS 445504, ISIS 445505, ISIS 445509, ISIS 445513, ISIS 445522, ISIS 445530, ISIS 445531 or ISIS 445532. A control group of five CD1 mice was injected subcutaneously with PBS twice per week for 6 weeks. Body weight measurements were taken before and at the end of the treatment period. Three days after the last dose, the mice were sacrificed, organ weights were measured, and blood was collected for further analysis.

### Body Weight and Organ Weight

The body weight changes in the mice are presented in Table 101 and are expressed increase in grams over the PBS control weight taken before the start of treatment. Liver, spleen and kidney weights were measured at the end of the study, and are also presented in Table 101 as percentage of the body weight. Those antisense oligonucleotides which did not affect more than six-fold increases in liver and spleen weight above the PBS control were selected for further studies.

TABLE 101
Change in body and organ weights of CD1 mice after antisense
oligonucleotide treatment

| | Body | Liver | Kidney | Spleen |
|---|---|---|---|---|
| | weight (g) | (%) | (%) | (%) |
| PBS | 10 | 5 | 1.6 | 0.3 |
| ISIS 416850 | 11 | 6 | 1.5 | 0.4 |
| ISIS 445498 | 10 | 6 | 1.6 | 0.5 |
| ISIS 445503 | 9 | 8 | 1.4 | 0.6 |
| ISIS 445504 | 11 | 6 | 1.6 | 0.4 |
| ISIS 445505 | 12 | 6 | 1.5 | 0.5 |
| ISIS 445509 | 10 | 6 | 1.6 | 0.5 |
| ISIS 445513 | 9 | 5 | 1.6 | 0.4 |
| ISIS 445522 | 11 | 6 | 1.7 | 0.4 |
| ISIS 445530 | 11 | 6 | 1.5 | 0.5 |
| ISIS 445531 | 10 | 6 | 1.5 | 0.5 |
| ISIS 445532 | 10 | 6 | 1.6 | 0.4 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Measurements of alanine transaminase (ALT) and aspartate transaminase (AST) are expressed in IU/L and the results are presented in Table 102. Those antisense oligonucleotides which did not affect an increase in ALT/AST levels above seven-fold of control levels were selected for further studies. Plasma levels of bilirubin and albumin were also measured and results are also presented in Table 102 and expressed in mg/dL. Those antisense oligonucleotides which did not affect an increase in levels of bilirubin more than two-fold of the control levels by antisense oligonucleotide treatment were selected for further studies.

TABLE 102
Effect of antisense oligonucleotide treatment on metabolic markers in the
liver of CD1 mice

| | ALT | AST | Bilirubin | Albumin |
|---|---|---|---|---|
| | (IU/L) | (IU/L) | (mg/dL) | (mg/dL) |
| PBS | 34 | 49 | 0.23 | 3.6 |
| ISIS 416850 | 90 | 115 | 0.20 | 3.2 |
| ISIS 445498 | 66 | 102 | 0.24 | 3.4 |
| ISIS 445503 | 1314 | 852 | 0.28 | 3.4 |
| ISIS 445504 | 71 | 107 | 0.17 | 3.4 |
| ISIS 445505 | 116 | 153 | 0.18 | 3.2 |
| ISIS 445509 | 80 | 117 | 0.17 | 3.1 |
| ISIS 445513 | 37 | 84 | 0.22 | 3.1 |
| ISIS 445522 | 51 | 110 | 0.19 | 3.4 |
| ISIS 445530 | 104 | 136 | 0.18 | 3.2 |
| ISIS 445531 | 60 | 127 | 0.16 | 3.2 |
| ISIS 445532 | 395 | 360 | 0.20 | 2.9 |

### Kidney Function

To evaluate the effect of ISIS oligonucleotides on kidney function, plasma concentrations of blood urea nitrogen (BUN) were measured using an automated clinical chemistry analyzer (Hitachi Olympus AU400e, Melville, N.Y.). Results are presented in Table 103, expressed in mg/dL. Those antisense oligonucleotides which did not affect more than a two-fold increase in BUN levels compared to the PBS control were selected for further studies.

TABLE 103
Effect of antisense oligonucleotide treatment on BUN levels (mg/dL) in
the kidney of CD1 mice

| | BUN |
|---|---|
| PBS | 29 |
| ISIS 416850 | 28 |
| ISIS 445498 | 28 |
| ISIS 445503 | 29 |
| ISIS 445504 | 29 |
| ISIS 445505 | 29 |
| ISIS 445509 | 29 |
| ISIS 445513 | 27 |
| ISIS 445522 | 28 |
| ISIS 445530 | 26 |
| ISIS 445531 | 27 |
| ISIS 445532 | 23 |

### Hematology Assays

Blood obtained from all mice groups were sent to Antech Diagnostics for hematocrit (HCT) measurements, as well as measurements of the various blood cells, such as WBC (neutrophils and lymphocytes), RBC, and platelets, and total hemoglobin content. The results are presented in Tables 104 and 105. Those antisense oligonucleotides which did not affect a decrease in platelet count of more than 50% and an increase in monocyte count of more than three-fold were selected for further studies.

TABLE 104
Effect of antisense oligonucleotide treatment on hematologic factors in
CD1 mice

| | RBC | Hemoglobin | HCT | WBC |
|---|---|---|---|---|
| | (10⁶/mL) | (g/dL) | (%) | (10³/mL) |
| PBS | 9.6 | 15.0 | 51 | 6 |
| ISIS 416850 | 9.8 | 14.8 | 50 | 6 |
| ISIS 445498 | 9.4 | 13.9 | 47 | 5 |
| ISIS 445503 | 9.2 | 13.6 | 46 | 8 |
| ISIS 445504 | 9.6 | 14.7 | 49 | 5 |
| ISIS 445505 | 9.6 | 14.6 | 49 | 5 |
| ISIS 445509 | 10.2 | 15.3 | 51 | 5 |
| ISIS 445513, | 9.8 | 15.0 | 50 | 7 |
| ISIS 445522 | 9.7 | 14.6 | 49 | 5 |
| ISIS 445530 | 10.0 | 15.1 | 50 | 7 |
| ISIS 445531 | 9.4 | 14.5 | 48 | 9 |
| ISIS 445532 | 9.7 | 14.8 | 48 | 7 |

TABLE 105
Effect of antisense oligonucleotide treatment on blood cell count in
CD1 mice

| | Neutrophil | Lymphocyte | Platelets |
|---|---|---|---|
| | (/mL) | (/mL) | (10³/mL) |
| PBS | 1356 | 4166 | 749 |
| ISIS 416850 | 1314 | 4710 | 614 |
| ISIS 445498 | 1197 | 3241 | 802 |
| ISIS 445503 | 1475 | 6436 | 309 |
| ISIS 445504 | 959 | 3578 | 826 |
| ISIS 445505 | 818 | 3447 | 725 |
| ISIS 445509 | 1104 | 3758 | 1085 |
| ISIS 445513 | 959 | 5523 | 942 |
| ISIS 445522 | 698 | 3997 | 1005 |
| ISIS 445530 | 930 | 5488 | 849 |
| ISIS 445531 | 2341 | 6125 | 996 |
| ISIS 445532 | 1116 | 5490 | 689 |

### Example 26

### Tolerability of Antisense Oligonucleotides Targeting Human Factor XI in Sprague-Dawley Rats

Eight antisense oligonucleotides which had been evaluated in CD1 mice (Example 25) were further evaluated in Sprague-Dawley rats for changes in the levels of various metabolic markers.

### Treatment

Groups of four Sprague Dawley rats each were injected subcutaneously twice per week for 6 weeks with 50 mg/kg of ISIS 445498, ISIS 445504, ISIS 445505, ISIS 445509, ISIS 445513, ISIS 445522, ISIS 445530 or ISIS 445531. A control group of Sprague Dawley rats was injected subcutaneously with PBS twice per week for 6 weeks. Body weight measurements were taken before and throughout the treatment period. Three days after the last dose, urine samples were collected and the rats were then sacrificed, organ weights were measured, and blood was collected for further analysis.

### Body Weight and Organ Weight

The body weights of the rats were measured at the onset of the study and subsequently twice per week. The body weights are presented in Table 106 and are expressed as percent increase over the PBS control weight taken before the start of treatment. Liver, spleen and kidney weights were measured at the end of the study, and are also presented in Table 106 as a percentage of the body weight. Those antisense oligonucleotides which did not affect more than six-fold increases in liver and spleen weight above the PBS control were selected for further studies.

TABLE 106
Change in body and organ weights of Sprague Dawley rats after antisense
oligonucleotide treatment (%)

| | Body | | | |
|---|---|---|---|---|
| | weight | Liver | Spleen | Kidney |
| ISIS 445498 | −17 | +26 | +107 | −10 |
| ISIS 445504 | −15 | +22 | +116 | +6 |
| ISIS 445505 | −21 | +12 | +146 | +2 |
| ISIS 445509 | −17 | +16 | +252 | +3 |
| ISIS 445513 | −13 | +25 | +194 | +15 |
| ISIS 445522 | −13 | +26 | +184 | +19 |
| ISIS 445530 | −7 | +24 | +99 | +4 |
| ISIS 445531 | −10 | +17 | +89 | +4 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Plasma concentrations of ALT (alanine transaminase) and AST (aspartate transaminase) were measured and the results are presented in Table 107 expressed in IU/L. Those antisense oligonucleotides which did not affect an increase in ALT/AST levels above seven-fold of control levels were selected for further studies. Plasma levels of bilirubin and albumin were also measured using the same clinical chemistry analyzer; results are presented in Table 107 and expressed in mg/dL. Those antisense oligonucleotides which did not affect an increase in levels of bilirubin more than two-fold of the control levels by antisense oligonucleotide treatment were selected for further studies.

TABLE 107
Effect of antisense oligonucleotide treatment on metabolic markers in the
liver of Sprague-Dawley rats

| | ALT | AST | Bilirubin | Albumin |
|---|---|---|---|---|
| | (IU/L) | (IU/L) | (mg/dL) | (mg/dL) |
| PBS | 102 | 36 | 0.13 | 3.7 |
| ISIS 445498 | 417 | 124 | 0.14 | 3.7 |
| ISIS 445504 | 206 | 86 | 0.11 | 3.5 |
| ISIS 445505 | 356 | 243 | 0.15 | 3.6 |
| ISIS 445509 | 676 | 291 | 0.14 | 3.5 |
| ISIS 445513 | 214 | 91 | 0.15 | 3.5 |
| ISIS 445522 | 240 | 138 | 0.47 | 3.6 |
| ISIS 445530 | 116 | 56 | 0.11 | 3.7 |
| ISIS 445531 | 272 | 137 | 0.12 | 3.7 |

### Kidney Function

To evaluate the effect of ISIS oligonucleotides on kidney function, plasma concentrations of blood urea nitrogen (BUN) and creatinine were measured using an automated clinical chemistry analyzer (Hitachi Olympus AU400e, Melville, N.Y.). Results are presented in Table 108, expressed in mg/dL. Those antisense oligonucleotides which did not affect more than a two-fold increase in BUN levels compared to the PBS control were selected for further studies. The total urine protein and ratio of urine protein to creatinine in total urine samples after antisense oligonucleotide treatment was calculated and is also presented in Table 108. Those antisense oligonucleotides which did not affect more than a five-fold increase in urine protein/creatinine ratios compared to the PBS control were selected for further studies.

TABLE 108
Effect of antisense oligonucleotide treatment on metabolic markers in the
kidney of Sprague-Dawley rats

| | | | Urine |
|---|---|---|---|
| | BUN | Creatinine | protein/creatinine |
| | (mg/dL) | (mg/dL) | ratio |
| PBS | 18 | 0.4 | 1.4 |
| ISIS 445498 | 25 | 0.5 | 3.1 |
| ISIS 445504 | 26 | 0.4 | 4.3 |
| ISIS 445505 | 24 | 0.4 | 3.8 |
| ISIS 445509 | 27 | 0.5 | 4.0 |
| ISIS 445513 | 24 | 0.4 | 4.6 |
| ISIS 445522 | 25 | 0.4 | 6.4 |
| ISIS 445530 | 22 | 0.4 | 4.2 |
| ISIS 445531 | 23 | 0.4 | 3.4 |

### Hematology Assays

Blood obtained from all rat groups were sent to Antech Diagnostics for hematocrit (HCT) measurements, as well as measurements of the various blood cells, such as WBC (neutrophils, lymphocytes, and monocytes), RBC, and platelets, and total hemoglobin content. The results are presented in Tables 109 and 110. Those antisense oligonucleotides which did not affect a decrease in platelet count of more than 50% and an increase in monocyte count of more than three-fold were selected for further studies.

TABLE 109
Effect of antisense oligonucleotide treatment on hematologic factors in
Sprague-Dawley rats

| | RBC | Hemoglobin | HCT | WBC |
|---|---|---|---|---|
| | (/pL) | (g/dL) | (%) | (/nL) |
| PBS | 8.8 | 16.0 | 55 | 13 |
| ISIS 445498 | 8.5 | 14.7 | 49 | 13 |
| ISIS 445504 | 8.9 | 14.7 | 50 | 16 |
| ISIS 445505 | 9.1 | 15.0 | 50 | 21 |
| ISIS 445509 | 8.4 | 14.1 | 47 | 17 |
| ISIS 445513 | 7.8 | 13.0 | 44 | 17 |
| ISIS 445522 | 7.7 | 13.6 | 47 | 18 |
| ISIS 445530 | 8.9 | 14.7 | 50 | 12 |
| ISIS 445531 | 8.8 | 14.8 | 50 | 13 |

TABLE 110
Effect of antisense oligonucleotide treatment on blood cell count in
Sprague-Dawley rats

| | Neutrophil | Lymphocyte | Monocytes | Platelets |
|---|---|---|---|---|
| | (%) | (%) | (%) | (/nL) |
| PBS | 14 | 82 | 2.0 | 1007 |
| ISIS 445498 | 9 | 89 | 2.0 | 1061 |
| ISIS 445504 | 10 | 87 | 2.0 | 776 |
| ISIS 445505 | 10 | 87 | 2.5 | 1089 |
| ISIS 445509 | 11 | 84 | 3.8 | 1115 |
| ISIS 445513 | 14 | 82 | 3.5 | 1051 |
| ISIS 445522 | 13 | 84 | 2.8 | 1334 |
| ISIS 445530 | 11 | 87 | 2.0 | 1249 |
| ISIS 445531 | 10 | 86 | 2.8 | 1023 |

### Example 27

### Tolerability of Antisense Oligonucleotides Targeting Human Factor XI in CD1 Mice

ISIS oligonucleotides with 4-8-4 MOE, 3-8-3 MOE, 2-10-2 MOE, 3-10-3 MOE, and 4-10-4

MOE motifs targeting human Factor XI were administered in CD1 mice evaluated for changes in the levels of various metabolic markers.

### Treatment

Groups of five CD1 mice each were injected subcutaneously twice per week for 6 weeks with 50 mg/kg of ISIS 449707, ISIS 449708, ISIS 449409, ISIS 449710, or ISIS 449711. A control group of five CD1 mice was injected subcutaneously with PBS twice per week for 6 weeks. Body weight measurements were taken before and at the end of the treatment period. Three days after the last dose, the mice were sacrificed, organ weights were measured, and blood was collected for further analysis.

### Body Weight and Organ Weight

The body weights of the mice taken at the end of the study are presented in Table 111 and are expressed in grams. Liver, spleen and kidney weights were also measured at the end of the study and are also presented in Table 111 as percentage of the body weight. Those antisense oligonucleotides which did not affect more than six-fold increases in liver and spleen weight above the PBS control were selected for further studies.

TABLE 111
Change in body and organ weights of CD1 mice after antisense
oligonucleotide treatment

| | Body | | | |
|---|---|---|---|---|
| | weight | Liver | Spleen | Kidney |
| | (g) | (%) | (%) | (%) |
| PBS | 39 | — | — | — |
| ISIS 449707 | 42 | +11 | +63 | −5 |
| ISIS 449708 | 40 | +17 | +66 | 0 |
| ISIS 449709 | 40 | +15 | +62 | −14 |
| ISIS 449710 | 42 | +6 | +43 | −7 |
| ISIS 449711 | 42 | +18 | +63 | −12 |

### Liver Function

To evaluate the effect of ISIS oligonucleotides on hepatic function, plasma concentrations of transaminases were measured using an automated clinical chemistry analyzer (Hitachi Olympus AU400e, Melville, N.Y.). Plasma concentrations of ALT (alanine transaminase) and AST (aspartate transaminase) were measured and the results are presented in Table 112 expressed in IU/L. Those antisense oligonucleotides which did not affect an increase in ALT/AST levels above seven-fold of control levels were selected for further studies. Plasma levels of bilirubin and albumin were also measured using the same clinical chemistry analyzer and results are presented in Table 112 and expressed in mg/dL. Those antisense oligonucleotides which did not affect an increase in levels of bilirubin more than two-fold of the control levels by antisense oligonucleotide treatment were selected for further studies.

TABLE 112
Effect of antisense oligonucleotide treatment on metabolic markers in the
liver of CD1 mice

| | ALT | AST | Bilirubin | Albumin |
|---|---|---|---|---|
| | (IU/L) | (IU/L) | (mg/dL) | (mg/dL) |
| PBS | 39 | 52 | 0.22 | 3.2 |
| ISIS 449707 | 41 | 62 | 0.19 | 2.3 |
| ISIS 449708 | 66 | 103 | 0.17 | 2.8 |
| ISIS 449709 | 62 | 83 | 0.18 | 2.8 |
| ISIS 449710 | 43 | 95 | 0.18 | 2.8 |
| ISIS 449711 | 52 | 83 | 0.22 | 2.8 |

### Kidney Function

To evaluate the effect of ISIS oligonucleotides on kidney function, plasma concentrations of blood urea nitrogen (BUN) and creatinine were measured using an automated clinical chemistry analyzer (Hitachi Olympus AU400e, Melville, N.Y.). Results are presented in Table 113, expressed in mg/dL. Those antisense oligonucleotides which did not affect more than a two-fold increase in BUN levels compared to the PBS control were selected for further studies.

TABLE 113
Effect of antisense oligonucleotide treatment on metabolic markers
(mg/dL) in the kidney of CD1 mice

| | BUN | Creatinine |
|---|---|---|
| PBS | 28 | 0.3 |
| ISIS 449707 | 27 | 0.2 |
| ISIS 449708 | 28 | 0.2 |
| ISIS 449709 | 34 | 0.3 |
| ISIS 449710 | 29 | 0.2 |
| ISIS 449711 | 26 | 0.2 |

### Hematology Assays

Blood obtained from all mice groups were sent to Antech Diagnostics for hematocrit (HCT), measurements, as well as measurements of the various blood cells, such as WBC (neutrophils, lymphocytes, and monocytes), RBC, and platelets, and total hemoglobin content. The results are presented in Tables 114 and 115. Those antisense oligonucleotides which did not affect a decrease in platelet count of more than 50% and an increase in monocyte count of more than three-fold were selected for further studies.

TABLE 114
Effect of antisense oligonucleotide treatment on hematologic factors in
CD1 mice

| | RBC | Hemoglobin | Hematocrit | WBC |
|---|---|---|---|---|
| | (/pL) | (g/dL) | (%) | (/nL) |
| PBS | 9.8 | 14.6 | 54 | 6 |
| ISIS 449707 | 8.4 | 12.4 | 45 | 6 |
| ISIS 449708 | 9.2 | 13.2 | 48 | 7 |
| ISIS 449709 | 9.2 | 13.2 | 49 | 5 |
| ISIS 449710 | 9.1 | 13.5 | 48 | 7 |
| ISIS 449711 | 9.0 | 13.3 | 48 | 6 |

TABLE 115
Effect of antisense oligonucleotide treatment on blood cell count in CD1
mice

| | Neutrophils | Lymphocytes | Monocytes | Platelets |
|---|---|---|---|---|
| | (%) | (%) | (%) | (/nL) |
| PBS | 15 | 80 | 3 | 1383 |
| ISIS 449707 | 11 | 85 | 3 | 1386 |
| ISIS 449708 | 17 | 77 | 5 | 1395 |
| ISIS 449709 | 19 | 76 | 4 | 1447 |
| ISIS 449710 | 15 | 81 | 3 | 1245 |
| ISIS 449711 | 15 | 79 | 6 | 1225 |

### Example 28

### Tolerability of Antisense Oligonucleotides Targeting Human Factor XI in Sprague-Dawley Rats

Five antisense oligonucleotides which had been evaluated in CD1 mice (Example 27) were further evaluated in Sprague-Dawley rats for changes in the levels of various metabolic markers.

### Treatment

Groups of four Sprague Dawley rats each were injected subcutaneously twice per week for 6 weeks with 50 mg/kg of ISIS 449707, ISIS 449708, ISIS 449709, ISIS 449710, or ISIS 449711. A control group of four Sprague Dawley rats was injected subcutaneously with PBS twice per week for 6 weeks. Body weight measurements were taken before and throughout the treatment period. Three days after the last dose, urine samples were collected and the rats were then sacrificed, organ weights were measured, and blood was collected for further analysis.

### Body Weight and Organ Weight

The body weights of the rats were measured at the onset of the study and at the end of the study. The body weight changes are presented in Table 116 and are expressed as increase in grams over the PBS control weight taken before the start of treatment. Liver, spleen and kidney weights were measured at the end of the study, and are also presented in Table 116 as a percentage of the body weight. Those antisense oligonucleotides which did not affect more than six-fold increases in liver and spleen weight above the PBS control were selected for further studies.

TABLE 116
Change in body and organ weights of Sprague Dawley rats after antisense
oligonucleotide treatment

| | Body | | | |
|---|---|---|---|---|
| | weight | Liver | Spleen | Kidney |
| | (g) | (%) | (%) | (%) |
| PBS | 478 | — | — | — |
| ISIS 449707 | 352 | +41 | +400 | +80 |
| ISIS 449708 | 382 | +31 | +259 | +40 |
| ISIS 449709 | 376 | +8 | +231 | +19 |
| ISIS 449710 | 344 | +82 | +302 | +50 |
| ISIS 449711 | 362 | +52 | +327 | +72 |

### Liver Function

To evaluate the impact of ISIS oligonucleotides on hepatic function, plasma concentrations of ALT and AST were measured using an automated clinical chemistry analyzer (Hitachi Olympus AU400e, Melville, N.Y.). Plasma concentrations of alanine transaminase (ALT) and aspartate transaminase (AST) were measured and the results are presented in Table 117 expressed in IU/L. Those antisense oligonucleotides which did not affect an increase in ALT/AST levels above seven-fold of control levels were selected for further studies. Plasma levels of bilirubin and albumin were also measured and results are presented in Table 117 and expressed in mg/dL. Those antisense oligonucleotides which did not affect an increase in levels of bilirubin more than two-fold of the control levels by antisense oligonucleotide treatment were selected for further studies.

TABLE 117
Effect of antisense oligonucleotide treatment on metabolic markers in the
liver of Sprague-Dawley rats

| | ALT | AST | Bilirubin | Albumin |
|---|---|---|---|---|
| | (IU/L) | (IU/L) | (mg/dL) | (mg/dL) |
| PBS | 41 | 107 | 0.1 | 3.4 |
| ISIS 449707 | 61 | 199 | 0.2 | 3.1 |
| ISIS 449708 | 25 | 90 | 0.1 | 3.2 |
| ISIS 449709 | 63 | 126 | 0.2 | 3.1 |
| ISIS 449710 | 36 | 211 | 0.1 | 2.9 |
| ISIS 449711 | 32 | 163 | 0.1 | 2.9 |

### Kidney Function

To evaluate the impact of ISIS oligonucleotides on kidney function, plasma concentrations of BUN and creatinine were measured using an automated clinical chemistry analyzer (Hitachi Olympus AU400e, Melville, N.Y.). Results are presented in Table 118, expressed in mg/dL. Those antisense oligonucleotides which did not affect more than a two-fold increase in BUN levels compared to the PBS control were selected for further studies. The total urine protein and ratio of urine protein to creatinine in total urine samples after antisense oligonucleotide treatment was calculated and is also presented in Table 118. Those antisense oligonucleotides which did not affect more than a five-fold increase in urine protein/creatinine ratios compared to the PBS control were selected for further studies.

TABLE 118
Effect of antisense oligonucleotide treatment on metabolic markers in the
kidney of Sprague-Dawley rats

| | | | Urine |
|---|---|---|---|
| | BUN | Creatinine | protein/creatinine |
| | (mg/dL) | (mg/dL) | ratio |
| PBS | 22 | 0.4 | 1.5 |
| ISIS 449707 | 24 | 0.4 | 3.2 |
| ISIS 449708 | 24 | 0.4 | 5.7 |
| ISIS 449709 | 24 | 0.4 | 3.4 |
| ISIS 449710 | 29 | 0.3 | 5.9 |
| ISIS 449711 | 28 | 0.4 | 7.3 |

### Hematology Assays

Blood obtained from all rat groups were sent to Antech Diagnostics for hematocrit (HCT) measurements, as well as measurements of the various blood cells, such as WBC (neutrophils, lymphocytes, and monocytes), RBC, and platelets, and total hemoglobin content. The results are presented in Tables 119 and 120. Those antisense oligonucleotides which did not affect a decrease in platelet count of more than 50% and an increase in monocyte count of more than three-fold were selected for further studies.

TABLE 119
Effect of antisense oligonucleotide treatment on hematologic factors in
Sprague-Dawley rats

| | RBC | Hemoglobin | Hematocrit | WBC |
|---|---|---|---|---|
| | (/pL) | (g/dL) | (%) | (/nL) |
| PBS | 8.2 | 15.1 | 50 | 16 |
| ISIS 449707 | 6.0 | 12.0 | 40 | 20 |
| ISIS 449708 | 6.6 | 12.2 | 40 | 22 |
| ISIS 449709 | 6.9 | 12.6 | 41 | 14 |
| ISIS 449710 | 6.3 | 12.5 | 41 | 13 |
| ISIS 449711 | 6.4 | 12.6 | 43 | 13 |

TABLE 120
Effect of antisense oligonucleotide treatment on blood cell count in
Sprague-Dawley rats

| | Neutrophils | Lymphocytes | Monocytes | Platelets |
|---|---|---|---|---|
| | (%) | (%) | (%) | (/nL) |
| PBS | 12 | 84 | 2 | 1004 |
| ISIS 449707 | 6 | 91 | 2 | 722 |
| ISIS 449708 | 6 | 92 | 2 | 925 |
| ISIS 449709 | 5 | 91 | 3 | 631 |
| ISIS 449710 | 6 | 91 | 2 | 509 |
| ISIS 449711 | 7 | 90 | 2 | 919 |

### Example 29

### Dose-Dependent Pharmacologic Effect of Antisense Oligonucleotides Targeting Human Factor XI in Cynomolgus Monkeys

Several antisense oligonucleotides were tested in cynomolgus monkeys to determine the pharmacologic effects of the oligonucleotides on Factor XI activity, anticoagulation and bleeding times, liver and kidney distributions, and tolerability. All the ISIS oligonucleotides used in this study target human Factor XI mRNA and are also fully cross-reactive with the rhesus monkey gene sequence (see Table 51). It is expected that the rhesus monkey ISIS oligonucleotides are fully cross-reactive with the cynomolgus monkey gene sequence as well. At the time the study was undertaken, the cynomolgus monkey genomic sequence was not available in the National Center for Biotechnology Information (NCBI) database; therefore, cross-reactivity with the cynomolgus monkey gene sequence could not be confirmed.

### Treatment

Groups, each consisting of two male and three female monkeys, were injected subcutaneously with ISIS 416838, ISIS 416850, ISIS 416858, ISIS 416864, or ISIS 417002 in escalating doses. Antisense oligonucleotide was administered to the monkeys at 5 mg/kg three times per a week for week 1; 5 mg/kg twice per week for weeks 2 and 3; 10 mg/kg three times per week for week 4; 10 mg/kg twice per week for weeks 5 and 6; 25 mg/kg three times per week for week 7; and 25 mg/kg twice per week for weeks 8, 9, 10, 11, and 12. One control group, consisting of two male and three female monkeys, was injected subcutaneously with PBS according to the same dosing regimen. An additional experimental group, consisting of two male and three female monkeys, was injected subcutaneously with ISIS 416850 in a chronic, lower dose regimen. Antisense oligonucleotide was administered to the monkeys at 5 mg/kg three times per week for week 1; 5 mg/kg twice per week for week 2 and 3; 10 mg/kg three times per week for week 4; and 10 mg/kg twice per week for weeks 5 to 12. Body weights were measured weekly. Blood samples were collected 14 days and 5 days before the start of treatment and subsequently once per week for Factor XI protein activity analysis in plasma and measurement of various hematologic factors. On day 85, the monkeys were euthanized by exsanguination while under deep anesthesia, and organs harvested for further analysis.

### RNA Analysis

On day 85, RNA was extracted from liver tissue for real-time PCR analysis of Factor XI using primer probe set LTS00301 (forward primer sequence ACACGCATTAAAAAGAGCAAAGC, designated herein as SEQ ID NO 271; reverse primer sequence CAGTGTCATGGTAAAATGAAGAATGG, designated herein as SEQ ID NO: 272; and probe sequence TGCAGGCACAGCATCCCAGTGTTCTX, wherein X is a fluorphore, designated herein as SEQ ID NO. 273). Results are presented as percent inhibition of Factor XI, relative to PBS control. As shown in Table 121, treatment with ISIS oligonucleotides resulted in significant reduction of Factor XI mRNA in comparison to the PBS control.

TABLE 121
Inhibition of Factor XI mRNA in the cynomolgus monkey liver relative to
the PBS control

| | % |
|---|---|
| ISIS No | inhibition |
| 416838 | 37 |
| 416850 | 84 |
| 416858 | 90 |
| 416864 | 44 |
| 417002 | 57 |

### Protein Analysis

Plasma samples from all monkey groups taken on different days were analyzed by a sandwich-style ELISA assay (Affinity Biologicals Inc.) using an affinity-purified polyclonal anti-Factor XI antibody as the capture antibody and a peroxidase-conjugated polyclonal anti-Factor XI antibody as the detecting antibody. Monkey plasma was diluted 1:50 for the assay. Peroxidase activity was expressed by incubation with the substrate o-phenylenediamine. The color produced was quantified using a microplate reader at 490 nm and was considered to be proportional to the concentration of Factor XI in the samples.

The results are presented in Table 122, expressed as percentage reduction relative to that of the PBS control. Treatment with ISIS 416850 and ISIS 416858 resulted in a time-dependent decrease in protein levels.

TABLE 122
Inhibition of Factor XI protein in the cynomolgus
monkey liver relative to the PBS control

| | ISIS | ISIS | ISIS | ISIS | ISIS | ISIS |
|---|---|---|---|---|---|---|
| Days | 416838 | 416850 | 416858 | 416864 | 417002 | 416850* |
| −14 | 0 | 0 | 0 | 0 | 0 | 0 |
| −5 | 0 | 0 | 0 | 5 | 0 | 1 |
| 8 | 3 | 8 | 6 | 7 | 0 | 6 |
| 15 | 4 | 4 | 16 | 9 | 4 | 13 |
| 22 | 5 | 11 | 23 | 7 | 2 | 12 |
| 29 | 8 | 15 | 28 | 10 | 8 | 20 |
| 36 | 11 | 17 | 35 | 9 | 8 | 22 |
| 43 | 5 | 23 | 39 | 9 | 9 | 24 |
| 50 | 8 | 42 | 49 | 10 | 13 | 30 |
| 57 | 10 | 49 | 60 | 7 | 24 | 34 |
| 64 | 11 | 55 | 68 | 5 | 26 | 37 |
| 71 | 12 | 57 | 71 | 10 | 30 | 41 |
| 78 | 10 | 63 | 73 | 9 | 22 | 42 |
| 85 | 10 | 64 | 78 | 8 | 23 | 34 |

### Body and Organ Weights

Body weights were taken once weekly throughout the dosing regimen. The measurements of each group are given in Table 123 expressed in grams. The results indicate that treatment with the antisense oligonucleotides did not cause any adverse changes in the health of the animals, which may have resulted in a significant alteration in weight compared to the PBS control. Organ weights were taken after the animals were euthanized and livers, kidneys and spleens were harvested and weighed. The results are presented in Table 124 and also show no significant alteration in weights compared to the PBS control, except for ISIS 416858, which shows increase in spleen weight. The ISIS oligonucleotide, ISIS 416850, given with the chronic dose regimen is distinguished from the other oligonucleotides with an asterisk (*).

TABLE 123
Weekly measurements of body weights
(g) of cynomolgus monkeys

| | | ISIS | ISIS | ISIS | ISIS | ISIS | ISIS |
|---|---|---|---|---|---|---|---|
| day | PBS | 416838 | 416850 | 416858 | 416864 | 417002 | 416850* |
| 1 | 2780 | 2720 | 2572 | 2912 | 2890 | 2640 | 2665 |
| 8 | 2615 | 2592 | 2430 | 2740 | 2784 | 2523 | 2579 |
| 15 | 2678 | 2642 | 2474 | 2760 | 2817 | 2571 | 2607 |
| 22 | 2715 | 2702 | 2514 | 2800 | 2857 | 2617 | 2661 |
| 29 | 2717 | 2689 | 2515 | 2763 | 2863 | 2622 | 2667 |
| 36 | 2738 | 2708 | 2545 | 2584 | 3327 | 2631 | 2656 |
| 43 | 2742 | 2700 | 2544 | 2607 | 3355 | 2630 | 2670 |
| 50 | 2764 | 2731 | 2613 | 2646 | 3408 | 2652 | 2679 |
| 57 | 2763 | 2737 | 2629 | 2617 | 3387 | 2654 | n.d. |
| 64 | 2781 | 2746 | 2642 | 2618 | 3384 | 2598 | n.d. |
| 71 | 2945 | 2869 | 2769 | 2865 | 2942 | 2727 | n.d. |
| 78 | 2815 | 2766 | 2660 | 2713 | 2822 | 2570 | n.d. |

n.d. = no data

TABLE 124
Organ weights (g) of cynomolgus monkeys after antisense oligonucleotide
treatment

| | Liver | Spleen | Kidney |
|---|---|---|---|
| PBS | 46 | 4 | 11 |
| ISIS 416838 | 63 | 5 | 12 |
| ISIS 416580 | 64 | 4 | 16 |
| ISIS 416858 | 60 | 12 | 13 |
| ISIS 416864 | 53 | 5 | 14 |
| ISIS 417002 | 51 | 5 | 15 |

### Liver Function

To evaluate the impact of ISIS oligonucleotides on hepatic function, plasma concentrations of transaminases were measured using an automated clinical chemistry analyzer (Hitachi Olympus AU400e, Melville, N.Y.). Plasma concentrations of ALT (alanine transaminase) and AST (aspartate transaminase) were measured and the results are presented in Tables 125 and 126 expressed in IU/L. Those antisense oligonucleotides which did not affect an increase in ALT/AST levels above seven-fold of control levels were selected for further studies. Plasma levels of bilirubin were also measured and results are presented in Table 127 expressed in mg/dL. Those antisense oligonucleotides which did not affect an increase in levels of bilirubin more than two-fold of the control levels by antisense oligonucleotide treatment were selected for further studies. The ISIS oligonucleotide, ISIS 416850, given with the chronic dose regimen is distinguished from the other oligonucleotides with an asterisk (*).

TABLE 125
Effect of antisense oligonucleotide treatment on
ALT (IU/L) in the liver of cynomolgus monkeys

| Days | | | | | | | |
|---|---|---|---|---|---|---|---|
| before/after | | ISIS | ISIS | ISIS | ISIS | ISIS | ISIS |
| treatment | PBS | 416838 | 416850 | 416858 | 416864 | 417002 | 416850* |
| −14 | 57 | 76 | 54 | 47 | 54 | 61 | 80 |
| 22 | 39 | 36 | 41 | 28 | 37 | 36 | 42 |
| 43 | 36 | 35 | 43 | 36 | 36 | 35 | 41 |
| 64 | 38 | 40 | 60 | 47 | 43 | 42 | n.d. |
| 85 | 34 | 41 | 75 | 50 | 43 | 116 | n.d. |

n.d. = no data

TABLE 126
Effect of antisense oligonucleotide treatment on
AST (IU/L) in the liver of cynomolgus monkeys

| Days | | | | | | | |
|---|---|---|---|---|---|---|---|
| before/after | | ISIS | ISIS | ISIS | ISIS | ISIS | ISIS |
| treatment | PBS | 416838 | 416850 | 416858 | 416864 | 417002 | 416850* |
| −14 | 71 | 139 | 81 | 58 | 76 | 114 | 100 |
| 22 | 43 | 39 | 45 | 38 | 41 | 44 | 39 |
| 43 | 38 | 32 | 50 | 39 | 40 | 42 | 40 |
| 64 | 35 | 33 | 56 | 50 | 46 | 37 | n.d. |
| 85 | 41 | 30 | 82. | 49 | 56 | 50 | n.d. |

n.d. = no data

TABLE 127
Effect of antisense oligonucleotide treatment on bilirubin
(mg/dL) in the liver of cynomolgus monkeys

| Days | | | | | | | |
|---|---|---|---|---|---|---|---|
| before/after | | ISIS | ISIS | ISIS | ISIS | ISIS | ISIS |
| treatment | PBS | 416838 | 416850 | 416858 | 416864 | 417002 | 416850* |
| −14 | 0.24 | 0.26 | 0.21 | 0.27 | 0.31 | 0.26 | 0.28 |
| 22 | 0.16 | 0.17 | 0.13 | 0.18 | 0.22 | 0.20 | 0.19 |
| 43 | 0.17 | 0.17 | 0.13 | 0.14 | 0.17 | 0.21 | 0.18 |
| 64 | 0.19 | 0.15 | 0.14 | 0.12 | 0.16 | 0.14 | n.d. |
| 85 | 0.20 | 0.13 | 0.14 | 0.14 | 0.17 | 0.12 | n.d. |

n.d. = no data

### Kidney Function

To evaluate the impact of ISIS oligonucleotides on kidney function, urine samples were collected. The ratio of urine protein to creatinine in urine samples after antisense oligonucleotide treatment was calculated and is presented in Table 128. Those antisense oligonucleotides which did not affect more than a five-fold increase in urine protein/creatinine ratios compared to the PBS control were selected for further studies.

TABLE 128
Effect of antisense oligonucleotide treatment on urine protein to creatinine
ratio in cynomolgus monkeys

| | Day 80 | Day 84 |
|---|---|---|
| PBS | 0.09 | 0.10 |
| ISIS 416838 | 0.13 | 0.13 |
| ISIS 416850 | 0.09 | 0.12 |
| ISIS 416858 | 0.10 | 0.07 |
| ISIS 416864 | 0.36 | 0.34 |
| ISIS 417002 | 0.18 | 0.24 |

### Measurement of Oligonucleotide Concentration

The concentration of the full-length oligonucleotide as well as the elapsed time oligonucleotide degradation and elimination from the liver and kidney were evaluated. The method used is a modification of previously published methods (Leeds et al., 1996; Geary et al., 1999) which consist of a phenol-chloroform (liquid-liquid) extraction followed by a solid phase extraction. An internal standard (ISIS 355868, a 27-mer 2′-O-methoxyethyl modified phosphorothioate oligonucleotide, GCGTTTGCTCTTCTTCTTGCGTTTTTT, designated herein as SEQ ID NO: 270) was added prior to extraction. Tissue sample concentrations were calculated using calibration curves, with a lower limit of quantitation (LLOQ) of approximately 1.14 μg/g. Half-lives were then calculated using WinNonlin software (PHARSIGHT). The results are presented in Tables 129 and 130, expressed as μg/g liver or kidney tissue.

TABLE 129
Full-length oligonucleotide concentration (μg/g) in the liver and kidney of
cynomolgus monkeys

| ISIS No. | Kidney | Liver |
|---|---|---|
| 416838 | 1339 | 1087 |
| 416850 | 2845 | 1225 |
| 416858 | 1772 | 1061 |
| 416864 | 2093 | 1275 |
| 417002 | 2162 | 1248 |

TABLE 130
Total oligonucleotide concentration (μg/g) in the liver and kidney of
cynomolgus monkeys

| ISIS No. | Kidney | Liver |
|---|---|---|
| 416838 | 1980 | 1544 |
| 416850 | 3988 | 1558 |
| 416858 | 2483 | 1504 |
| 416864 | 3522 | 1967 |
| 417002 | 3462 | 1757 |

### Hematology Assays

Blood obtained from all monkey groups were sent to Korea Institute of Toxicology (KIT) for HCT, MCV, MCH, and MCHC analysis, as well as measurements of the various blood cells, such as WBC (neutrophils, lymphocytes, monocytes, eosinophils, basophils, reticulocytes), RBC, platelets and total hemoglobin content. The results are presented in Tables 131-144. Those antisense oligonucleotides which did not affect a decrease in platelet count of more than 50% and an increase in monocyte count of more than three-fold were selected for further studies. The ISIS oligonucleotide, ISIS 416850, given with the chronic dose regimen is distinguished from the other oligonucleotides with an asterisk (*).

TABLE 131
Effect of antisense oligonucleotide treatment on WBC
count (×10³/μL) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 14 | 12 | 13 | 14 | 13 | 13 | 15 |
| day −5 | 13 | 12 | 13 | 14 | 13 | 14 | 15 |
| day 8 | 10 | 10 | 10 | 12 | 11 | 10 | 13 |
| day 15 | 10 | 10 | 9 | 11 | 10 | 10 | 16 |
| day 22 | 12 | 11 | 10 | 11 | 10 | 10 | 15 |
| day 29 | 11 | 11 | 11 | 12 | 10 | 10 | 14 |
| day 36 | 10 | 10 | 10 | 12 | 10 | 11 | 16 |
| day 43 | 10 | 10 | 9 | 11 | 10 | 10 | 15 |
| day 50 | 12 | 11 | 11 | 13 | 12 | 13 | 15 |
| day 57 | 11 | 12 | 11 | 13 | 12 | 12 | n.d. |
| day 64 | 11 | 13 | 11 | 12 | 11 | 11 | n.d. |
| day 71 | 15 | 15 | 15 | 13 | 14 | 12 | n.d. |
| day 78 | 10 | 11 | 12 | 11 | 11 | 9 | n.d. |
| day 85 | 10 | 12 | 15 | 11 | 12 | 10 | n.d. |

n.d. = no data

TABLE 132
Effect of antisense oligonucleotide treatment on RBC
count (×10⁶/μL) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 5.7 | 5.6 | 5.3 | 5.6 | 5.5 | 5.6 | 5.5 |
| day −5 | 5.7 | 5.6 | 5.5 | 5.6 | 5.6 | 5.6 | 5.5 |
| day 8 | 5.7 | 5.7 | 5.4 | 5.6 | 5.7 | 5.6 | 5.5 |
| day 15 | 5.6 | 5.6 | 5.3 | 5.4 | 5.7 | 5.4 | 5.3 |
| day 22 | 5.5 | 5.4 | 5 | 5.3 | 5.3 | 5.2 | 5.1 |
| day 29 | 5.6 | 5.3 | 4.9 | 5.3 | 5.3 | 5.2 | 5.2 |
| day 36 | 5.7 | 5.5 | 5.3 | 5.5 | 5.6 | 5.4 | 5.3 |
| day 43 | 5.7 | 5.6 | 5.2 | 5.5 | 5.5 | 5.4 | 5.2 |
| day 50 | 5.8 | 5.5 | 5.2 | 5.5 | 5.6 | 5.4 | 5.3 |
| day 57 | 5.7 | 5.5 | 5.2 | 5.6 | 5.5 | 4.9 | n.d. |
| day 64 | 5.8 | 5.6 | 5.4 | 5.7 | 5.6 | 5.4 | n.d. |
| day 71 | 5.6 | 5.5 | 5.4 | 5.6 | 5.6 | 5.5 | n.d. |
| day 78 | 5.6 | 5.4 | 5.3 | 5.4 | 5.3 | 5.4 | n.d. |
| day 85 | 5.6 | 5.5 | 5.5 | 5.5 | 5.4 | 5.4 | n.d. |

n.d. = no data

TABLE 133
Effect of antisense oligonucleotide treatment
on hemoglobin (g/dL) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 13.2 | 12.9 | 12.4 | 13.2 | 12.7 | 13.0 | 12.8 |
| day −5 | 13.1 | 13.1 | 12.7 | 13.2 | 13.0 | 13.2 | 12.8 |
| day 8 | 13.1 | 12.9 | 12.4 | 12.8 | 12.7 | 12.8 | 12.5 |
| day 15 | 12.9 | 12.9 | 12.1 | 12.6 | 12.8 | 12.3 | 12.2 |
| day 22 | 12.7 | 12.5 | 11.6 | 12.4 | 12.1 | 12.1 | 11.7 |
| day 29 | 12.8 | 12.4 | 11.5 | 12.3 | 12.1 | 12.0 | 12.0 |
| day 36 | 13.0 | 12.8 | 12.2 | 12.6 | 12.5 | 12.5 | 12.3 |
| day 43 | 12.9 | 12.7 | 11.8 | 12.4 | 12.2 | 12.3 | 11.8 |
| day 50 | 12.6 | 12.3 | 11.8 | 12.2 | 12.1 | 12.3 | 11.9 |
| day 57 | 13.1 | 12.6 | 12.1 | 12.7 | 12.3 | 11.3 | n.d. |
| day 64 | 13.1 | 12.6 | 12.3 | 12.8 | 12.1 | 12.2 | n.d. |
| day 71 | 12.9 | 12.7 | 12.3 | 12.7 | 12.2 | 12.5 | n.d. |
| day 78 | 13.0 | 12.5 | 12.2 | 12.4 | 11.9 | 12.4 | n.d. |
| day 85 | 13.2 | 12.4 | 12.7 | 11.9 | 12.3 | 12.2 | n.d. |

n.d. = no data

TABLE 134
Effect of antisense oligonucleotide treatment
on hematocrit (%) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 46 | 42 | 41 | 43 | 43 | 44 | 44 |
| day −5 | 44 | 42 | 43 | 42 | 44 | 45 | 43 |
| day 8 | 44 | 43 | 43 | 43 | 44 | 44 | 43 |
| day 15 | 44 | 42 | 40 | 40 | 42 | 40 | 40 |
| day 22 | 45 | 43 | 41 | 41 | 42 | 41 | 40 |
| day 29 | 46 | 43 | 41 | 41 | 43 | 42 | 42 |
| day 36 | 46 | 43 | 42 | 40 | 42 | 42 | 41 |
| day 43 | 46 | 43 | 40 | 40 | 42 | 41 | 40 |
| day 50 | 48 | 44 | 42 | 41 | 44 | 43 | 42 |
| day 57 | 46 | 43 | 42 | 41 | 42 | 38 | n.d. |
| day 64 | 47 | 44 | 43 | 42 | 42 | 41 | n.d. |
| day 71 | 46 | 44 | 43 | 42 | 44 | 43 | n.d. |
| day 78 | 43 | 41 | 41 | 39 | 39 | 40 | n.d. |
| day 85 | 43 | 42 | 42 | 39 | 40 | 41 | n.d. |

n.d. = no data

TABLE 135
Effect of antisense oligonucleotide treatment
on MCV (fL) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 81 | 77 | 78 | 77 | 79 | 79 | 81 |
| day −5 | 78 | 76 | 77 | 75 | 79 | 80 | 78 |
| day 8 | 77 | 77 | 80 | 77 | 78 | 79 | 79 |
| day 15 | 78 | 75 | 76 | 74 | 74 | 76 | 75 |
| day 22 | 84 | 80 | 83 | 77 | 79 | 79 | 79 |
| day 29 | 83 | 81 | 83 | 78 | 80 | 81 | 82 |
| day 36 | 81 | 78 | 80 | 75 | 76 | 78 | 76 |
| day 43 | 80 | 78 | 79 | 74 | 77 | 77 | 77 |
| day 50 | 84 | 80 | 83 | 76 | 79 | 80 | 80 |
| day 57 | 82 | 79 | 80 | 74 | 77 | 80 | n.d. |
| day 64 | 81 | 79 | 79 | 73 | 75 | 76 | n.d. |
| day 71 | 84 | 80 | 80 | 75 | 79 | 78 | n.d. |
| day 78 | 78 | 76 | 79 | 72 | 74 | 75 | n.d. |
| day 85 | 77 | 77 | 77 | 72 | 74 | 76 | n.d. |

n.d. = no data

TABLE 136
Effect of antisense oligonucleotide treatment
on MCH (pg) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 23 | 23 | 23 | 24 | 23 | 24 | 24 |
| day −5 | 23 | 23 | 23 | 23 | 23 | 24 | 23 |
| day 8 | 23 | 23 | 23 | 23 | 23 | 23 | 23 |
| day 15 | 23 | 23 | 23 | 23 | 23 | 23 | 23 |
| day 22 | 23 | 23 | 24 | 24 | 23 | 23 | 23 |
| day 29 | 23 | 23 | 23 | 23 | 23 | 23 | 23 |
| day 36 | 23 | 23 | 23 | 23 | 23 | 23 | 23 |
| day 43 | 23 | 23 | 23 | 23 | 22 | 23 | 23 |
| day 50 | 22 | 23 | 23 | 23 | 22 | 23 | 23 |
| day 57 | 23 | 23 | 23 | 22 | 23 | 23 | n.d. |
| Day 64 | 23 | 23 | 22 | 22 | 23 | 22 | n.d. |
| Day 71 | 23 | 23 | 23 | 22 | 23 | 23 | n.d. |
| Day 78 | 23 | 23 | 23 | 23 | 23 | 23 | n.d. |
| Day 85 | 23 | 23 | 22 | 22 | 23 | 23 | n.d. |

n.d. = no data

TABLE 137
Effect of antisense oligonucleotide treatment
on MCHC (g/dL) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 29 | 30 | 30 | 31 | 29 | 30 | 29 |
| day −5 | 30 | 31 | 30 | 31 | 29 | 30 | 30 |
| day 8 | 30 | 30 | 29 | 30 | 29 | 29 | 29 |
| day 15 | 30 | 31 | 30 | 31 | 30 | 31 | 30 |
| day 22 | 28 | 29 | 28 | 30 | 29 | 29 | 29 |
| day 29 | 28 | 29 | 28 | 30 | 29 | 29 | 28 |
| day 36 | 28 | 30 | 29 | 31 | 30 | 30 | 30 |
| day 43 | 28 | 30 | 29 | 31 | 29 | 30 | 30 |
| day 50 | 26 | 28 | 28 | 30 | 28 | 29 | 29 |
| day 57 | 29 | 29 | 29 | 31 | 29 | 29 | n.d. |
| day 64 | 28 | 29 | 29 | 30 | 29 | 30 | n.d. |
| day 71 | 28 | 29 | 28 | 30 | 28 | 29 | n.d. |
| day 78 | 30 | 30 | 29 | 32 | 30 | 31 | n.d. |
| day 85 | 31 | 30 | 30 | 31 | 30 | 30 | n.d. |

n.d. = no data

TABLE 138
Effect of antisense oligonucleotide treatment on platelet
count (×10³/μL) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 349 | 377 | 528 | 419 | 434 | 442 | 387 |
| day −5 | 405 | 425 | 573 | 463 | 456 | 466 | 434 |
| day 8 | 365 | 387 | 548 | 391 | 438 | 435 | 401 |
| day 15 | 375 | 387 | 559 | 400 | 439 | 410 | 396 |
| day 22 | 294 | 319 | 466 | 316 | 364 | 377 | 347 |
| day 29 | 311 | 337 | 475 | 336 | 397 | 410 | 370 |
| day 36 | 326 | 370 | 505 | 371 | 428 | 415 | 379 |
| day 43 | 336 | 365 | 490 | 342 | 351 | 393 | 391 |
| day 50 | 379 | 372 | 487 | 331 | 419 | 389 | 351 |
| day 57 | 345 | 371 | 528 | 333 | 409 | 403 | n.d. |
| day 64 | 329 | 358 | 496 | 295 | 383 | 436 | n.d. |
| day 71 | 322 | 365 | 465 | 286 | 394 | 490 | n.d. |
| day 78 | 309 | 348 | 449 | 262 | 366 | 432 | n.d. |
| day 85 | 356 | 344 | 458 | 267 | 387 | 418 | n.d. |

n.d. = no data

TABLE 139
Effect of antisense oligonucleotide treatment
on reticulocytes (%) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 1.4 | 1.0 | 1.7 | 1.0 | 0.9 | 0.9 | 1.1 |
| day −5 | 1.0 | 0.9 | 1.2 | 0.9 | 0.9 | 0.8 | 0.8 |
| day 8 | 1.0 | 1.2 | 1.2 | 1.2 | 0.8 | 1.1 | 1.1 |
| day 15 | 1.5 | 1.2 | 1.9 | 1.6 | 0.8 | 1.1 | 1.0 |
| day 22 | 1.2 | 1.2 | 1.9 | 1.3 | 0.9 | 1.2 | 1.0 |
| day 29 | 1.6 | 1.6 | 2.5 | 1.5 | 1.3 | 1.6 | 1.4 |
| day 36 | 1.7 | 1.6 | 2.2 | 1.6 | 1.3 | 1.3 | 1.3 |
| day 43 | 1.3 | 1.2 | 1.6 | 1.3 | 1.1 | 1.1 | 1.0 |
| day 50 | 1.6 | 1.6 | 2.7 | 1.5 | 1.3 | 1.6 | 1.2 |
| day 57 | 1.8 | 1.5 | 2.0 | 1.4 | 1.0 | 4.6 | n.d. |
| day 64 | 1.3 | 1.3 | 1.7 | 1.0 | 0.8 | 1.3 | n.d. |
| day 71 | 1.6 | 1.3 | 1.8 | 1.3 | 1.0 | 1.3 | n.d. |
| day 78 | 1.5 | 1.4 | 1.8 | 1.2 | 1.2 | 1.3 | n.d. |
| day 85 | 1.5 | 1.5 | 2.3 | 1.3 | 1.5 | 1.4 | n.d. |

n.d. = no data

TABLE 140
Effect of antisense oligonucleotide treatment
on neutrophils (%) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 40 | 36 | 49 | 37 | 53 | 43 | 48 |
| day −5 | 37 | 35 | 52 | 46 | 51 | 43 | 53 |
| day 8 | 54 | 42 | 57 | 51 | 52 | 46 | 53 |
| day 15 | 49 | 43 | 58 | 54 | 59 | 57 | 73 |
| day 22 | 41 | 37 | 57 | 47 | 59 | 55 | 64 |
| day 29 | 44 | 36 | 53 | 43 | 44 | 45 | 42 |
| day 36 | 37 | 39 | 57 | 47 | 58 | 61 | 72 |
| day 43 | 40 | 30 | 50 | 45 | 57 | 57 | 61 |
| day 50 | 36 | 31 | 45 | 46 | 49 | 61 | 62 |
| day 57 | 41 | 32 | 49 | 44 | 57 | 54 | n.d. |
| day 64 | 40 | 30 | 41 | 37 | 49 | 55 | n.d. |
| day 71 | 38 | 28 | 27 | 26 | 42 | 34 | n.d. |
| day 78 | 42 | 35 | 42 | 39 | 48 | 51 | n.d. |
| day 85 | 30 | 22 | 60 | 40 | 39 | 36 | n.d. |

n.d. = no data

TABLE 141
Effect of antisense oligonucleotide treatment
on lymphocytes (%) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 54 | 59 | 47 | 58 | 42 | 53 | 47 |
| day −5 | 56 | 59 | 43 | 49 | 44 | 53 | 43 |
| day 8 | 43 | 54 | 39 | 45 | 45 | 50 | 44 |
| day 15 | 47 | 53 | 38 | 43 | 38 | 40 | 24 |
| day 22 | 54 | 59 | 39 | 49 | 37 | 41 | 33 |
| day 29 | 51 | 59 | 43 | 51 | 51 | 50 | 53 |
| day 36 | 58 | 57 | 39 | 49 | 38 | 35 | 26 |
| day 43 | 55 | 65 | 45 | 51 | 39 | 39 | 36 |
| day 50 | 59 | 64 | 49 | 48 | 46 | 34 | 35 |
| day 57 | 55 | 63 | 45 | 51 | 39 | 40 | n.d. |
| day 64 | 56 | 64 | 53 | 56 | 46 | 39 | n.d. |
| day 71 | 56 | 65 | 61 | 66 | 52 | 59 | n.d. |
| day 78 | 53 | 60 | 51 | 54 | 46 | 41 | n.d. |
| day 85 | 63 | 72 | 34 | 52 | 54 | 56 | n.d. |

n.d. = no data

TABLE 142
Effect of antisense oligonucleotide treatment
on eosinophils (%) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 1.3 | 0.6 | 1.0 | 0.7 | 1.0 | 0.3 | 0.5 |
| day −5 | 1.5 | 0.6 | 1.6 | 1.3 | 0.9 | 0.3 | 0.7 |
| day 8 | 0.9 | 0.4 | 1.1 | 0.3 | 0.7 | 0.2 | 0.5 |
| day 15 | 0.7 | 0.3 | 1.0 | 0.3 | 0.5 | 0.1 | 0.2 |
| day 22 | 0.9 | 0.5 | 0.7 | 0.6 | 0.9 | 0.3 | 0.5 |
| day 29 | 0.9 | 0.3 | 1.2 | 0.6 | 0.9 | 0.3 | 0.8 |
| day 36 | 0.9 | 0.5 | 1.7 | 0.4 | 0.6 | 0.2 | 0.4 |
| day 43 | 0.9 | 0.6 | 1.2 | 0.3 | 0.6 | 0.2 | 0.4 |
| day 50 | 1.2 | 0.8 | 1.2 | 0.4 | 0.7 | 0.1 | 0.3 |
| day 57 | 0.7 | 0.6 | 1.0 | 0.3 | 0.4 | 0.2 | n.d. |
| day 64 | 1.0 | 0.7 | 1.3 | 0.4 | 0.7 | 0.2 | n.d. |
| day 71 | 1.6 | 0.8 | 1.8 | 0.9 | 1.1 | 0.3 | n.d. |
| day 78 | 1.0 | 0.9 | 1.0 | 0.5 | 1.2 | 0.1 | n.d. |
| day 85 | 1.3 | 1.5 | 1.2 | 0.6 | 1.6 | 0.2 | n.d. |

n.d. = no data

TABLE 143
Effect of antisense oligonucleotide treatment
on monocytes (%) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 3.3 | 3.1 | 2.3 | 2.8 | 2.8 | 3.0 | 2.9 |
| day −5 | 3.8 | 3.6 | 2.8 | 2.8 | 3.3 | 3.2 | 2.4 |
| day 8 | 2.3 | 2.5 | 1.8 | 2.7 | 2.1 | 3.3 | 1.8 |
| day 15 | 2.7 | 2.4 | 2.0 | 2.2 | 2.4 | 2.3 | 1.5 |
| day 22 | 3.4 | 2.9 | 2.4 | 2.8 | 2.8 | 3.1 | 1.9 |
| day 29 | 3.3 | 3.2 | 2.7 | 3.8 | 3.4 | 3.5 | 2.7 |
| day 36 | 3.1 | 2.5 | 2.1 | 2.9 | 2.3 | 2.6 | 1.5 |
| day 43 | 3.5 | 3.3 | 2.6 | 3.1 | 2.1 | 2.8 | 1.8 |
| day 50 | 2.6 | 3.2 | 3.7 | 4.6 | 2.9 | 3.1 | 1.8 |
| day 57 | 2.6 | 3.2 | n.d. 3.2 | 3.8 | 2.4 | 3.6 | n.d. |
| day 64 | 2.6 | 3.5 | n.d. 3.5 | 4.4 | 2.8 | 4.0 | n.d. |
| day 71 | 3.4 | 4.3 | n.d. 4.7 | 4.9 | 3.7 | 4.7 | n.d. |
| day 78 | 3.3 | 3.6 | n.d. 4.5 | 4.9 | 3.7 | 4.7 | n.d. |
| day 85 | 4.4 | 3.7 | n.d. 3.5 | 6.1 | 3.7 | 5.3 | n.d. |

n.d. = no data

TABLE 144
Effect of antisense oligonucleotide treatment
on basophils (%) in cynomolgus monkeys

| | | ISIS | ISIS | ISIS | ISIS | ISIS | ISIS |
|---|---|---|---|---|---|---|---|
| | PBS | 416838 | 416850 | 416858 | 416864 | 417002 | 416850* |
| day −14 | 0.3 | 0.2 | 0.2 | 0.3 | 0.2 | 0.3 | 0.2 |
| day −5 | 0.3 | 0.3 | 0.2 | 0.3 | 0.2 | 0.3 | 0.3 |
| day 8 | 0.2 | 0.2 | 0.2 | 0.3 | 0.2 | 0.3 | 0.3 |
| day 15 | 0.3 | 0.3 | 0.2 | 0.2 | 0.2 | 0.2 | 0.2 |
| day 22 | 0.2 | 0.2 | 0.2 | 0.2 | 0.2 | 0.2 | 0.1 |
| day 29 | 0.3 | 0.2 | 0.2 | 0.2 | 0.3 | 0.2 | 0.3 |
| day 36 | 0.3 | 0.4 | 0.3 | 0.3 | 0.3 | 0.2 | 0.1 |
| day 43 | 0.3 | 0.4 | 0.3 | 0.3 | 0.4 | 0.3 | 0.2 |
| day 50 | 0.4 | 0.3 | 0.3 | 0.4 | 0.4 | 0.3 | 0.2 |
| day 57 | 0.2 | 0.3 | 0.4 | 0.2 | 0.3 | 0.3 | n.d. |
| day 64 | 0.3 | 0.4 | 0.4 | 0.4 | 0.4 | 0.2 | n.d. |
| day 71 | 0.2 | 0.5 | 0.3 | 0.4 | 0.4 | 0.3 | n.d. |
| day 78 | 0.2 | 0.4 | 0.3 | 0.4 | 0.3 | 0.3 | n.d. |
| day 85 | 0.3 | 0.3 | 0.3 | 0.3 | 0.4 | 0.3 | n.d. |

n.d. = no data

### Cytokine and Chemokine Assays

Blood samples obtained from the monkey groups treated with PBS, ISIS 416850 and ISIS 416858 administered in the escalating dose regimen were sent to Pierce Biotechnology (Woburn, Mass.) for measurement of chemokine and cytokine levels. Levels of IL-1β, IL-6, IFN-γ, and TNF-α were measured using the respective primate antibodies and levels of IL-8, MIP-1α, MCP-1, MIP-1β and RANTES were measured using the respective cross-reacting human antibodies. Measurements were taken 14 days before the start of treatment and on day 85, when the monkeys were euthanized. The results are presented in Tables 145 and 146.

TABLE 145
Effect of antisense oligonucleotide treatment on cytokine/chemokine
levels (pg/mL) in cynomolgus monkeys on day −14

| | IL-1β | IL-6 | IFN-γ | TNF-α | IL-8 | MIP-1α | MCP-1 | MIP-1β | RANTES |
|---|---|---|---|---|---|---|---|---|---|
| PBS | 16 | 10 | 114 | 7 | 816 | 54 | 1015 | 118 | 72423 |
| ISIS 416850 | 3 | 30 | 126 | 14 | 1659 | 28 | 1384 | 137 | 75335 |
| ISIS 416858 | 5 | 9 | 60 | 9 | 1552 | 36 | 1252 | 122 | 112253 |

TABLE 146
Effect of antisense oligonucleotide treatment on cytokine/chemokine
levels (pg/mL) in cynomolgus monkeys on day 85

| | IL-1β | IL-6 | IFN-γ | TNF-α | IL-8 | MIP-1α | MCP-1 | MIP-1β | RANTES |
|---|---|---|---|---|---|---|---|---|---|
| PBS | 7 | 4 | 102 | 34 | 87 | 23 | 442 | 74 | 84430 |
| ISIS 416850 | 13 | 17 | 18 | 27 | 172 | 41 | 2330 | 216 | 83981 |
| ISIS 416858 | 5 | 25 | 18 | 45 | 303 | 41 | 1752 | 221 | 125511 |

### Example 30

### Pharmacologic Effect of Antisense Oligonucleotides Targeting Human Factor XI in Cynomolgus Monkeys

Several antisense oligonucleotides chosen from the rodent tolerability studies (Examples 25-28) were tested in cynomolgus monkeys to determine their pharmacologic effects, relative efficacy on Factor XI activity and tolerability in a cynomolgus monkey model. The antisense oligonucleotides were also compared to ISIS 416850 and ISIS 416858 selected from the monkey study described earlier (Example 29). All the ISIS oligonucleotides used in this study target human Factor XI mRNA and are also fully cross-reactive with the rhesus monkey gene sequence (see Tables 51 and 53). It is expected that the rhesus monkey ISIS oligonucleotides are fully cross-reactive with the cynomolgus monkey gene sequence as well. At the time the study was undertaken, the cynomolgus monkey genomic sequence was not available in the National Center for Biotechnology Information (NCBI) database; therefore, cross-reactivity with the cynomolgus monkey gene sequence could not be confirmed.

### Treatment

Groups, each consisting of two male and two female monkeys, were injected subcutaneously with 25 mg/kg of ISIS 416850, ISIS 449709, ISIS 445522, ISIS 449710, ISIS 449707, ISIS 449711, ISIS 449708, 416858 and ISIS 445531. Antisense oligonucleotide was administered to the monkeys at 25 mg/kg three times per week for week 1 and 25 mg/kg twice per week for weeks 2 to 8. A control group, consisting of two male and two female monkeys was injected subcutaneously with PBS according to the same dosing regimen. Body weights were taken 14 days and 7 days before the start of treatment and were then measured weekly throughout the treatment period. Blood samples were collected 14 days and 5 days before the start of treatment and subsequently several times during the dosing regimen for measurement of various hematologic factors. On day 55, the monkeys were euthanized by exsanguination while under deep anesthesia, and organs harvested for further analysis.

### RNA Analysis

On day 55, RNA was extracted from liver tissue for real-time PCR analysis of Factor XI using primer probe set LTS00301. Results are presented as percent inhibition of Factor XI, relative to PBS control. As shown in Table 147, treatment with ISIS 416850, ISIS 449709, ISIS 445522, ISIS 449710, ISIS 449707, ISIS 449708, ISIS 416858 and ISIS 445531 resulted in significant reduction of Factor XI mRNA in comparison to the PBS control.

TABLE 147
Inhibition of Factor XI mRNA in the cynomolgus monkey liver relative
to the PBS control

| | % |
|---|---|
| Oligo ID | inhibition |
| 416850 | 68 |
| 449709 | 69 |
| 445522 | 89 |
| 449710 | 52 |
| 449707 | 47 |
| 449711 | 0 |
| 449708 | 46 |
| 416858 | 89 |
| 445531 | 66 |

### Protein Analysis

Plasma samples from all monkey groups taken on different days were analyzed by a sandwich-style ELISA assay (Affinity Biologicals Inc.) using an affinity-purified polyclonal anti-Factor XI antibody as the capture antibody and a peroxidase-conjugated polyclonal anti-Factor XI antibody as the detecting antibody. Monkey plasma was diluted 1:50 for the assay. Peroxidase activity was expressed by incubation with the substrate o-phenylenediamine. The color produced was quantified using a microplate reader at 490 nm and was considered to be proportional to the concentration of Factor XI in the samples.

The results are presented in Table 148, expressed as percentage reduction relative to that of the PBS control. Treatment with ISIS 416850, ISIS 449709, ISIS 445522, and ISIS 416858 resulted in a time-dependent decrease in protein levels.

TABLE 148
Inhibition of Factor XI protein in the cynomolgus monkey liver relative to the PBS control

| ISIS No. | Day −14 | Day −5 | Day 10 | Day 17 | Day 24 | Day 31 | Day 38 | Day 45 | Day 52 | Day 55 |
|---|---|---|---|---|---|---|---|---|---|---|
| 416850 | 0 | 0 | 20 | 31 | 38 | 52 | 51 | 53 | 53 | 58 |
| 449709 | 1 | 0 | 27 | 35 | 44 | 45 | 46 | 48 | 47 | 50 |
| 445522 | 2 | 0 | 36 | 50 | 61 | 70 | 73 | 77 | 80 | 82 |
| 449710 | 1 | 0 | 10 | 14 | 17 | 25 | 20 | 23 | 4 | 24 |
| 449707 | 0 | 0 | 16 | 19 | 21 | 29 | 28 | 35 | 29 | 32 |
| 449711 | 0 | 1 | 5 | 3 | 6 | 9 | 2 | 4 | 3 | 5 |
| 449708 | 1 | 0 | 7 | 15 | 3 | 14 | 9 | 2 | 6 | 6 |
| 416858 | 4 | 0 | 36 | 49 | 62 | 68 | 74 | 79 | 81 | 81 |
| 445531 | 0 | 1 | 9 | 22 | 23 | 27 | 29 | 32 | 32 | 37 |

### Body and Organ Weights

Body weights of each group are given in Table 149 expressed in grams. The results indicate that treatment with the antisense oligonucleotides did not cause any adverse changes in the health of the animals, which may have resulted in a significant alteration in weight compared to the PBS control. Organ weights were taken after the animals were euthanized on day 55, and livers, kidneys and spleens were harvested. The results are presented in Table 150 expressed as a percentage of the body weight and also show no significant alteration in weights compared to the PBS control, with the exception of ISIS 449711, which caused increase in spleen weight.

TABLE 149
Weekly measurements of body weights (g) of cynomolgus monkeys

| | | ISIS | ISIS | ISIS | ISIS | ISIS | ISIS | ISIS | ISIS | ISIS |
|---|---|---|---|---|---|---|---|---|---|---|
| Days | PBS | 416850 | 449709 | 445522 | 449710 | 449707 | 449711 | 449708 | 416858 | 445531 |
| −14 | 2069 | 2061 | 2044 | 2050 | 2097 | 2072 | 2049 | 2096 | 2073 | 2079 |
| −7 | 2107 | 2074 | 2093 | 2042 | 2114 | 2083 | 2105 | 2163 | 2092 | 2092 |
| 1 | 2131 | 2083 | 2112 | 2047 | 2131 | 2107 | 2123 | 2130 | 2115 | 2125 |
| 8 | 2186 | 2072 | 2075 | 2094 | 2120 | 2088 | 2123 | 2148 | 2149 | 2119 |
| 15 | 2201 | 2147 | 2085 | 2092 | 2145 | 2120 | 2103 | 2125 | 2162 | 2109 |
| 22 | 2206 | 2139 | 2117 | 2114 | 2177 | 2142 | 2171 | 2110 | 2188 | 2143 |
| 29 | 2204 | 2159 | 2068 | 2125 | 2149 | 2155 | 2203 | 2095 | 2196 | 2148 |
| 36 | 2246 | 2136 | 2064 | 2121 | 2180 | 2158 | 2227 | 2100 | 2210 | 2191 |
| 43 | 2304 | 2186 | 2106 | 2142 | 2227 | 2197 | 2251 | 2125 | 2238 | 2233 |
| 50 | 2274 | 2143 | 2147 | 2127 | 2201 | 2185 | 2227 | 2076 | 2225 | 2197 |

TABLE 150
Organ weights (g) of cynomolgus monkeys after antisense oligonucleotide
treatment

| | Liver | Spleen | Kidney |
|---|---|---|---|
| PBS | 2.3 | 0.16 | 0.48 |
| ISIS 416850 | 2.5 | 0.17 | 0.51 |
| ISIS 449709 | 2.6 | 0.21 | 0.57 |
| ISIS 445522 | 2.6 | 0.23 | 0.55 |
| ISIS 449710 | 2.6 | 0.24 | 0.58 |
| ISIS 449707 | 2.5 | 0.24 | 0.53 |
| ISIS 449711 | 2.6 | 0.32 | 0.54 |
| ISIS 449708 | 2.6 | 0.19 | 0.60 |
| ISIS 416858 | 2.6 | 0.24 | 0.47 |
| ISIS 445531 | 2.8 | 0.24 | 0.49 |

### Liver Function

To evaluate the impact of ISIS oligonucleotides on hepatic function, plasma concentrations of ALT and AST were measured using an automated clinical chemistry analyzer (Hitachi Olympus AU400e, Melville, N.Y.). Plasma concentrations of alanine transaminase (ALT) and aspartate transaminase (AST) were measured and the results are presented in Tables 151 and 152 expressed in IU/L. Plasma levels of bilirubin were also measured and results are presented in Table 153 expressed in mg/dL. As observed in Tables 151-153, there were no significant increases in any of the liver metabolic markers after antisense oligonucleotide treatment.

TABLE 151
Effect of antisense oligonucleotide treatment on ALT (IU/L) in the liver of
cynomolgus monkeys

| | Day −14 | Day −5 | Day 31 | Day 55 |
|---|---|---|---|---|
| PBS | 57 | 55 | 53 | 57 |
| ISIS 416850 | 48 | 42 | 45 | 55 |
| ISIS 449709 | 73 | 77 | 65 | 102 |
| ISIS 445522 | 43 | 45 | 40 | 60 |
| ISIS 449710 | 37 | 42 | 37 | 45 |
| ISIS 449707 | 54 | 56 | 52 | 63 |
| ISIS 449711 | 49 | 137 | 48 | 54 |
| ISIS 449708 | 48 | 54 | 44 | 46 |
| ISIS 416858 | 43 | 66 | 46 | 58 |
| ISIS 445531 | 84 | 73 | 57 | 73 |

TABLE 152
Effect of antisense oligonucleotide treatment on AST (IU/L) in the liver of
cynomolgus monkeys

| | Day −14 | Day −5 | Day 31 | Day 55 |
|---|---|---|---|---|
| PBS | 65 | 45 | 44 | 47 |
| ISIS 416850 | 62 | 45 | 46 | 57 |
| ISIS 449709 | 62 | 51 | 45 | 71 |
| ISIS 445522 | 62 | 47 | 46 | 79 |
| ISIS 449710 | 52 | 38 | 37 | 64 |
| ISIS 449707 | 64 | 53 | 50 | 52 |
| ISIS 449711 | 58 | 78 | 47 | 47 |
| ISIS 449708 | 74 | 53 | 56 | 50 |
| ISIS 416858 | 64 | 100 | 60 | 69 |
| ISIS 445531 | 78 | 46 | 47 | 49 |

TABLE 153
Effect of antisense oligonucleotide treatment on bilirubin (mg/dL) in the
liver of cynomolgus monkeys

| | Day −14 | Day −5 | Day 31 | Day 55 |
|---|---|---|---|---|
| PBS | 0.25 | 0.20 | 0.20 | 0.17 |
| ISIS 416850 | 0.26 | 0.22 | 0.26 | 0.17 |
| ISIS 449709 | 0.24 | 0.19 | 0.15 | 0.18 |
| ISIS 445522 | 0.24 | 0.20 | 0.14 | 0.18 |
| ISIS 449710 | 0.24 | 0.19 | 0.15 | 0.22 |
| ISIS 449707 | 0.27 | 0.19 | 0.13 | 0.16 |
| ISIS 449711 | 0.23 | 0.16 | 0.13 | 0.13 |
| ISIS 449708 | 0.27 | 0.21 | 0.14 | 0.14 |
| ISIS 416858 | 0.25 | 0.23 | 0.16 | 0.16 |
| ISIS 445531 | 0.22 | 0.18 | 0.13 | 0.11 |

### Kidney Function

To evaluate the impact of ISIS oligonucleotides on kidney function, urine samples were collected on different days. BUN levels were measured at various time points using an automated clinical chemistry analyzer (Hitachi Olympus AU400e, Melville, N.Y.) and the results are presented in Table 154. The ratio of urine protein to creatinine in urine samples after antisense oligonucleotide treatment was also calculated for day 49 and results are presented in Table 155. As observed in Tables 154 and 155, there were no significant increases in any of the kidney metabolic markers after antisense oligonucleotide treatment.

TABLE 154
Effect of antisense oligonucleotide treatment on BUN levels (mg/dL) in
cynomolgus monkeys

| | Day −14 | Day −5 | Day 31 | Day 55 |
|---|---|---|---|---|
| PBS | 22 | 21 | 22 | 22 |
| ISIS 416850 | 24 | 23 | 21 | 26 |
| ISIS 449709 | 22 | 21 | 20 | 28 |
| ISIS 445522 | 23 | 22 | 22 | 22 |
| ISIS 449710 | 19 | 19 | 19 | 23 |
| ISIS 449707 | 25 | 21 | 21 | 20 |
| ISIS 449711 | 26 | 22 | 20 | 23 |
| ISIS 449708 | 25 | 23 | 23 | 23 |
| ISIS 416858 | 25 | 24 | 23 | 24 |
| ISIS 445531 | 22 | 18 | 20 | 22 |

TABLE 155
Effect of antisense oligonucleotide treatment on urine protein to creatinine
ratio in cynomolgus monkeys

| | Urine |
|---|---|
| | protein/creatinine |
| | ratio |
| PBS | 0.02 |
| ISIS 416850 | 0.08 |
| ISIS 449709 | 0.05 |
| ISIS 445522 | 0.01 |
| ISIS 449710 | 0.00 |
| ISIS 449707 | 0.03 |
| ISIS 449711 | 0.01 |
| ISIS 449708 | 0.00 |
| ISIS 416858 | 0.05 |
| ISIS 445531 | 0.08 |

### Hematology Assays

Blood obtained from all the monkey groups on different days were sent to Korea Institute of Toxicology (KIT) for HCT, MCV, MCH, and MCHC measurements, as well as measurements of the various blood cells, such as WBC (neutrophils and monocytes), RBC and platelets, as well as total hemoglobin content. The results are presented in Tables 156-165.

TABLE 156
Effect of antisense oligonucleotide treatment
on HCT (%) in cynomolgus monkeys

| | Day −14 | Day −5 | Day 17 | Day 31 | Day 45 | Day 55 |
|---|---|---|---|---|---|---|
| PBS | 40 | 42 | 43 | 43 | 41 | 40 |
| ISIS 416850 | 41 | 44 | 42 | 42 | 42 | 40 |
| ISIS 449709 | 41 | 42 | 43 | 42 | 41 | 40 |
| ISIS 445522 | 42 | 42 | 41 | 43 | 41 | 39 |
| ISIS 449710 | 41 | 44 | 43 | 44 | 43 | 41 |
| ISIS 449707 | 40 | 43 | 42 | 43 | 43 | 42 |
| ISIS 449711 | 41 | 41 | 42 | 39 | 39 | 38 |
| ISIS 449708 | 41 | 44 | 44 | 43 | 44 | 42 |
| ISIS 416858 | 41 | 44 | 43 | 43 | 41 | 39 |
| ISIS 445531 | 41 | 42 | 43 | 41 | 41 | 41 |

TABLE 157
Effect of antisense oligonucleotide treatment on platelet
count (×100/μL) in cynomolgus monkeys

| | Day −14 | Day −5 | Day 17 | Day 31 | Day 45 | Day 55 |
|---|---|---|---|---|---|---|
| PBS | 361 | 441 | 352 | 329 | 356 | 408 |
| ISIS 416850 | 462 | 517 | 467 | 507 | 453 | 396 |
| ISIS 449709 | 456 | 481 | 449 | 471 | 418 | 441 |
| ISIS 445522 | 433 | 512 | 521 | 425 | 403 | 333 |
| ISIS 449710 | 411 | 463 | 382 | 422 | 313 | 360 |
| ISIS 449707 | 383 | 464 | 408 | 408 | 424 | 399 |
| ISIS 449711 | 410 | 431 | 325 | 309 | 257 | 259 |
| ISIS 449708 | 387 | 517 | 444 | 378 | 381 | 348 |
| ISIS 416858 | 369 | 433 | 358 | 289 | 287 | 257 |
| ISIS 445531 | 379 | 416 | 380 | 376 | 345 | 319 |

TABLE 158
Effect of antisense oligonucleotide treatment
on neutrophils (%) in cynomolgus monkeys

| | Day −14 | Day −5 | Day 17 | Day 31 | Day 45 | Day 55 |
|---|---|---|---|---|---|---|
| PBS | 81 | 84 | 75 | 75 | 91 | 118 |
| ISIS 416850 | 88 | 109 | 95 | 100 | 85 | 108 |
| ISIS 449709 | 73 | 101 | 89 | 81 | 77 | 115 |
| ISIS 445522 | 61 | 84 | 81 | 66 | 69 | 125 |
| ISIS 449710 | 93 | 86 | 80 | 94 | 97 | 132 |
| ISIS 449707 | 85 | 106 | 80 | 89 | 89 | 98 |
| ISIS 449711 | 64 | 71 | 52 | 58 | 45 | 70 |
| ISIS 449708 | 73 | 84 | 61 | 57 | 61 | 75 |
| ISIS 416858 | 65 | 84 | 54 | 54 | 61 | 73 |
| ISIS 445531 | 60 | 80 | 85 | 116 | 93 | 91 |

TABLE 159
Effect of antisense oligonucleotide treatment
on monocytes (%) in cynomolgus monkeys

| | Day −14 | Day −5 | Day 17 | Day 31 | Day 45 | Day 55 |
|---|---|---|---|---|---|---|
| PBS | 1.9 | 2.8 | 3.1 | 2.8 | 3.9 | 2.2 |
| ISIS 416850 | 1.9 | 2.9 | 3.2 | 3.7 | 3.8 | 3.4 |
| ISIS 449709 | 4.0 | 2.0 | 3.0 | 2.8 | 3.6 | 3.4 |
| ISIS 445522 | 2.1 | 2.3 | 3.6 | 3.9 | 4.4 | 3.0 |
| ISIS 449710 | 1.3 | 2.0 | 2.5 | 2.4 | 3.4 | 1.6 |
| ISIS 449707 | 1.3 | 2.3 | 3.2 | 4.2 | 4.0 | 4.8 |
| ISIS 449711 | 1.2 | 2.3 | 5.9 | 6.9 | 7.6 | 7.8 |
| ISIS 449708 | 1.7 | 2.6 | 5.4 | 5.8 | 7.0 | 6.2 |
| ISIS 416858 | 2.0 | 2.7 | 4.0 | 4.7 | 4.6 | 4.6 |
| ISIS 445531 | 1.3 | 2.2 | 3.4 | 4.1 | 4.4 | 4.1 |

TABLE 160
Effect of antisense oligonucleotide treatment on
hemoglobin content (g/dL) in cynomolgus monkeys

| | Day −14 | Day −5 | Day 17 | Day 31 | Day 45 | Day 55 |
|---|---|---|---|---|---|---|
| PBS | 12.3 | 12.5 | 12.9 | 12.7 | 12.4 | 12.1 |
| ISIS 416850 | 13.0 | 13.5 | 13.3 | 13.1 | 13.1 | 12.7 |
| ISIS 449709 | 12.8 | 12.8 | 13.2 | 13.1 | 12.6 | 12.5 |
| ISIS 445522 | 13.3 | 12.7 | 12.7 | 12.9 | 12.6 | 12.0 |
| ISIS 449710 | 13.0 | 13.2 | 13.4 | 13.1 | 13.0 | 12.7 |
| ISIS 449707 | 12.7 | 12.8 | 12.7 | 12.7 | 12.9 | 12.6 |
| ISIS 449711 | 12.7 | 12.7 | 12.5 | 11.8 | 11.5 | 11.3 |
| ISIS 449708 | 13.0 | 13.2 | 13.5 | 13.0 | 13.3 | 13.0 |
| ISIS 416858 | 12.8 | 13.0 | 13.0 | 12.8 | 12.3 | 12.0 |
| ISIS 445531 | 12.6 | 12.6 | 12.7 | 12.3 | 12.0 | 12.1 |

TABLE 161
Effect of antisense oligonucleotide treatment on WBC
count (×10³/μL) in cynomolgus monkeys

| | Day −14 | Day −5 | Day 17 | Day 31 | Day 45 | Day 55 |
|---|---|---|---|---|---|---|
| PBS | 10 | 10 | 11 | 12 | 11 | 12 |
| ISIS 416850 | 12 | 13 | 11 | 12 | 12 | 10 |
| ISIS 449709 | 11 | 10 | 11 | 11 | 11 | 10 |
| ISIS 445522 | 10 | 9 | 11 | 13 | 10 | 11 |
| ISIS 449710 | 11 | 11 | 12 | 12 | 11 | 15 |
| ISIS 449707 | 13 | 11 | 12 | 11 | 12 | 8 |
| ISIS 449711 | 13 | 12 | 10 | 9 | 9 | 7 |
| ISIS 449708 | 14 | 10 | 11 | 11 | 10 | 10 |
| ISIS 416858 | 10 | 11 | 10 | 9 | 8 | 9 |
| ISIS 445531 | 20 | 15 | 17 | 17 | 20 | 15 |

TABLE 162
Effect of antisense oligonucleotide treatment on RBC
count (×10⁶/μL) in cynomolgus monkeys

| | Day −14 | Day −5 | Day 17 | Day 31 | Day 45 | Day 55 |
|---|---|---|---|---|---|---|
| PBS | 5.6 | 5.6 | 5.8 | 5.8 | 5.6 | 5.5 |
| ISIS 416850 | 5.5 | 5.7 | 5.6 | 5.6 | 5.7 | 5.6 |
| ISIS 449709 | 5.8 | 5.8 | 5.9 | 5.9 | 5.7 | 5.7 |
| ISIS 445522 | 5.9 | 5.6 | 5.6 | 5.8 | 5.7 | 5.4 |
| ISIS 449710 | 5.6 | 5.8 | 5.8 | 5.8 | 5.7 | 5.6 |
| ISIS 449707 | 5.7 | 5.8 | 5.7 | 5.7 | 5.9 | 5.8 |
| ISIS 449711 | 5.6 | 5.7 | 5.6 | 5.4 | 5.4 | 5.3 |
| ISIS 449708 | 5.7 | 5.9 | 5.9 | 5.8 | 6.0 | 5.8 |
| ISIS 416858 | 5.5 | 5.5 | 5.6 | 5.6 | 5.5 | 5.3 |
| ISIS 445531 | 5.7 | 5.7 | 5.8 | 5.6 | 5.5 | 5.6 |

TABLE 163
Effect of antisense oligonucleotide treatment
on MCV (fL) in cynomolgus monkeys

| | Day −14 | Day −5 | Day 17 | Day 31 | Day 45 | Day 55 |
|---|---|---|---|---|---|---|
| PBS | 72 | 74 | 75 | 73 | 73 | 73 |
| ISIS 416850 | 74 | 77 | 76 | 75 | 75 | 73 |
| ISIS 449709 | 72 | 74 | 73 | 73 | 71 | 71 |
| ISIS 445522 | 72 | 74 | 74 | 75 | 73 | 72 |
| ISIS 449710 | 75 | 77 | 75 | 75 | 75 | 73 |
| ISIS 449707 | 71 | 75 | 74 | 74 | 73 | 73 |
| ISIS 449711 | 73 | 74 | 75 | 73 | 73 | 73 |
| ISIS 449708 | 73 | 75 | 75 | 75 | 74 | 74 |
| ISIS 416858 | 75 | 79 | 78 | 76 | 75 | 75 |
| ISIS 445531 | 72 | 74 | 75 | 75 | 75 | 74 |

TABLE 164
Effect of antisense oligonucleotide treatment
on MCH (pg) in cynomolgus monkeys

| | Day −14 | Day −5 | Day 17 | Day 31 | Day 45 | Day 55 |
|---|---|---|---|---|---|---|
| PBS | 22.1 | 22.4 | 22.3 | 22.1 | 22.0 | 22.0 |
| ISIS 416850 | 23.7 | 23.7 | 23.7 | 23.3 | 22.7 | 22.9 |
| ISIS 449709 | 22.4 | 22.3 | 22.5 | 22.2 | 21.0 | 22.0 |
| ISIS 445522 | 22.6 | 22.5 | 22.8 | 22.4 | 22.4 | 22.2 |
| ISIS 449710 | 23.0 | 22.8 | 23.1 | 22.6 | 21.8 | 22.7 |
| ISIS 449707 | 22.2 | 22.2 | 22.1 | 22.1 | 22.6 | 21.9 |
| ISIS 449711 | 22.6 | 22.7 | 22.2 | 22.1 | 21.7 | 21.3 |
| ISIS 449708 | 22.9 | 22.7 | 22.9 | 22.7 | 22.2 | 22.5 |
| ISIS 416858 | 23.2 | 23.5 | 23.1 | 23.0 | 22.2 | 22.8 |
| ISIS 445531 | 22.2 | 22.2 | 22.1 | 22.0 | 21.6 | 21.7 |

TABLE 165
Effect of antisense oligonucleotide treatment
on MCHC (g/dL) in cynomolgus monkeys

| | Day −14 | Day −5 | Day 17 | Day 31 | Day 45 | Day 55 |
|---|---|---|---|---|---|---|
| PBS | 30.8 | 30.0 | 30.1 | 29.9 | 30.3 | 30.2 |
| ISIS 416850 | 32.0 | 30.7 | 31.3 | 31.0 | 31.0 | 30.9 |
| ISIS 449709 | 31.4 | 30.3 | 30.7 | 30.7 | 31.1 | 31.2 |
| ISIS 445522 | 31.4 | 30.4 | 30.9 | 30.0 | 30.7 | 31.0 |
| ISIS 449710 | 31.2 | 29.7 | 30.7 | 30.1 | 30.4 | 31.1 |
| ISIS 449707 | 31.4 | 29.8 | 30.0 | 29.8 | 29.8 | 30.0 |
| ISIS 449711 | 31.0 | 30.7 | 29.9 | 29.8 | 29.6 | 29.5 |
| ISIS 449708 | 31.4 | 30.2 | 30.7 | 29.9 | 30.6 | 31.8 |
| ISIS 416858 | 31.1 | 29.8 | 29.9 | 31.0 | 30.3 | 30.4 |
| ISIS 445531 | 30.9 | 30.0 | 29.5 | 29.7 | 29.0 | 29.6 |

### Cytokine and Chemokine Assays

Blood samples obtained from all monkey groups were sent to Pierce Biotechnology (Woburn, Mass.) for measurements of chemokine and cytokine levels. Levels of IL-1β, IL-6, IFN-γ, and TNF-α were measured using the respective primate antibodies and levels of IL-8, MIP-1α, MCP-1, MIP-10 and RANTES were measured using the respective cross-reacting human antibodies. Measurements were taken 14 days before the start of treatment and on day 55, when the monkeys were euthanized. The results are presented in Tables 166 and 167.

TABLE 166
Effect of antisense oligonucleotide treatment on cytokine/chemokine
levels (pg/mL) in cynomolgus monkeys on day −14

| | IL-1β | IL-6 | IFN-γ | TNF-α | IL-8 | MIP-1α | MCP-1 | MIP-1β | RANTES |
|---|---|---|---|---|---|---|---|---|---|
| PBS | 350 | 3 | 314 | 32 | 82 | 27 | 277 | 8 | 297 |
| ISIS 416850 | 215 | 1 | 115 | 4 | 45 | 14 | 434 | 31 | 4560 |
| ISIS 449409 | 137 | 1 | 37 | 9 | 34 | 13 | 290 | 14 | 2471 |
| ISIS 445522 | 188 | 5 | 172 | 16 | 32 | 22 | 297 | 27 | 3477 |
| ISIS 449710 | 271 | 7 | 1115 | 72 | 29 | 20 | 409 | 18 | 1215 |
| ISIS 449707 | 115 | 1 | 34 | 6 | 106 | 16 | 294 | 13 | 3014 |
| ISIS 449711 | 79 | 2 | 29 | 6 | 156 | 20 | 264 | 24 | 3687 |
| ISIS 449708 | 35 | 1 | 27 | 12 | 184 | 11 | 361 | 19 | 11666 |
| ISIS 416858 | 103 | 0 | 32 | 4 | 224 | 11 | 328 | 37 | 6521 |
| ISIS 445531 | 101 | 2 | 68 | 9 | 83 | 25 | 317 | 22 | 7825 |

TABLE 167
Effect of antisense oligonucleotide treatment on cytokine/chemokine
levels (pg/mL) in cynomolgus monkeys on day 55

| | IL-1β | IL-6 | IFN-γ | TNF-α | IL-8 | MIP-1α | MCP-1 | MIP-1β | RANTES |
|---|---|---|---|---|---|---|---|---|---|
| PBS | 453 | 3 | 232 | 191 | 68 | 21 | 237 | 34 | 775 |
| ISIS 416850 | 106 | 1 | 19 | 16 | 620 | 17 | 887 | 50 | 27503 |
| ISIS 449409 | 181 | 0 | 25 | 8 | 254 | 17 | 507 | 47 | 8958 |
| ISIS 445522 | 341 | 2 | 83 | 18 | 100 | 22 | 592 | 63 | 16154 |
| ISIS 449710 | 286 | 2 | 176 | 26 | 348 | 27 | 474 | 53 | 22656 |
| ISIS 449707 | 97 | 1 | 24 | 16 | 48 | 12 | 264 | 49 | 1193 |
| ISIS 449711 | 146 | 7 | 22 | 31 | 110 | 17 | 469 | 91 | 3029 |
| ISIS 449708 | 131 | 0 | 18 | 17 | 85 | 23 | 409 | 128 | 4561 |
| ISIS 416858 | 28 | 1 | 9 | 15 | 167 | 11 | 512 | 47 | 5925 |
| ISIS 445531 | 155 | 1 | 15 | 16 | 293 | 12 | 339 | 84 | 5935 |

### Example 31

### Measurement of Viscosity of Isis Antisense Oligonucleotides Targeting Human Factor XI

The viscosity of antisense oligonucleotides targeting human Factor XI was measured with the aim of screening out antisense oligonucleotides which have a viscosity more than 40 cP at a concentration of 165-185 mg/mL.

ISIS oligonucleotides (32-35 mg) were weighed into a glass vial, 120 μL of water was added and the antisense oligonucleotide was dissolved into solution by heating the vial at 50° C. Part of (75 μL) the pre-heated sample was pipetted to a micro-viscometer (Cambridge). The temperature of the micro-viscometter was set to 25° C. and the viscosity of the sample was measured. Another part (20 μL) of the pre-heated sample was pipetted into 10 mL of water for UV reading at 260 nM at 85° C. (Cary UV instrument). The results are presented in Table 168.

TABLE 168
Viscosity and concentration of ISIS antisense
oligonucleotides targeting human Factor XI

| | Viscosity | Concentration |
|---|---|---|
| ISIS No. | (cP) | (mg/mL) |
| 412223 | 8 | 163 |
| 412224 | 98 | 186 |
| 412225 | >100 | 162 |
| 413481 | 23 | 144 |
| 413482 | 16. | 172 |
| 416848 | 6 | 158 |
| 416850 | 67 | 152 |
| 416851 | 26 | 187 |
| 416852 | 29 | 169 |
| 416856 | 18 | 175 |
| 416858 | 10 | 166 |
| 416859 | 10 | 161 |
| 416860 | >100 | 154 |
| 416861 | 14 | 110 |
| 416863 | 9 | 165 |
| 416866 | >100 | 166 |
| 416867 | 8 | 168 |
| 445498 | 21 | 157 |
| 445504 | 20 | 139 |
| 445505 | 9 | 155 |
| 445509 | >100 | 167 |
| 445513 | 34 | 167 |
| 445522 | 63 | 173 |
| 445522 | 58 | 174 |
| 445530 | 25 | 177 |
| 445531 | 15 | 155 |
| 445531 | 20 | 179 |
| 449707 | 7 | 166 |
| 449708 | 9 | 188 |
| 449709 | 65 | 171 |
| 449710 | 7 | 186 |
| 449711 | 6 | 209 |
| 451541 | 10 | 168 |

1. A method for modulating an inflammatory response by administering a compound to an animal, wherein the compound comprises a Factor XI modulator and whereby the inflammatory response is modulated in the animal.

2. The method of claim 1, wherein the Factor XI modulator is a modified oligonucleotide targeting Factor XI.

3. A method for ameliorating or reducing the risk of an inflammatory disease, disorder or condition in an animal, or for treating an animal at risk for an inflammatory disease, disorder or condition, comprising administering a compound targeting Factor XI to the animal, wherein the compound administered to the animal ameliorates or reduces the risk of the inflammatory disease, disorder or condition in the animal, or treats the animal at risk for the inflammatory disease, disorder or condition.

4. (canceled)

5. A method for inhibiting Factor XI expression in an animal suffering from an inflammatory disease, disorder or condition, comprising administering a compound targeting Factor XI to the animal, wherein the compound administered to the animal inhibits Factor XI expression in the animal suffering from the inflammatory disease, disorder or condition.

6. (canceled)

7. The method of claim 1, wherein Factor XI has a sequence as shown in SEQ ID NO: 1 or SEQ ID NO: 2.

8. The method of claim 3, wherein the compound targeting Factor XI is a modified oligonucleotide.

9. The method of claim 8, wherein the modified oligonucleotide has a nucleobase sequence comprising at least 8 contiguous nucleobases of a nucleobase sequence selected from among the nucleobase sequences recited in SEQ ID NOs: 15-269.

10. The method of claim 9, wherein the nucleobase sequence of the modified oligonucleotide is 80% complementary to a nucleobase sequence of SEQ ID NO: 1, SEQ ID NO: 2, SEQ ID NO: 6 or SEQ ID NO: 274.

11. The method of claim 8, wherein the modified oligonucleotide consists of a single-stranded modified oligonucleotide.

12. The method of claim 8, wherein the modified oligonucleotide consists of 12 to 30 linked nucleosides or wherein the modified oligonucleotide consists of 20 linked nucleosides.

13. (canceled)

14. The method of claim 12, wherein at least one internucleoside linkage is a modified internucleoside linkage, wherein at least one nucleoside comprises a modified sugar and/or wherein at least one nucleoside comprises a modified nucleobase.

15. The method of claim 14, wherein each modified internucleoside linkage is a phosphorothioate internucleoside linkage.

16. (canceled)

17. The method of claim 14, wherein at least one modified sugar is a bicyclic sugar or a 2′-O-methoxyethyl.

18.-19. (canceled)

20. The method of claim 14, wherein the modified nucleobase is a 5-methylcytosine.

21. The method of claim 8, wherein the modified oligonucleotide comprises:
(a) a gap segment consisting of linked deoxynucleosides;
(b) a 5′ wing segment consisting of linked nucleosides;
(c) a 3′ wing segment consisting of linked nucleosides;
wherein the gap segment is positioned immediately adjacent to and between the 5′ wing segment and the 3′ wing segment and wherein each nucleoside of each wing segment comprises a modified sugar.

22. The method of claim 8, wherein the modified oligonucleotide comprises:
(a) a gap segment consisting of ten linked deoxynucleosides;
(b) a 5′ wing segment consisting of five linked nucleosides;
(c) a 3′ wing segment consisting of five linked nucleosides;
wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

23.-28. (canceled)

29. The method of claim 1, wherein the animal is a human.

30. The method of claim 3, wherein the inflammatory disease is arthritis, colitis, diabetes, sepsis, allergic inflammation, asthma, immunoproliferative disease, antiphospholipid syndrome or graft-related disorder, and/or an autoimmune disease.

31. (canceled)

32. The method of claim 30, wherein the arthritis is selected from rheumatoid arthritis, juvenile rheumatoid arthritis, arthritis uratica, gout, chronic polyarthritis, periarthritis humeroscapularis, cervical arthritis, lumbosacral arthritis, osteoarthritis, psoriatic arthritis, enteropathic arthritis and ankylosing spondylitis and wherein the colitis is selected from ulcerative colitis, Inflammatory Bowel Disease (IBD) and Crohn's Disease.

33. (canceled)

34. The method of claim 3, wherein the inflammatory disease disorder or condition is Th1 mediated and/or Th2 mediated.

35. The method of claim 34, wherein a marker for the Th1 mediated and/or Th2 mediated inflammatory disease, disorder or condition is decreased.

36. The method of claim 35, wherein the marker for Th1 is any of the cytokines IL-1, IL-6, INF-γ, TNF-α or KC.

37.-38. (canceled)

39. The method of claim 35, wherein the marker for Th2 is any of IL-4, IL-5, eosinophil infiltration or mucus production.

40.-41. (canceled)

42. The method of claim 1, further comprising a second agent.

43. (canceled)

44. The method of claim 1, wherein the compound is a salt form.

45. The method of claim 1, further comprising a pharmaceutically acceptable carrier or diluent.

46.-49. (canceled)
